(19) United States
(12) Patent Application Publication
(10) Pub. No.: US 2022/0137050 A1
(43) Pub. Date: May 5, 2022

(54) METHOD FOR DIAGNOSING HUMAN T-CELL LEUKEMIA VIRUS TYPE 1 (HTLV-1) ASSOCIATED DISEASES

(71) Applicant: NEC CORPORATION, Tokyo (JP)

(72) Inventors: Yoshiko YOSHIHARA, Tokyo (JP); Takuya FUKUSHIMA, Okinawa (JP); Megumi MIYARA, Okinawa (JP)

(73) Assignee: NEC CORPORATION, Tokyo (JP)

(21) Appl. No.: 17/429,206

(22) PCT Filed: Feb. 6, 2020
(86) PCT No.: PCT/JP2020/004458

§ 371 Date: Aug. 6, 2021

(30) Foreign Application Priority Data
Feb. 8, 2019 (JP) 2019-021539
Nov. 5, 2019 (JP) 2019-200955

(51) Int. Cl. G01N 33/569

(52) U.S. Cl. CPC G01N 33/56988

(57) ABSTRACT

As a technique enabling to simply and accurately diagnose human T cell leukemia virus type 1 (HTLV-1) related disease, there is provided a diagnostic method for an HTLV-1 related disease, comprising the steps of: measuring the amount of a marker protein in a blood sample taken from a subject, wherein the marker protein is at least one selected from the proteins listed in Tables 1 and 2; and, if a measured value is a predetermined reference value or more or the predetermined reference value or less, determining that the subject suffers from the HTLV-1 related disease or determining that the subject is in remission of the HTLV-1 related disease.

### TECHNICAL FIELD

The present invention relates to a method for diagnosing human T-cell leukemia virus type 1 (HTLV-1) associated diseases, more specifically, a method and kit for diagnosing HTLV-1 related diseases using a blood marker protein.

### BACKGROUND ART

Human T-cell leukemia virus type 1 (HTLV-1) is a retrovirus principally infects CD4-positive T cells to cause adult T-cell leukemia (ATL). HTLV-1 also causes HTLV-1 associated myelopathy (HAM) and HTLV-1 uveitis (HU). Hereinafter ATL, HAM and HU will be collectively referred to as “HTLV-1 related diseases”.

ATL is a malignant hematological tumor that invades various organs throughout the body and categorized into 4 disease forms: acute form, lymphoma-form, chronic form and smoldering form. The acute and lymphoma forms, which are also referred to “aggressive ATL”, are hematopoietic malignancies with the poorest prognosis. Whereas, the chronic and smoldering forms, which are also referred to as “indolent ATL”, mostly develop into blast crisis during the time course of the disease. The prognosis of these forms is poor.

As a treatment for ATL, a combination chemotherapy is known. However, even in a current reports, the median survival time of “aggressive ATL” still remains 13 months, which and not satisfactory. Patients with “Indolent ATL”, in some cases, survive for a long time without treatment and are mostly monitored without treatment until the disease worsens due to blast crisis and the like.

Currently, a main infection route of HTLV-1 is considered as a mother-to-child transmission, particularly through mother milk. HTLV-1 related diseases are developed from individuals infected with HTLV-1 (HTLV-1 careers) but most of the careers are asymptomatic. Japan is only one country having a high prevalence of HTLV-1 among the developed countries. HTLV-1 careers and HTLV-1 related diseases are often seen in the southwest of Japan including Kyushu and Okinawa districts.

In connection with the present invention, Patent Literature 1 discloses “a diagnostic agent for adult T-cell leukemia, containing a reagent that can detect the presence or absence of a transcript of TSLC1 gene or TSLC1 protein in cells or serum”. The TSLC1 gene is considered as a gene specifically expressed in leukemia cells taken from ATL patients. Owing to the technique disclosed there, it is reported that T cell leukemia can be highly precisely diagnosed.

### CITATION LIST

### Patent Literature

Patent Literature 1: Japanese Patent Laid-Open No. 2005-147798

### Non Patent Literature

Non Patent Literature 1: “Measurement of Cetuximab and Panitumumab-Unbound Serum EGFR Extracellular Domain Using an Assay Based on Slow Off-Rate Modified Aptamer (SOMAmer) Reagents”, N J Park et al., PLOS ONE, 2013, 8 (4): e61002.

### SUMMARY OF INVENTION

### Technical Problem

Compared to Kyushu and Okinawa districts where many experienced ATL specialist physicians and core hospitals are present, in metropolitan areas such as Kanto and Kinki districts, even though the real numbers of HTLV-1 careers and ATL patients are equal to those in a high prevalence area of HTLV-1, the number of specialist physicians capable of diagnosing ATL is low at present.

In the circumstances, a main object of the present invention is to provide a technique that can realize a simple and accurate diagnosis of an HTLV-1 related disease.

### Solution to Problem

To attain the object, the present invention provides the following [1] to [33].

[1] A diagnostic method for an HTLV-1 related disease, comprising the steps of:

measuring an amount of a marker protein in a blood sample taken from a subject, wherein the marker protein is at least one selected from the proteins listed in Tables 1 and 2; and

if a measured value is a predetermined reference value or more or the predetermined reference value or less, determining that when the subject is not definitely diagnosed with the human T-cell leukemia virus type 1 (HTLV-1) related disease, the subject suffers from, or is likely to develop, the HTLV-1 related disease; and determining that when the subject is definitely diagnosed with the HTLV-1 related disease, the subject is in remission, or is likely to be in remission, of the HTLV-1 related disease.

[1A] A diagnostic method for an HTLV-1 related disease, comprising the steps of:

measuring an amount of a marker protein in a blood sample taken from a subject, wherein the marker protein is at least one selected from the proteins listed in Tables 1 and 2; and

if a measured value is a predetermined reference value or more or the predetermined reference value or less, determining that when the subject is a carrier of human T-cell leukemia virus type 1 (HTLV-1) or an individual in remission of an HTLV-1 related disease, the subject suffers from, or is likely to develop, the HTLV-1 related disease; and determining that when the subject is a patient with the HTLV-1 related disease, the subject is in remission, or is likely to be in remission, of the HTLV-1 related disease.

[2] The diagnostic method according to [1], wherein the HTLV-1 related disease is adult T-cell leukemia (ATL).

[2A] The diagnostic method according to [1] or [2], wherein

the marker protein is at least one selected from the proteins represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 126, 128, 129, 131, 139, 141, 155, 156, 161, 164, 165, 167, 170, 172, 173, 174, 175, 181, 185, 187, 189, 195, 198, 199, 200, 203, 205, 207, 209, 211, 220, 221, 222, 223, 224, 225, 226, 230, 231, 232, 235, 236, 238, 240, 241, 242, 243, 244, 246, 247, 250, 251, 252, 253, 256, 259, 260, 269, 270, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609, 621 and 629 of Table 1, and

when the subject is an HTLV-1 carrier or an individual in remission of an HTLV-1 related disease, an increase of the measured value to the reference value or more or a decrease of the measured value to the reference value or less indicates that the subject suffers from, or is likely to develop, the HTLV-1 related disease, and

thereafter, when the subject is a patient with the HTLV-1 related disease, a decrease of the measured value to the reference value or less or an increase of the measured value to the reference or more indicates that the subject is in remission, or is likely to be in remission, of the HTLV-1 related disease.

[2B] The diagnostic method according to [2A], wherein

the marker protein is at least one selected from the proteins represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 128, 129, 131, 139, 141, 155, 156, 161, 164, 165, 167, 172, 173, 174, 175, 181, 185, 187, 189, 195, 198, 199, 200, 203, 205, 207, 209, 211, 220, 221, 222, 223, 224, 225, 226, 230, 231, 232, 236, 238, 240, 241, 242, 243, 244, 246, 247, 250, 251, 252, 253, 256, 259, 260, 269, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609 and 629 of Table 1, and

when the subject is an HTLV-1 carrier or an individual in remission of an HTLV-1 related disease, an increase of the measured value to the reference value or more indicates that the subject suffers from, or is likely to develop, the HTLV-1 related disease, and

thereafter, when the subject is a patient with the HTLV-1 related disease, a decrease of the measured value to the reference value or less indicates that the subject is in remission, or is likely to be in remission, of the HTLV-1 related disease.

[2C] The diagnostic method according to [2A], wherein

the marker protein is at least one selected from the proteins represented by Nos. 126, 170, 235, 270, and 621 of Table 1, and

when the subject is an HTLV-1 carrier or an individual in remission of an HTLV-1 related disease, a decrease of the measured value to the reference value or less indicates that the subject suffers from, or is likely to develop, the HTLV-1 related disease, and

thereafter, when the subject is a patient with an HTLV-1 related disease, an increase of the measured value to the reference value or more indicates that the subject is in remission, or is likely to be in remission, of the HTLV-1 related disease.

[3] The diagnostic method according to [1] or [2], wherein the subject is an HTLV-1 career, and it is determined that the subject suffers from, or is likely to develop, the HTLV-1 related disease.

[4] The diagnostic method according to [3], wherein the marker protein is at least one selected from the proteins represented by Nos. 1 to 61 and 63 to 482 of Table 1.

[4A] The diagnostic method according to [1] or [2], wherein

the marker protein is at least one selected from the proteins represented by Nos. 1 to 61 and 63 to 482 of Table 1, and

the subject is an HTLV-1 carrier or an individual in remission of an HTLV-1 related disease, and

an increase of the measured value to the reference value or more or a decrease of the measured value to the reference value or less indicates that the subject suffers from, or is likely to develop, an HTLV-1 related disease.

[5] The diagnostic method according to [3] or [4A], wherein the marker protein is at least one selected from the proteins represented by 1 to 61 and 63 to 333 of Table 1.

[6] The diagnostic method according to [3] or [4A], wherein the marker protein is at least one of the proteins represented by Nos. 1 and/or 2 of Table 1.

[7] The diagnostic method according to any one of [3] to [6], wherein the reference value is an average, median or mode of measured values in blood samples of an HTLV-1 carrier group or a healthy subject group (non HTLV-1 careers).

[8] The diagnostic method according to [1] or [2], wherein the subject is a patient with an HTLV-1 related disease, and it is determined that the subject is in remission, or is likely to be in remission, of the HTLV-1 related disease.

[9] The diagnostic method according to [8], wherein the marker protein is at least one selected from the proteins represented by Nos. 1 to 633 of Table 2.

[9A] The diagnostic method according to [1] or [2], wherein

the marker protein is at least one selected from the proteins represented by Nos. 1 to 633 of Table 2, and

the subject is a patient with an HTLV-1 related disease, and

an increase of the measured value to the reference value or more or a decrease of the measured value to the reference value or less indicates that the subject is in remission, or is likely to be in remission, of the HTLV-1 related disease.

[10] The diagnostic method according to [8] or [9A], wherein the marker protein is at least one selected from the proteins represented by Nos. 1 to 440 of Table 2.

[11] The diagnostic method according to any one of [8] to [10], wherein the reference value is an average, median or mode of measured values in blood samples of an HTLV-1 related disease group.

[12] The diagnostic method according to any one of [1] to [11], wherein the blood sample is plasma.

[13] A diagnostic kit for an HTLV-1 related disease, comprising a reagent for detecting at least one protein selected from the proteins listed in Table 1 (except the protein represented by No. 62) and the proteins listed in Table 2.

[13A] The diagnostic kit according to [13], comprising a reagent for detecting at least one protein selected from the proteins represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 126, 128, 129, 131, 139, 141, 155, 156, 161, 164, 165, 167, 170, 172, 173, 174, 175, 181, 185, 187, 189, 195, 198, 199, 200, 203, 205, 207, 209, 211, 220, 221, 222, 223, 224, 225, 226, 230, 231, 232, 235, 236, 238, 240, 241, 242, 243, 244, 246, 247, 250, 251, 252, 253, 256, 259, 260, 269, 270, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609, 621, 629 of Table 1.

[13B] The diagnostic kit according to [13A], comprising a reagent for detecting at least one protein selected from the proteins represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 128, 129, 131, 139, 141, 155, 156, 161, 164, 165, 167, 172, 173, 174, 175, 181, 185, 187, 189, 195, 198, 199, 200, 203, 205, 207, 209, 211, 220, 221, 222, 223, 224, 225, 226, 230, 231, 232, 236, 238, 240, 241, 242, 243, 244, 246, 247, 250, 251, 252, 253, 256, 259, 260, 269, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609 and 629 of Table 1.

[13C] The diagnostic kit according to [13A], comprising a reagent for detecting at least one protein selected from the proteins represented by Nos. 126, 170, 235, 270 and 621 of Table 1.

[14] The diagnostic kit according to [13], comprising a reagent for detecting at least one protein selected from the proteins represented by Nos. 1 to 61 and 63 to 482 of Table 1 and Nos. 1 to 633 of Table 2.

[15] The diagnostic kit according to [13], comprising a reagent for detecting at least one protein selected from the proteins represented by Nos. 1 to 61 and 63 to 333 of Table 1 and Nos. 1 to 440 of Table 2.

[16] The diagnostic kit according to [13], comprising a reagent for detecting a protein represented by No. 1 or 2 of Table 1.

[17] The diagnostic kit according to any one of [13] to [16], wherein the HTLV-1 related disease is adult T-cell leukemia (ATL).

[18] The diagnostic kit according to any one of [13] to [17], wherein the detection reagent is an antibody, nucleic acid or aptamer that specifically binds to the protein.

[19] Use of at least one protein selected from the proteins listed in Table 1 (except the protein represented by No. 62) and the proteins listed in Table 2, for diagnosing an HTLV-1 related disease.

[20] Use according to [19], wherein the HTLV-1 related disease is adult T-cell leukemia (ATL).

[21] A method for treating an HTLV-1 related disease, comprising the steps of:

measuring an amount of a marker protein in a blood sample taken from a subject, wherein the marker protein is at least one selected from the proteins listed in Table 1 (except the protein represented by No. 62);

selecting the subject as a subject to be treated if the measured value is a predetermined reference value or more or the predetermined reference value or less; and applying a chemotherapy and/or a non-chemotherapy to the selected subject to be treated.

[22] The method according to [21], wherein the subject is an HTLV-1 career.

[23] The method according to [22], wherein the HTLV-1 related disease is adult T-cell leukemia (ATL).

[24] The method according to any one of [21] to [23], wherein the non-chemotherapy is a cancer vaccine therapy

[25] A method for treating an HTLV-1 related disease, comprising the steps of:

measuring an amount of a marker protein in a blood sample taken from a subject, wherein the marker protein is at least one selected from the proteins listed in Table 2; and

increasing, reducing, stopping or terminating application of a chemotherapy and/or non-chemotherapy to the subject if the measured value is a predetermined reference value or more or the predetermined reference or less.

[26] The method according to [25], wherein the subject is a patient with an HTLV-1 related disease.

[27] The method according to [25], wherein the HTLV-1 related disease is adult T-cell leukemia (ATL).

[28] The method according to [25] to [27], wherein the non-chemotherapy is a cancer vaccine therapy.

[28A] A method for treating an HTLV-1 related disease, comprising the steps of:

measuring an amount of a marker protein in a blood sample taken from a subject, wherein the marker protein is at least one selected from the proteins represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 126, 128, 129, 131, 139, 141, 155, 156, 161, 164, 165, 167, 170, 172, 173, 174, 175, 181, 185, 187, 189, 195, 198, 199, 200, 203, 205, 207, 209, 211, 220, 221, 222, 223, 224, 225, 226, 230, 231, 232, 235, 236, 238, 240, 241, 242, 243, 244, 246, 247, 250, 251, 252, 253, 256, 259, 260, 269, 270, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609, 621 and 629 of Table 1;

selecting the subject as a subject to be treated if the measured value is a predetermined reference value or more or the predetermined reference value or less;

applying a chemotherapy and/or a non-chemotherapy to the selected subject to be treated; and

thereafter, increasing, reducing, stopping or terminating application of a chemotherapy and/or non-chemotherapy to the subject if the measured value reaches a predetermined reference value or less or the predetermined reference or more.

[28B] The method according to [28A], wherein the subject is a patient with an HTLV-1 related disease.

[28C] The method according to [28A], wherein the HTLV-1 related disease is adult T-cell leukemia (ATL).

[28D] The method according to any one of [28A] to [28C], wherein the non-chemotherapy is a cancer vaccine therapy.

[29] A method for determining effectiveness of a treatment for an HTLV-1 related disease, comprising the steps of:

measuring an amount of a marker protein in a blood sample taken from a subject, wherein the marker protein is at least one selected from the proteins listed in Table 2; and

determining that the treatment is effective if the measured value is a predetermined reference value or more or the predetermined reference value or less.

[30] The method according to [29], wherein the subject is a patient with an HTLV-1 related disease.

[31] The method according to [30], wherein the HTLV-1 related disease is adult T-cell leukemia (ATL).

[32] The method according to any one of [29] to [31], wherein the treatment is a chemotherapy and/or non-chemotherapy.

[33] The method according to [32], wherein the non-chemotherapy is a cancer vaccine therapy.

### Advantageous Effects of Invention

The present invention provides a technique that can realize a simple and accurate diagnosis of an HTLV-1 related disease.

### BRIEF DESCRIPTION OF DRAWINGS

FIG. 1 The figure is a graph showing measurement results of the amount of a protein in plasmas of HTLV-1 careers and ATL patients.

FIG. 2 The figure is a graph showing measurement results of the amount of a protein in plasmas of HTLV-1 careers and ATL patients.

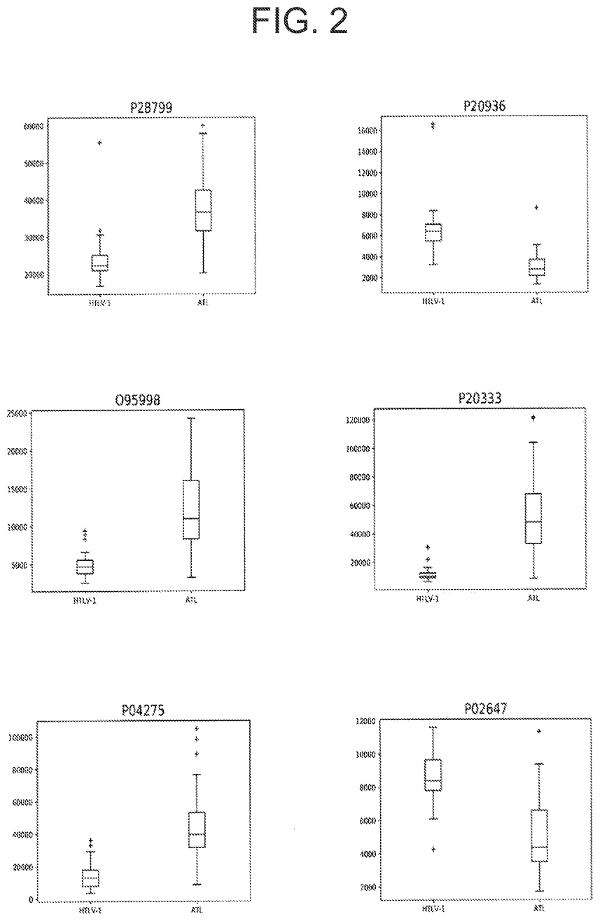

FIG. 3 The figure is a graph showing measurement results of the amount of a protein in plasmas of HTLV-1 careers and ATL patients.

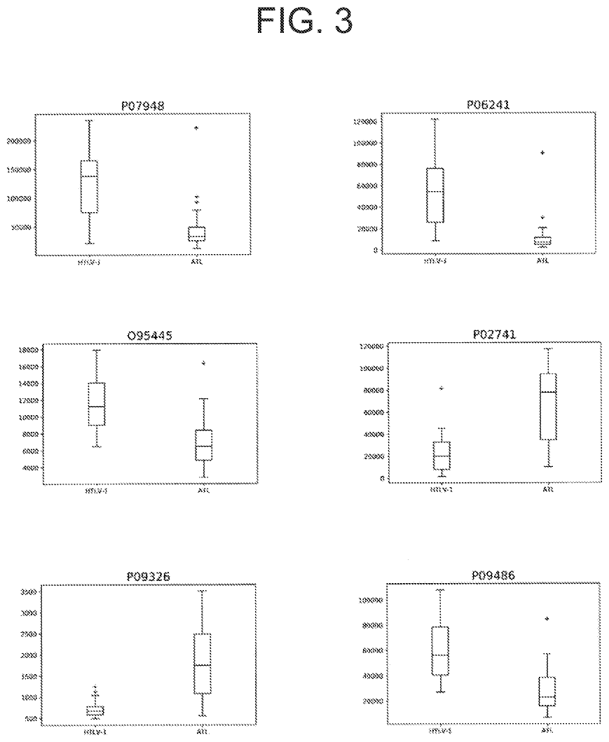

FIG. 4 The figure is a graph showing measurement results of the amount of a protein in plasmas of HTLV-1 careers and ATL patients.

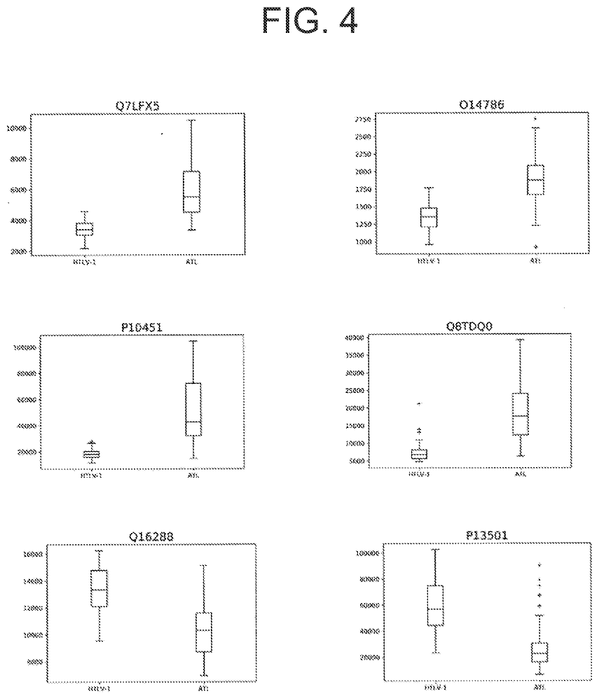

FIG. 5 The figure is a graph showing measurement results of the amount of a protein in plasmas of HTLV-1 careers and ATL patients.

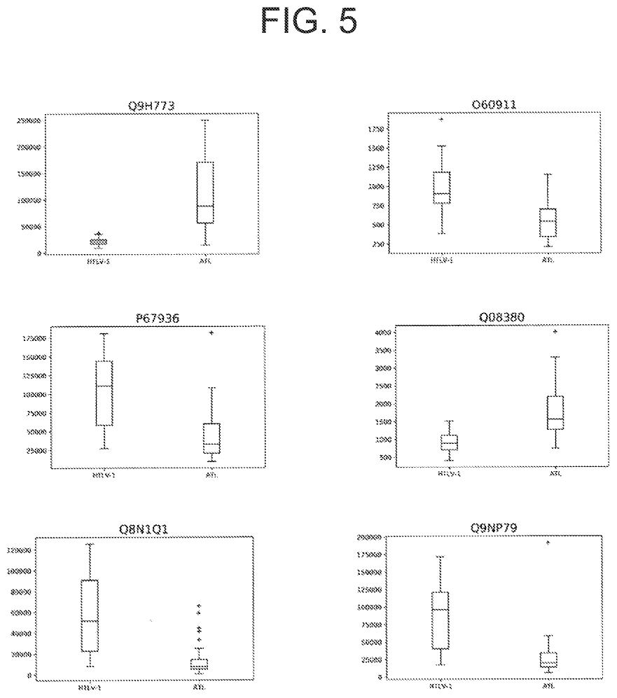

FIG. 6 The figure is a graph showing measurement results of the amount of a protein in plasmas of HTLV-1 careers and ATL patients.

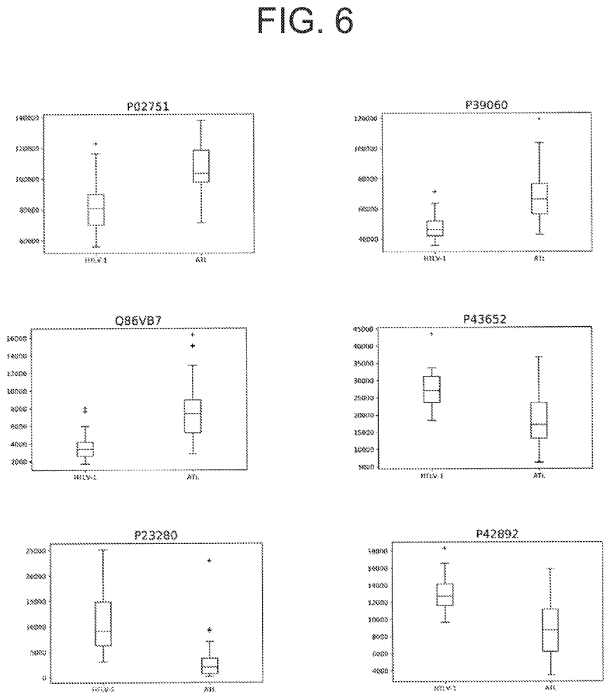

FIG. 7 The figure is a graph showing measurement results of the amount of a protein in plasmas of HTLV-1 careers and ATL patients.

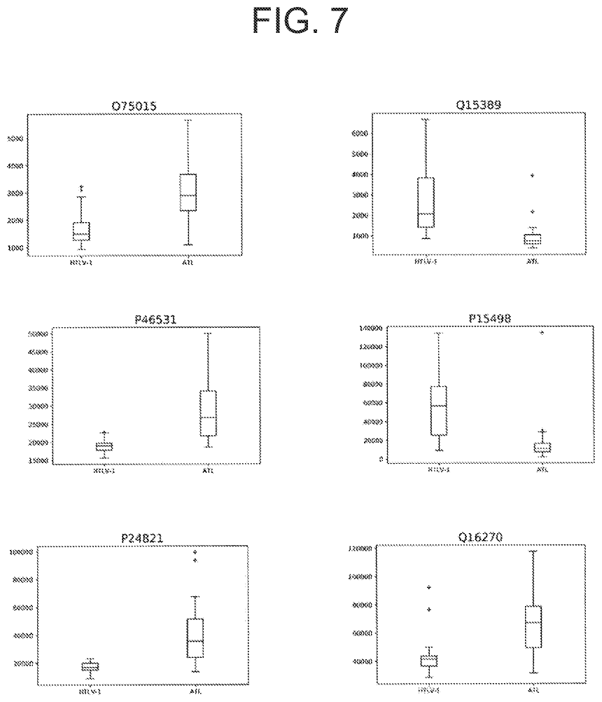

FIG. 8 The figure is a graph showing measurement results of the amount of a protein in plasmas of HTLV-1 careers and ATL patients.

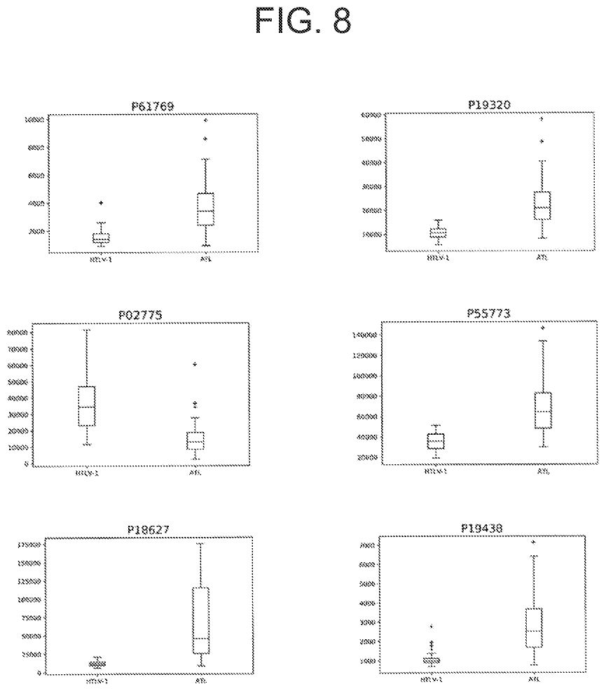

FIG. 9 The figure is a graph showing measurement results of the amount of a protein in plasmas of HTLV-1 careers and ATL patients.

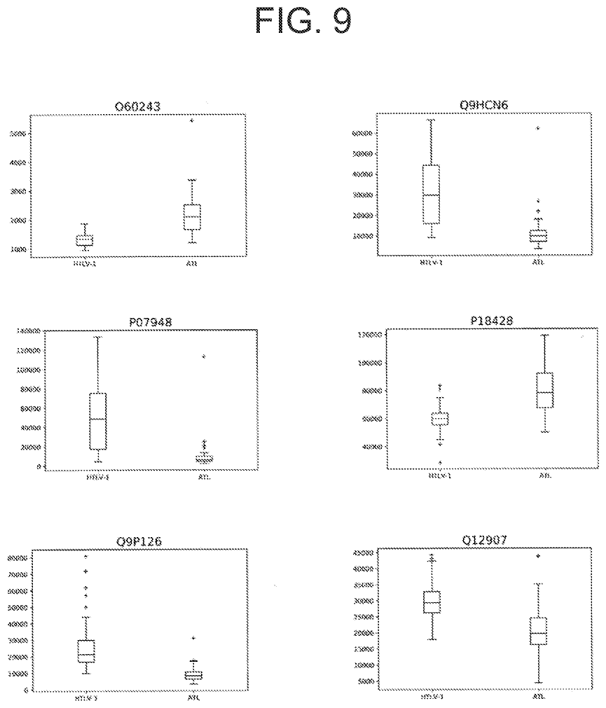

FIG. 10 The figure is a graph showing measurement results of the amount of a protein in plasmas of HTLV-1 careers and ATL patients.

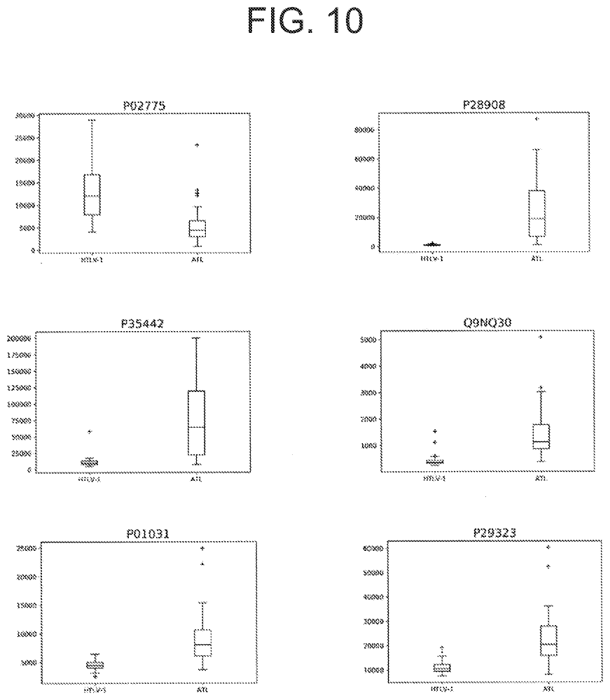

FIG. 11 The figure is a graph showing measurement results of the amount of a protein in plasmas of HTLV-1 careers and ATL patients.

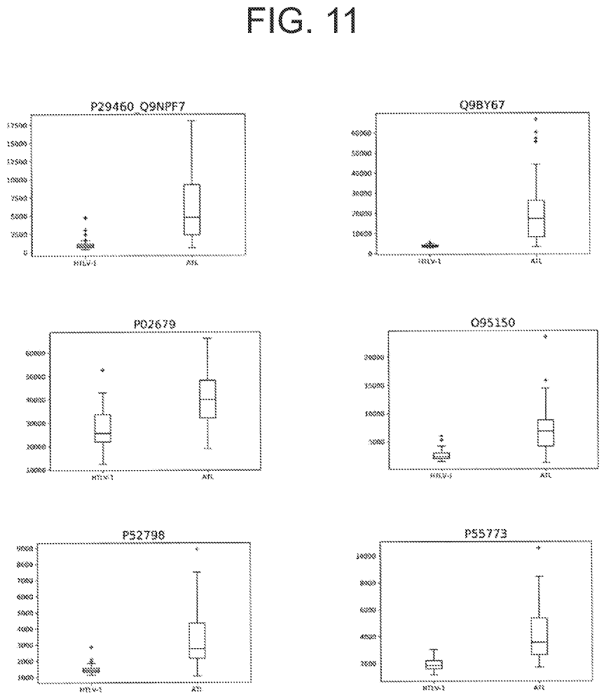

FIG. 12 The figure is a graph showing measurement results of the amount of a protein in plasmas of HTLV-1 careers and ATL patients.

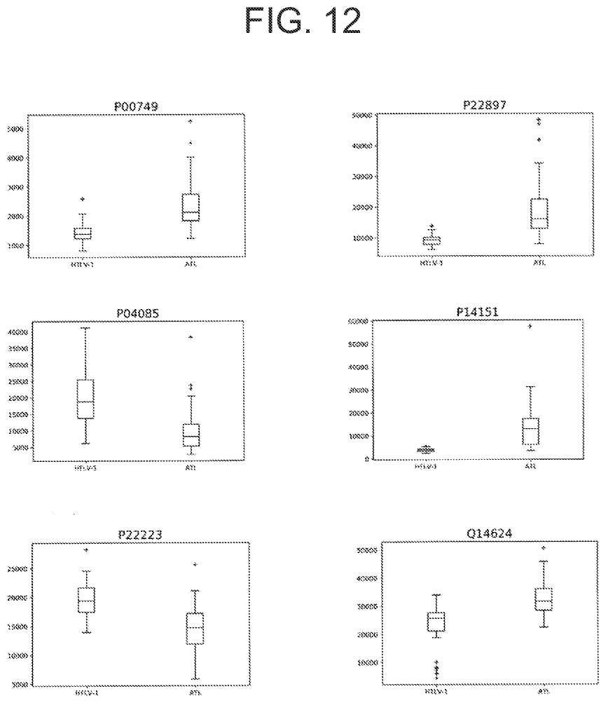

FIG. 13 The figure is a graph showing measurement results of the amount of a protein in plasmas of HTLV-1 careers and ATL patients.

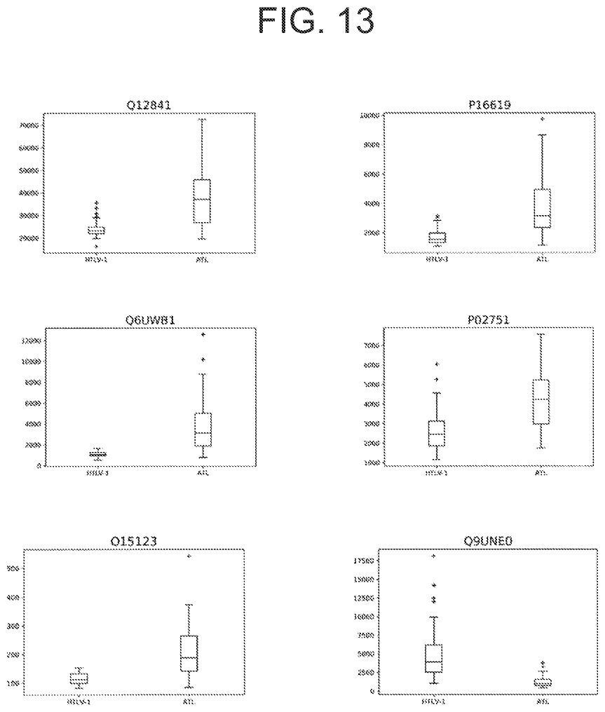

FIG. 14 The figure is a graph showing measurement results of the amount of a protein in plasmas of HTLV-1 careers and ATL patients.

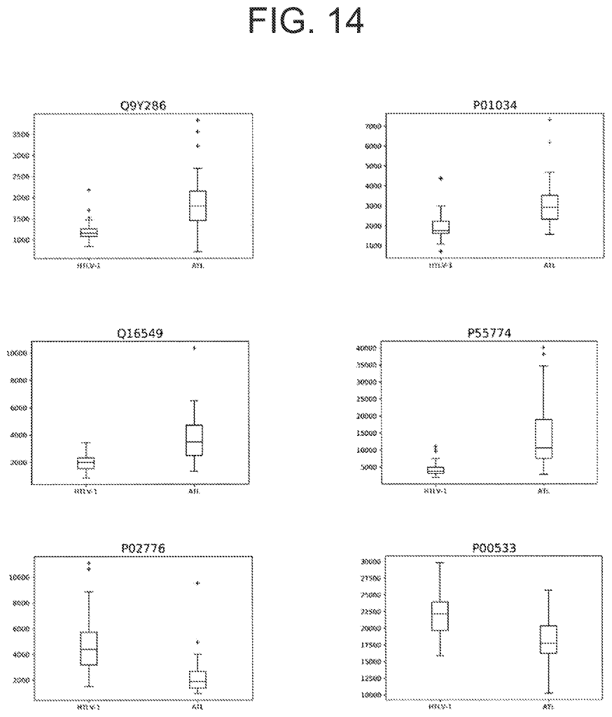

FIG. 15 The figure is a graph showing measurement results of the amount of a protein in plasmas of HTLV-1 careers and ATL patients.

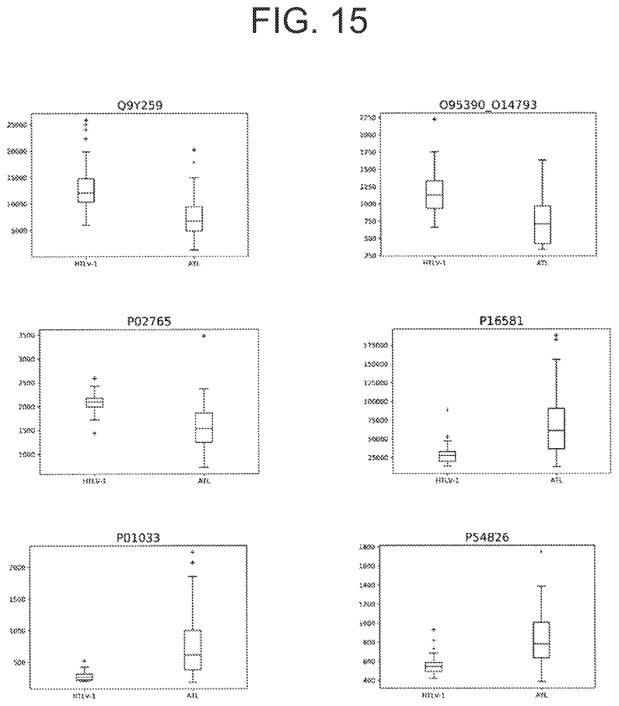

FIG. 16 The figure is a graph showing measurement results of the amount of a protein in plasmas of HTLV-1 careers and ATL patients.

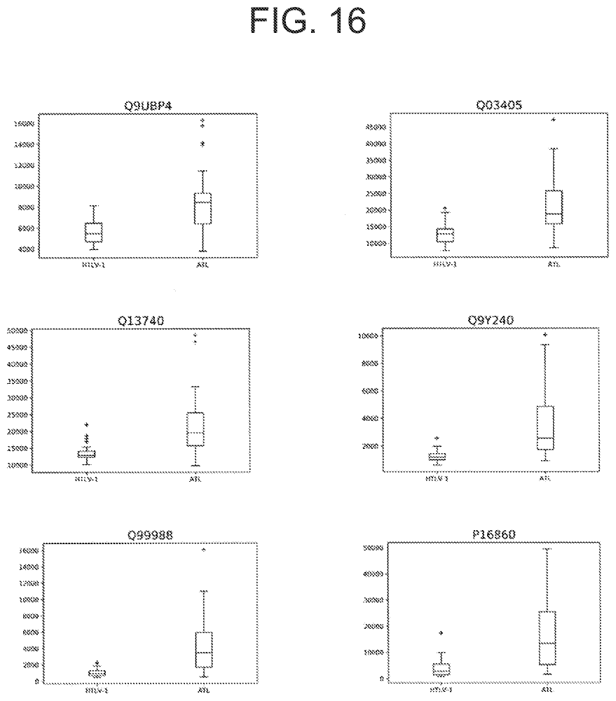

FIG. 17 The figure is a graph showing measurement results of the amount of a protein in plasmas of HTLV-1 careers and ATL patients.

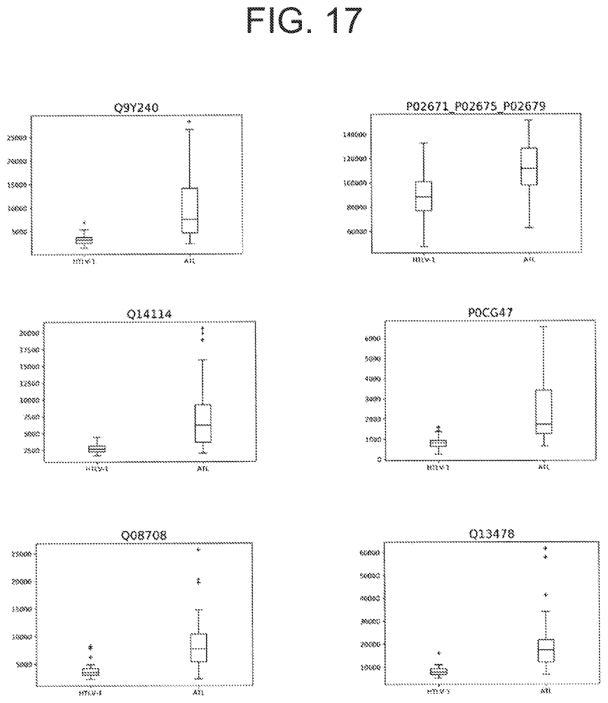

FIG. 18 The figure is a graph showing measurement results of the amount of a protein in plasmas of HTLV-1 careers and ATL patients.

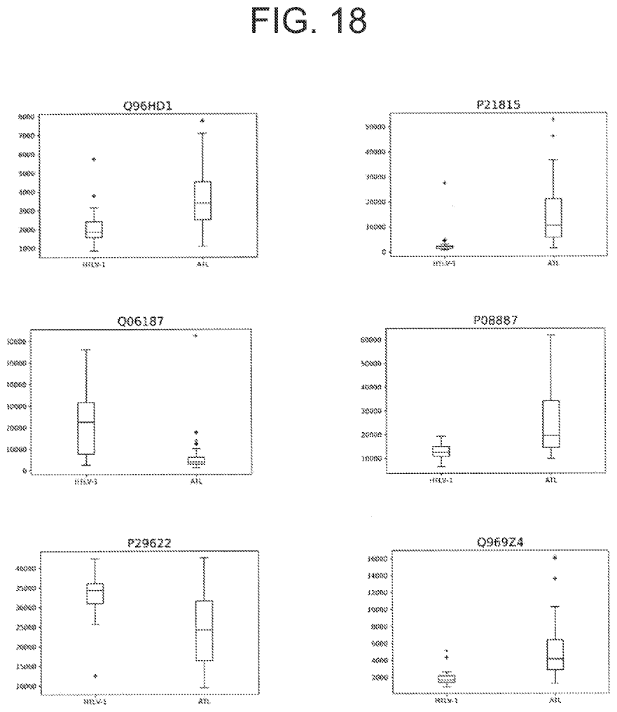

FIG. 19 The figure is a graph showing measurement results of the amount of a protein in plasmas of HTLV-1 careers and ATL patients.

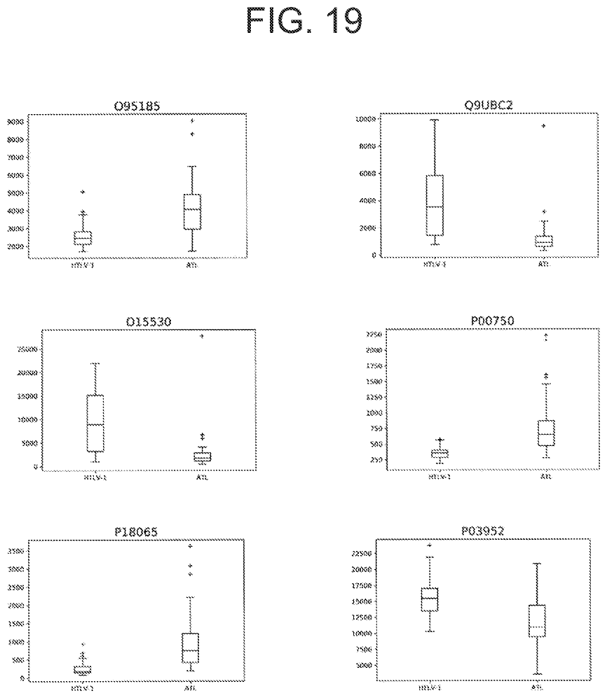

FIG. 20 The figure is a graph showing measurement results of the amount of a protein in plasmas of HTLV-1 careers and ATL patients.

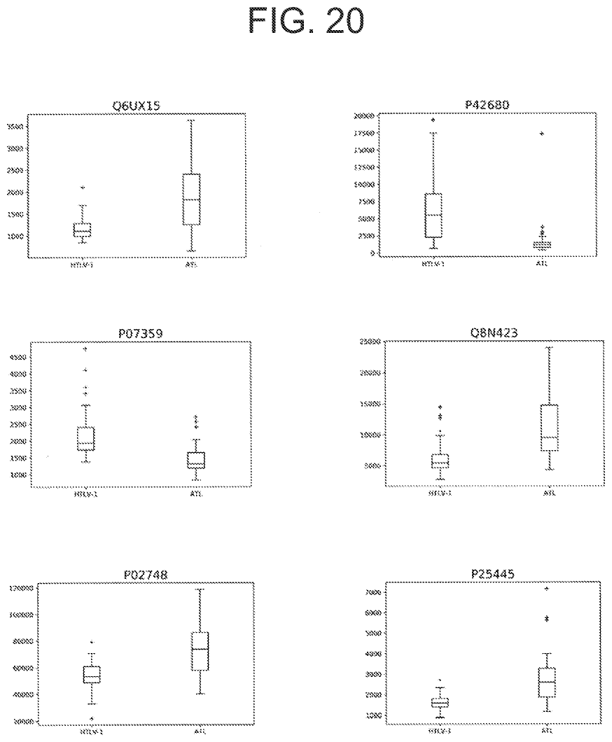

FIG. 21 The figure is a graph showing measurement results of the amount of a protein in plasmas of HTLV-1 careers and ATL patients.

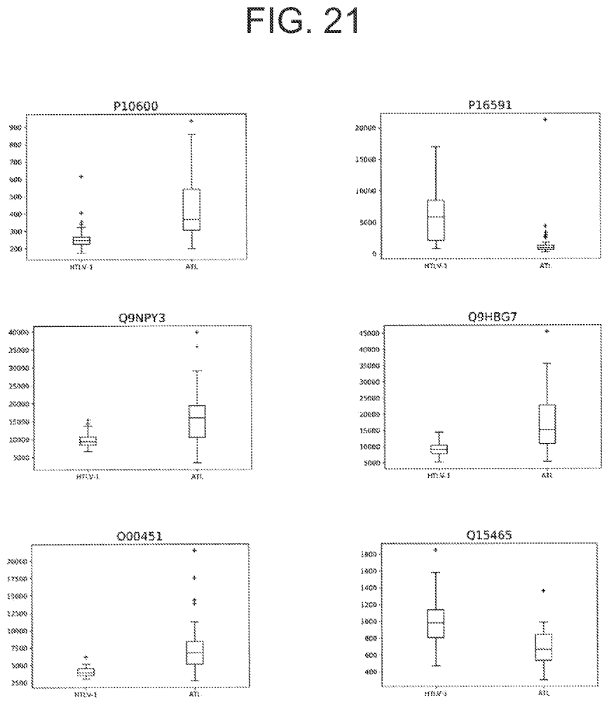

FIG. 22 The figure is a graph showing measurement results of the amount of a protein in plasmas of HTLV-1 careers and ATL patients.

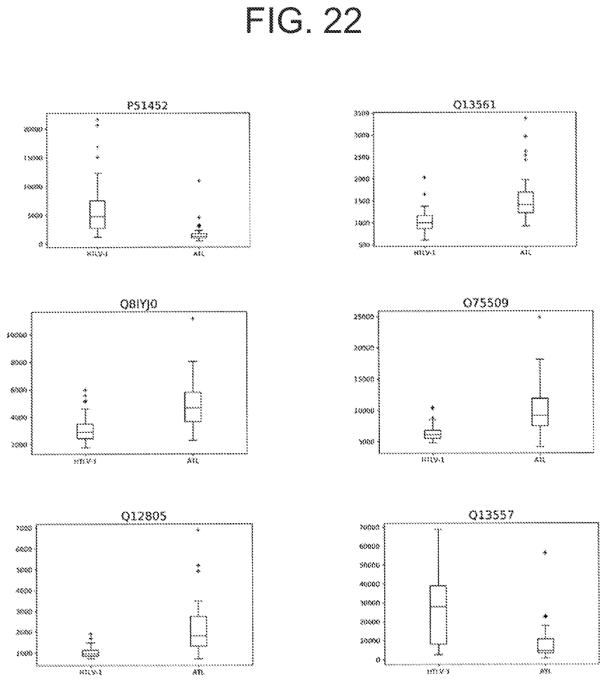

FIG. 23 The figure is a graph showing measurement results of the amount of a protein in plasmas of HTLV-1 careers and ATL patients.

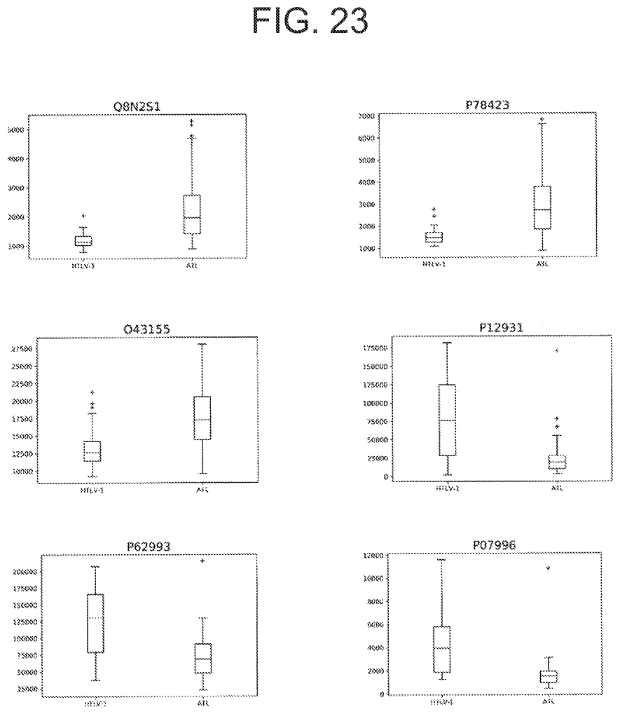

FIG. 24 The figure is a graph showing measurement results of the amount of a protein in plasmas of HTLV-1 careers and ATL patients.

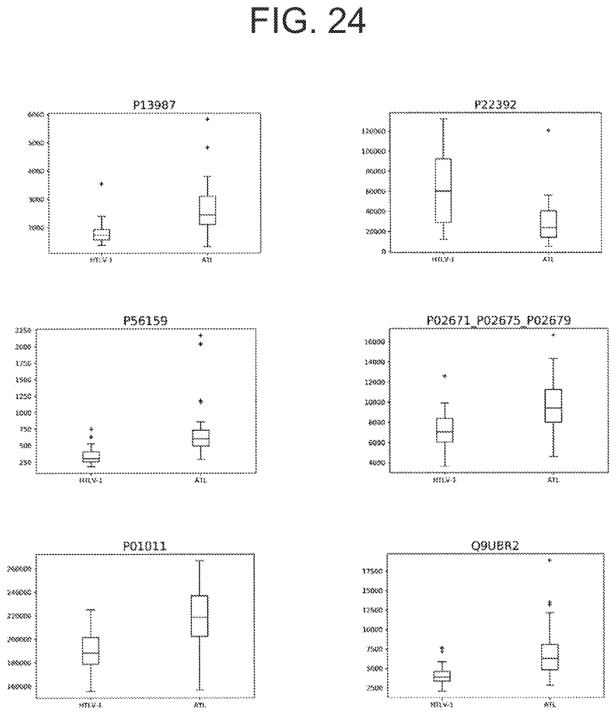

FIG. 25 The figure is a graph showing measurement results of the amount of a protein in plasmas of HTLV-1 careers and ATL patients.

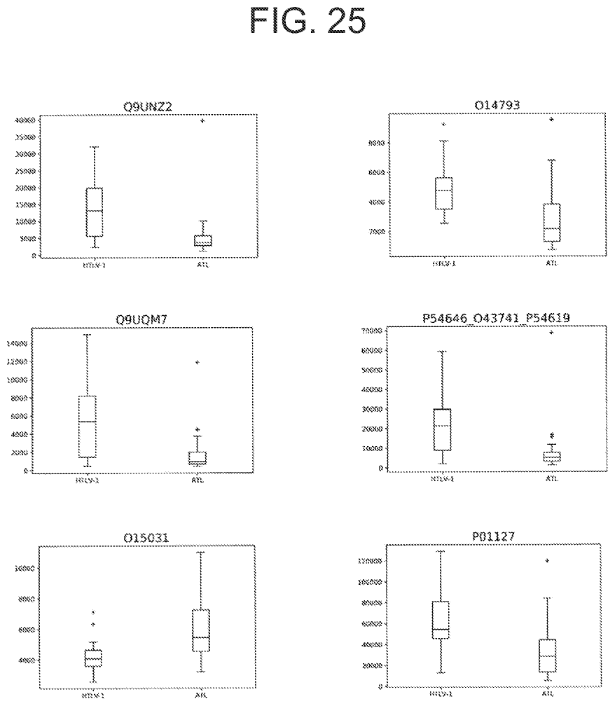

FIG. 26 The figure is a graph showing measurement results of the amount of a protein in plasmas of HTLV-1 careers and ATL patients.

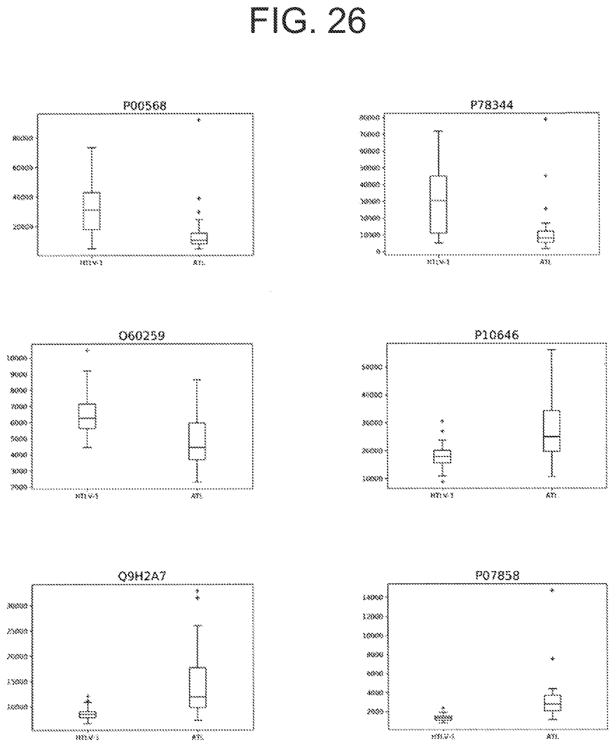

FIG. 27 The figure is a graph showing measurement results of the amount of a protein in plasmas of HTLV-1 careers and ATL patients.

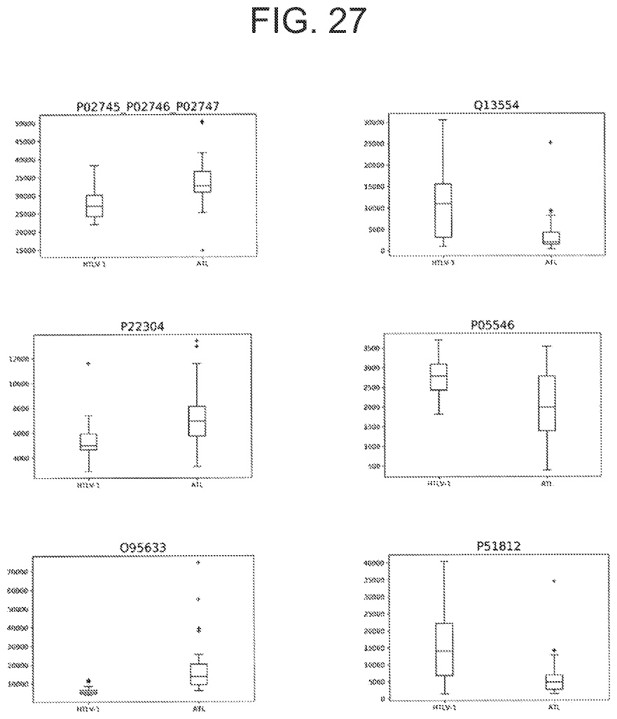

FIG. 28 The figure is a graph showing measurement results of the amount of a protein in plasmas of HTLV-1 careers and ATL patients.

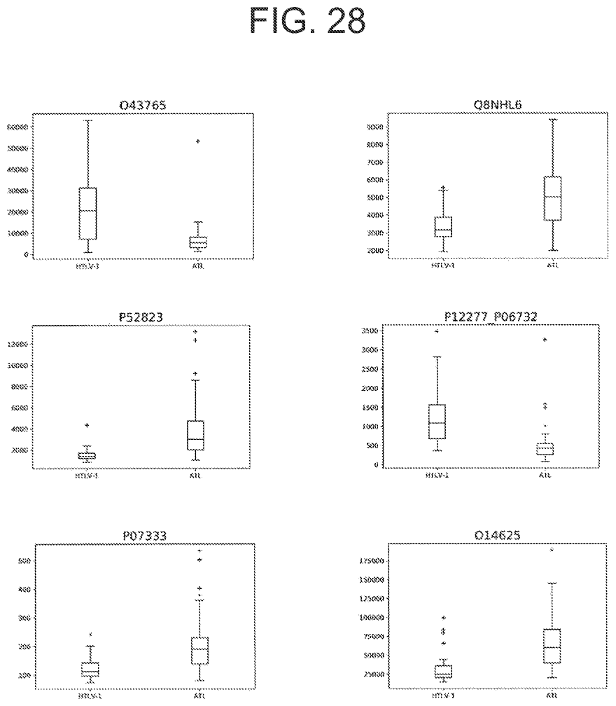

FIG. 29 The figure is a graph showing measurement results of the amount of a protein in plasmas of HTLV-1 careers and ATL patients.

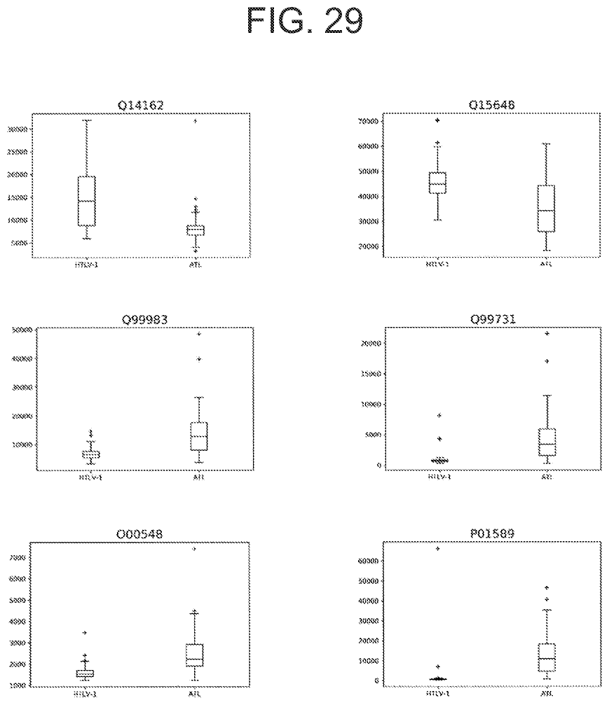

FIG. 30 The figure is a graph showing measurement results of the amount of a protein in plasmas of HTLV-1 careers and ATL patients.

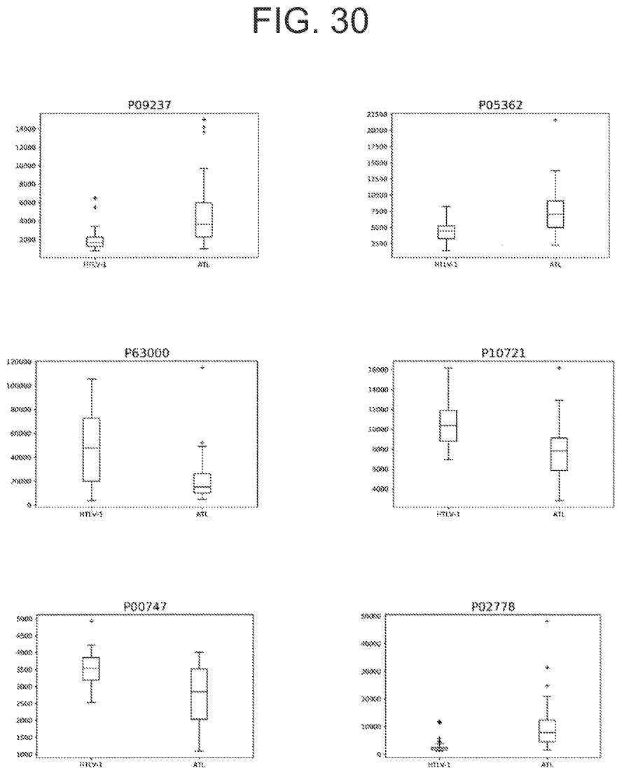

FIG. 31 The figure is a graph showing measurement results of the amount of a protein in plasmas of HTLV-1 careers and ATL patients.

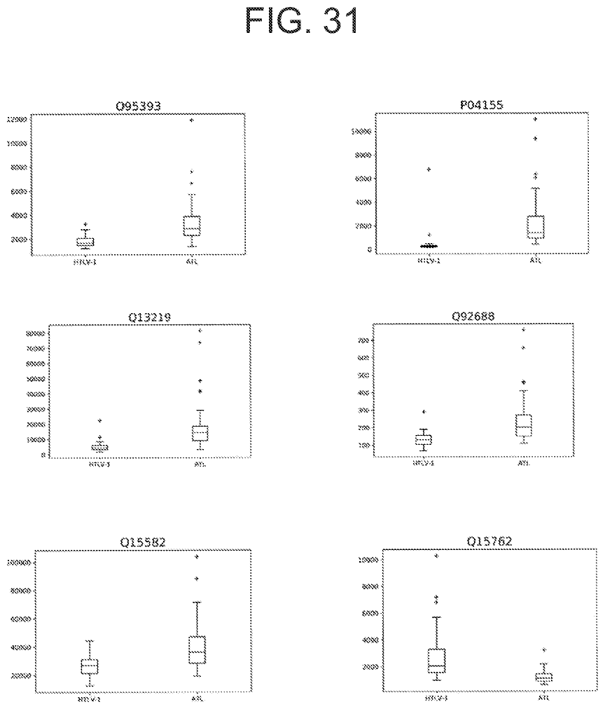

FIG. 32 The figure is a graph showing measurement results of the amount of a protein in plasmas of HTLV-1 careers and ATL patients.

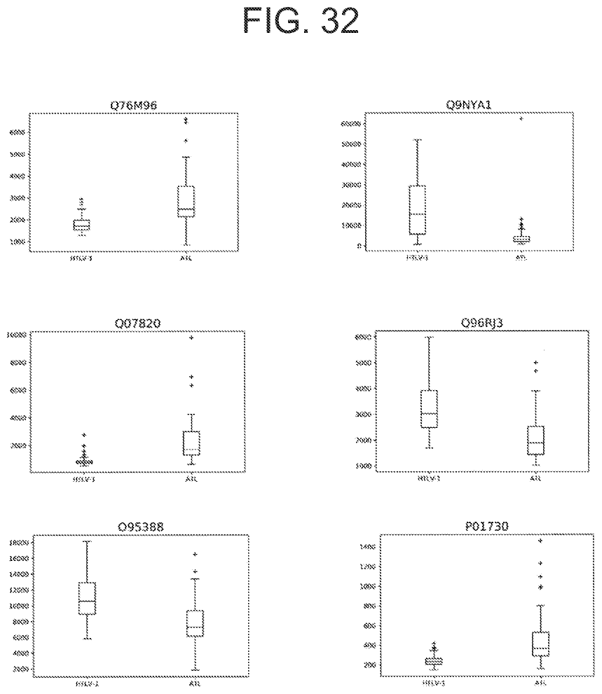

FIG. 33 The figure is a graph showing measurement results of the amount of a protein in plasmas of HTLV-1 careers and ATL patients.

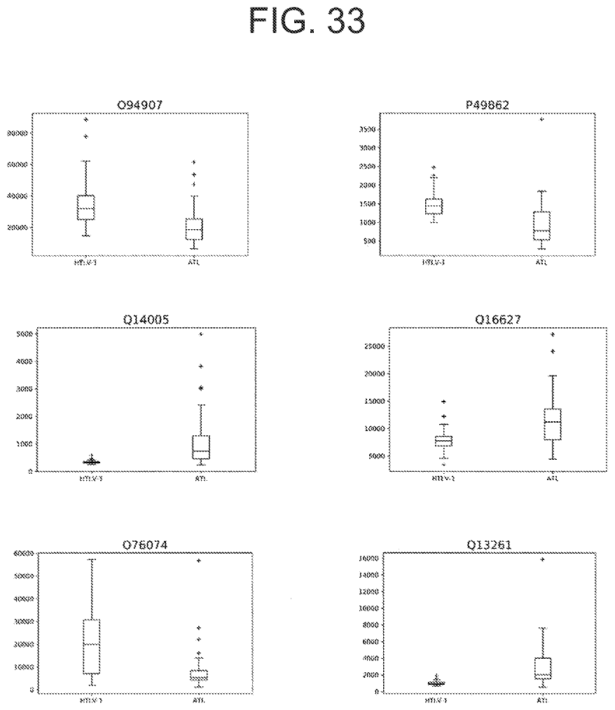

FIG. 34 The figure is a graph showing measurement results of the amount of a protein in plasmas of HTLV-1 careers and ATL patients.

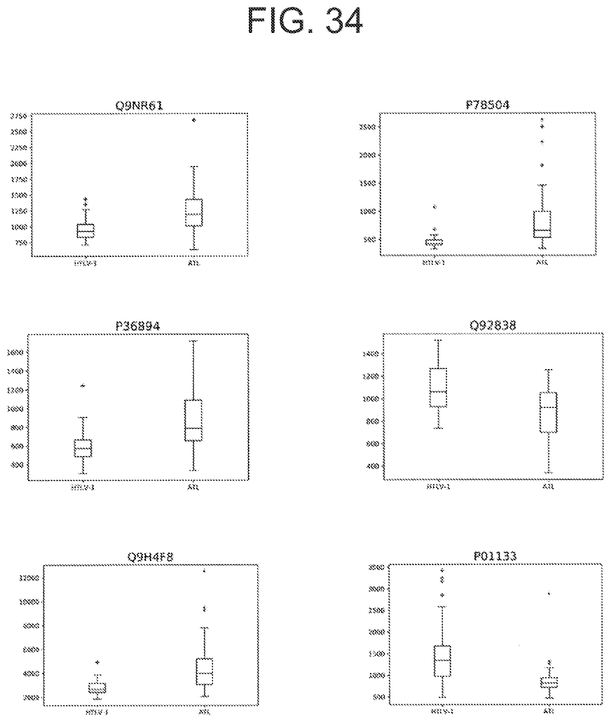

FIG. 35 The figure is a graph showing measurement results of the amount of a protein in plasmas of HTLV-1 careers and ATL patients.

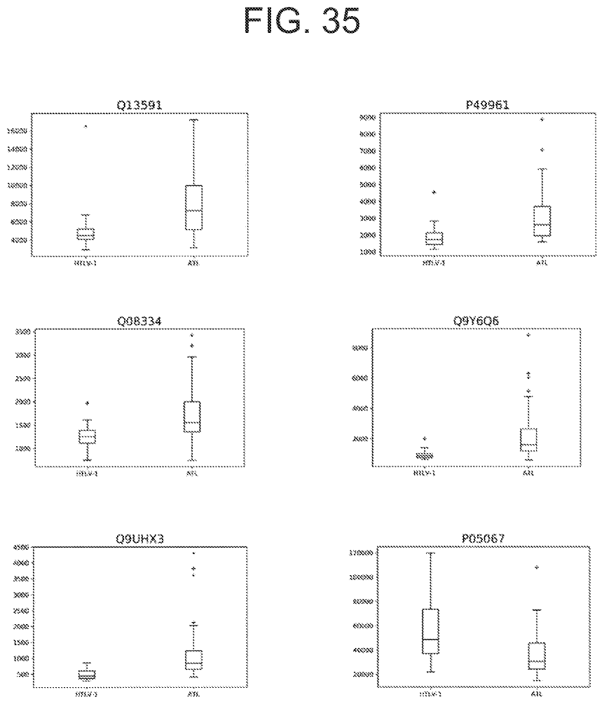

FIG. 36 The figure is a graph showing measurement results of the amount of a protein in plasmas of HTLV-1 careers and ATL patients.

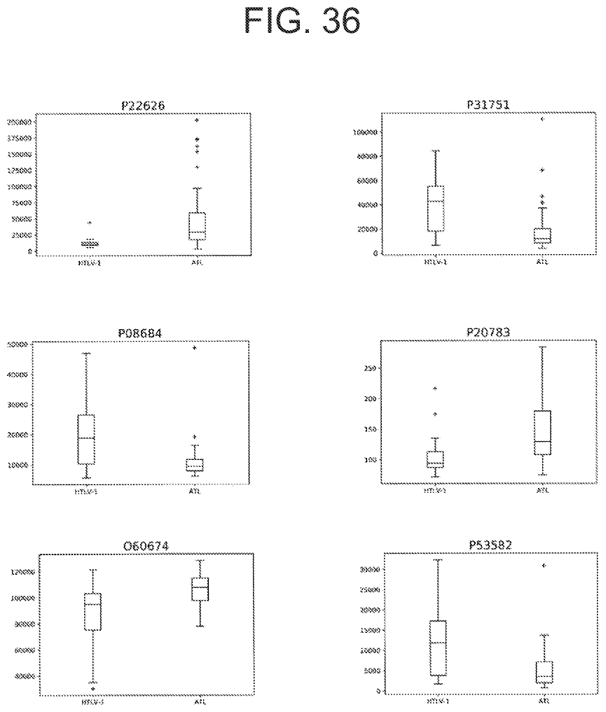

FIG. 37 The figure is a graph showing measurement results of the amount of a protein in plasmas of HTLV-1 careers and ATL patients.

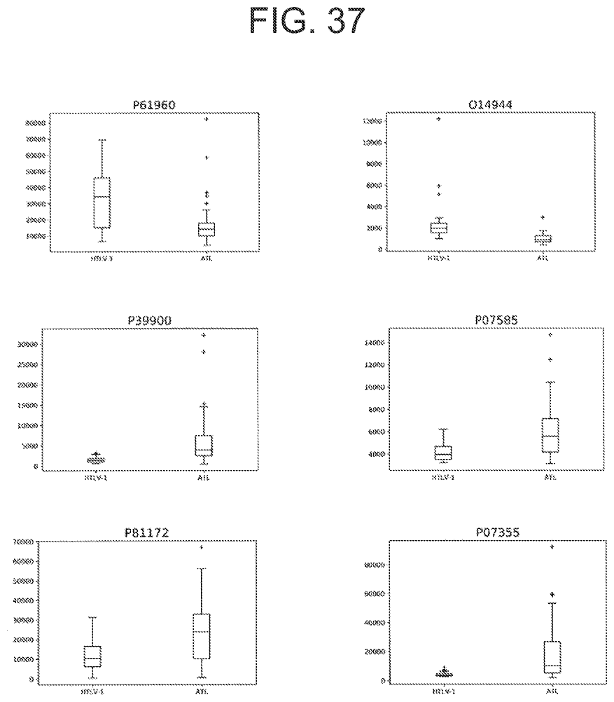

FIG. 38 The figure is a graph showing measurement results of the amount of a protein in plasmas of HTLV-1 careers and ATL patients.

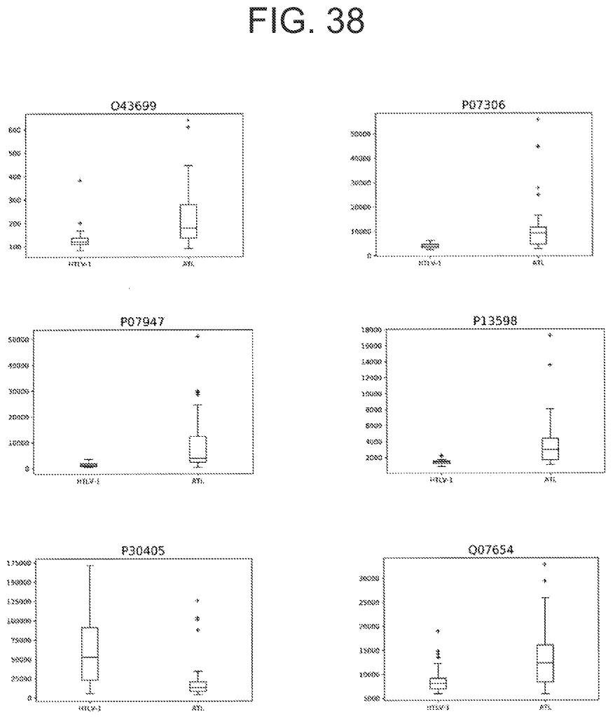

FIG. 39 The figure is a graph showing measurement results of the amount of a protein in plasmas of HTLV-1 careers and ATL patients.

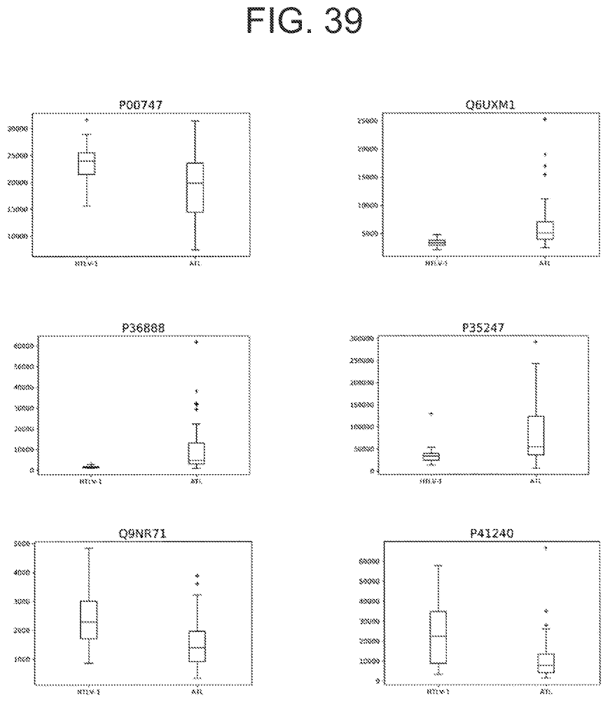

FIG. 40 The figure is a graph showing measurement results of the amount of a protein in plasmas of HTLV-1 careers and ATL patients.

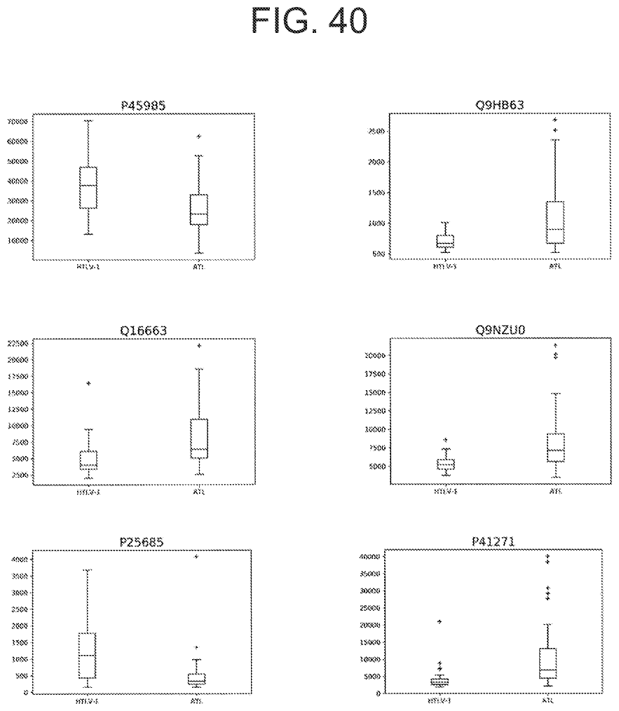

FIG. 41 The figure is a graph showing measurement results of the amount of a protein in plasmas of HTLV-1 careers and ATL patients.

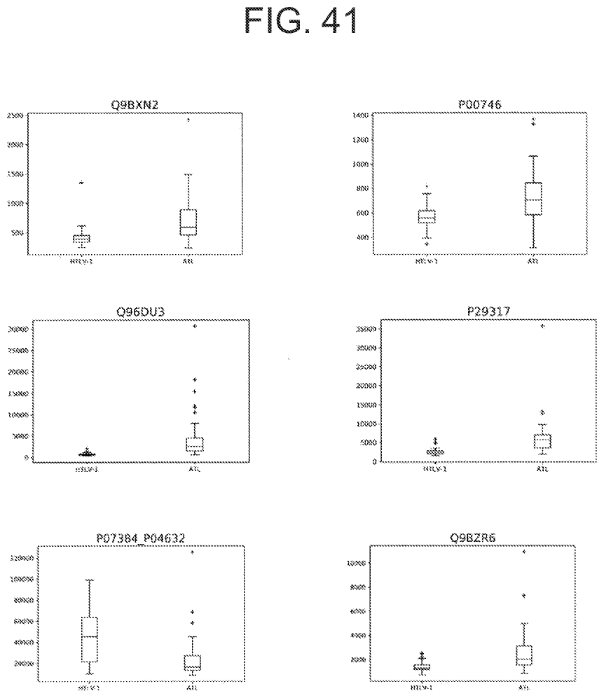

FIG. 42 The figure is a graph showing measurement results of the amount of a protein in plasmas of HTLV-1 careers and ATL patients.

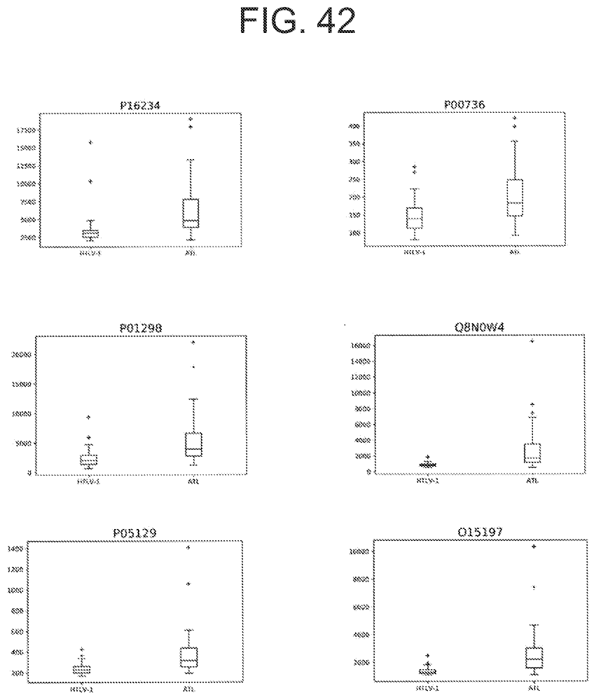

FIG. 43 The figure is a graph showing measurement results of the amount of a protein in plasmas of HTLV-1 careers and ATL patients.

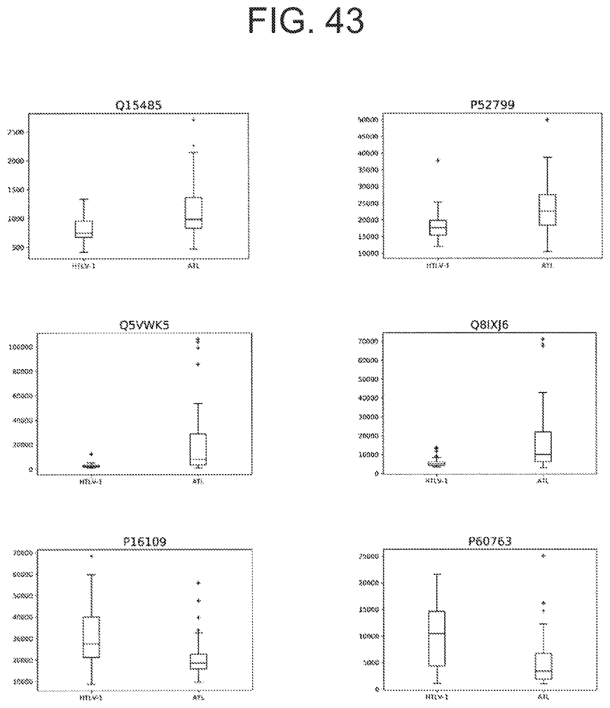

FIG. 44 The figure is a graph showing measurement results of the amount of a protein in plasmas of HTLV-1 careers and ATL patients.

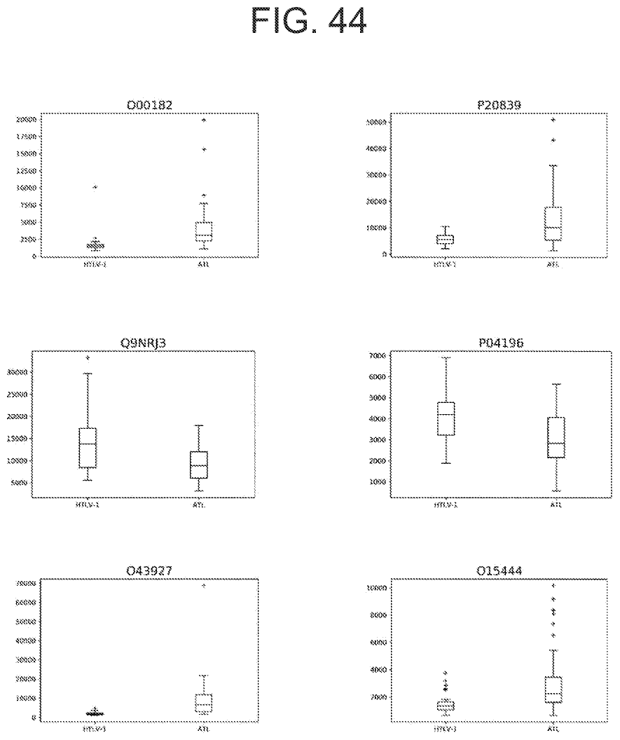

FIG. 45 The figure is a graph showing measurement results of the amount of a protein in plasmas of HTLV-1 careers and ATL patients.

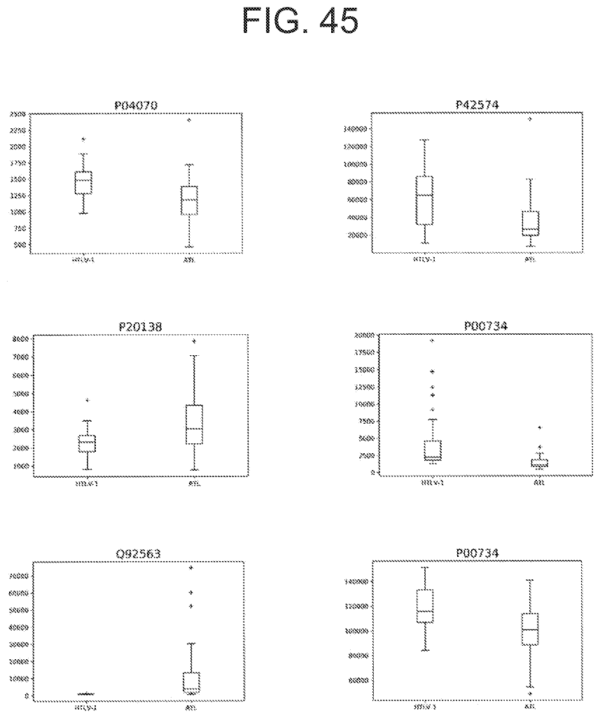

FIG. 46 The figure is a graph showing measurement results of the amount of a protein in plasmas of HTLV-1 careers and ATL patients.

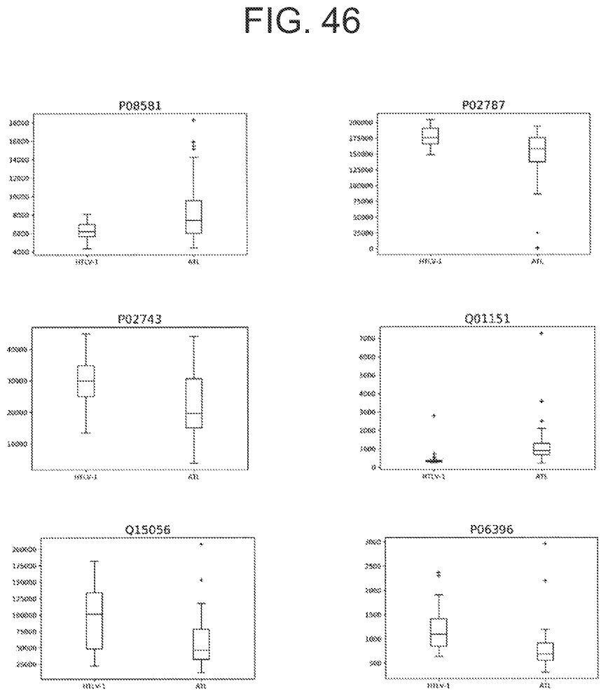

FIG. 47 The figure is a graph showing measurement results of the amount of a protein in plasmas of HTLV-1 careers and ATL patients.

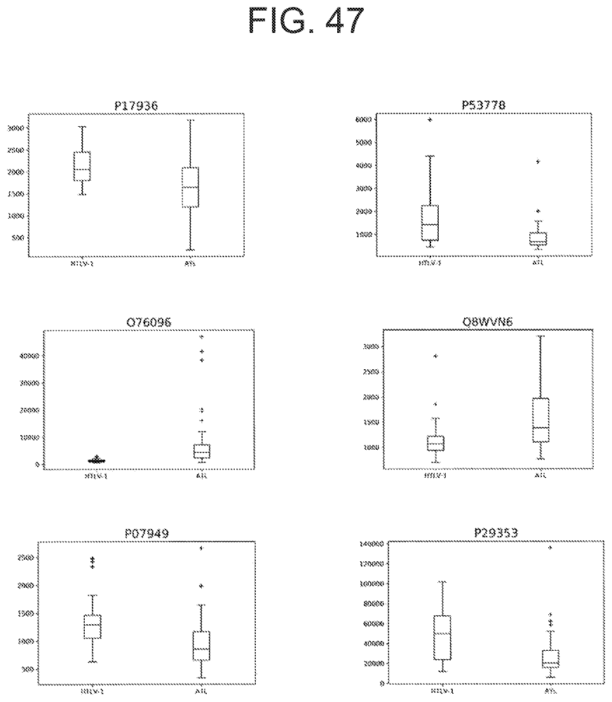

FIG. 48 The figure is a graph showing measurement results of the amount of a protein in plasmas of HTLV-1 careers and ATL patients.

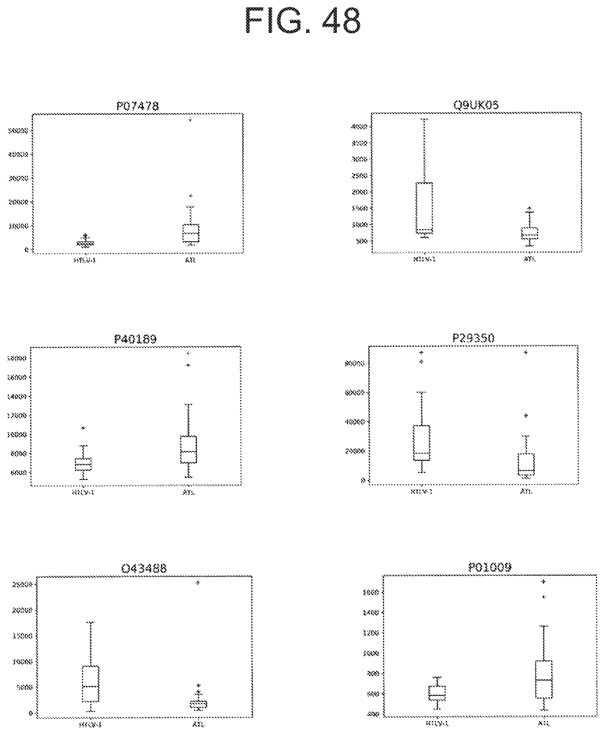

FIG. 49 The figure is a graph showing measurement results of the amount of a protein in plasmas of HTLV-1 careers and ATL patients.

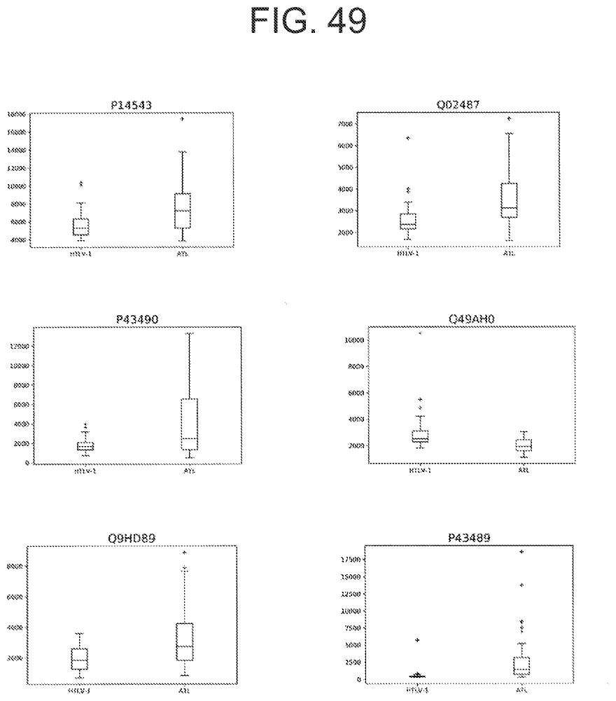

FIG. 50 The figure is a graph showing measurement results of the amount of a protein in plasmas of HTLV-1 careers and ATL patients.

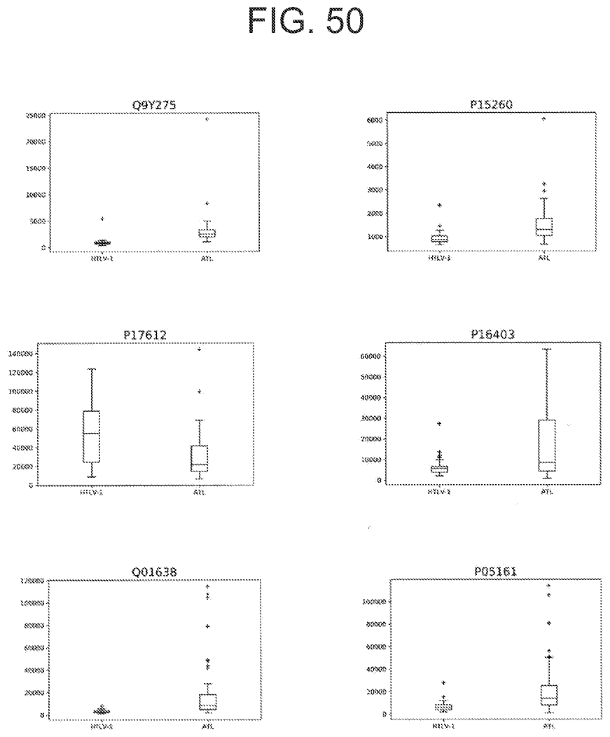

FIG. 51 The figure is a graph showing measurement results of the amount of a protein in plasmas of HTLV-1 careers and ATL patients.

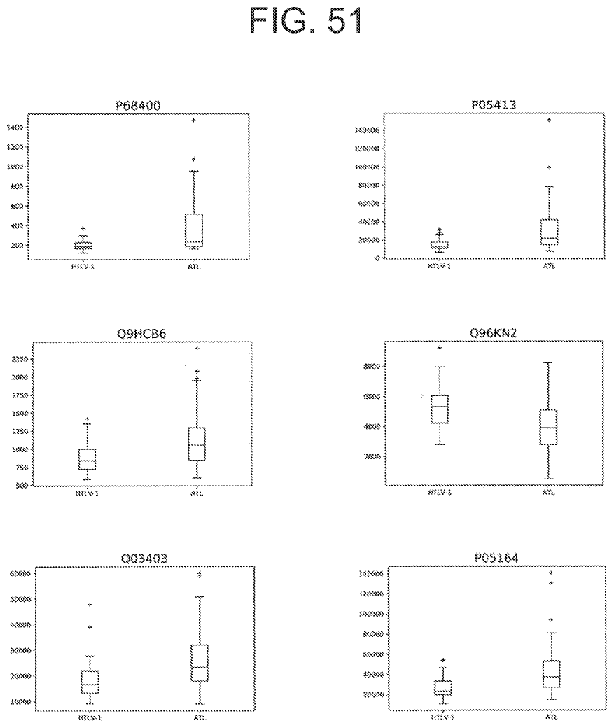

FIG. 52 The figure is a graph showing measurement results of the amount of a protein in plasmas of HTLV-1 careers and ATL patients.

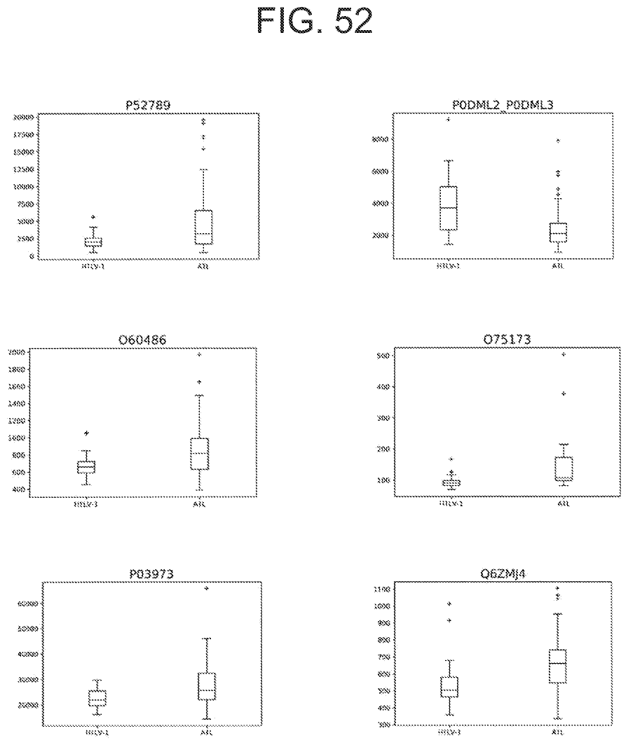

FIG. 53 The figure is a graph showing measurement results of the amount of a protein in plasmas of HTLV-1 careers and ATL patients.

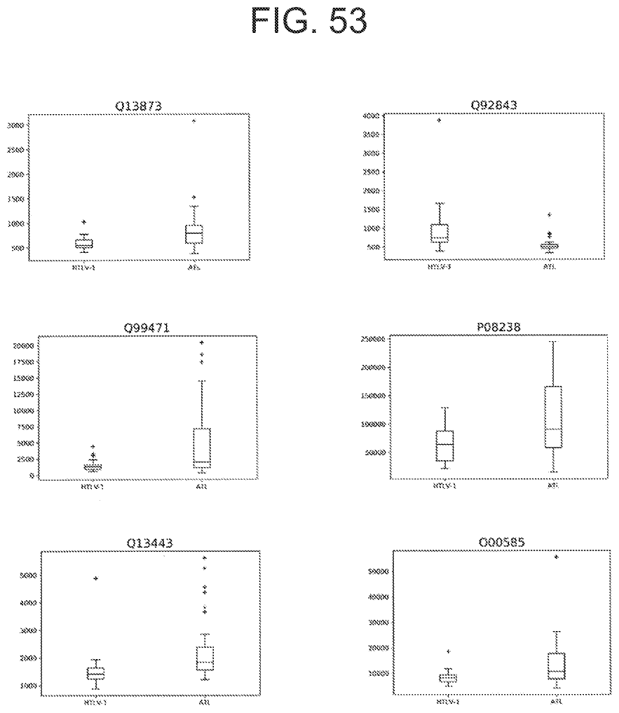

FIG. 54 The figure is a graph showing measurement results of the amount of a protein in plasmas of HTLV-1 careers and ATL patients.

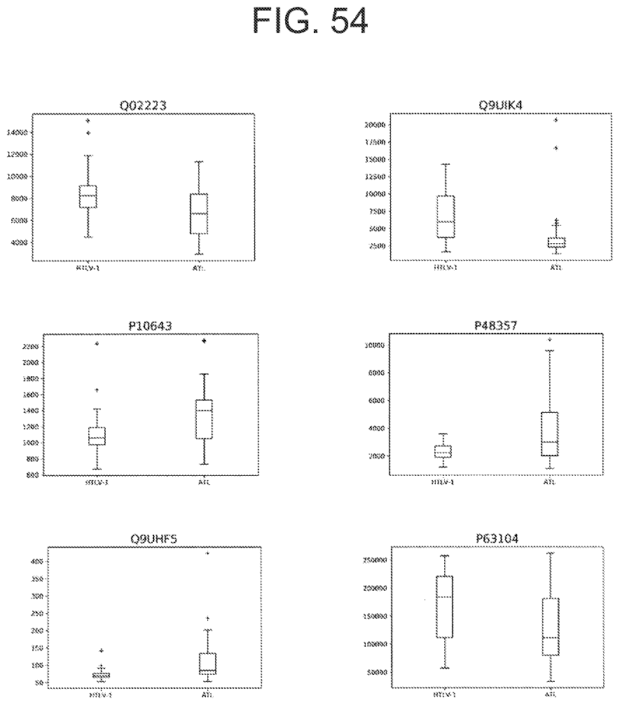

FIG. 55 The figure is a graph showing measurement results of the amount of a protein in plasmas of HTLV-1 careers and ATL patients.

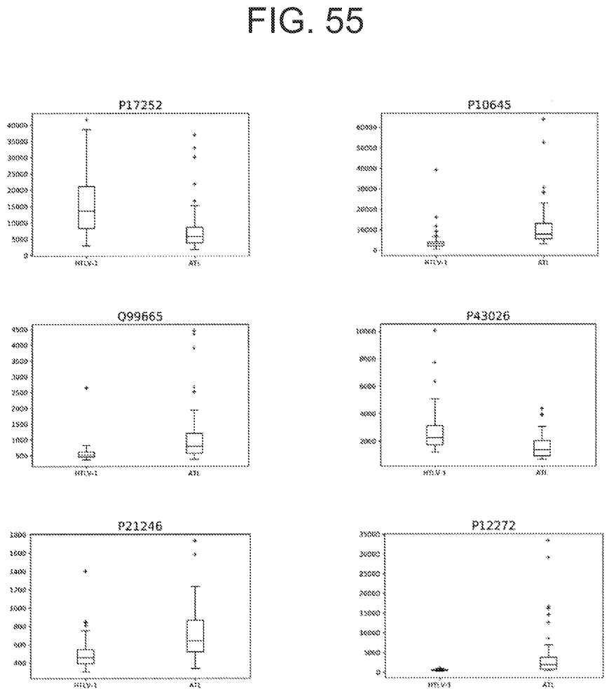

FIG. 56 The figure is a graph showing measurement results of the amount of a protein in plasmas of HTLV-1 careers and ATL patients.

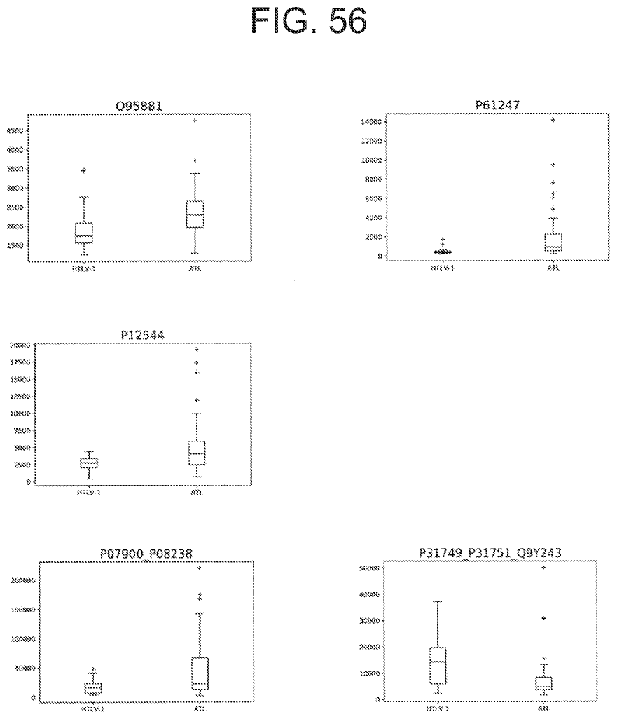

FIG. 57 The figure is a graph showing measurement results of the amount of a protein in plasmas of HTLV-1 careers and ATL patients.

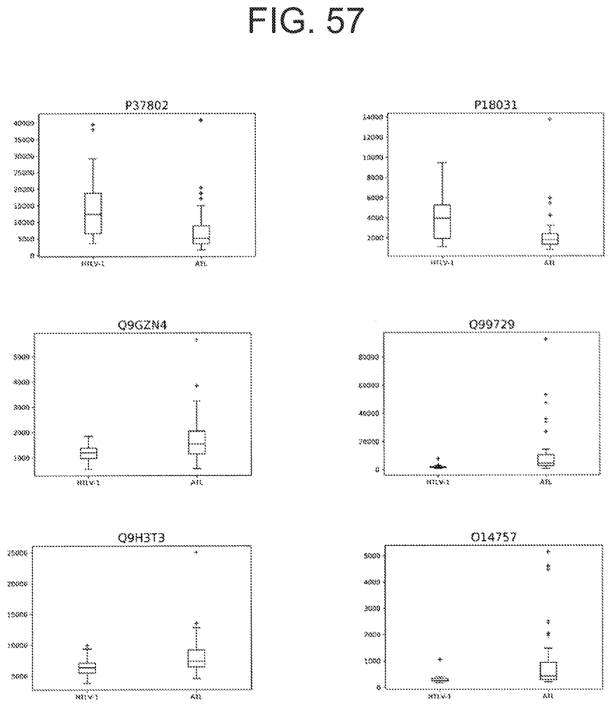

FIG. 58 The figure is a graph showing measurement results of the amount of a protein in plasmas of HTLV-1 careers and ATL patients.

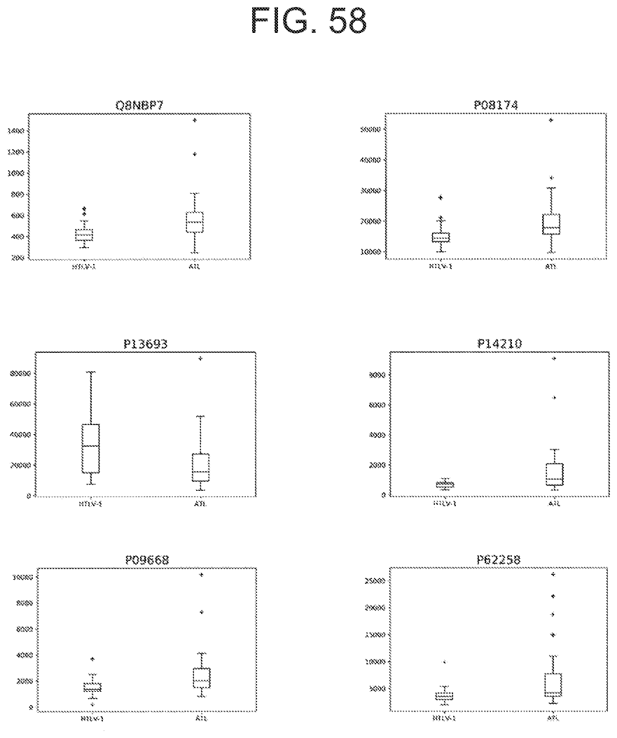

FIG. 59 The figure is a graph showing measurement results of the amount of a protein in plasmas of HTLV-1 careers and ATL patients.

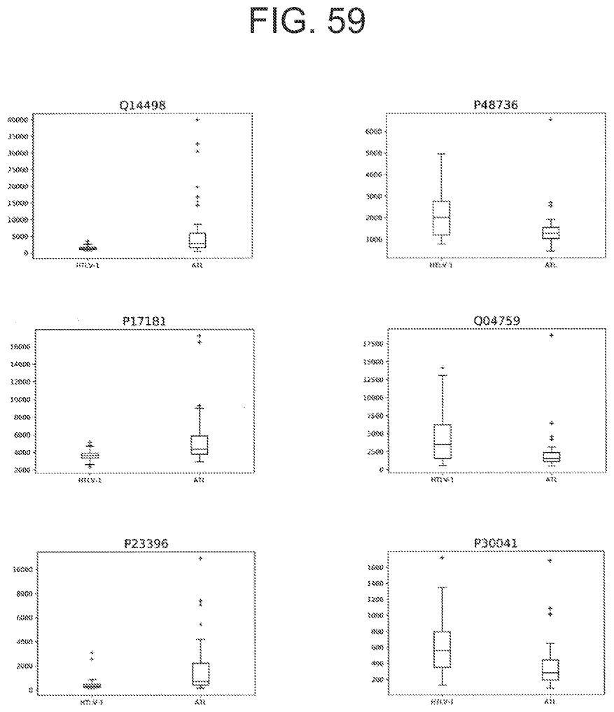

FIG. 60 The figure is a graph showing measurement results of the amount of a protein in plasmas of HTLV-1 careers and ATL patients.

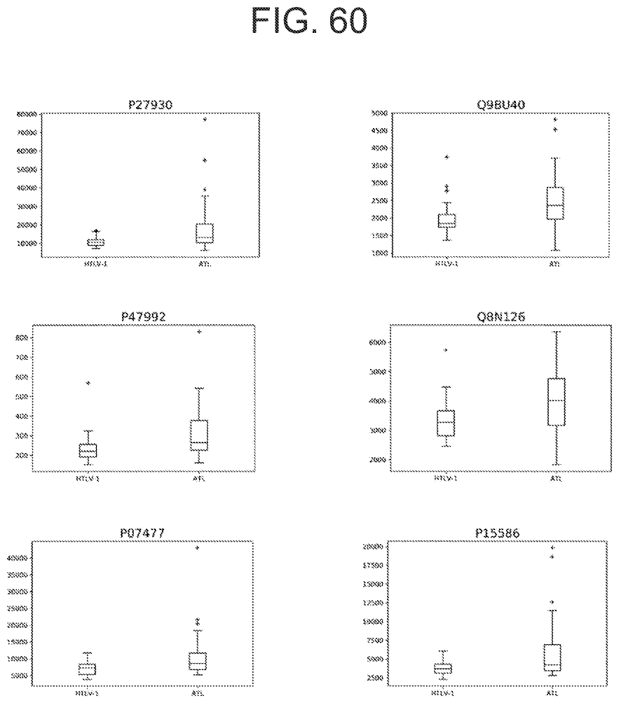

FIG. 61 The figure is a graph showing measurement results of the amount of a protein in plasmas of HTLV-1 careers and ATL patients.

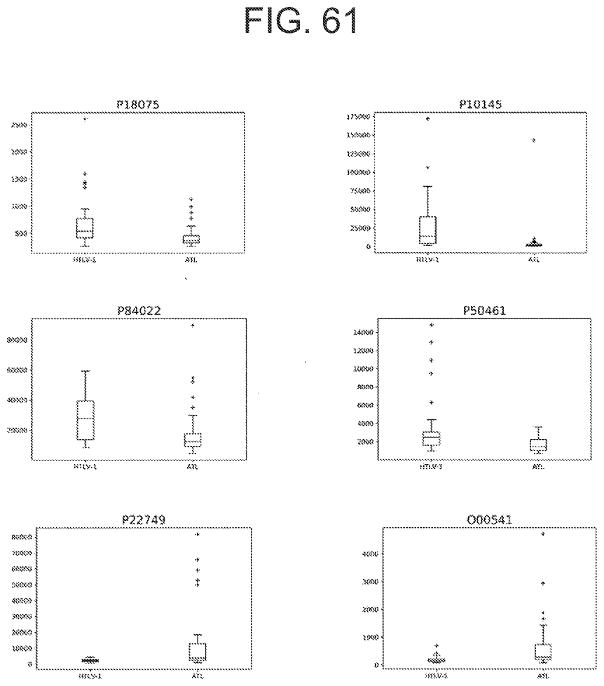

FIG. 62 The figure is a graph showing measurement results of the amount of a protein in plasmas of HTLV-1 careers and ATL patients.

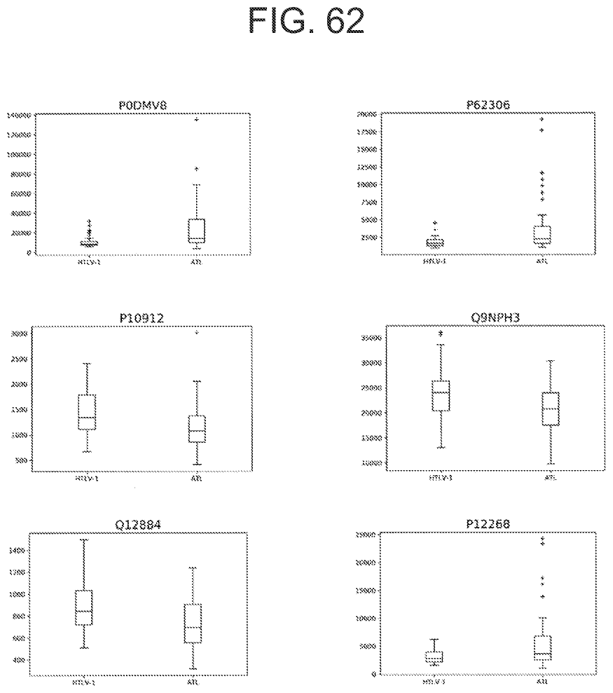

FIG. 63 The figure is a graph showing measurement results of the amount of a protein in plasmas of HTLV-1 careers and ATL patients.

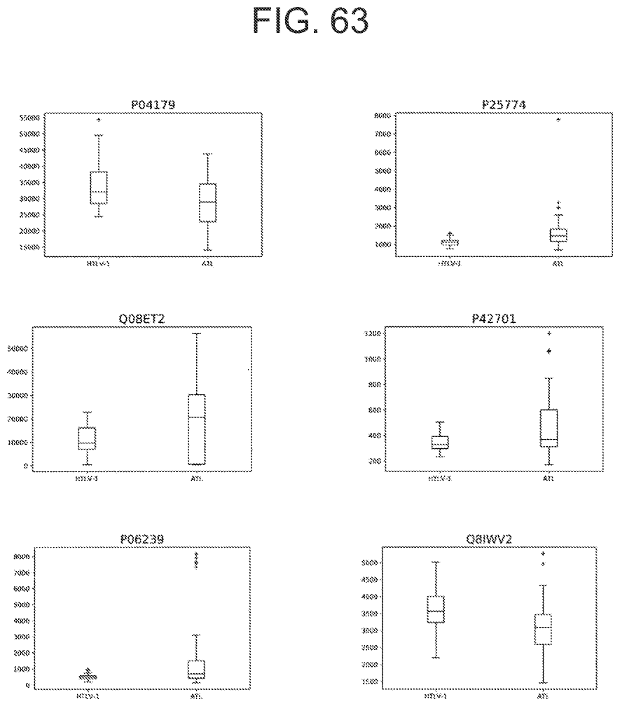

FIG. 64 The figure is a graph showing measurement results of the amount of a protein in plasmas of HTLV-1 careers and ATL patients.

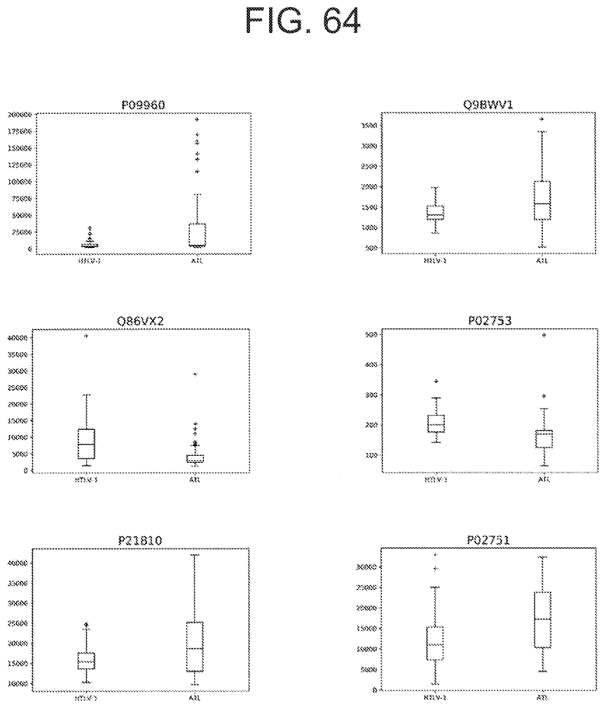

FIG. 65 The figure is a graph showing measurement results of the amount of a protein in plasmas of HTLV-1 careers and ATL patients.

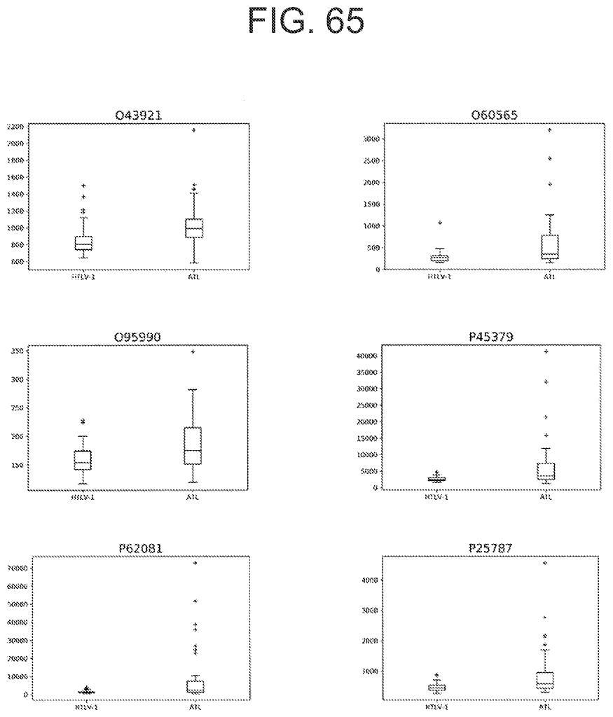

FIG. 66 The figure is a graph showing measurement results of the amount of a protein in plasmas of HTLV-1 careers and ATL patients.

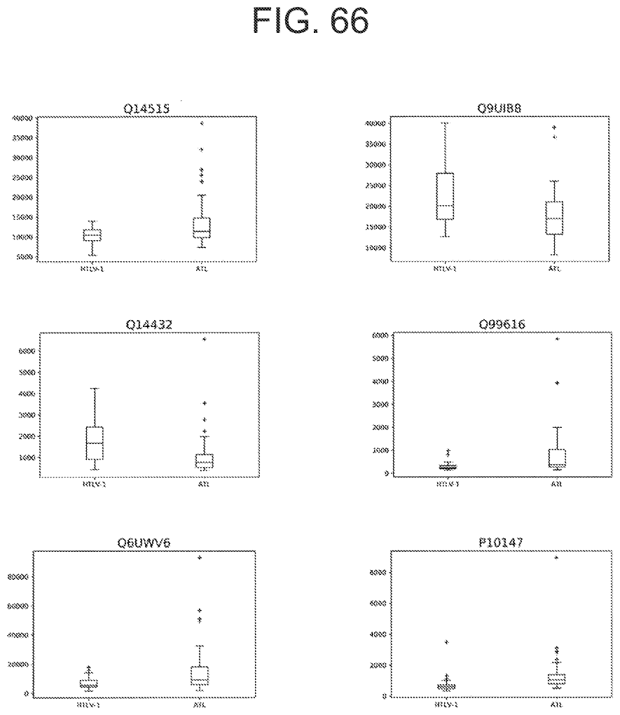

FIG. 67 The figure is a graph showing measurement results of the amount of a protein in plasmas of HTLV-1 careers and ATL patients.

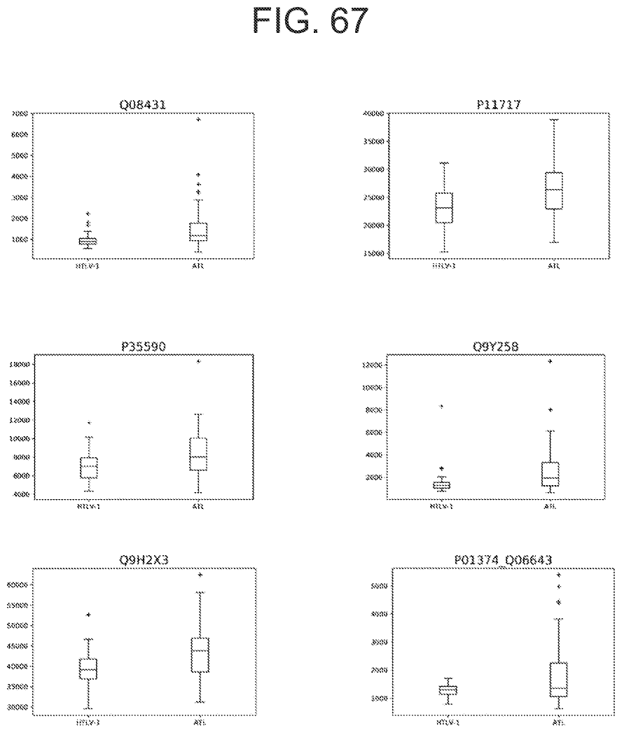

FIG. 68 The figure is a graph showing measurement results of the amount of a protein in plasmas of HTLV-1 careers and ATL patients.

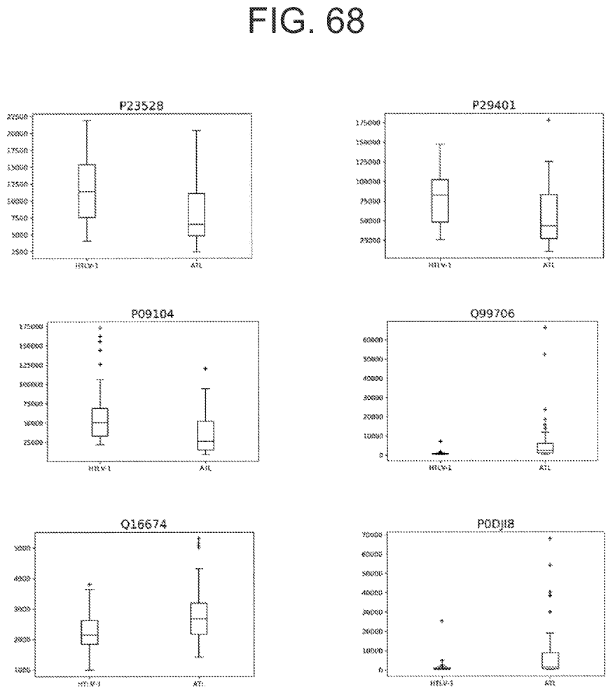

FIG. 69 The figure is a graph showing measurement results of the amount of a protein in plasmas of HTLV-1 careers and ATL patients.

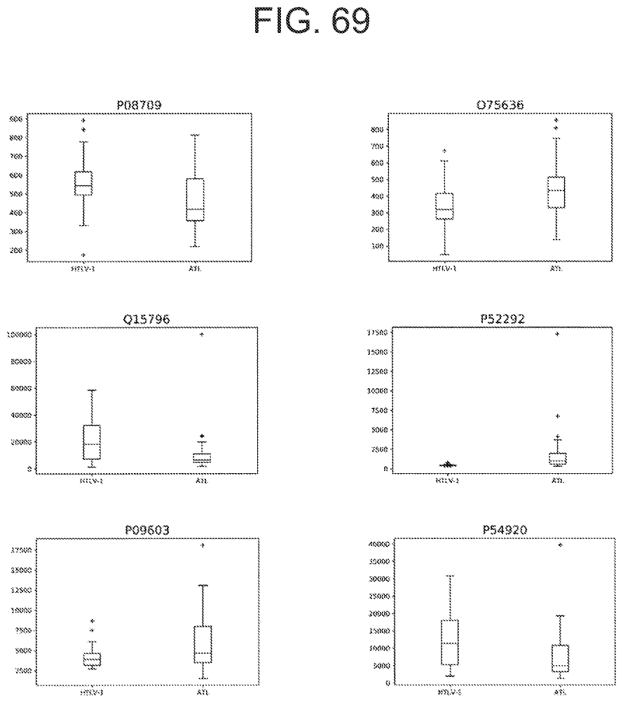

FIG. 70 The figure is a graph showing measurement results of the amount of a protein in plasmas of HTLV-1 careers and ATL patients.

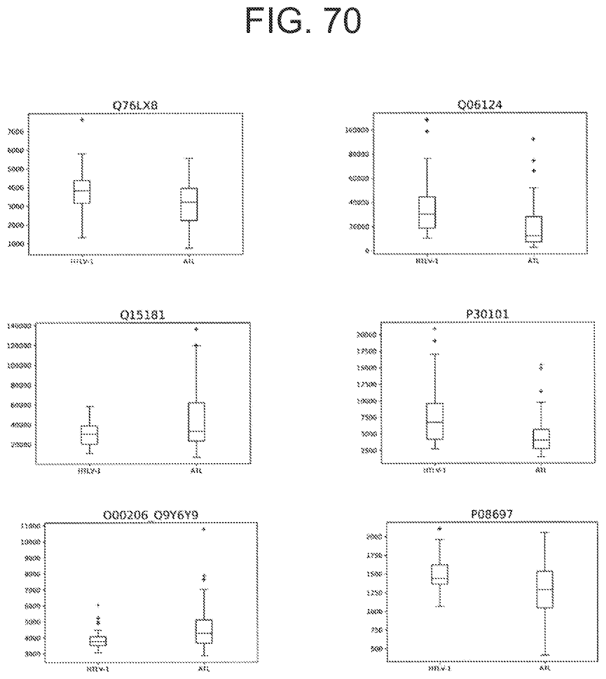

FIG. 71 The figure is a graph showing measurement results of the amount of a protein in plasmas of HTLV-1 careers and ATL patients.

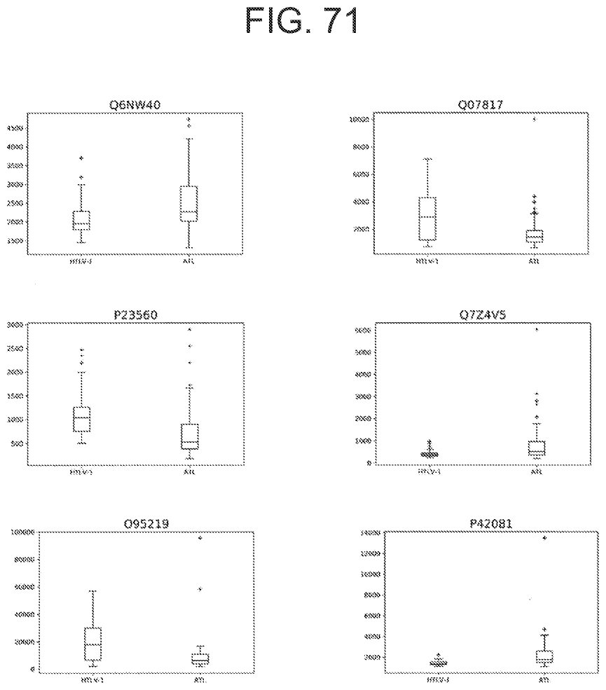

FIG. 72 The figure is a graph showing measurement results of the amount of a protein in plasmas of HTLV-1 careers and ATL patients.

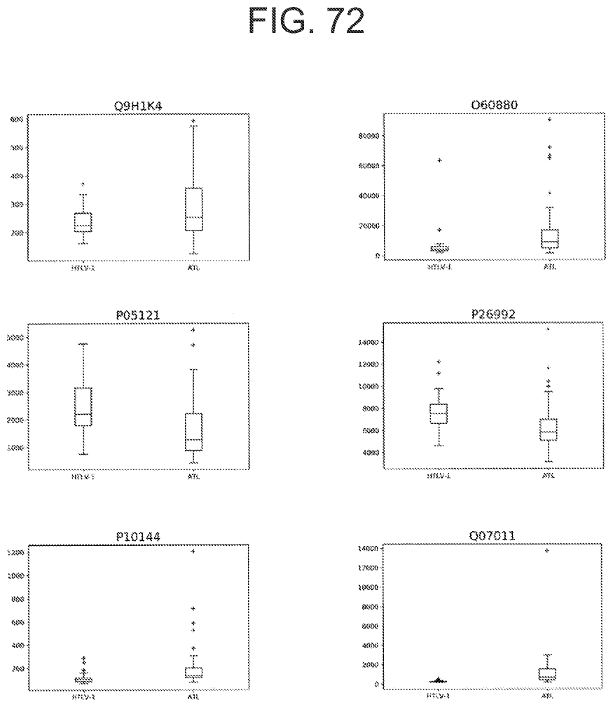

FIG. 73 The figure is a graph showing measurement results of the amount of a protein in plasmas of HTLV-1 careers and ATL patients.

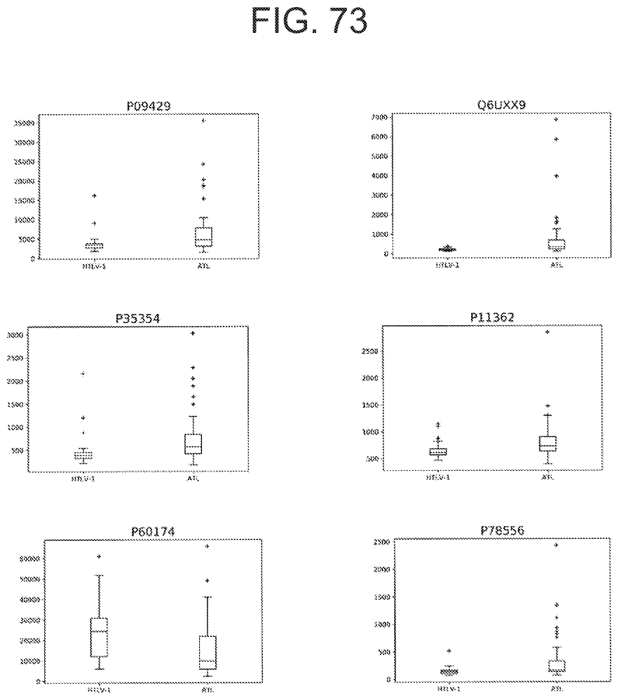

FIG. 74 The figure is a graph showing measurement results of the amount of a protein in plasmas of HTLV-1 careers and ATL patients.

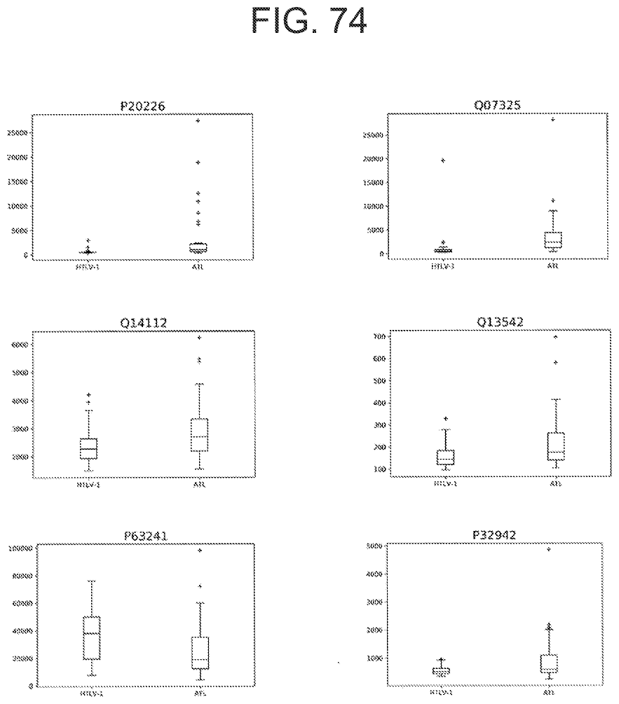

FIG. 75 The figure is a graph showing measurement results of the amount of a protein in plasmas of HTLV-1 careers and ATL patients.

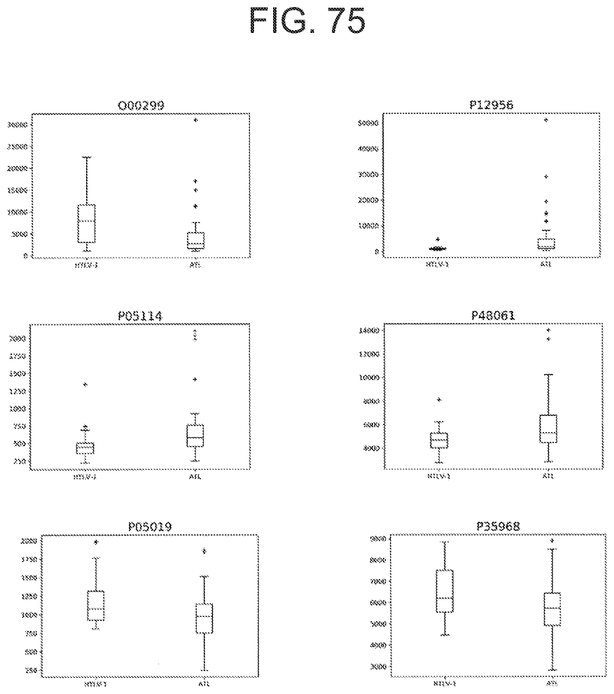

FIG. 76 The figure is a graph showing measurement results of the amount of a protein in plasmas of HTLV-1 careers and ATL patients.

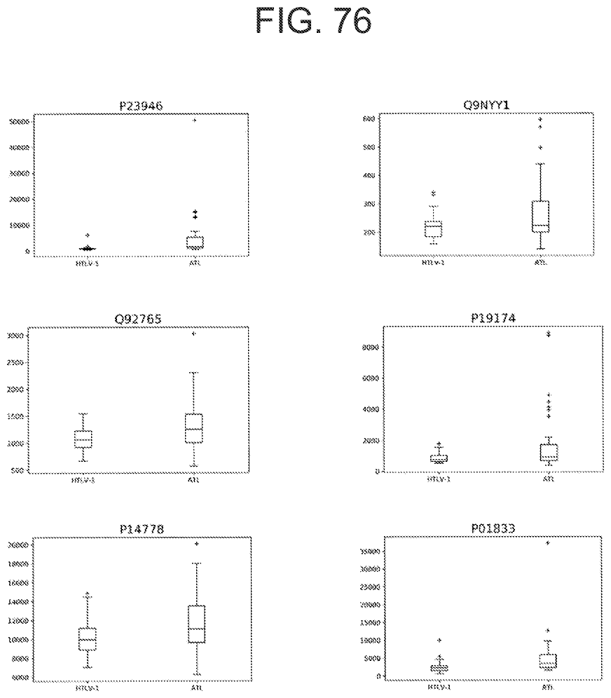

FIG. 77 The figure is a graph showing measurement results of the amount of a protein in plasmas of HTLV-1 careers and ATL patients.

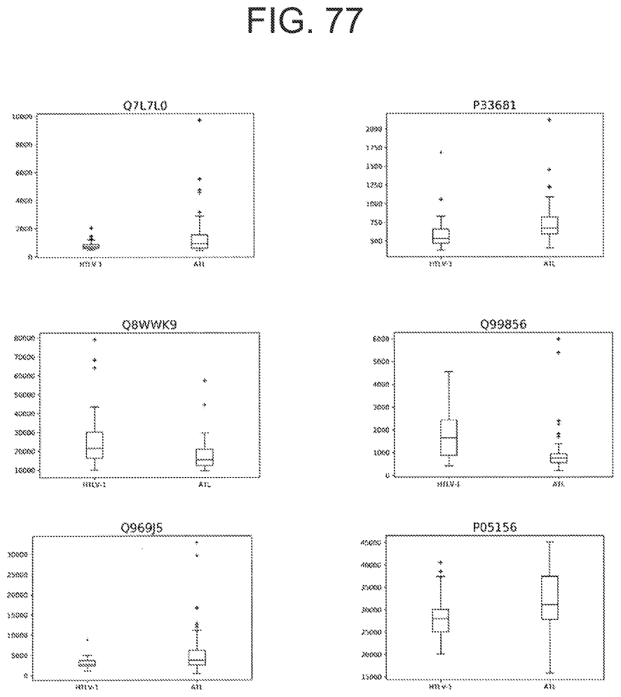

FIG. 78 The figure is a graph showing measurement results of the amount of a protein in plasmas of HTLV-1 careers and ATL patients.

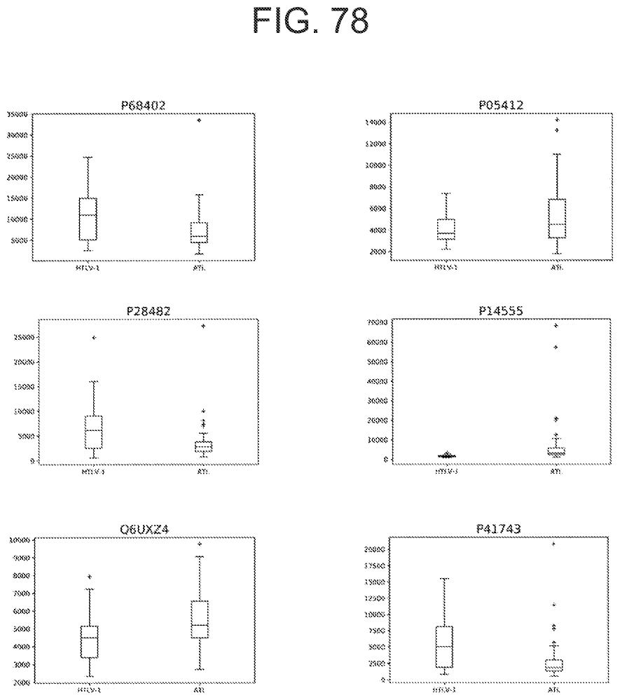

FIG. 79 The figure is a graph showing measurement results of the amount of a protein in plasmas of HTLV-1 careers and ATL patients.

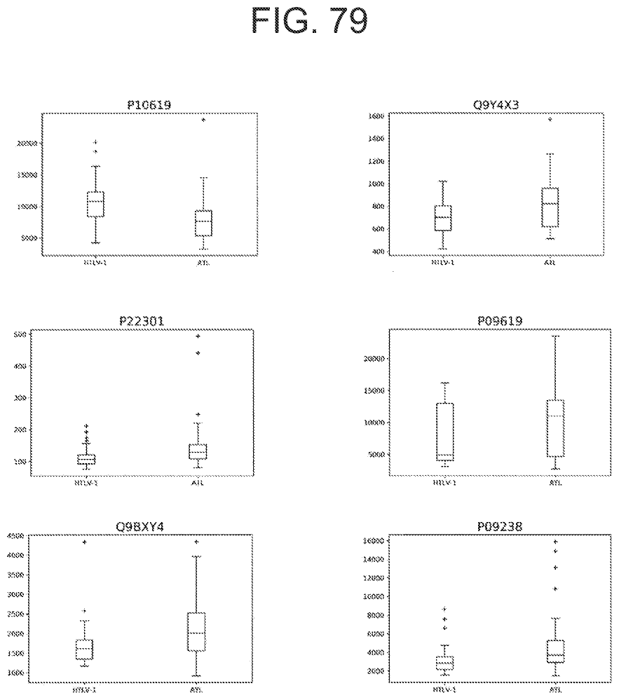

FIG. 80 The figure is a graph showing measurement results of the amount of a protein in plasmas of HTLV-1 careers and ATL patients.

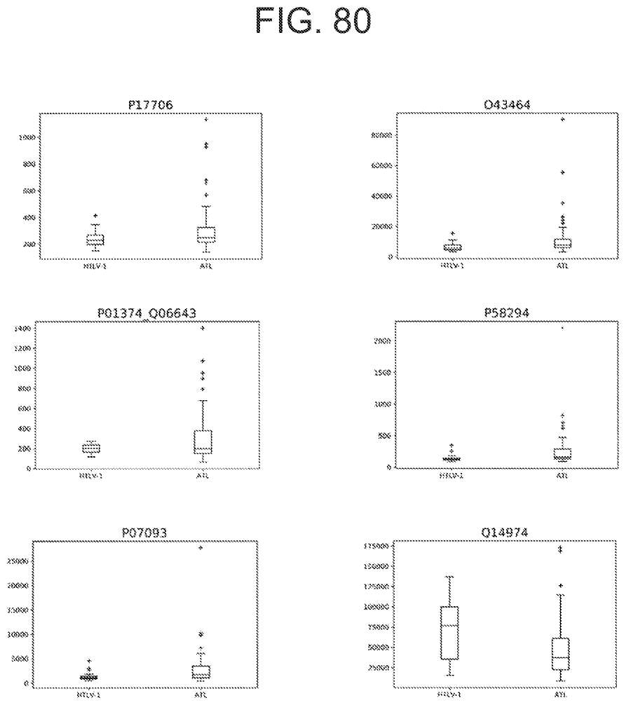

FIG. 81 The figure is a graph showing measurement results of the amount of a protein in plasmas of HTLV-1 careers and ATL patients.

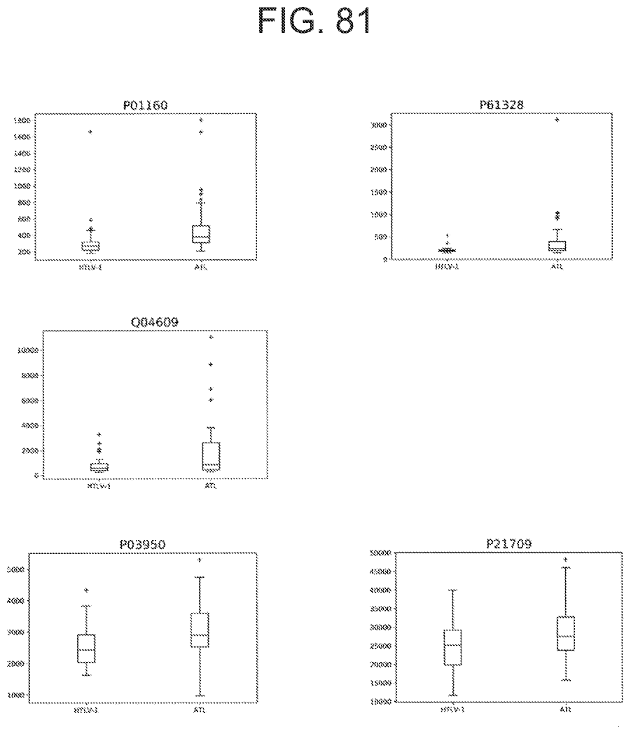

FIG. 82 The figure is a graph showing measurement results of the amount of a protein in plasmas of HTLV-1 careers and ATL patients.

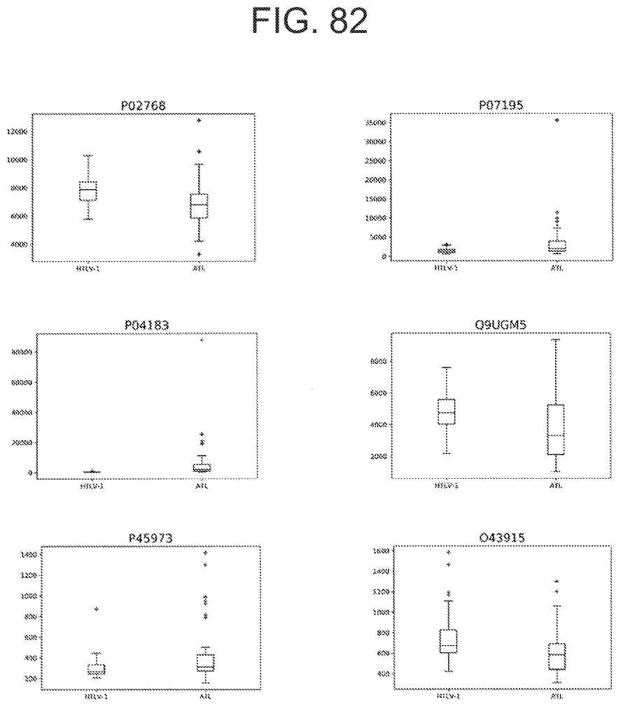

FIG. 83 The figure is a graph showing measurement results of the amount of a protein in plasmas of HTLV-1 careers and ATL patients.

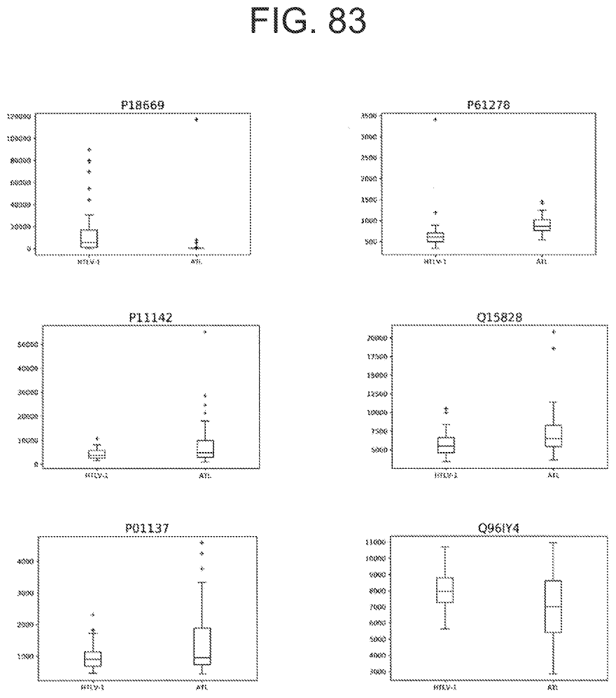

FIG. 84 The figure is a graph showing measurement results of the amount of a protein in plasmas of HTLV-1 careers and ATL patients.

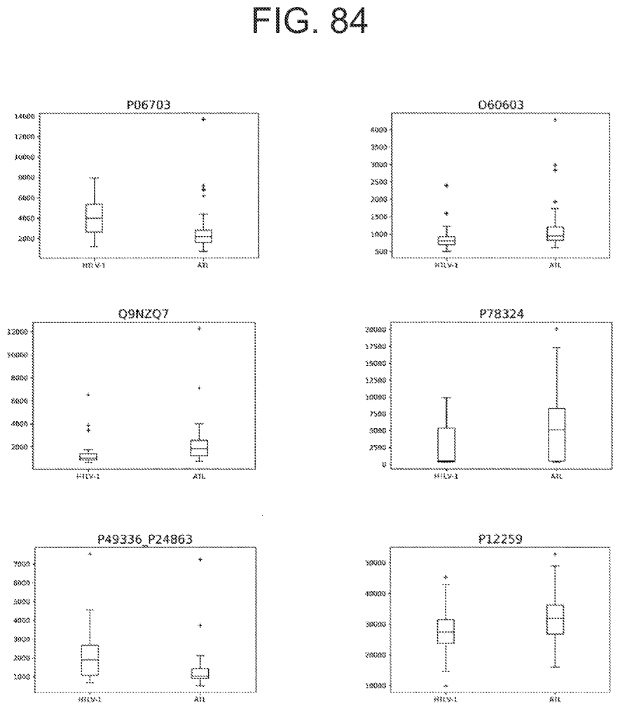

FIG. 85 The figure is a graph showing measurement results of the amount of a protein in plasmas of HTLV-1 careers and ATL patients.

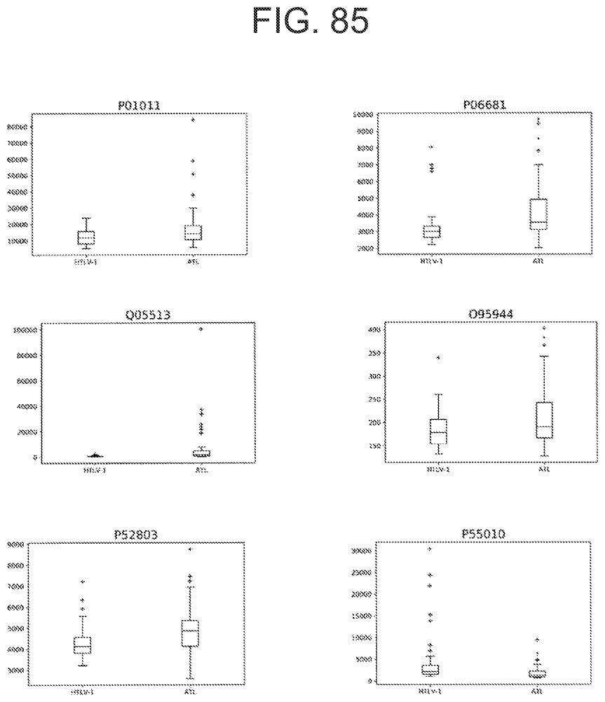

FIG. 86 The figure is a graph showing measurement results of the amount of a protein in plasmas of HTLV-1 careers and ATL patients.

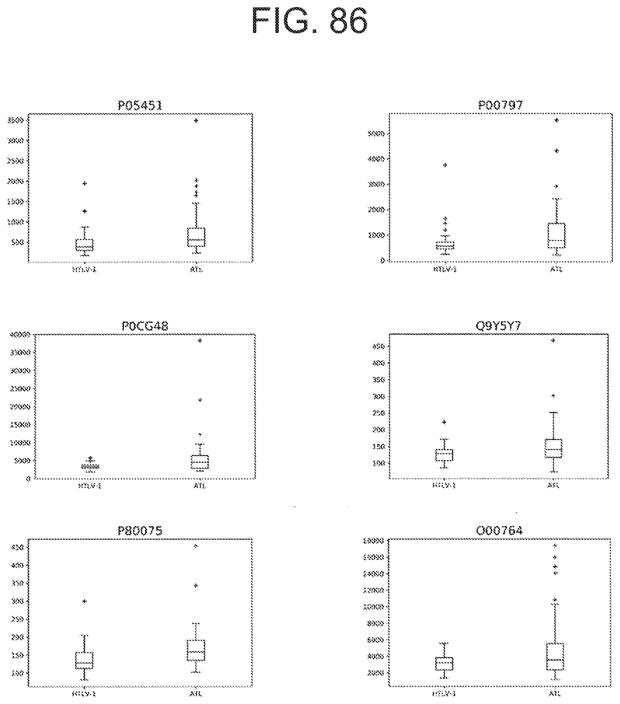

FIG. 87 The figure is a graph showing measurement results of the amount of a protein in plasmas of HTLV-1 careers and ATL patients.

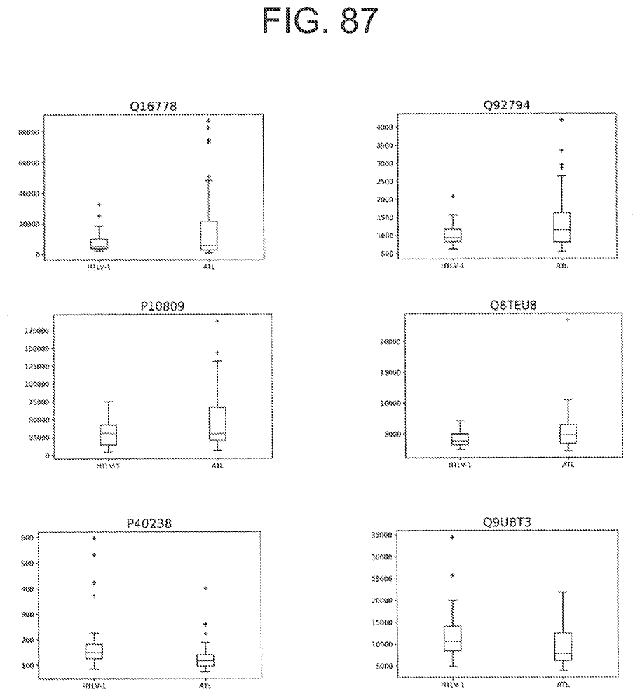

FIG. 88 The figure is a graph showing measurement results of the amount of a protein in plasmas of HTLV-1 careers and ATL patients.

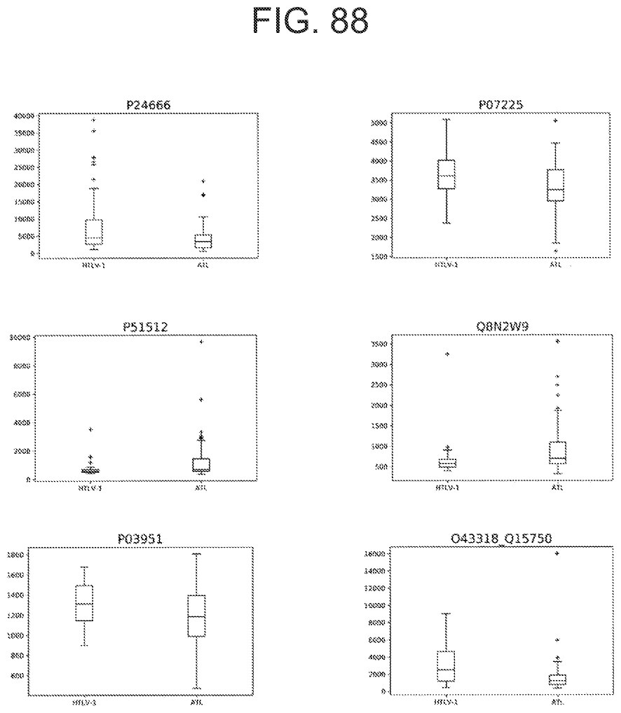

FIG. 89 The figure is a graph showing measurement results of the amount of a protein in plasmas of HTLV-1 careers and ATL patients.

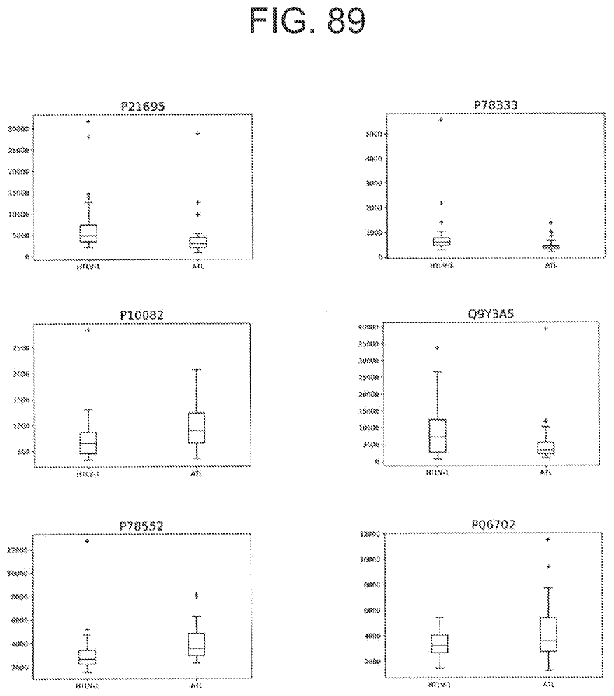

FIG. 90 The figure is a graph showing measurement results of the amount of a protein in plasmas of HTLV-1 careers and ATL patients.

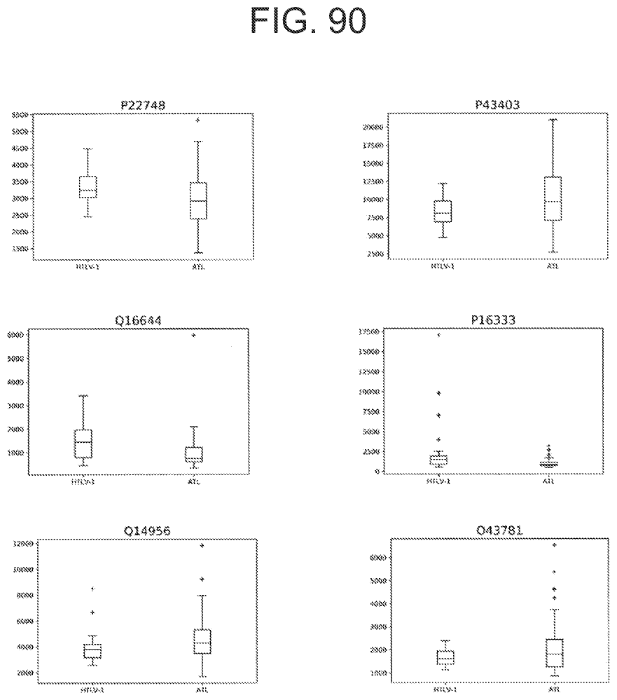

FIG. 91 The figure is a graph showing measurement results of the amount of a protein in plasmas of HTLV-1 careers and ATL patients.

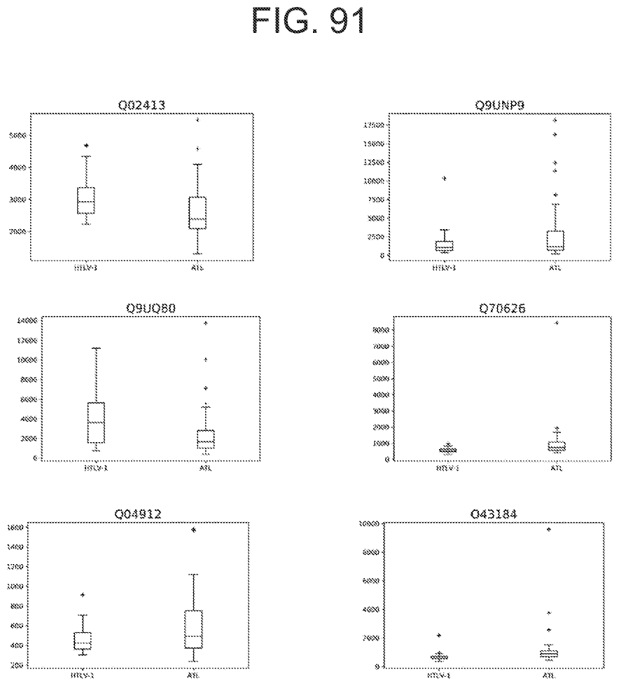

FIG. 92 The figure is a graph showing measurement results of the amount of a protein in plasmas of HTLV-1 careers and ATL patients.

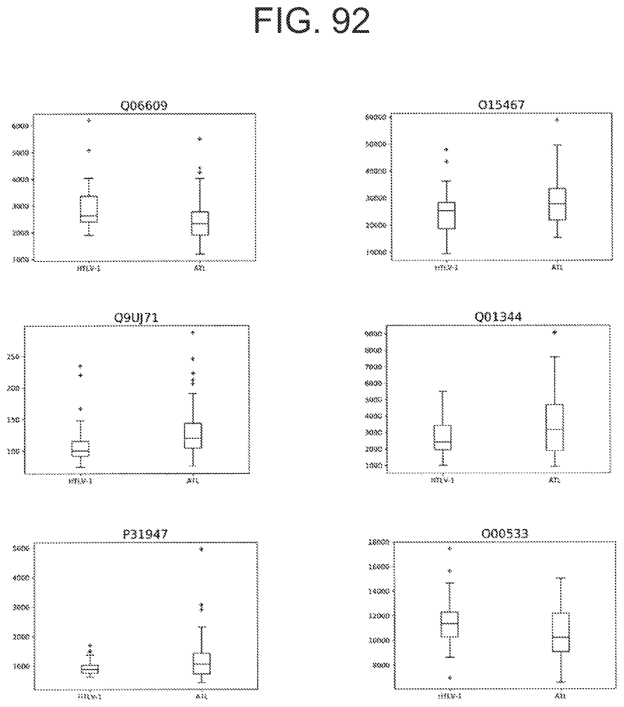

FIG. 93 The figure is a graph showing measurement results of the amount of a protein in plasmas of HTLV-1 careers and ATL patients.

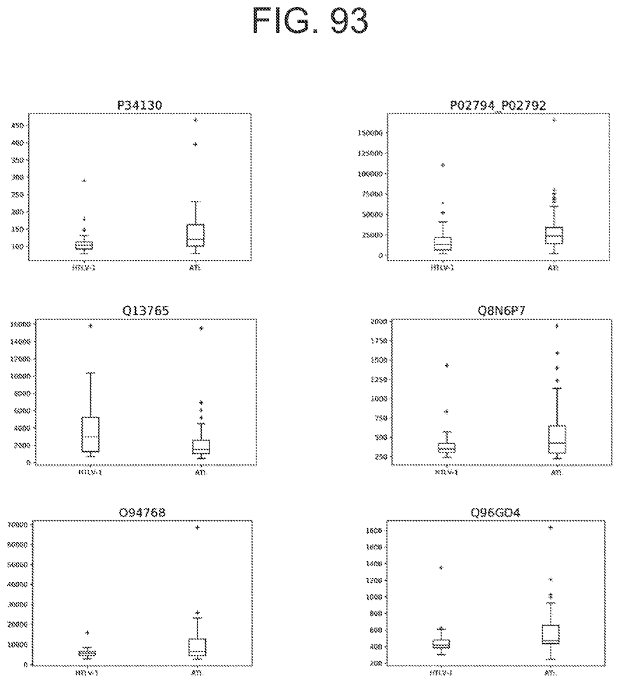

FIG. 94 The figure is a graph showing measurement results of the amount of a protein in plasmas of HTLV-1 careers and ATL patients.

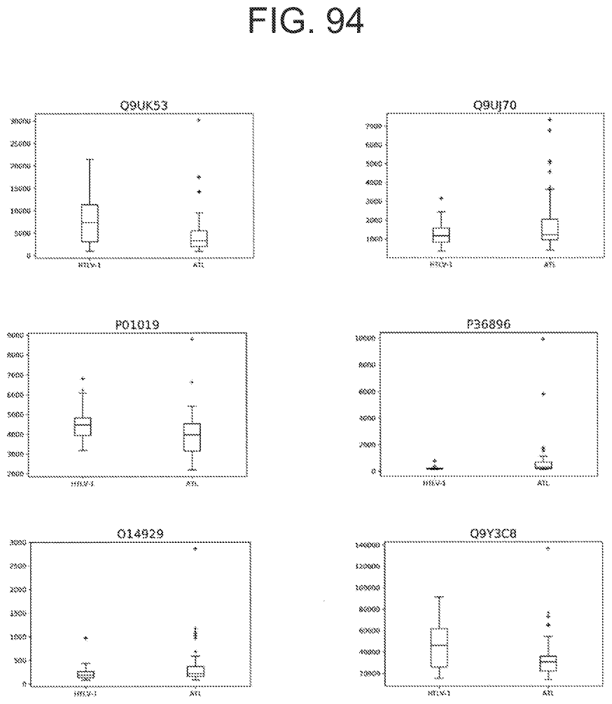

FIG. 95 The figure is a graph showing measurement results of the amount of a protein in plasmas of HTLV-1 careers and ATL patients.

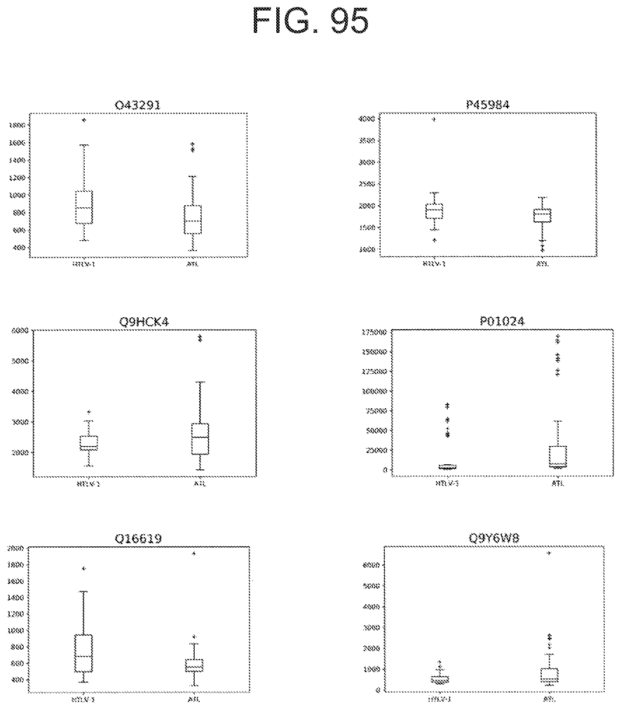

FIG. 96 The figure is a graph showing measurement results of the amount of a protein in plasmas of HTLV-1 careers and ATL patients.

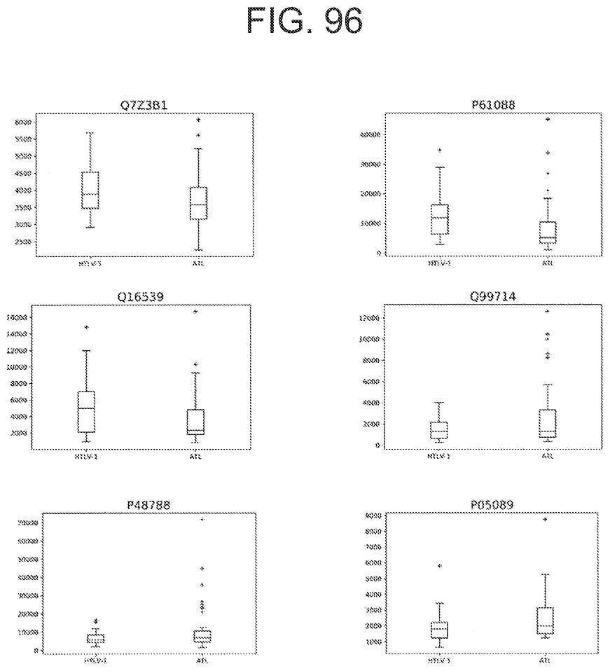

FIG. 97 The figure is a graph showing measurement results of the amount of a protein in plasmas of HTLV-1 careers and ATL patients.

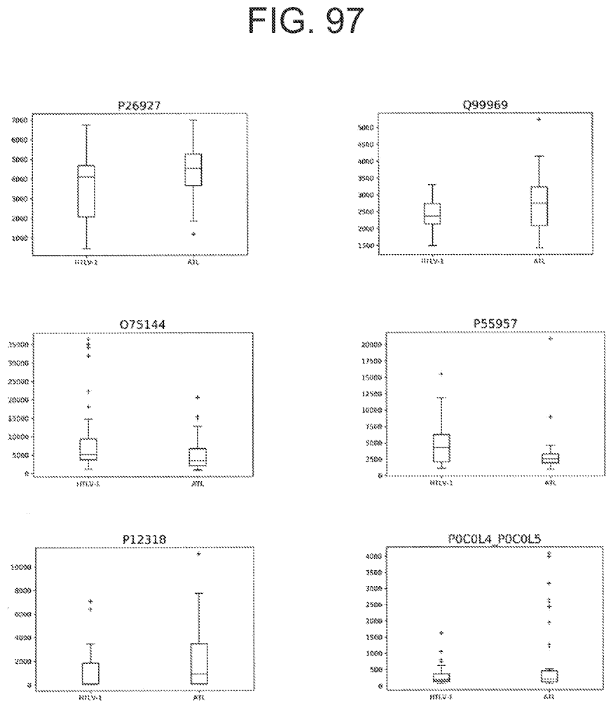

FIG. 98 The figure is a graph showing measurement results of the amount of a protein in plasmas of HTLV-1 careers and ATL patients.

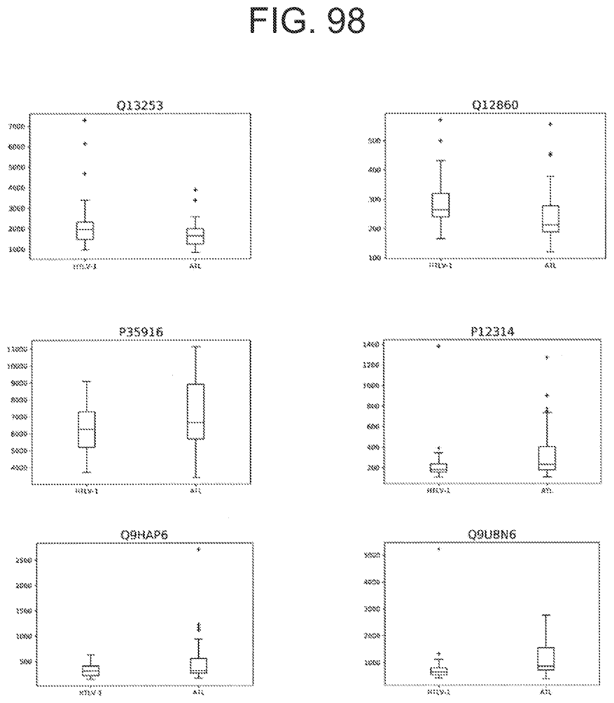

FIG. 99 The figure is a graph showing measurement results of the amount of a protein in plasmas of HTLV-1 careers and ATL patients.

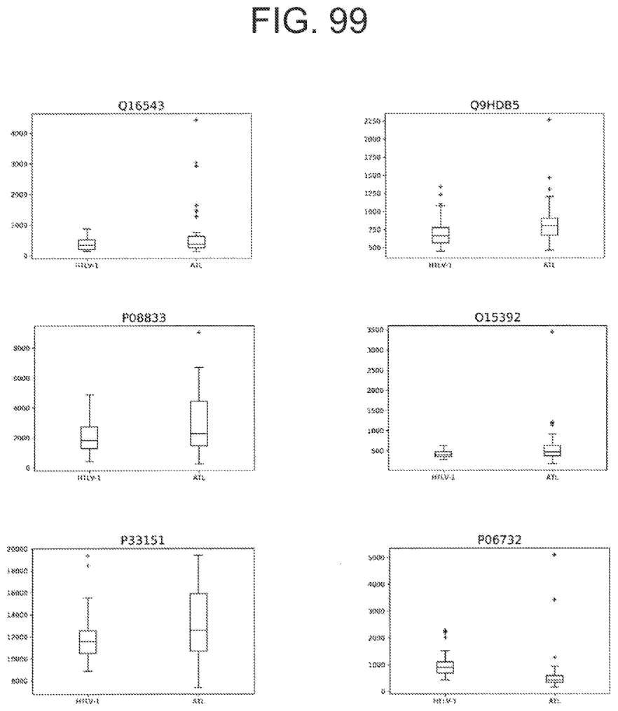

FIG. 100 The figure is a graph showing measurement results of the amount of a protein in plasmas of HTLV-1 careers and ATL patients.

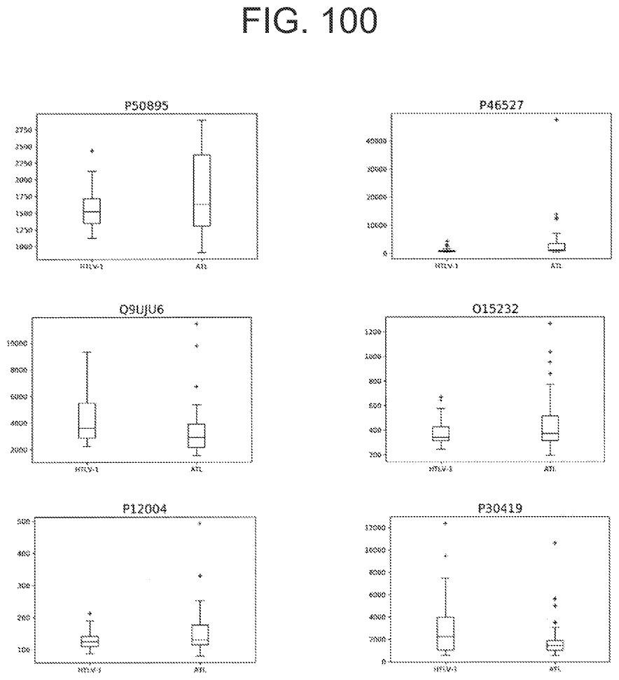

FIG. 101 The figure is a graph showing measurement results of the amount of a protein in plasmas of HTLV-1 careers and ATL patients.

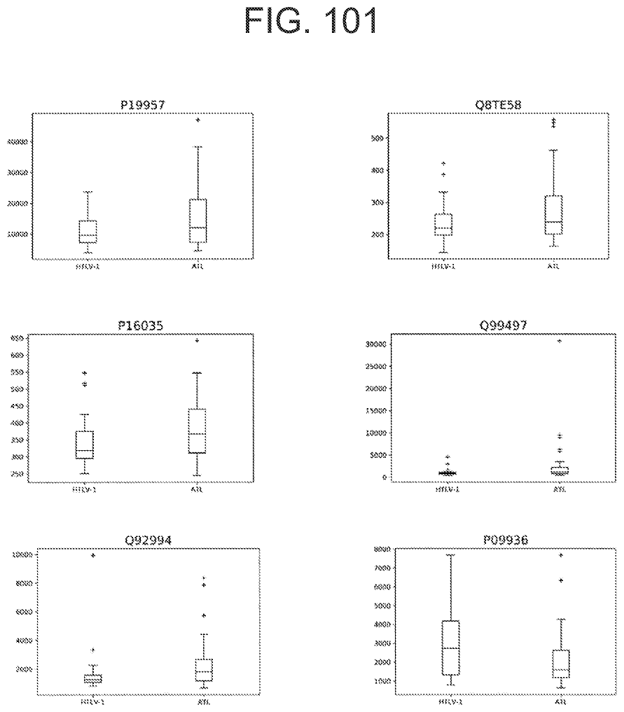

FIG. 102 The figure is a graph showing measurement results of the amount of a protein in plasmas of HTLV-1 careers and ATL patients.

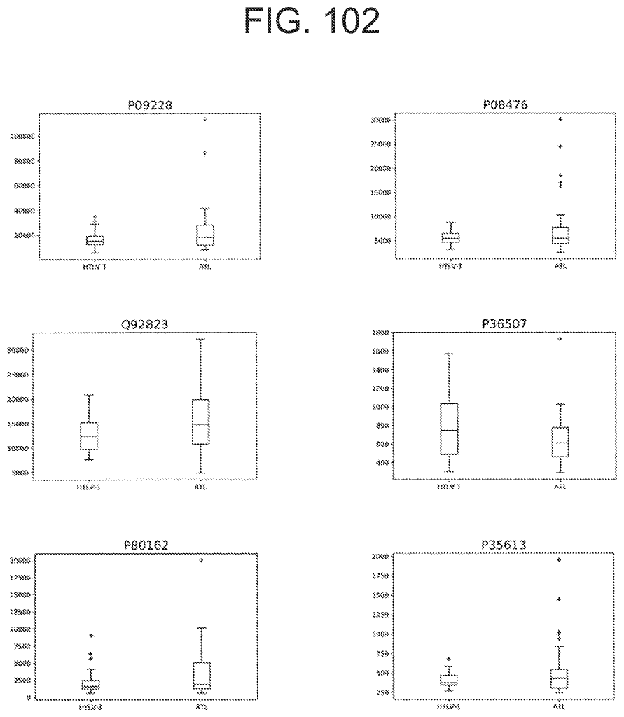

FIG. 103 The figure is a graph showing measurement results of the amount of a protein in plasmas of HTLV-1 careers and ATL patients.

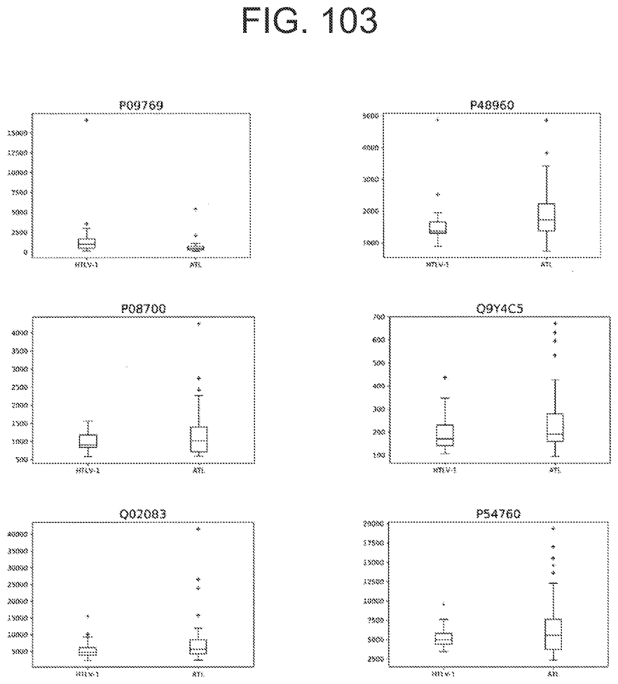

FIG. 104 The figure is a graph showing measurement results of the amount of a protein in plasmas of HTLV-1 careers and ATL patients.

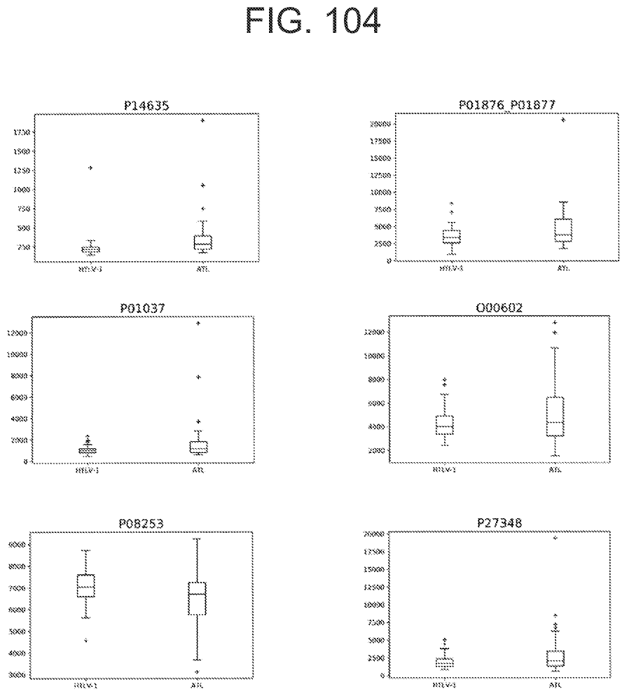

FIG. 105 The figure is a graph showing measurement results of the amount of a protein in plasmas of HTLV-1 careers and ATL patients.

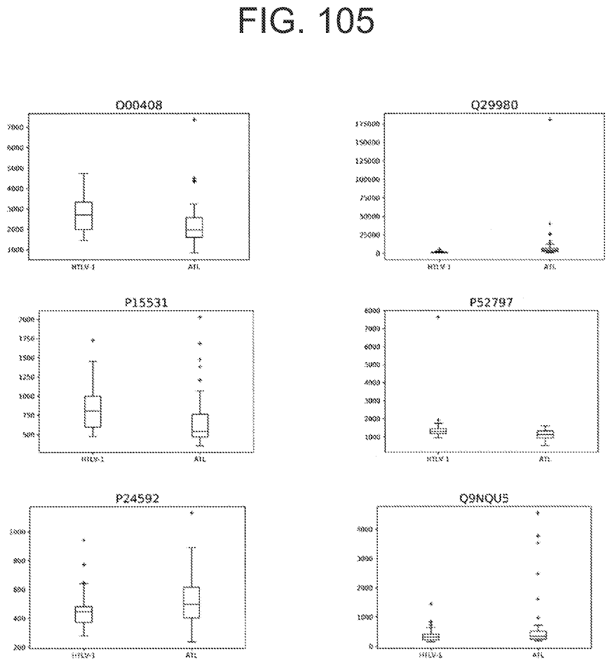

FIG. 106 The figure is a graph showing measurement results of the amount of a protein in plasmas of HTLV-1 careers and ATL patients.

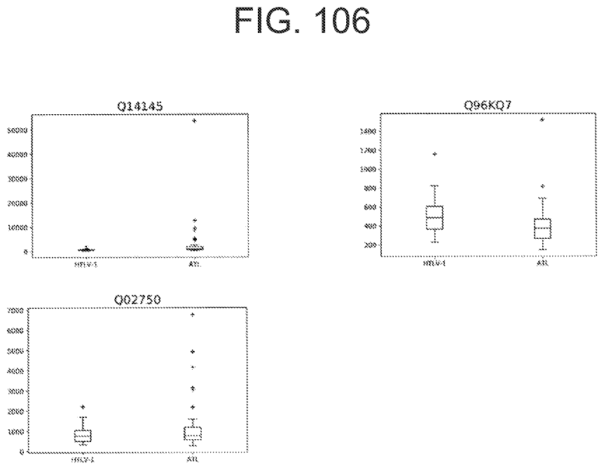

FIG. 107 The figure is a graph showing measurement results of the amount of a protein in plasmas of ATL patients and ATL patients in remission.

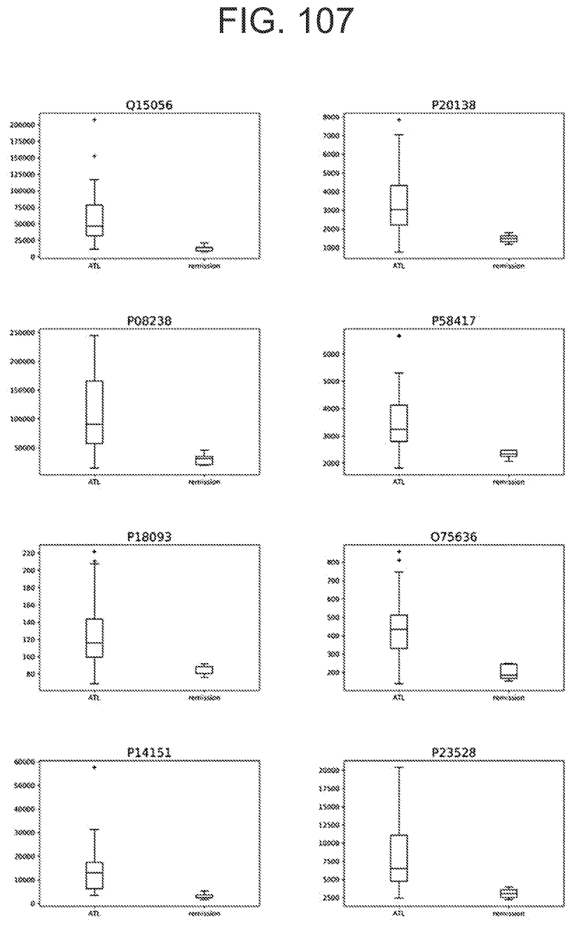

FIG. 108 The figure is a graph showing measurement results of the amount of a protein in plasmas of ATL patients and ATL patients in remission.

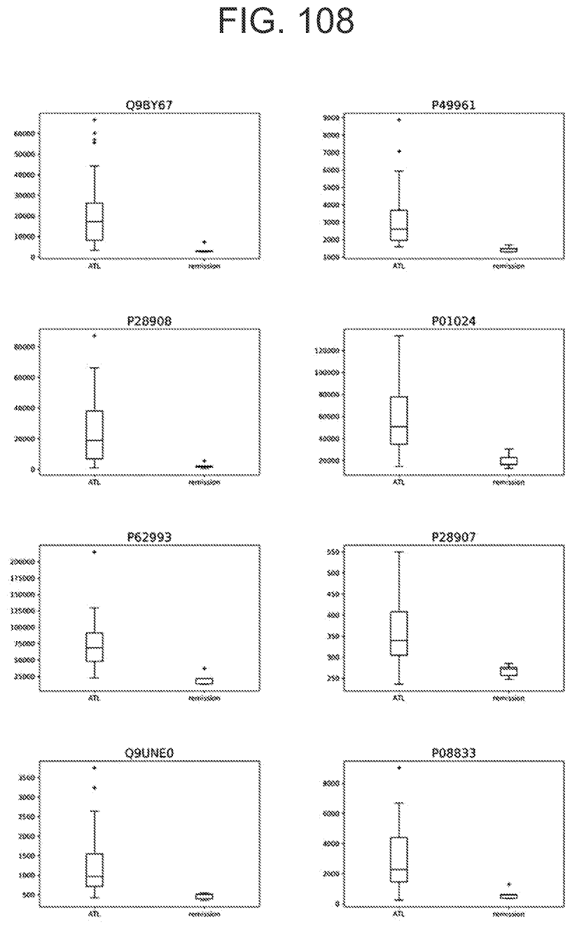

FIG. 109 The figure is a graph showing measurement results of the amount of a protein in plasmas of ATL patients and ATL patients in remission.

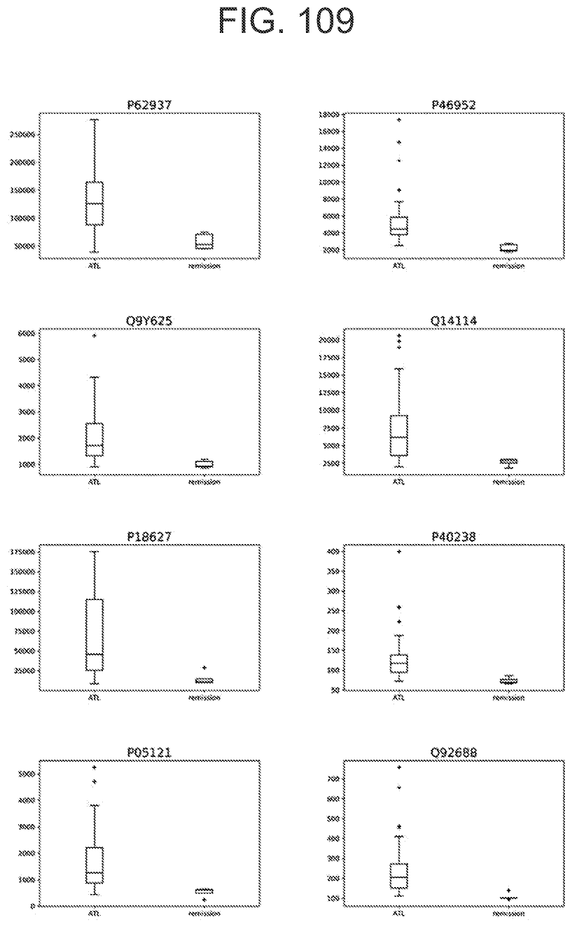

FIG. 110 The figure is a graph showing measurement results of the amount of a protein in plasmas of ATL patients and ATL patients in remission.

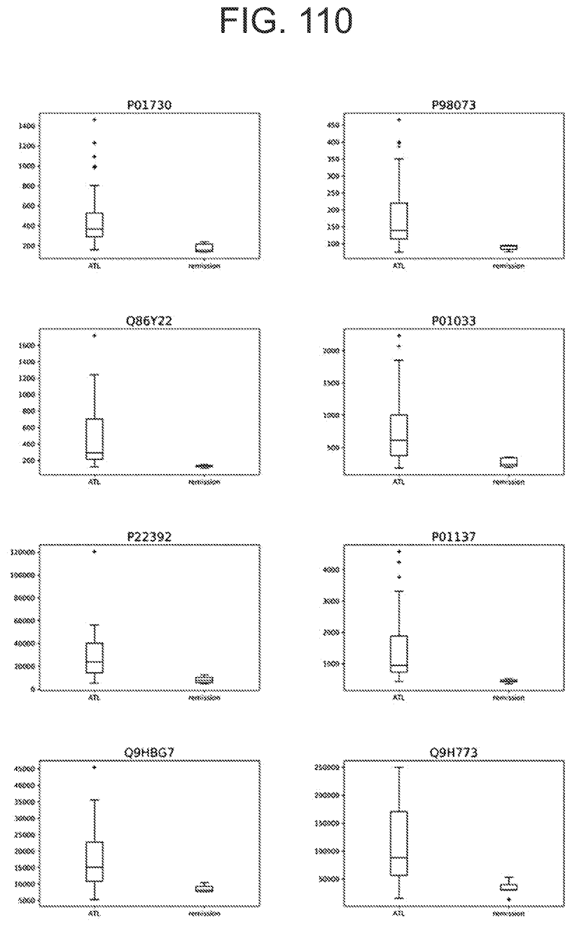

FIG. 111 The figure is a graph showing measurement results of the amount of a protein in plasmas of ATL patients and ATL patients in remission.

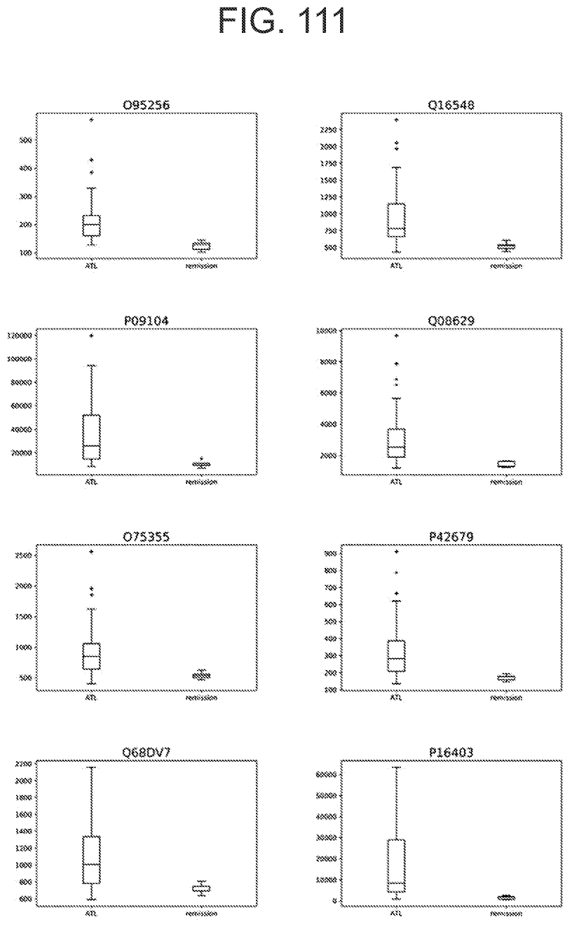

FIG. 112 The figure is a graph showing measurement results of the amount of a protein in plasmas of ATL patients and ATL patients in remission.

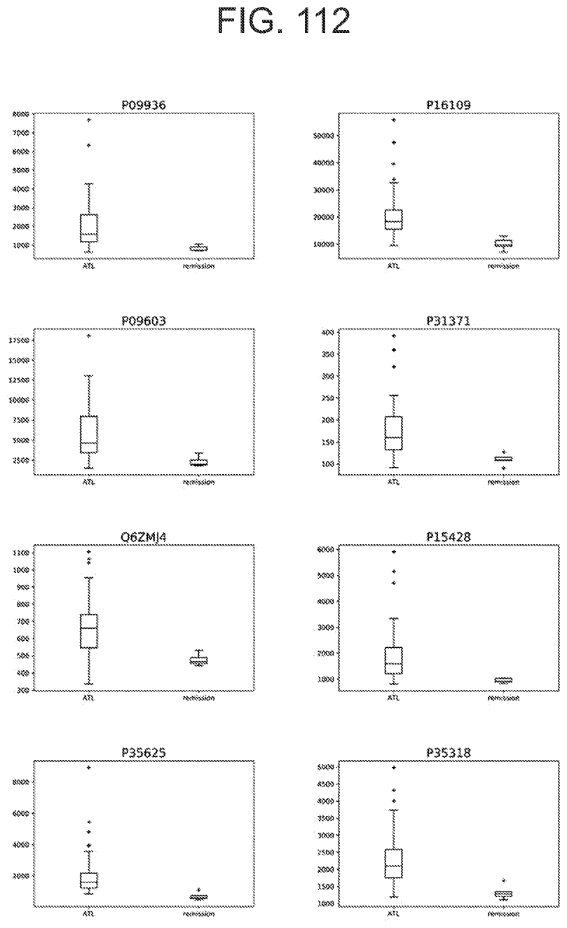

FIG. 113 The figure is a graph showing measurement results of the amount of a protein in plasmas of ATL patients and ATL patients in remission.

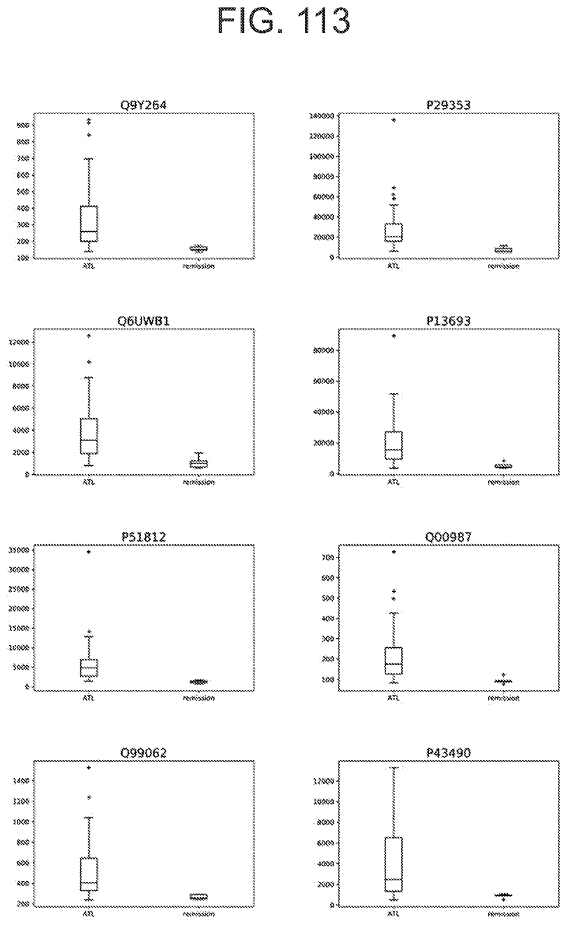

FIG. 114 The figure is a graph showing measurement results of the amount of a protein in plasmas of ATL patients and ATL patients in remission.

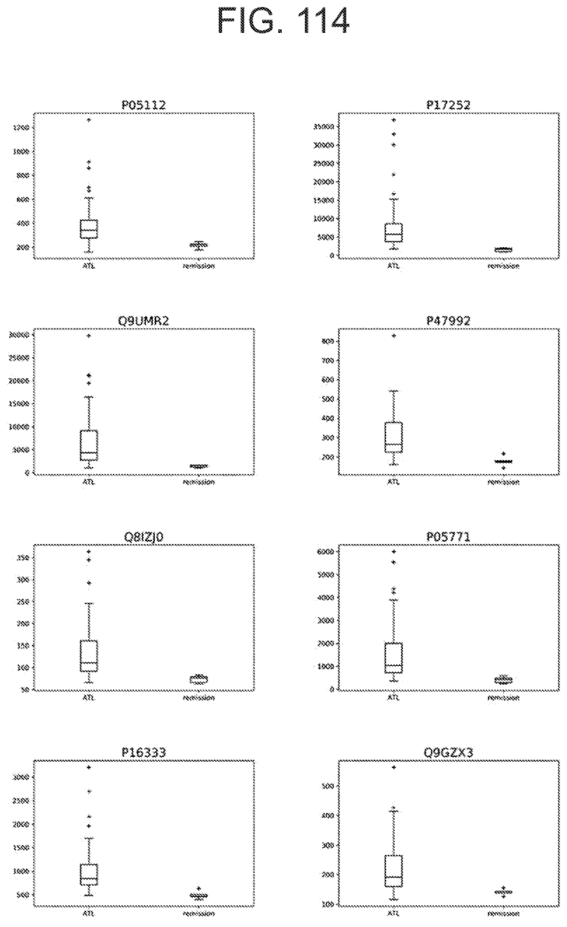

FIG. 115 The figure is a graph showing measurement results of the amount of a protein in plasmas of ATL patients and ATL patients in remission.

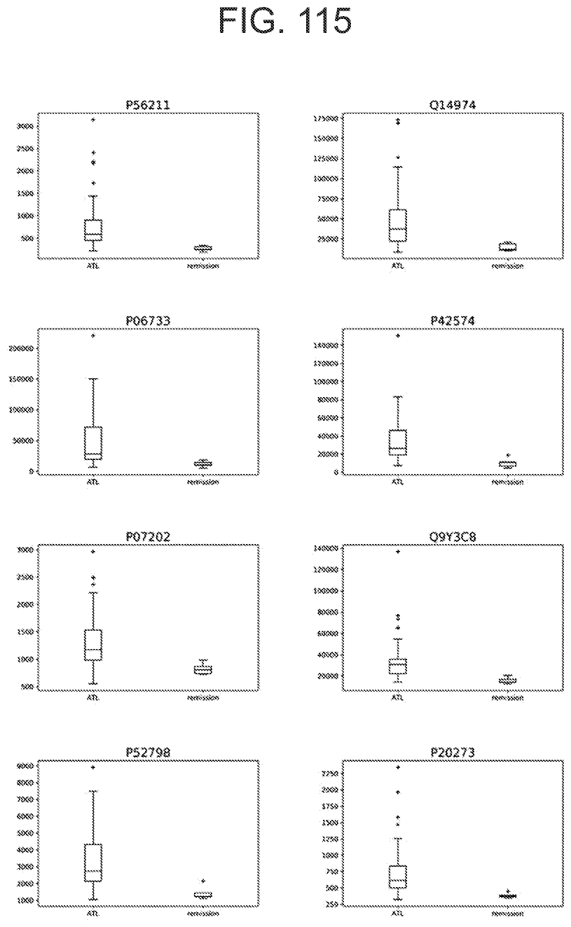

FIG. 116 The figure is a graph showing measurement results of the amount of a protein in plasmas of ATL patients and ATL patients in remission.

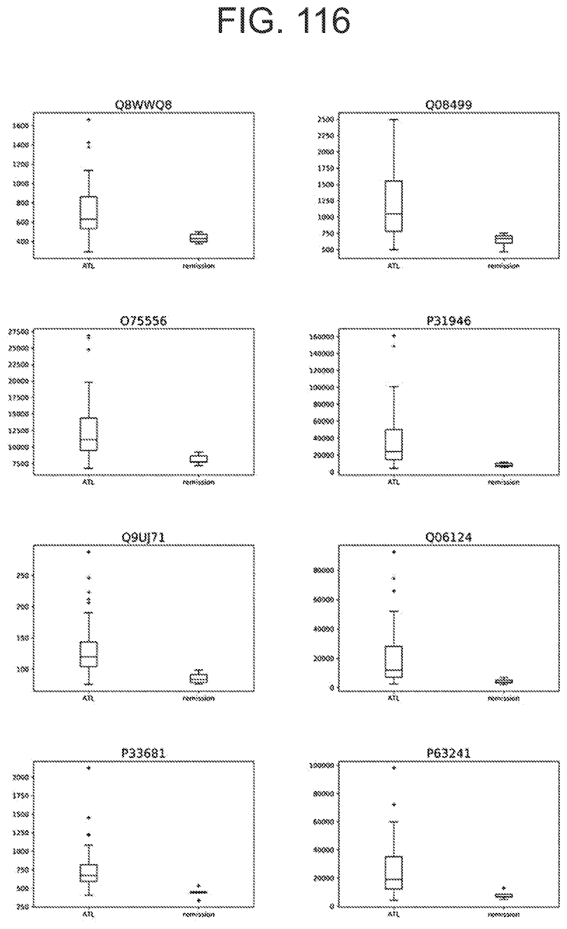

FIG. 117 The figure is a graph showing measurement results of the amount of a protein in plasmas of ATL patients and ATL patients in remission.

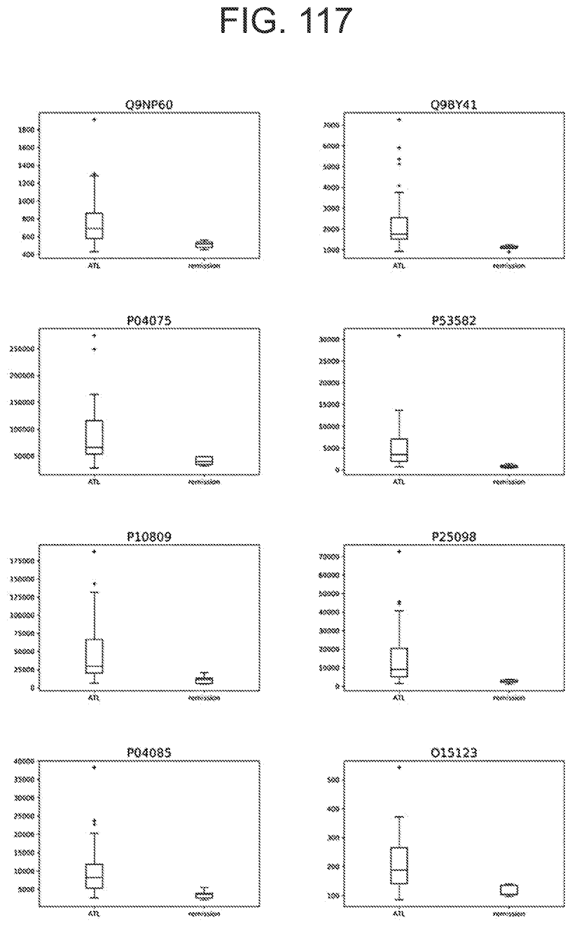

FIG. 118 The figure is a graph showing measurement results of the amount of a protein in plasmas of ATL patients and ATL patients in remission.

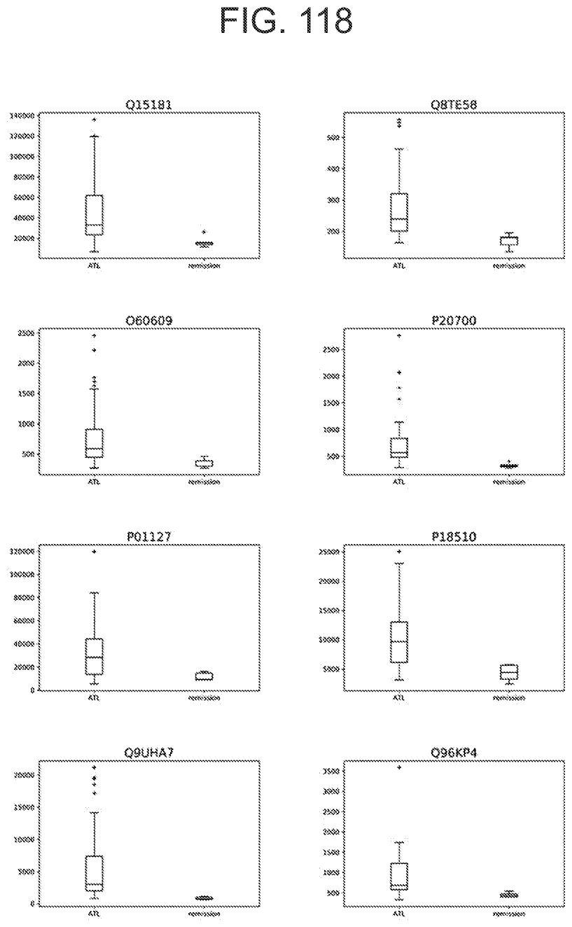

FIG. 119 The figure is a graph showing measurement results of the amount of a protein in plasmas of ATL patients and ATL patients in remission.

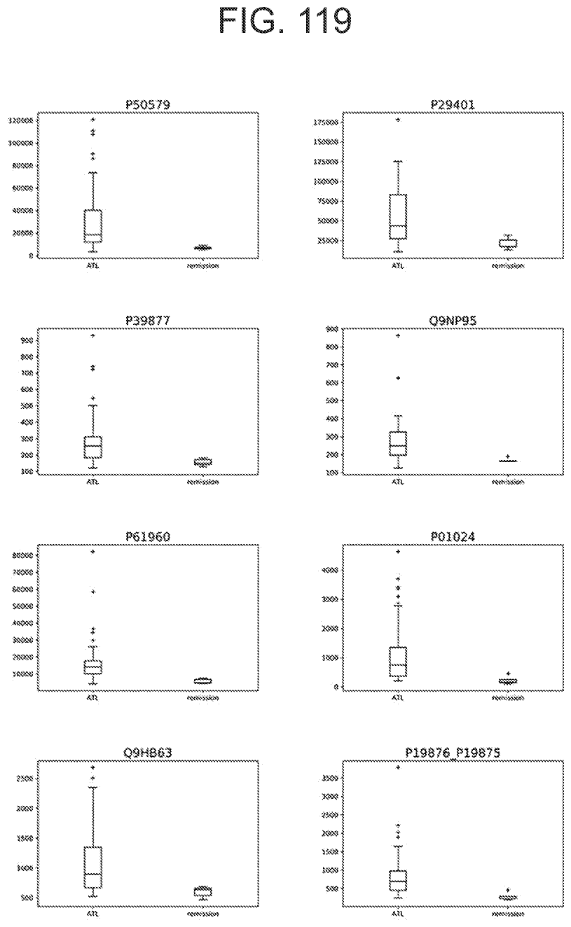

FIG. 120 The figure is a graph showing measurement results of the amount of a protein in plasmas of ATL patients and ATL patients in remission.

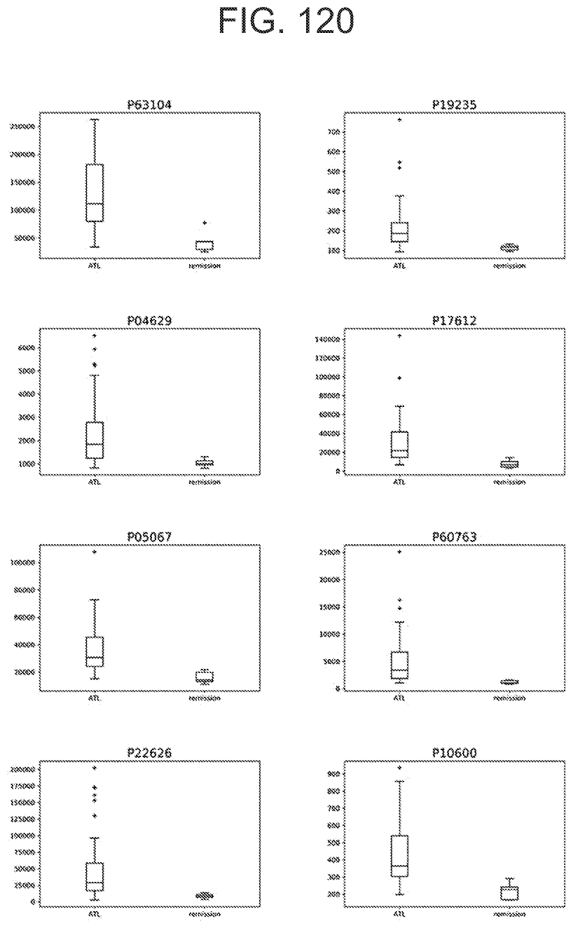

FIG. 121 The figure is a graph showing measurement results of the amount of a protein in plasmas of ATL patients and ATL patients in remission.

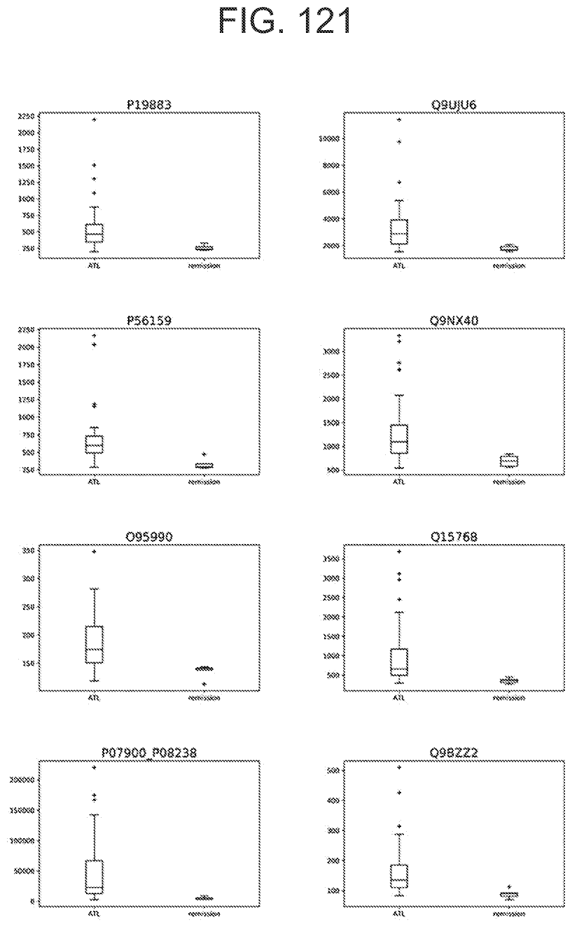

FIG. 122 The figure is a graph showing measurement results of the amount of a protein in plasmas of ATL patients and ATL patients in remission.

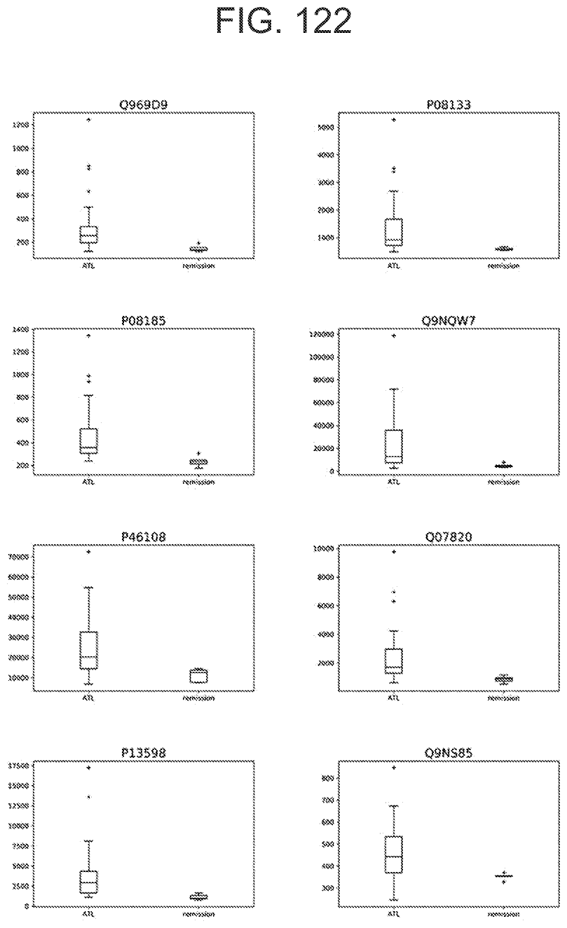

FIG. 123 The figure is a graph showing measurement results of the amount of a protein in plasmas of ATL patients and ATL patients in remission.

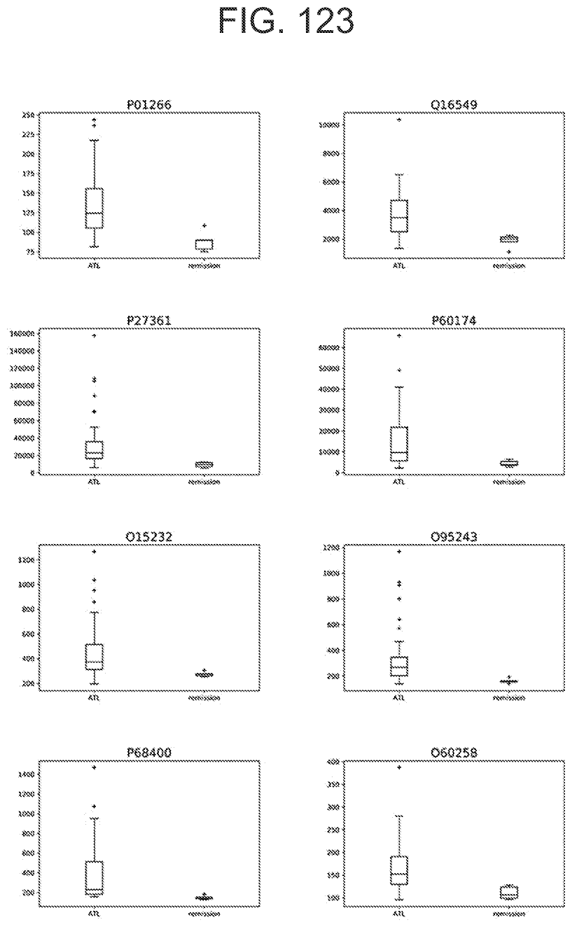

FIG. 124 The figure is a graph showing measurement results of the amount of a protein in plasmas of ATL patients and ATL patients in remission.

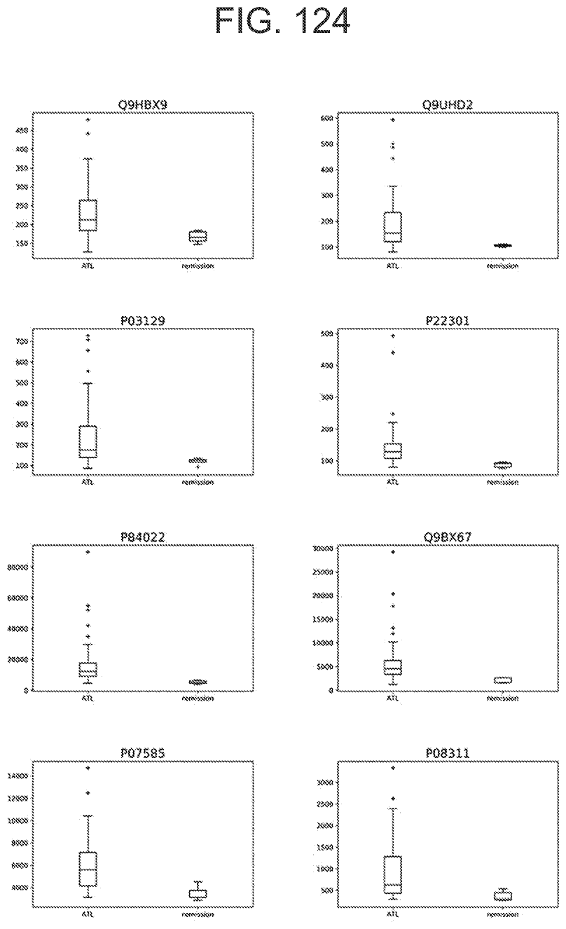

FIG. 125 The figure is a graph showing measurement results of the amount of a protein in plasmas of ATL patients and ATL patients in remission.

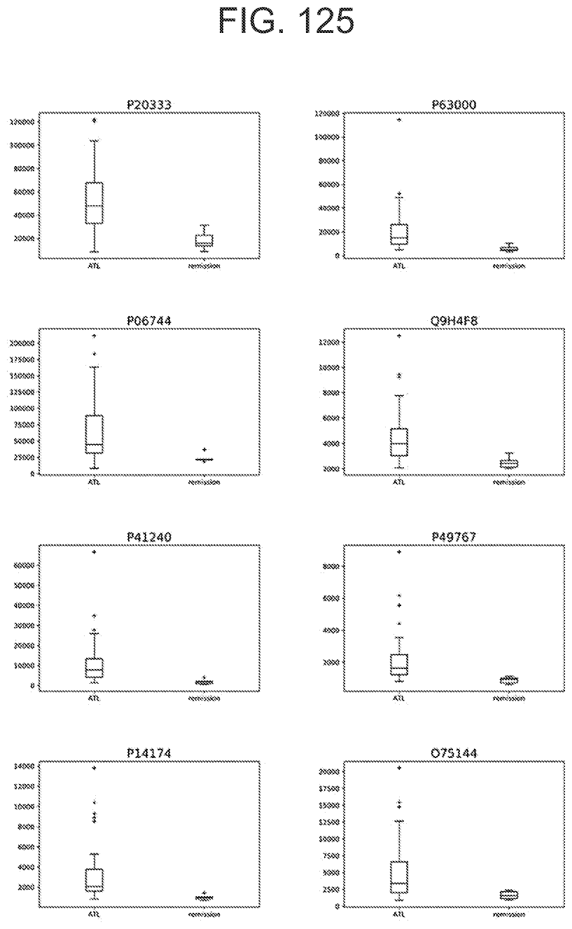

FIG. 126 The figure is a graph showing measurement results of the amount of a protein in plasmas of ATL patients and ATL patients in remission.

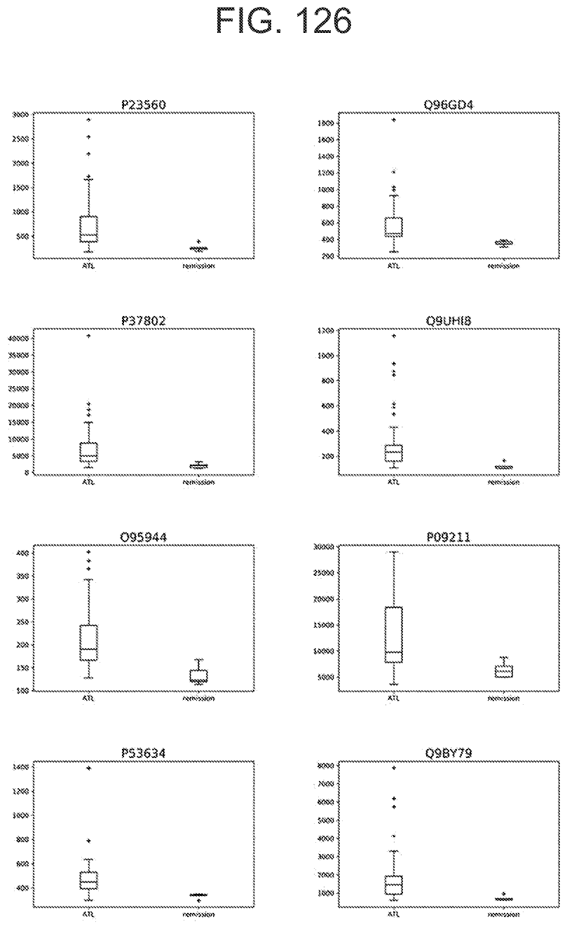

FIG. 127 The figure is a graph showing measurement results of the amount of a protein in plasmas of ATL patients and ATL patients in remission.

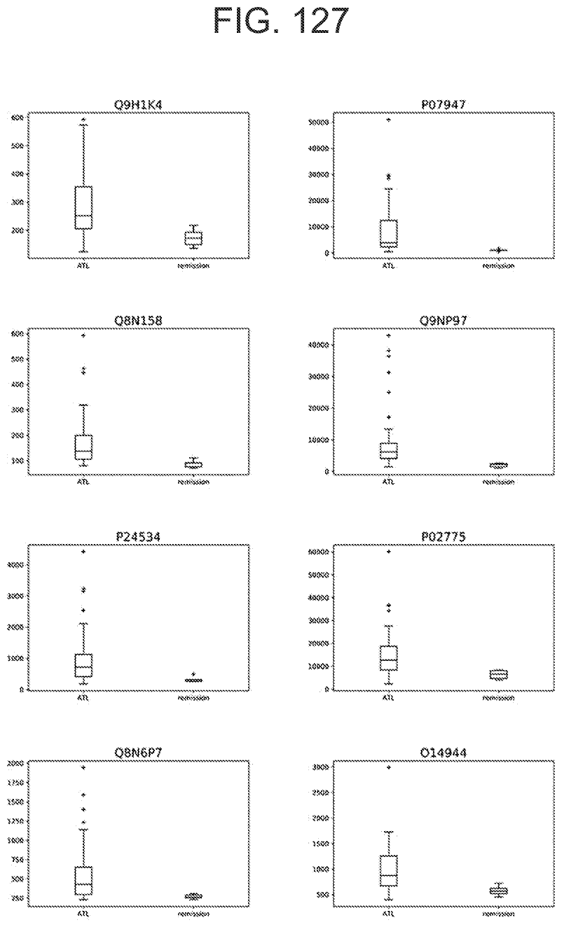

FIG. 128 The figure is a graph showing measurement results of the amount of a protein in plasmas of ATL patients and ATL patients in remission.

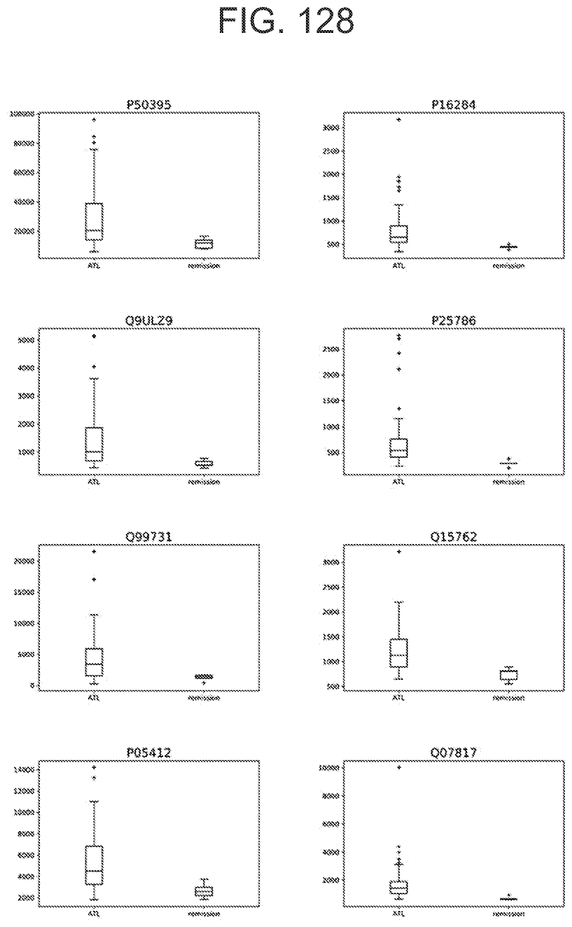

FIG. 129 The figure is a graph showing measurement results of the amount of a protein in plasmas of ATL patients and ATL patients in remission.

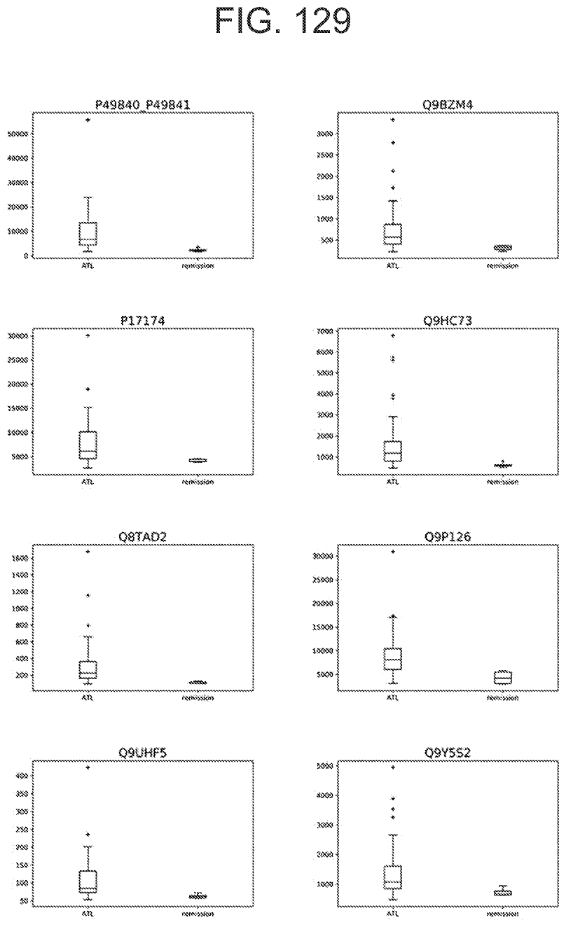

FIG. 130 The figure is a graph showing measurement results of the amount of a protein in plasmas of ATL patients and ATL patients in remission.

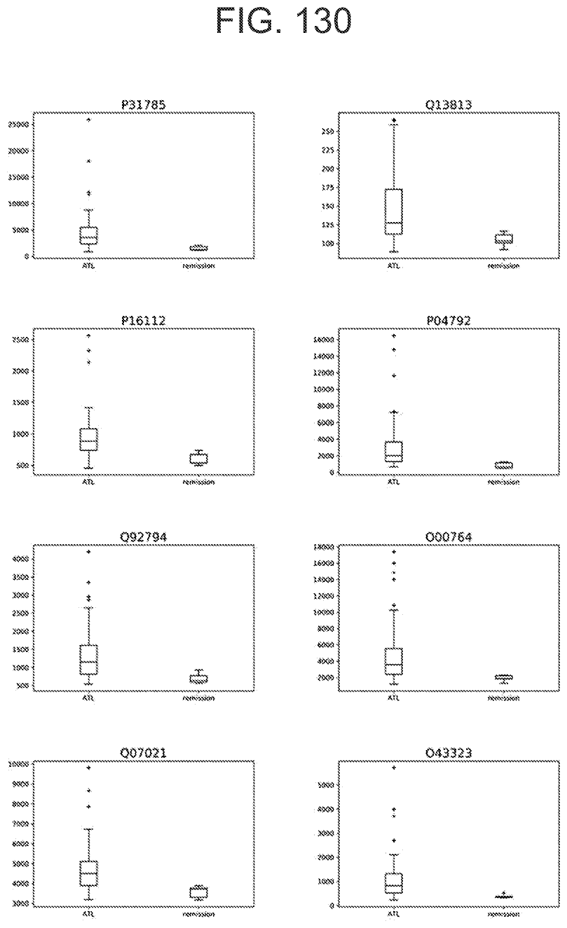

FIG. 131 The figure is a graph showing measurement results of the amount of a protein in plasmas of ATL patients and ATL patients in remission.

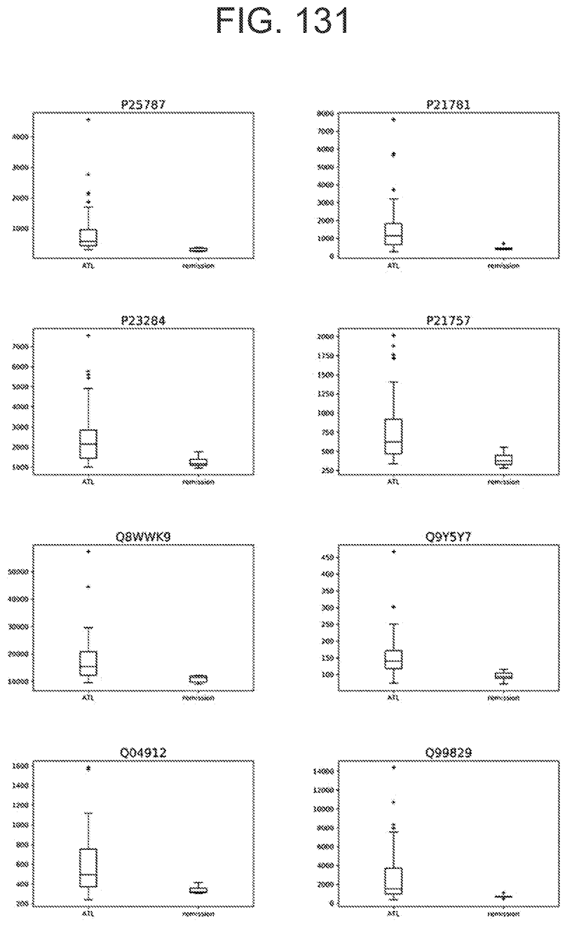

FIG. 132 The figure is a graph showing measurement results of the amount of a protein in plasmas of ATL patients and ATL patients in remission.

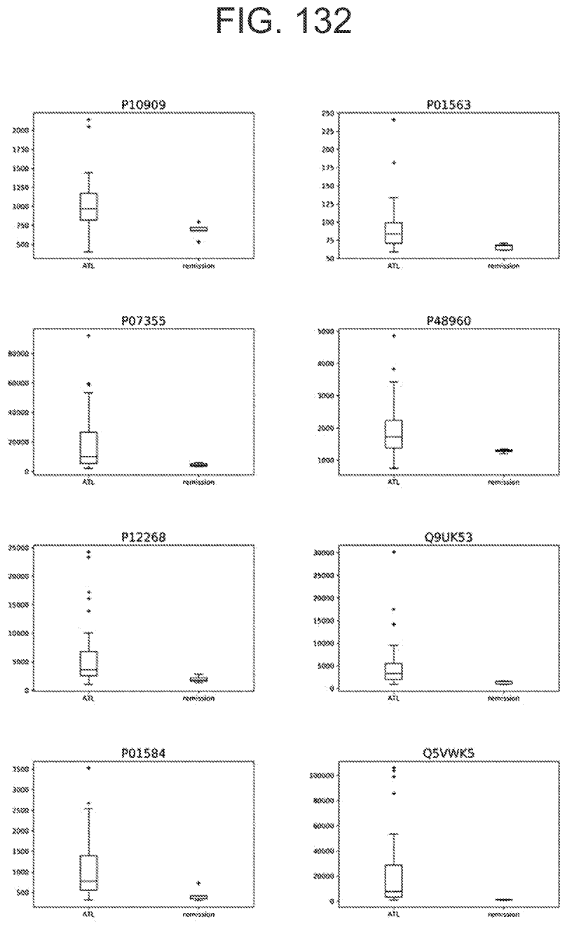

FIG. 133 The figure is a graph showing measurement results of the amount of a protein in plasmas of ATL patients and ATL patients in remission.

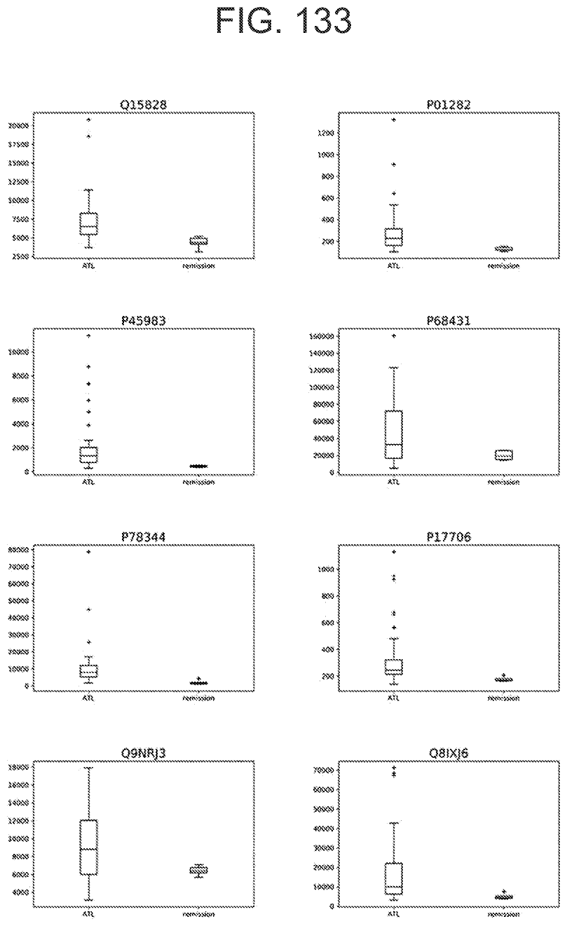

FIG. 134 The figure is a graph showing measurement results of the amount of a protein in plasmas of ATL patients and ATL patients in remission.

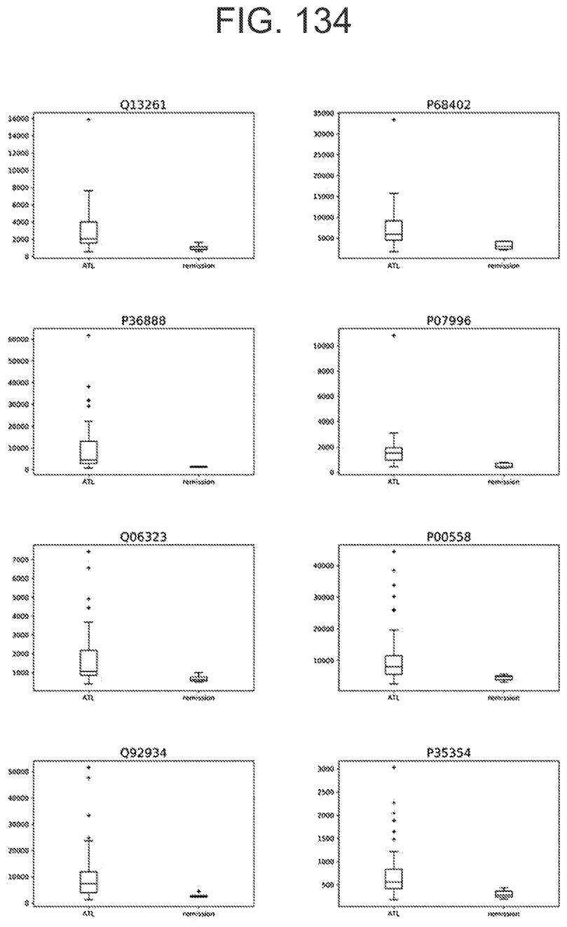

FIG. 135 The figure is a graph showing measurement results of the amount of a protein in plasmas of ATL patients and ATL patients in remission.

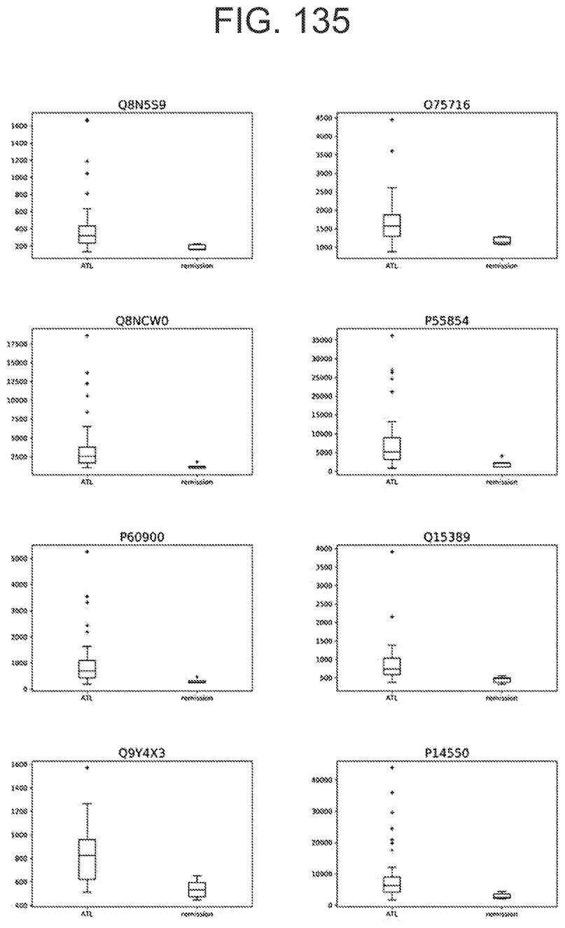

FIG. 136 The figure is a graph showing measurement results of the amount of a protein in plasmas of ATL patients and ATL patients in remission.

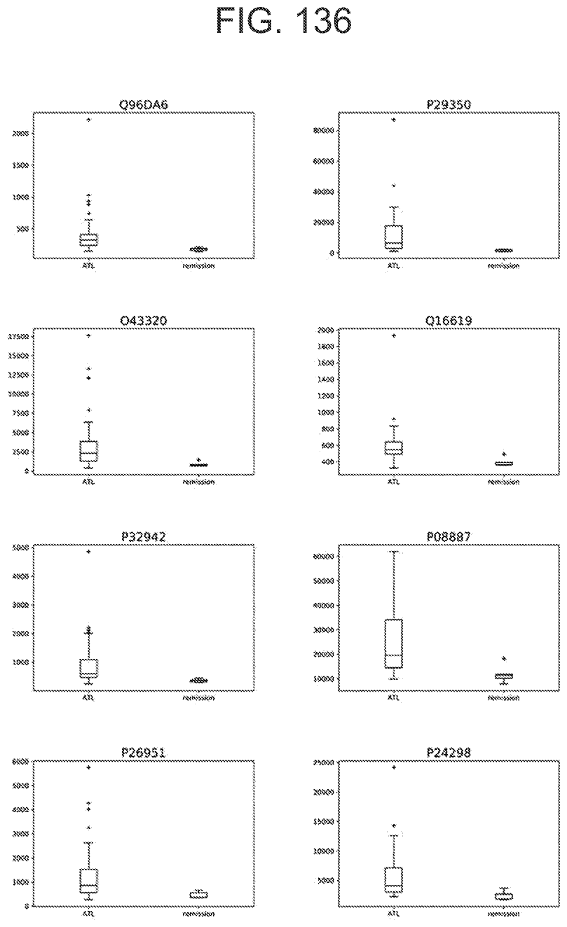

FIG. 137 The figure is a graph showing measurement results of the amount of a protein in plasmas of ATL patients and ATL patients in remission.

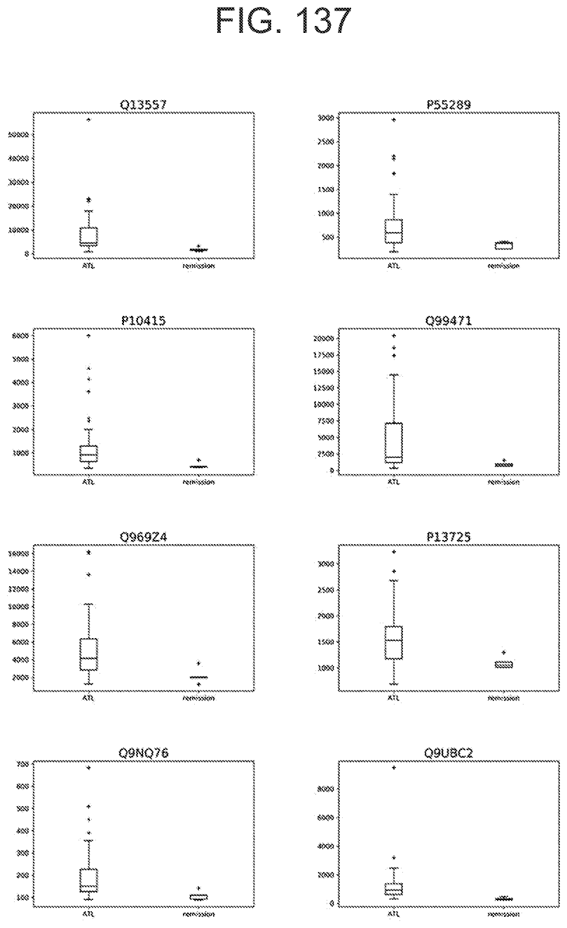

FIG. 138 The figure is a graph showing measurement results of the amount of a protein in plasmas of ATL patients and ATL patients in remission.

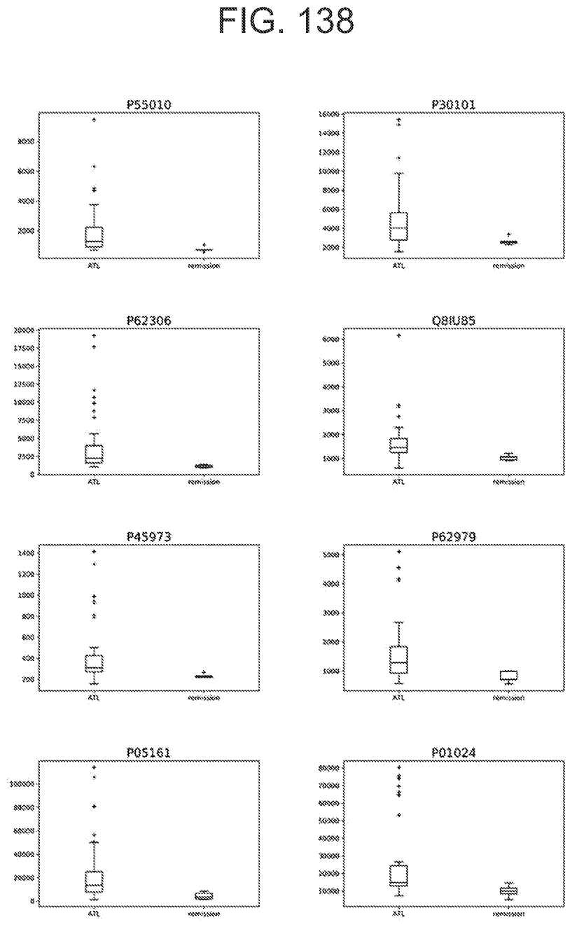

FIG. 139 The figure is a graph showing measurement results of the amount of a protein in plasmas of ATL patients and ATL patients in remission.

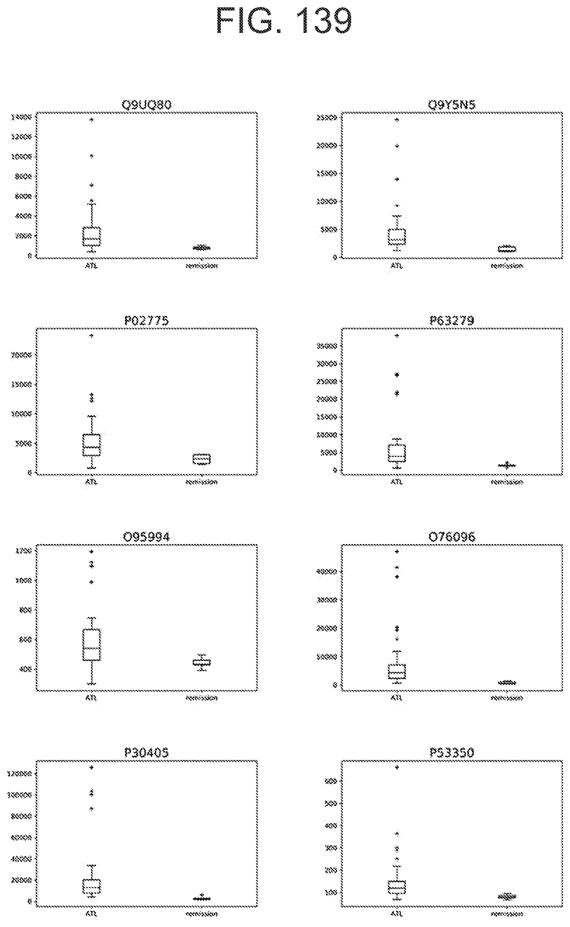

FIG. 140 The figure is a graph showing measurement results of the amount of a protein in plasmas of ATL patients and ATL patients in remission.

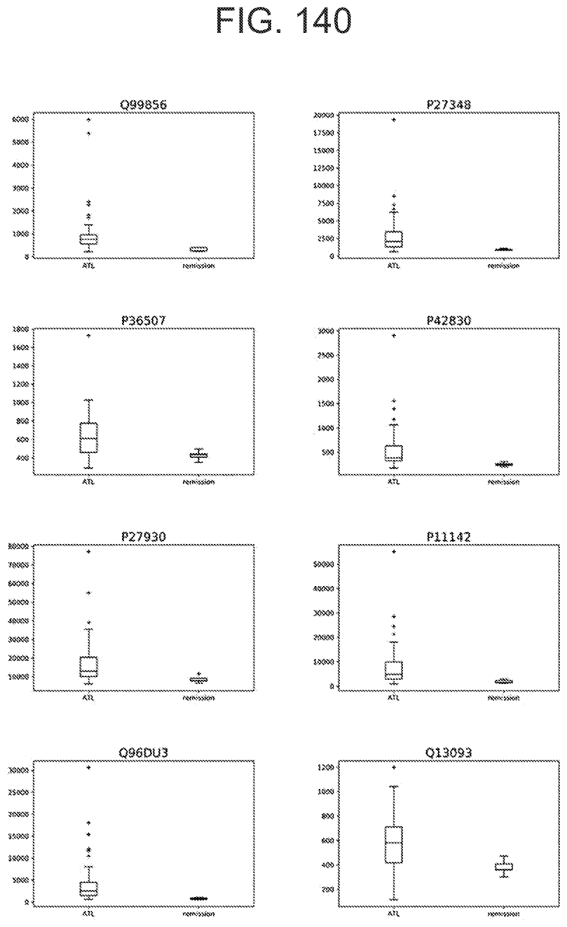

FIG. 141 The figure is a graph showing measurement results of the amount of a protein in plasmas of ATL patients and ATL patients in remission.

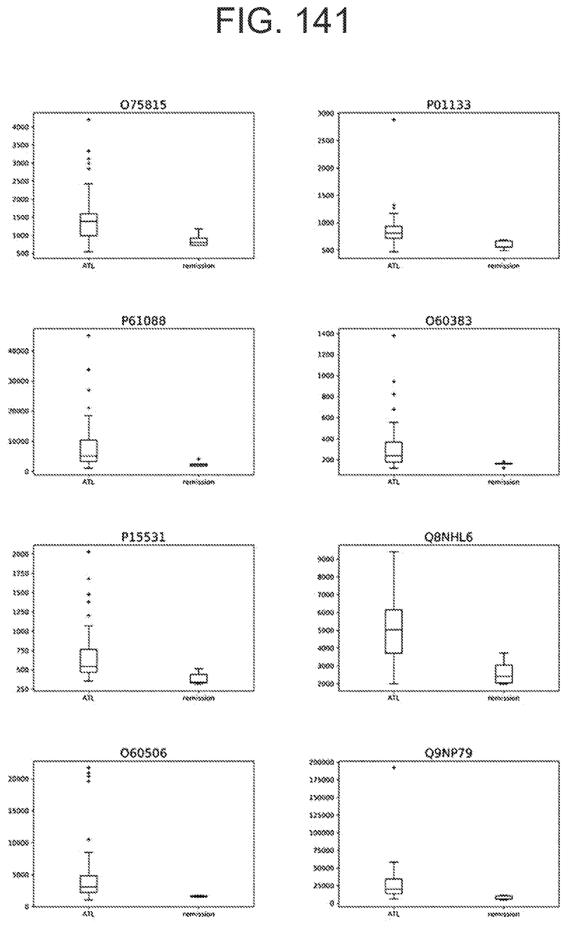

FIG. 142 The figure is a graph showing measurement results of the amount of a protein in plasmas of ATL patients and ATL patients in remission.

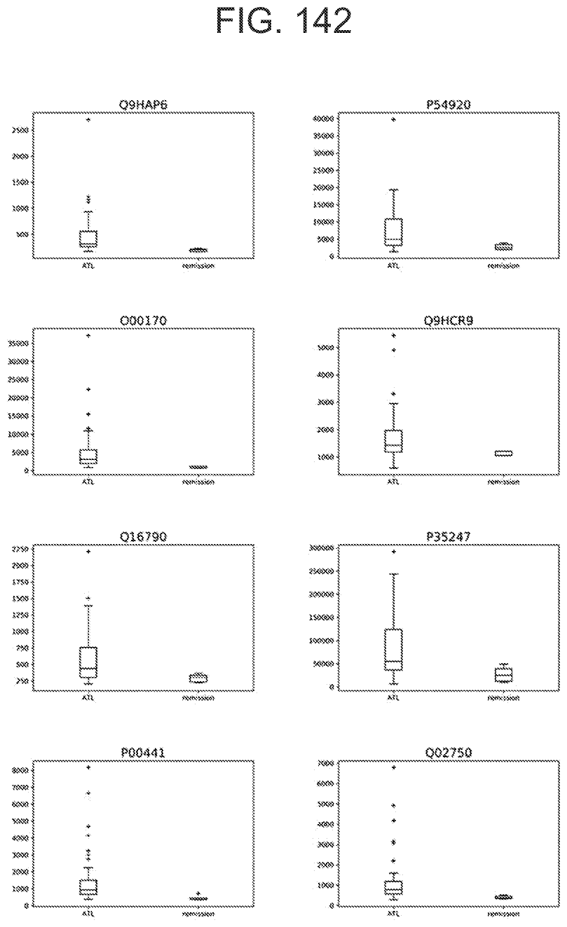

FIG. 143 The figure is a graph showing measurement results of the amount of a protein in plasmas of ATL patients and ATL patients in remission.

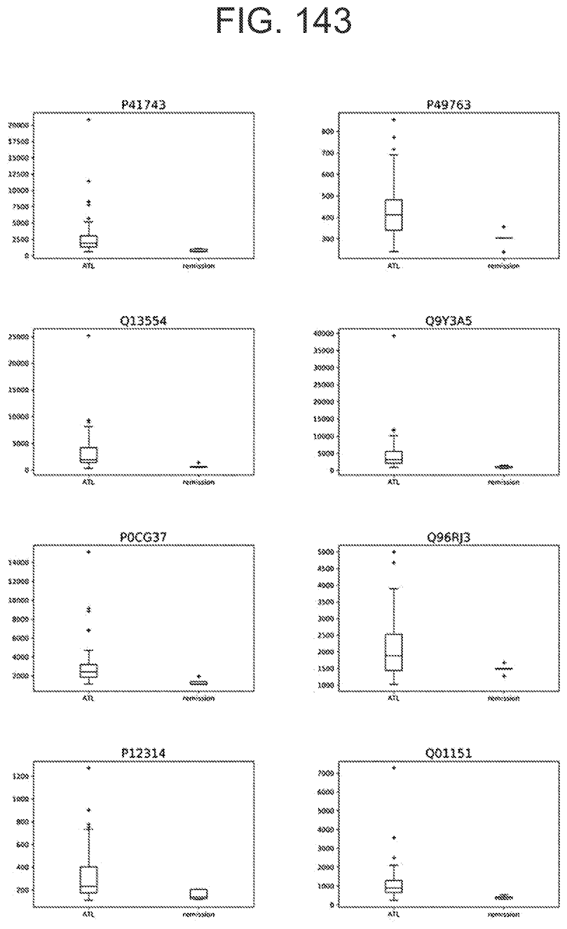

FIG. 144 The figure is a graph showing measurement results of the amount of a protein in plasmas of ATL patients and ATL patients in remission.

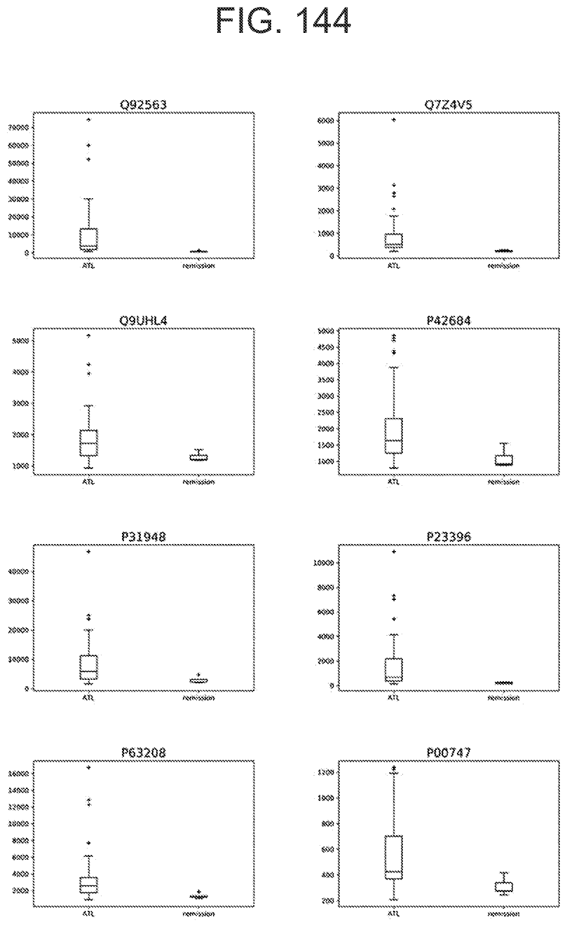

FIG. 145 The figure is a graph showing measurement results of the amount of a protein in plasmas of ATL patients and ATL patients in remission.

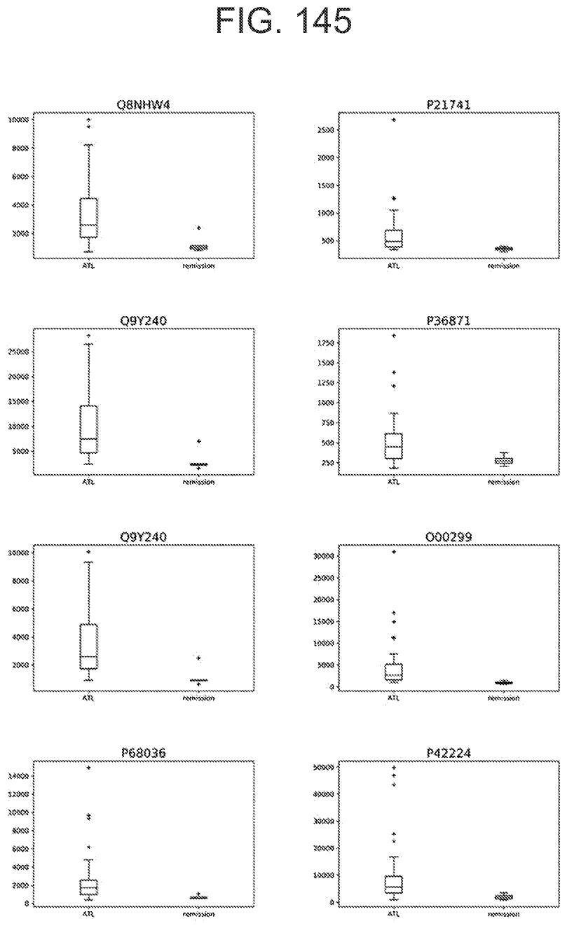

FIG. 146 The figure is a graph showing measurement results of the amount of a protein in plasmas of ATL patients and ATL patients in remission.

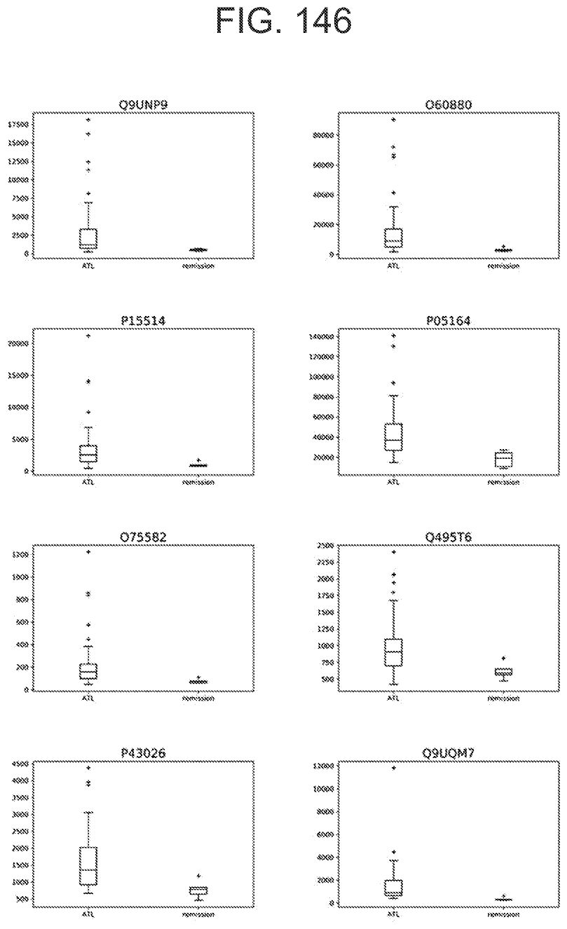

FIG. 147 The figure is a graph showing measurement results of the amount of a protein in plasmas of ATL patients and ATL patients in remission.

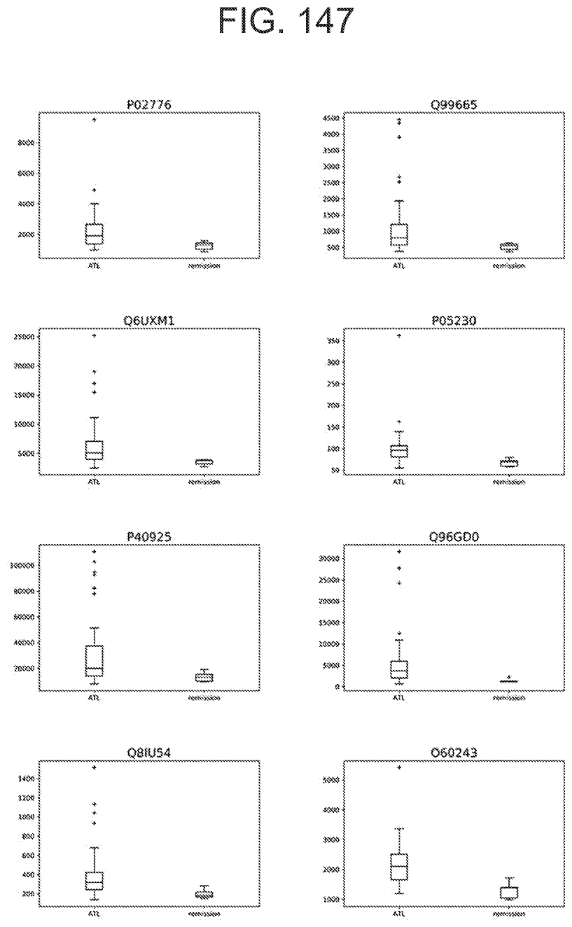

FIG. 148 The figure is a graph showing measurement results of the amount of a protein in plasmas of ATL patients and ATL patients in remission.

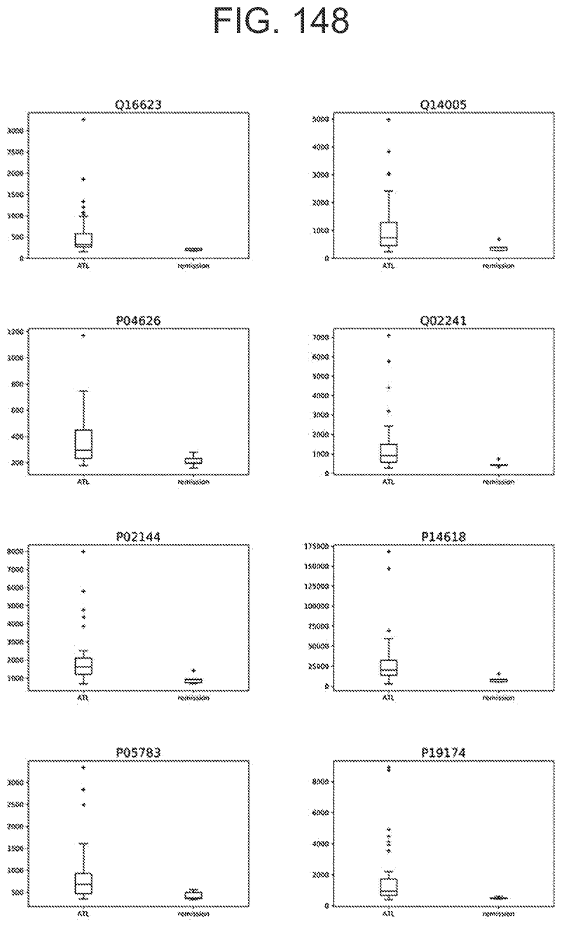

FIG. 149 The figure is a graph showing measurement results of the amount of a protein in plasmas of ATL patients and ATL patients in remission.

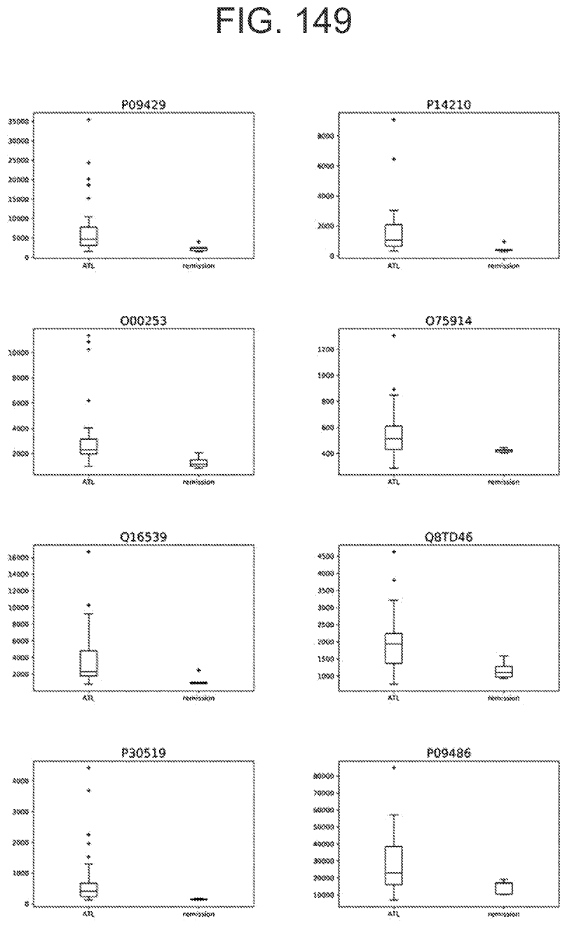

FIG. 150 The figure is a graph showing measurement results of the amount of a protein in plasmas of ATL patients and ATL patients in remission.

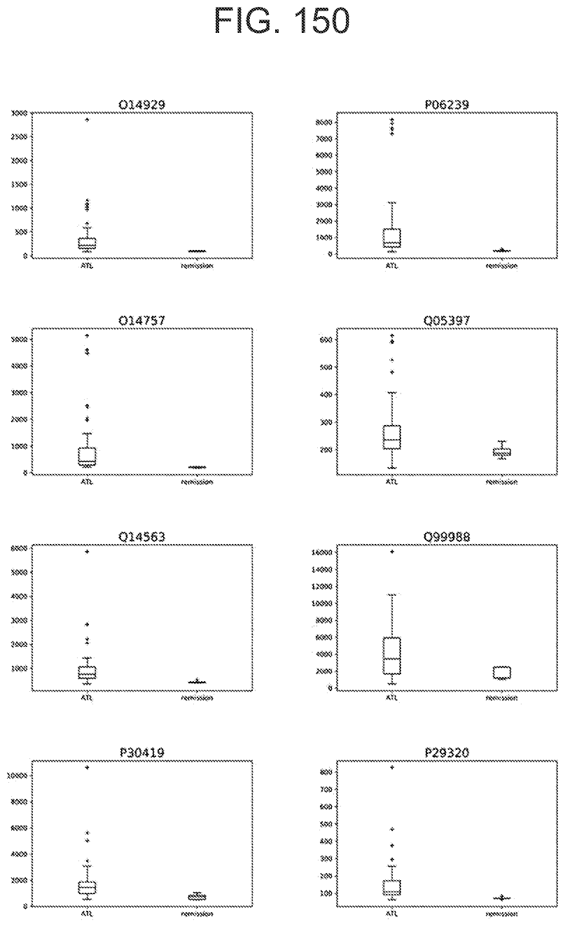

FIG. 151 The figure is a graph showing measurement results of the amount of a protein in plasmas of ATL patients and ATL patients in remission.

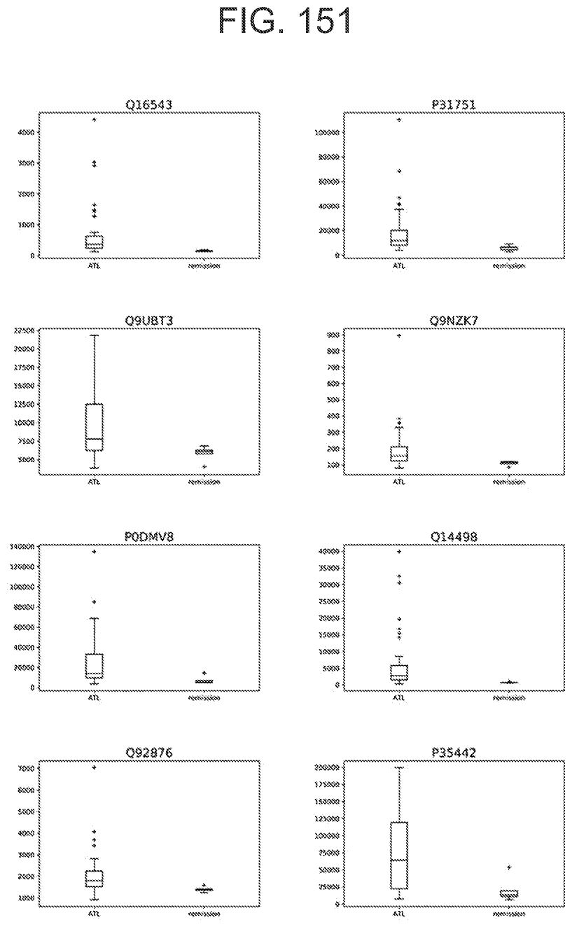

FIG. 152 The figure is a graph showing measurement results of the amount of a protein in plasmas of ATL patients and ATL patients in remission.

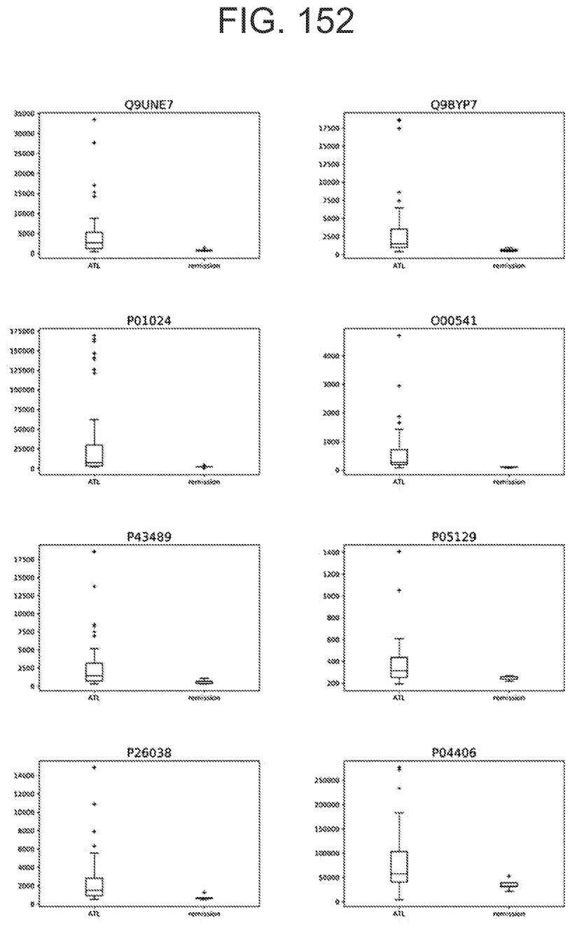

FIG. 153 The figure is a graph showing measurement results of the amount of a protein in plasmas of ATL patients and ATL patients in remission.

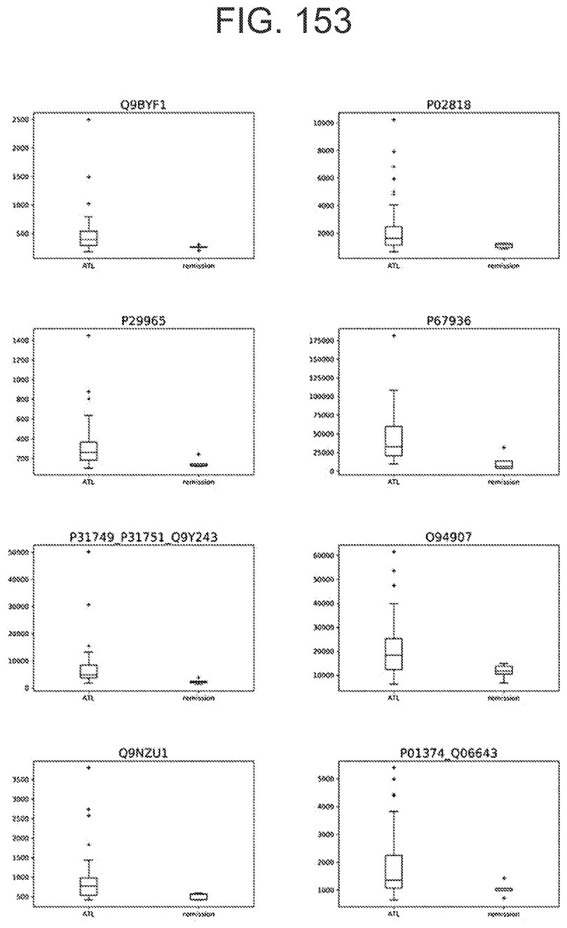

FIG. 154 The figure is a graph showing measurement results of the amount of a protein in plasmas of ATL patients and ATL patients in remission.

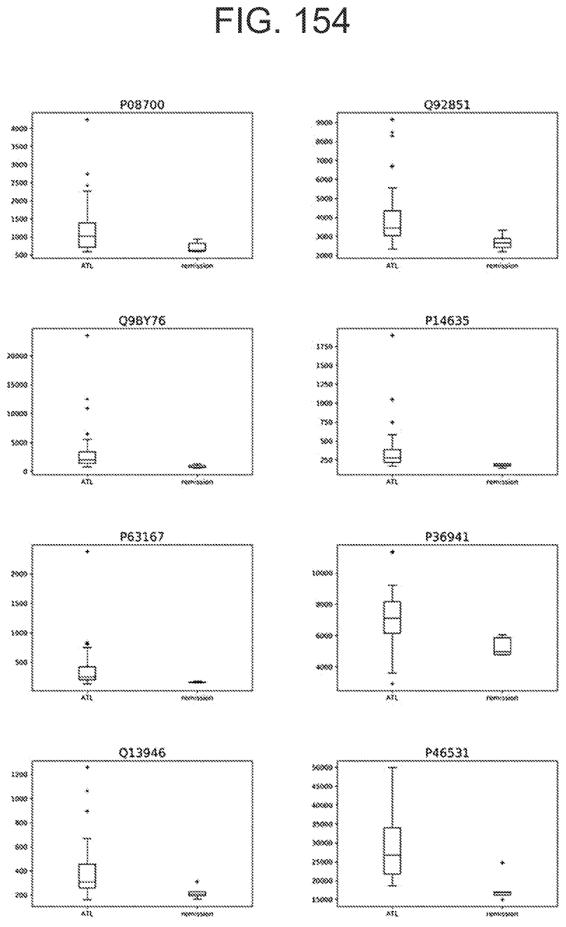

FIG. 155 The figure is a graph showing measurement results of the amount of a protein in plasmas of ATL patients and ATL patients in remission.

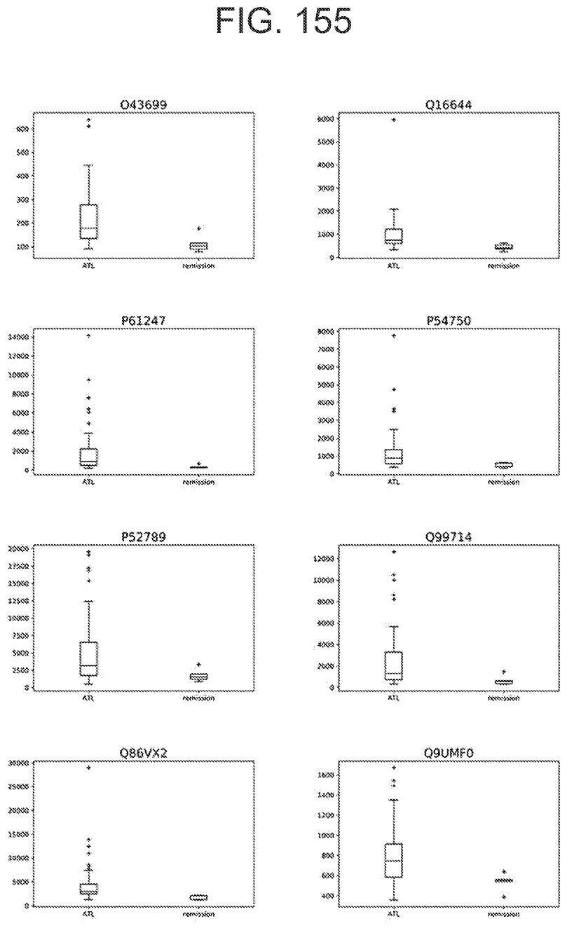

FIG. 156 The figure is a graph showing measurement results of the amount of a protein in plasmas of ATL patients and ATL patients in remission.

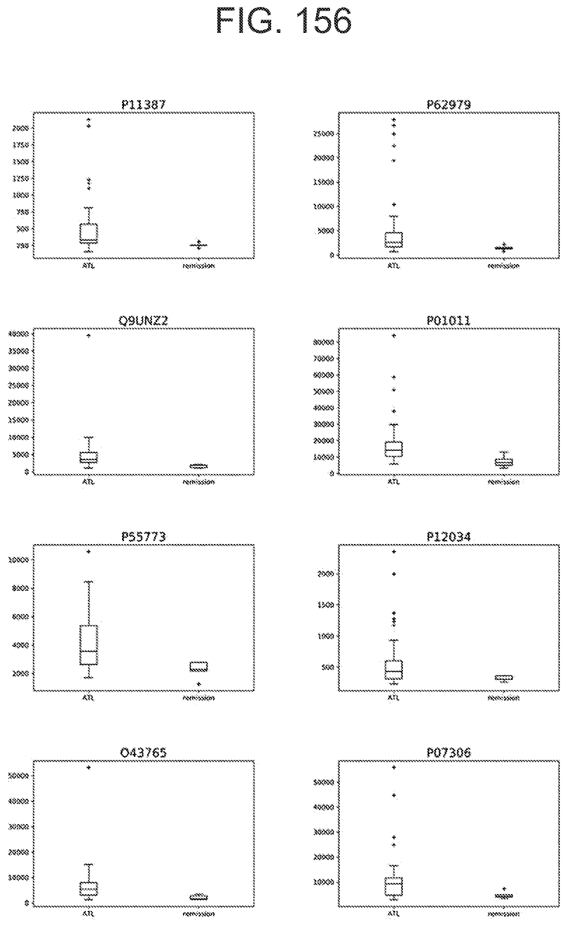

FIG. 157 The figure is a graph showing measurement results of the amount of a protein in plasmas of ATL patients and ATL patients in remission.

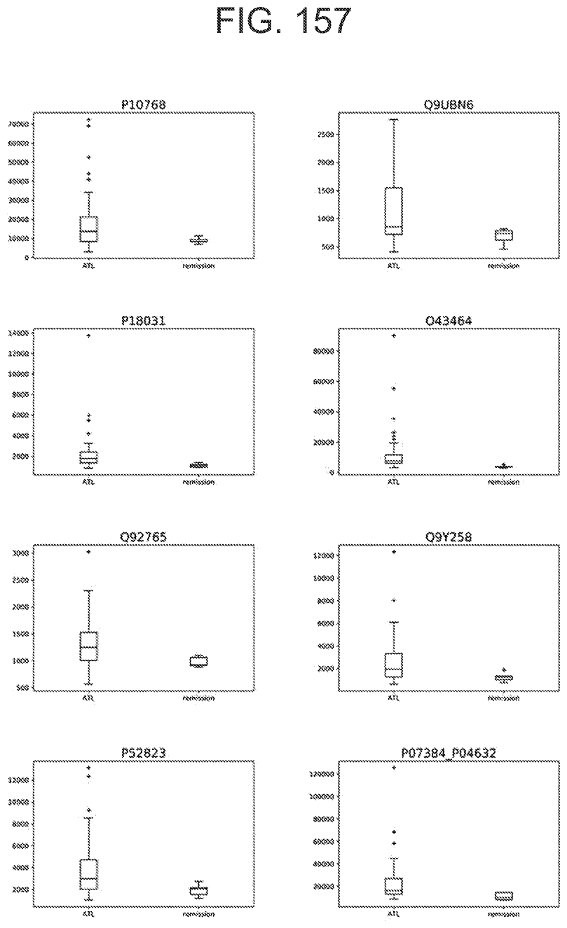

FIG. 158 The figure is a graph showing measurement results of the amount of a protein in plasmas of ATL patients and ATL patients in remission.

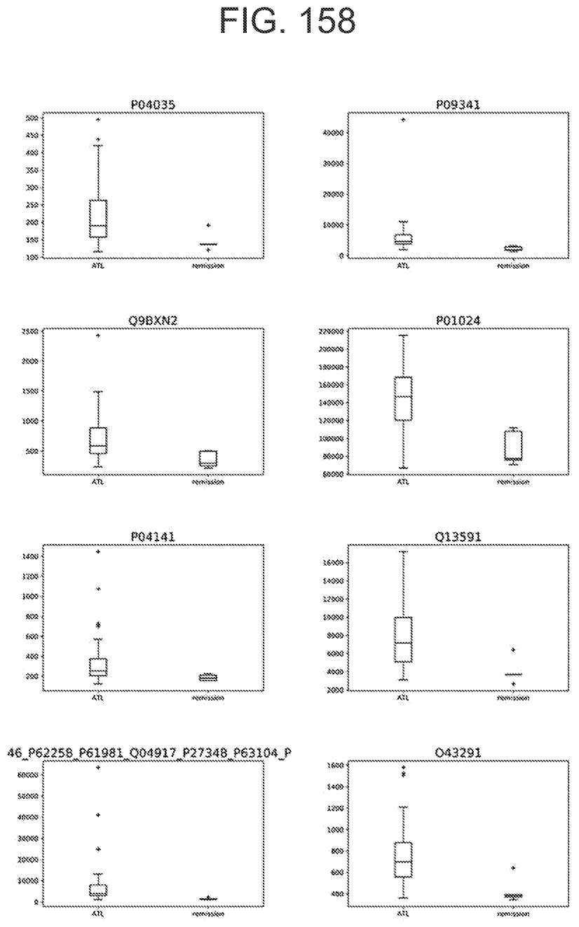

FIG. 159 The figure is a graph showing measurement results of the amount of a protein in plasmas of ATL patients and ATL patients in remission.

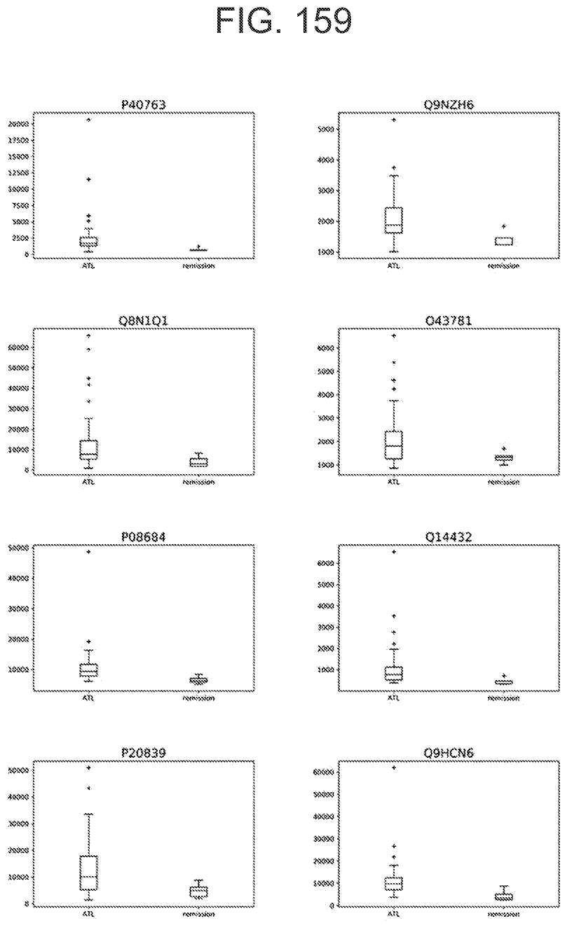

FIG. 160 The figure is a graph showing measurement results of the amount of a protein in plasmas of ATL patients and ATL patients in remission.

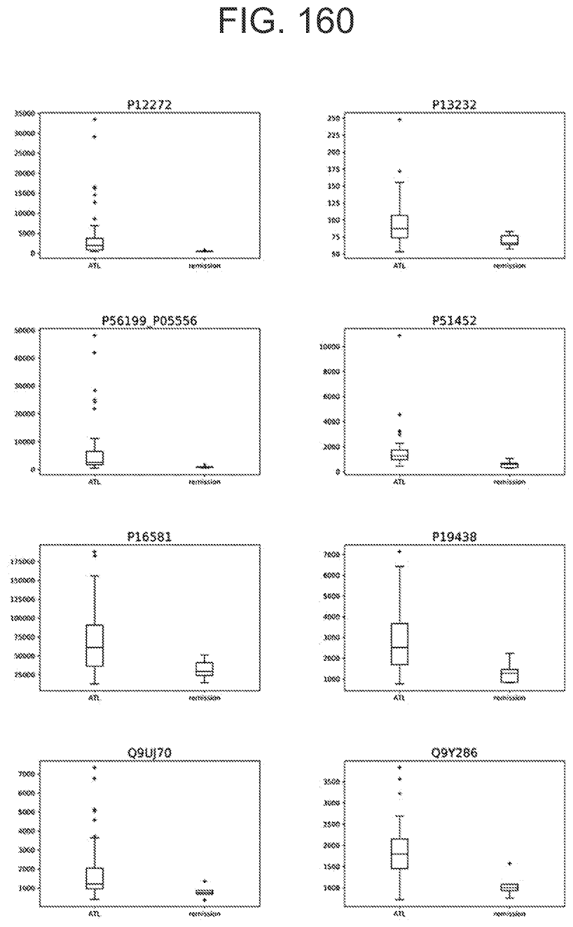

FIG. 161 The figure is a graph showing measurement results of the amount of a protein in plasmas of ATL patients and ATL patients in remission.

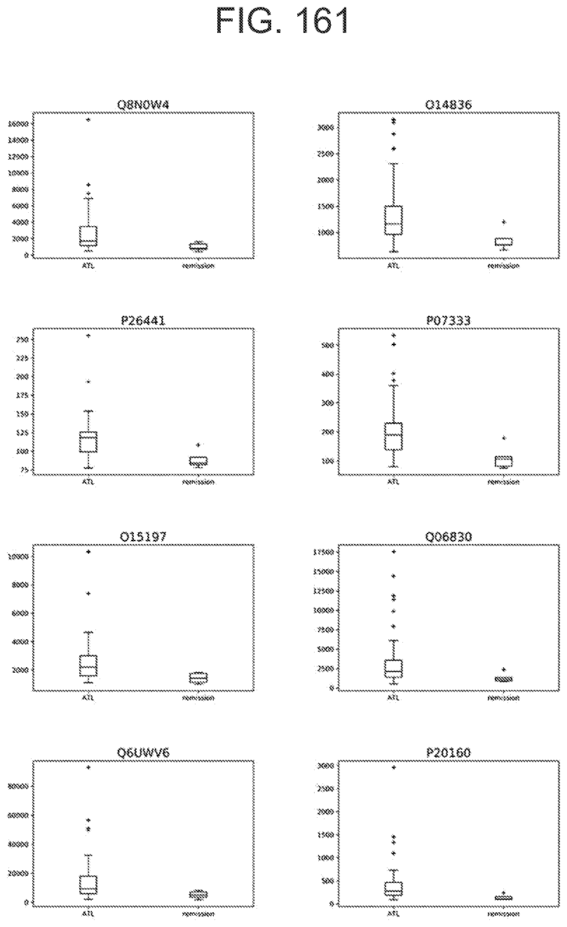

FIG. 162 The figure is a graph showing measurement results of the amount of a protein in plasmas of ATL patients and ATL patients in remission.

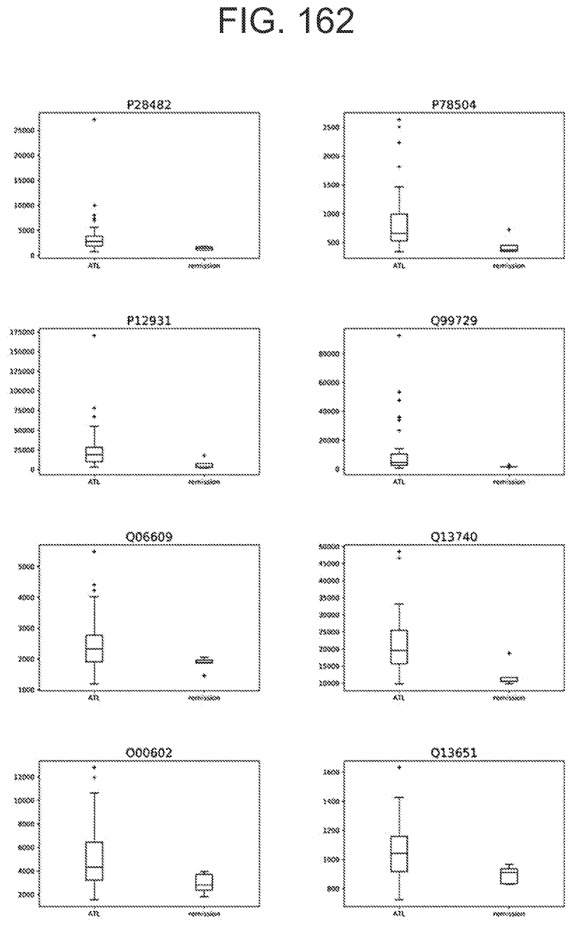

FIG. 163 The figure is a graph showing measurement results of the amount of a protein in plasmas of ATL patients and ATL patients in remission.

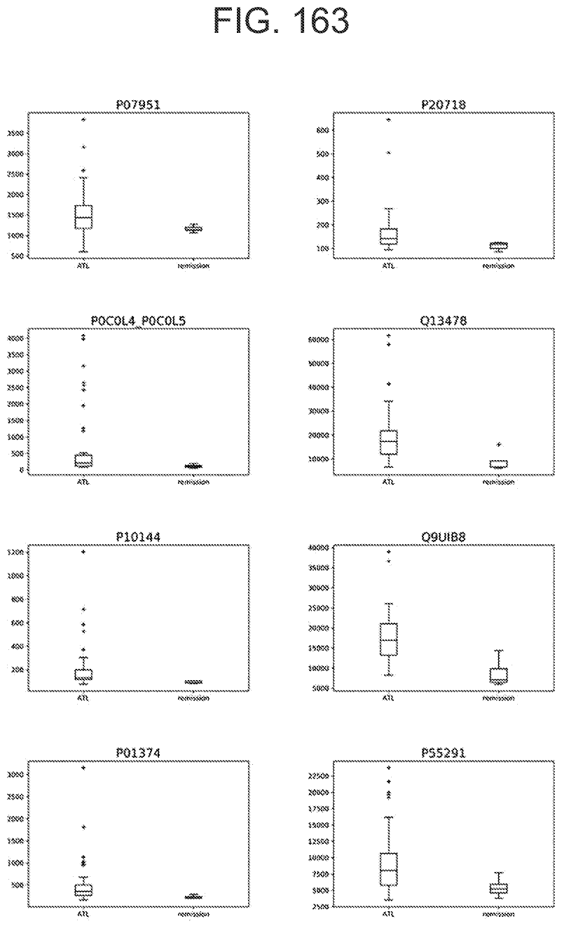

FIG. 164 The figure is a graph showing measurement results of the amount of a protein in plasmas of ATL patients and ATL patients in remission.

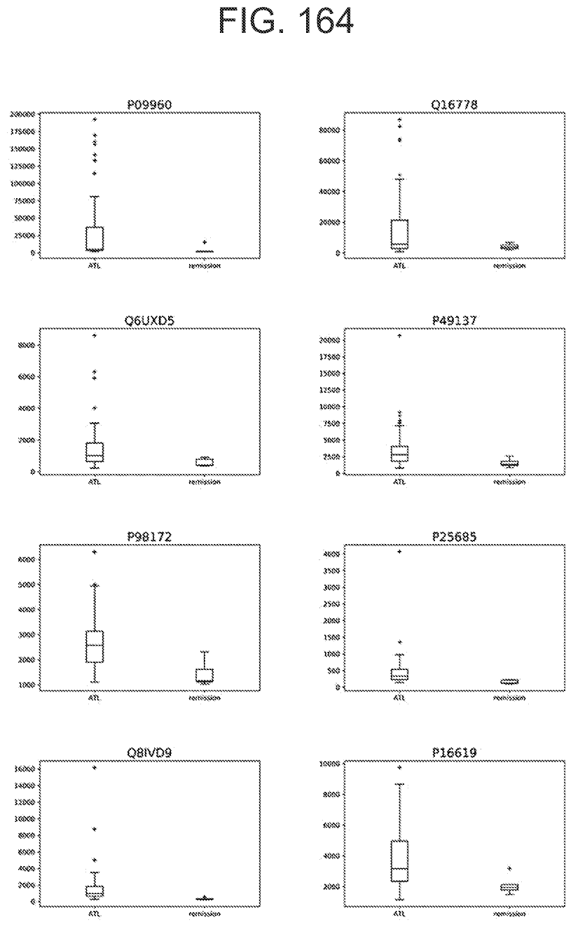

FIG. 165 The figure is a graph showing measurement results of the amount of a protein in plasmas of ATL patients and ATL patients in remission.

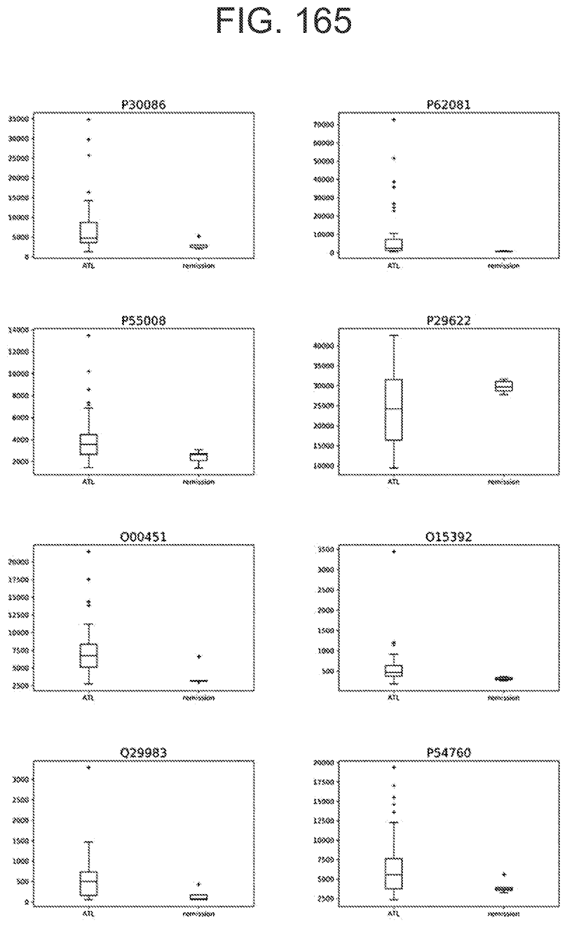

FIG. 166 The figure is a graph showing measurement results of the amount of a protein in plasmas of ATL patients and ATL patients in remission.

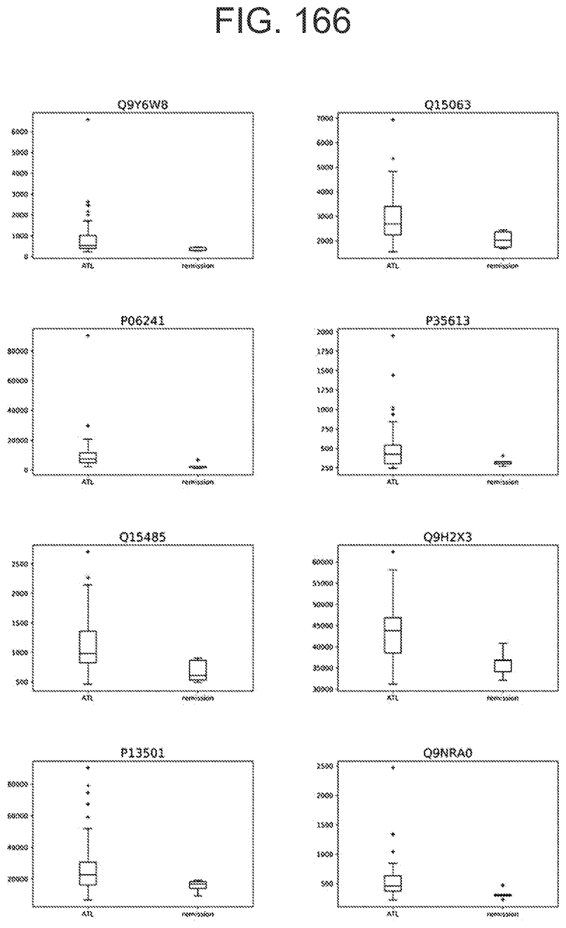

FIG. 167 The figure is a graph showing measurement results of the amount of a protein in plasmas of ATL patients and ATL patients in remission.

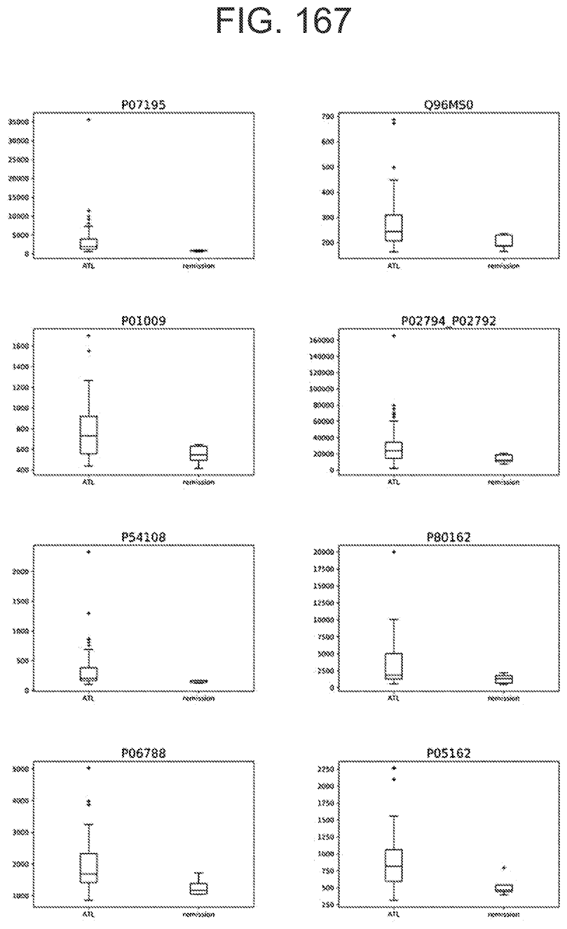

FIG. 168 The figure is a graph showing measurement results of the amount of a protein in plasmas of ATL patients and ATL patients in remission.

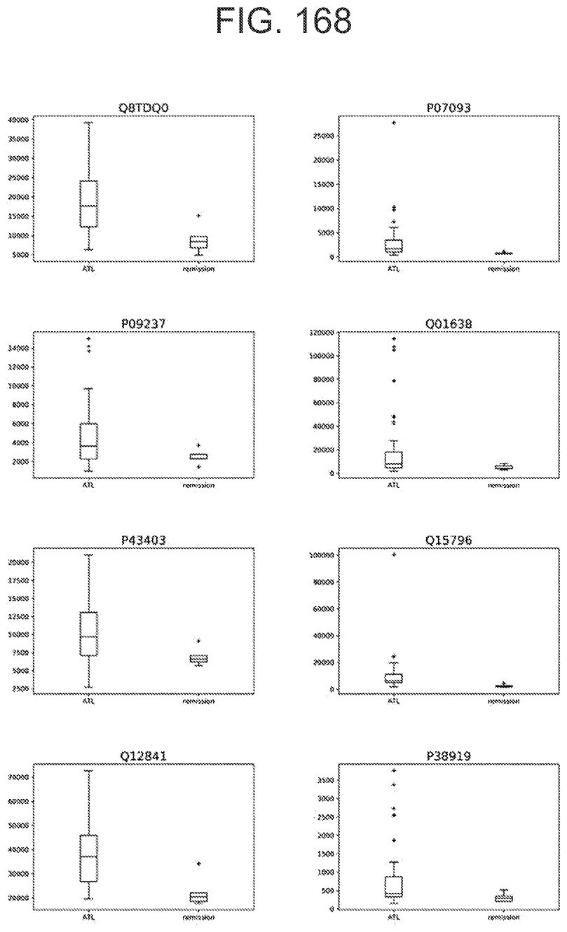

FIG. 169 The figure is a graph showing measurement results of the amount of a protein in plasmas of ATL patients and ATL patients in remission.

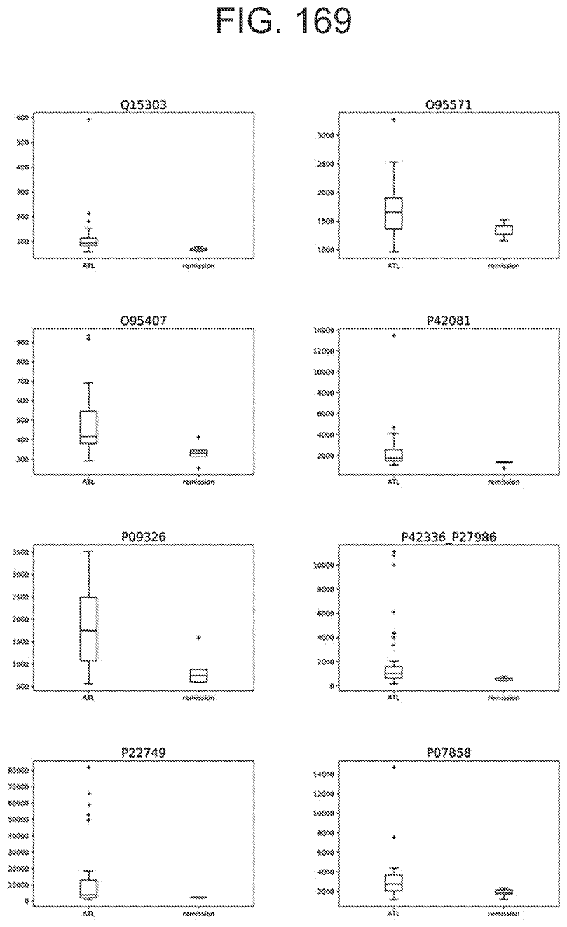

FIG. 170 The figure is a graph showing measurement results of the amount of a protein in plasmas of ATL patients and ATL patients in remission.

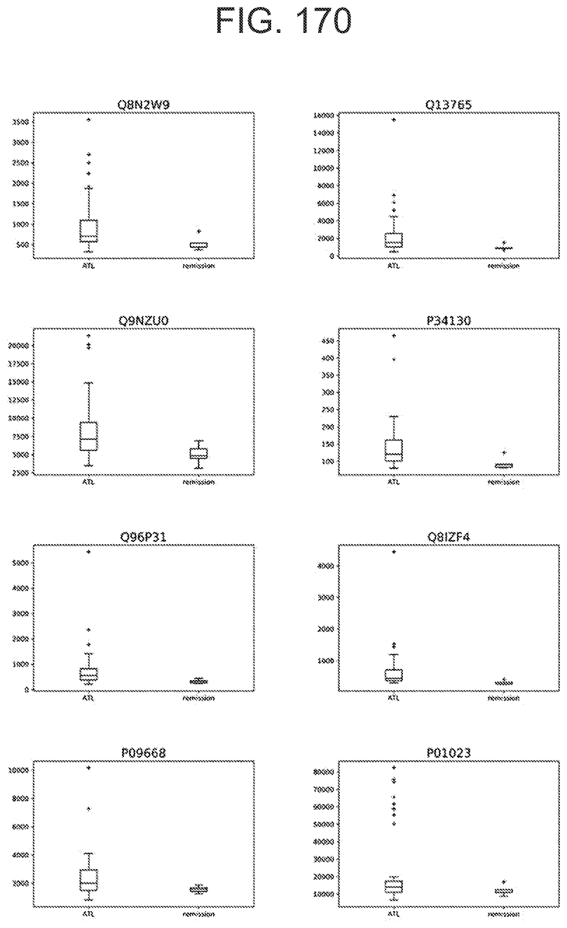

FIG. 171 The figure is a graph showing measurement results of the amount of a protein in plasmas of ATL patients and ATL patients in remission.

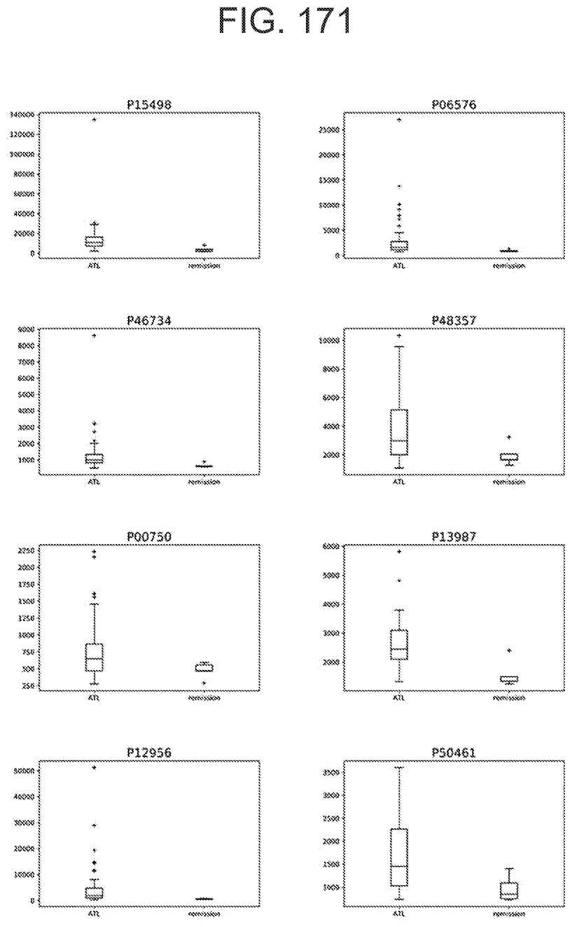

FIG. 172 The figure is a graph showing measurement results of the amount of a protein in plasmas of ATL patients and ATL patients in remission.

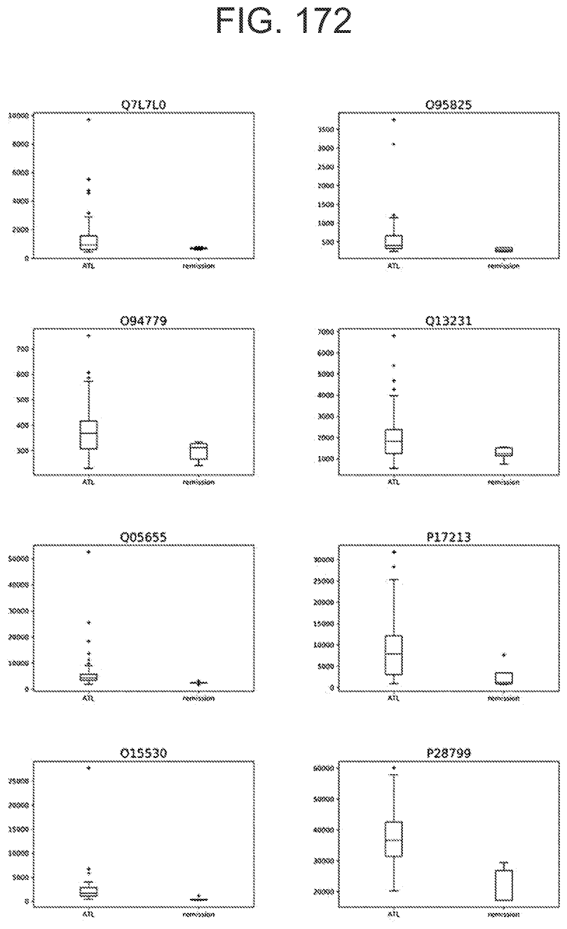

FIG. 173 The figure is a graph showing measurement results of the amount of a protein in plasmas of ATL patients and ATL patients in remission.

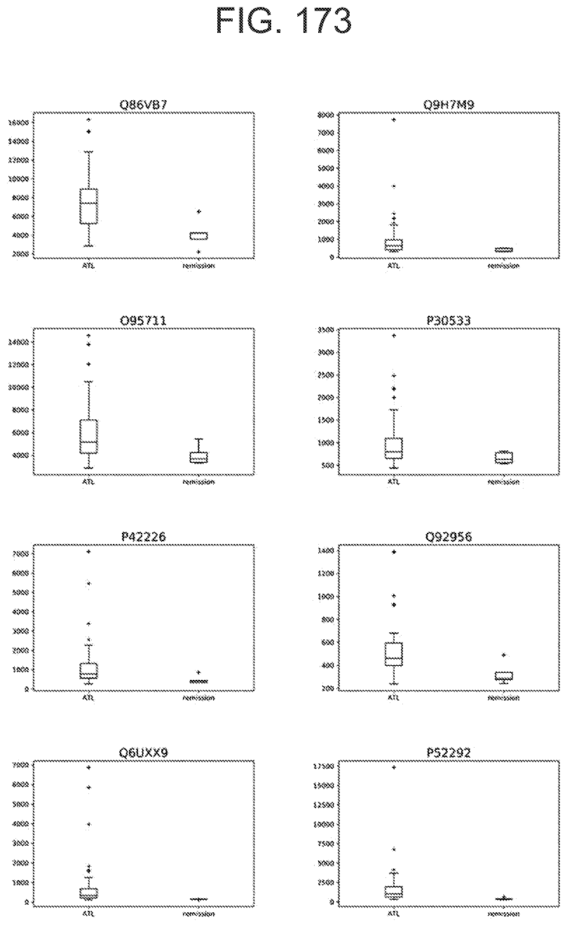

FIG. 174 The figure is a graph showing measurement results of the amount of a protein in plasmas of ATL patients and ATL patients in remission.

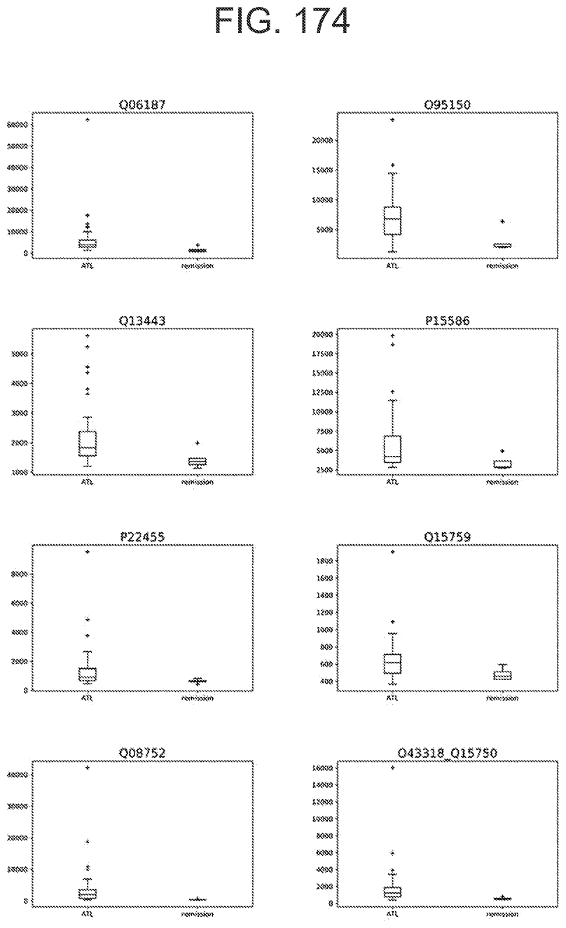

FIG. 175 The figure is a graph showing measurement results of the amount of a protein in plasmas of ATL patients and ATL patients in remission.

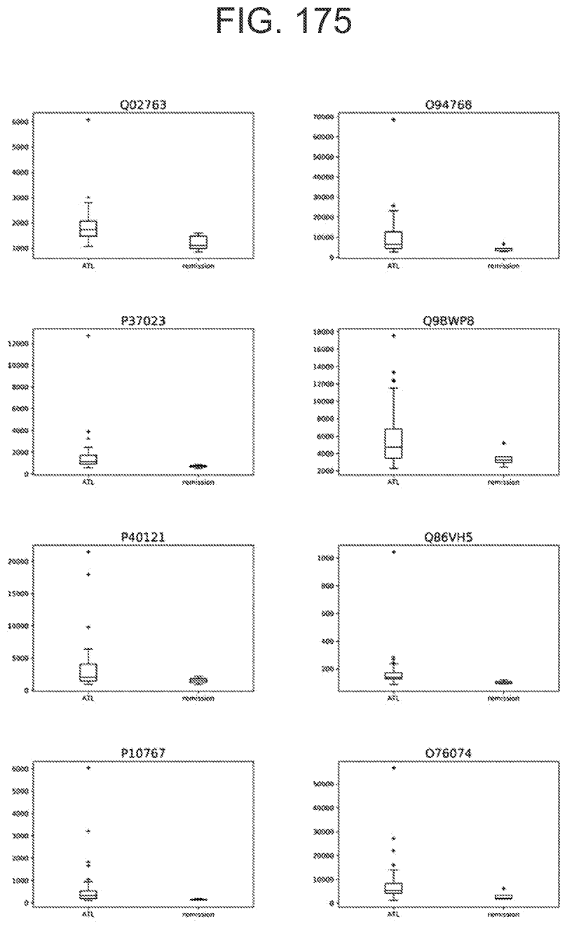

FIG. 176 The figure is a graph showing measurement results of the amount of a protein in plasmas of ATL patients and ATL patients in remission.

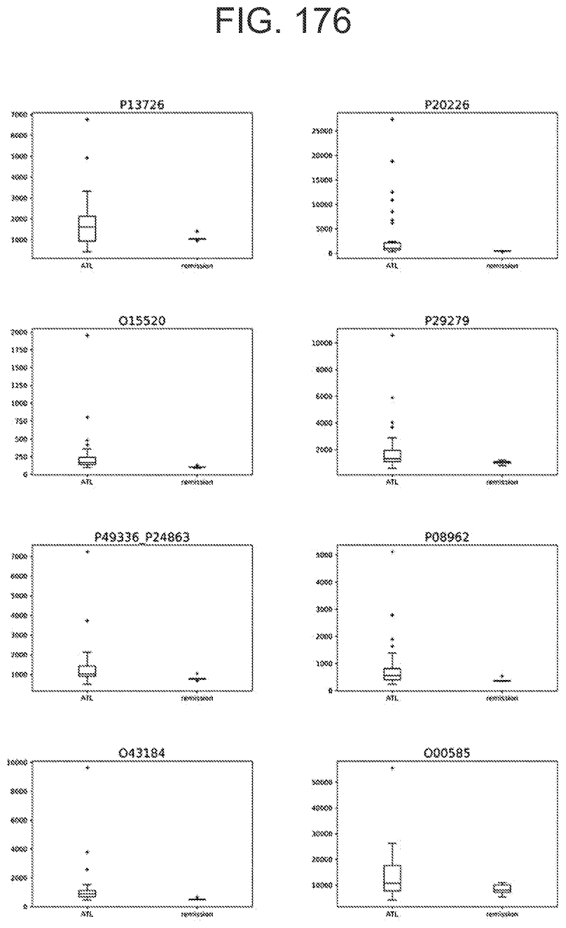

FIG. 177 The figure is a graph showing measurement results of the amount of a protein in plasmas of ATL patients and ATL patients in remission.

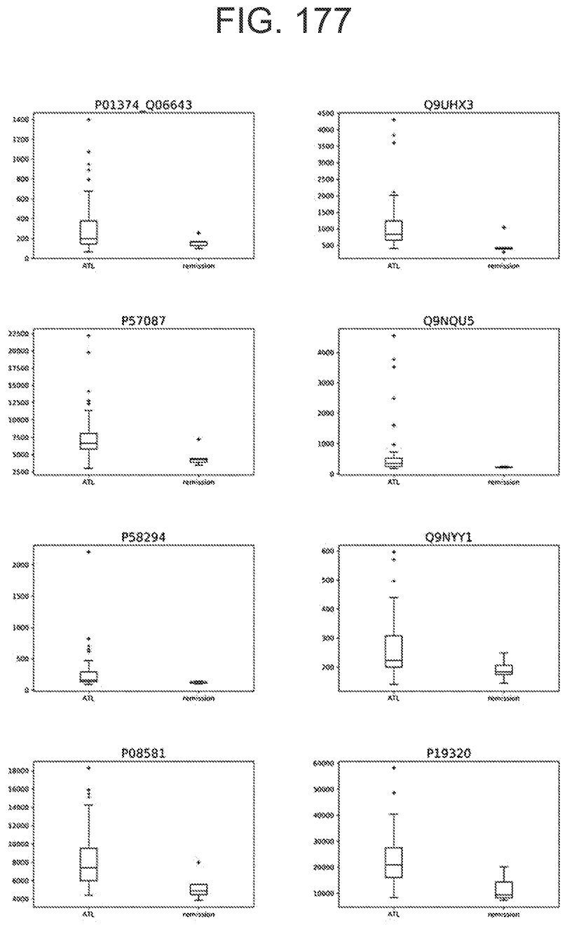

FIG. 178 The figure is a graph showing measurement results of the amount of a protein in plasmas of ATL patients and ATL patients in remission.

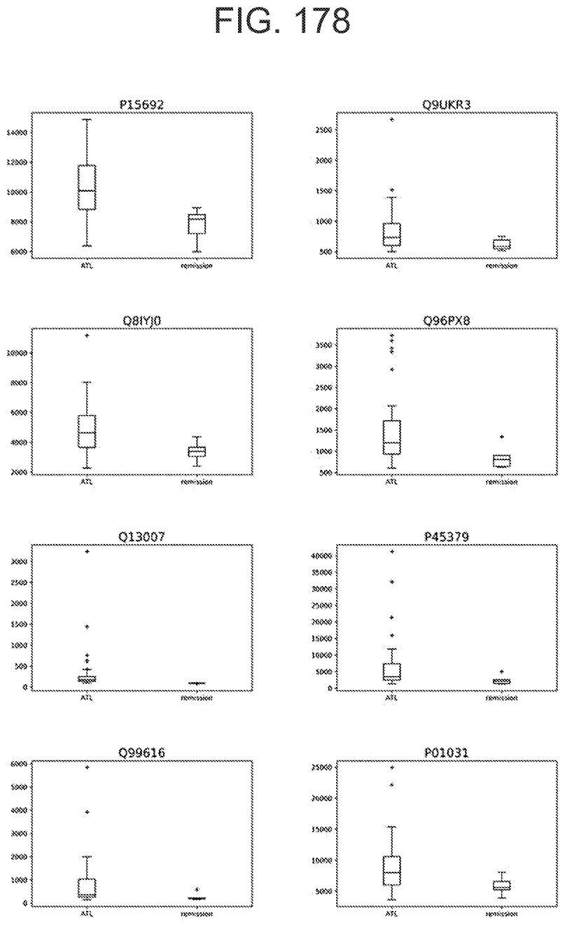

FIG. 179 The figure is a graph showing measurement results of the amount of a protein in plasmas of ATL patients and ATL patients in remission.

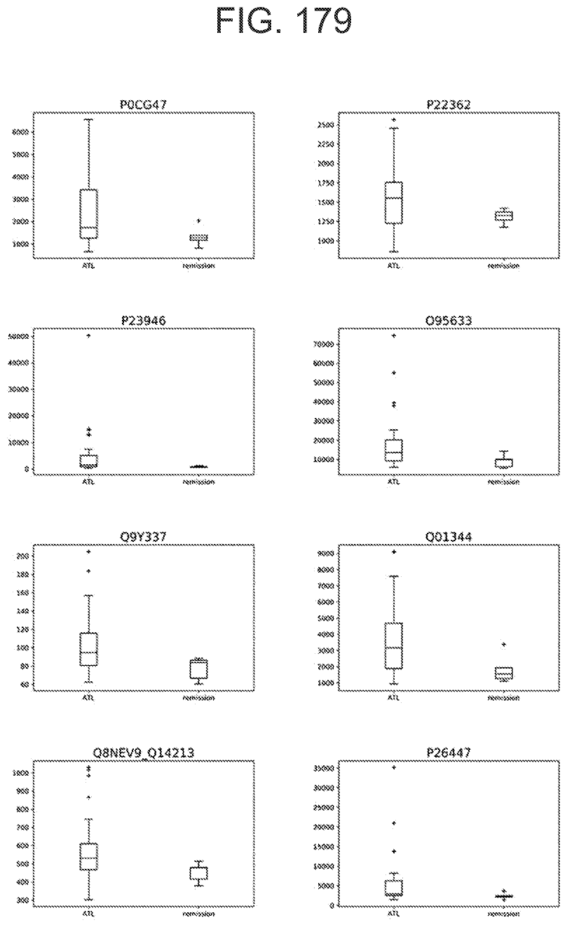

FIG. 180 The figure is a graph showing measurement results of the amount of a protein in plasmas of ATL patients and ATL patients in remission.

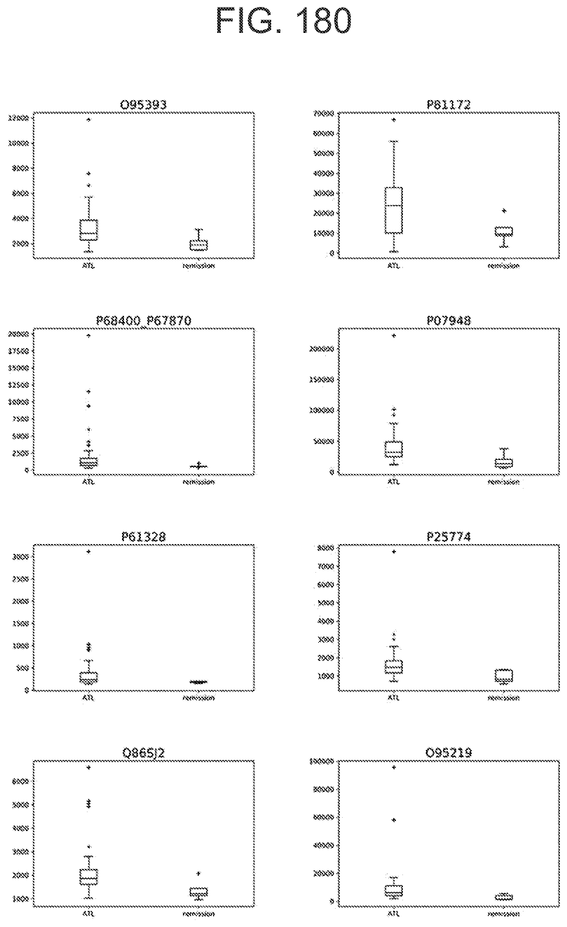

FIG. 181 The figure is a graph showing measurement results of the amount of a protein in plasmas of ATL patients and ATL patients in remission.

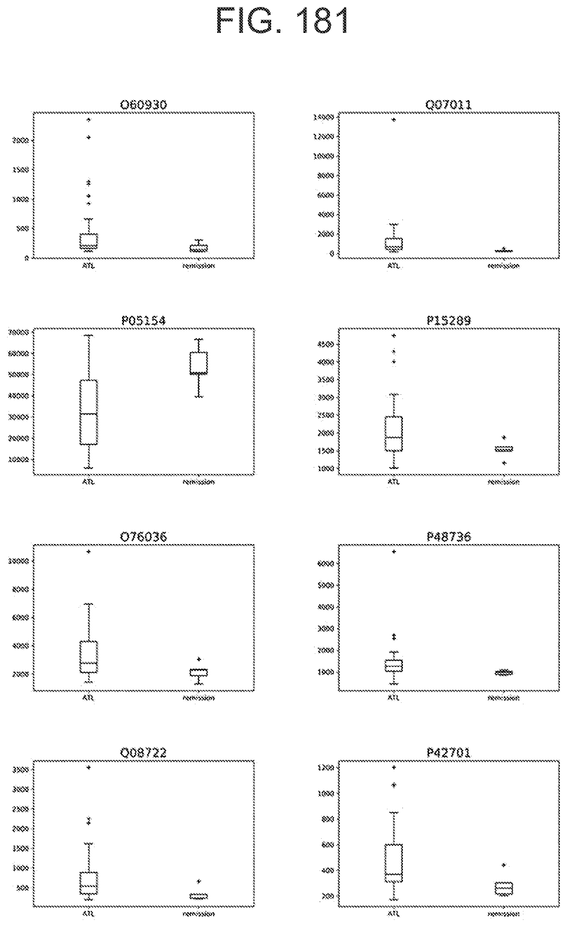

FIG. 182 The figure is a graph showing measurement results of the amount of a protein in plasmas of ATL patients and ATL patients in remission.

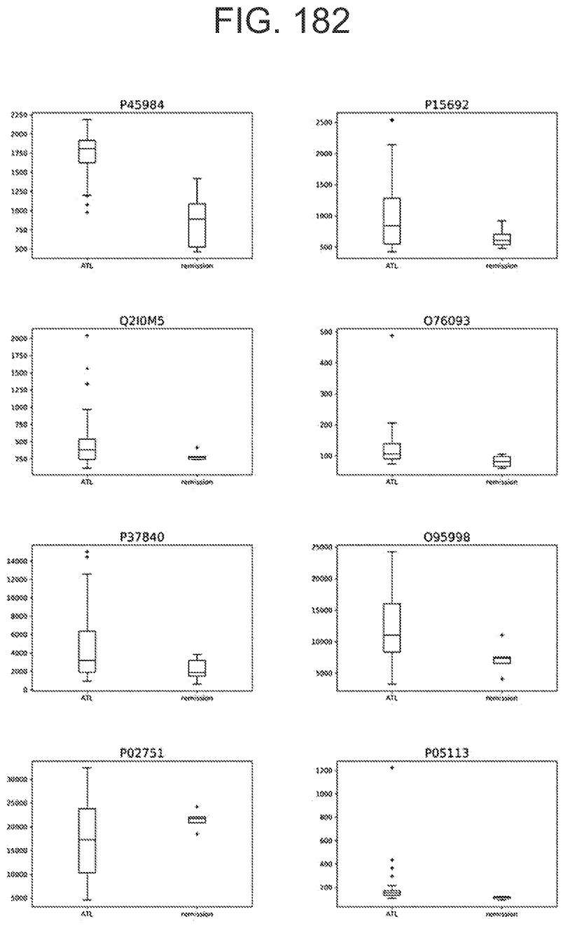

FIG. 183 The figure is a graph showing measurement results of the amount of a protein in plasmas of ATL patients and ATL patients in remission.

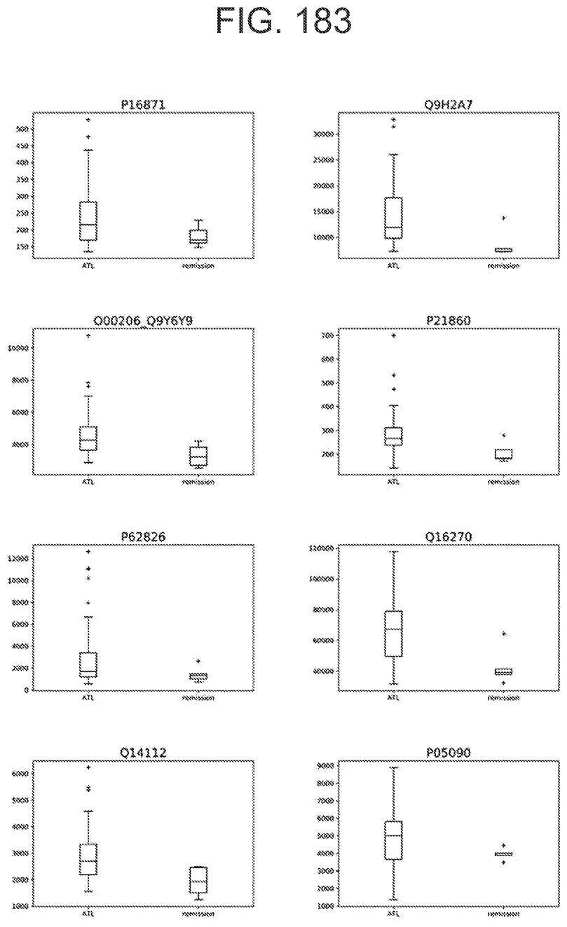

FIG. 184 The figure is a graph showing measurement results of the amount of a protein in plasmas of ATL patients and ATL patients in remission.

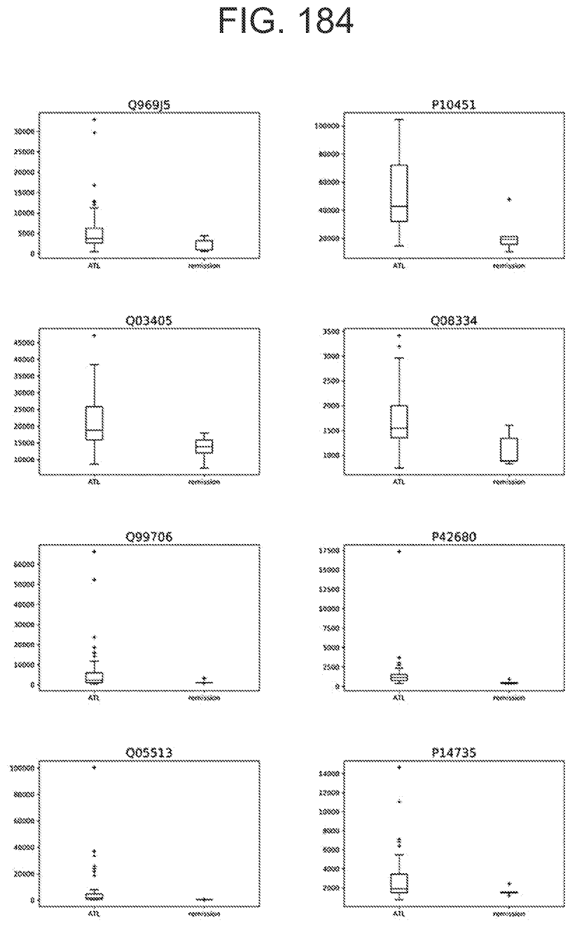

FIG. 185 The figure is a graph showing measurement results of the amount of a protein in plasmas of ATL patients and ATL patients in remission.

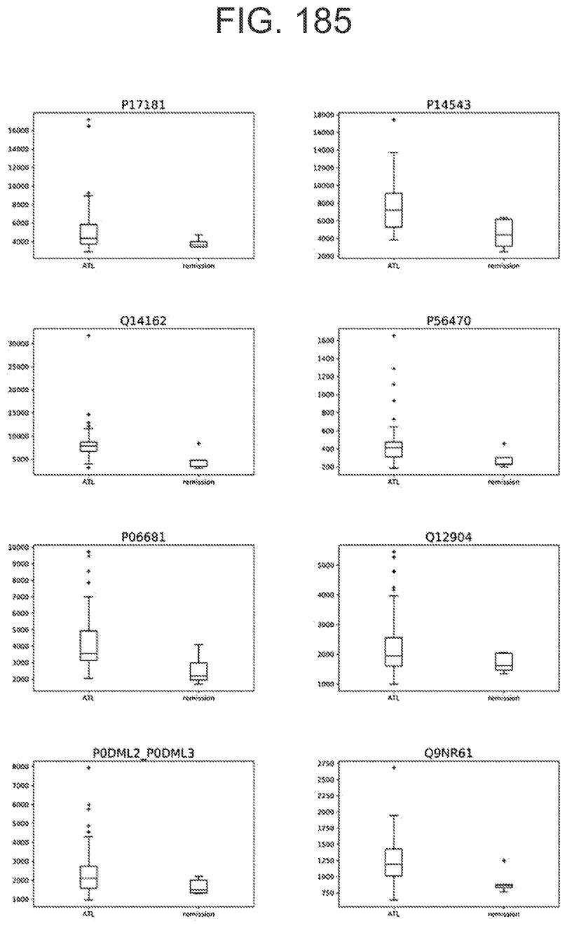

FIG. 186 The figure is a graph showing measurement results of the amount of a protein in plasmas of ATL patients and ATL patients in remission.

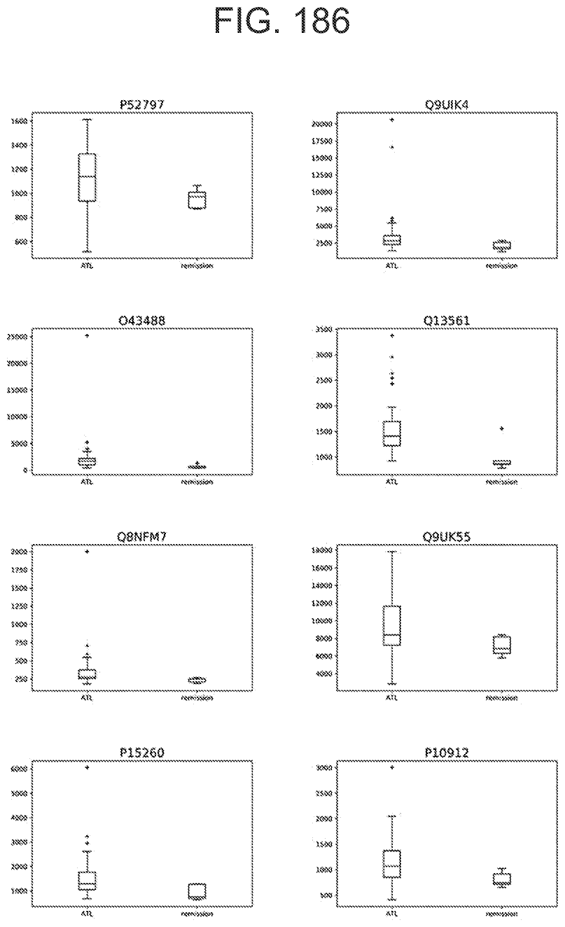

FIG. 187 The figure is a graph showing measurement results of the amount of a protein in plasmas of ATL patients and ATL patients in remission.

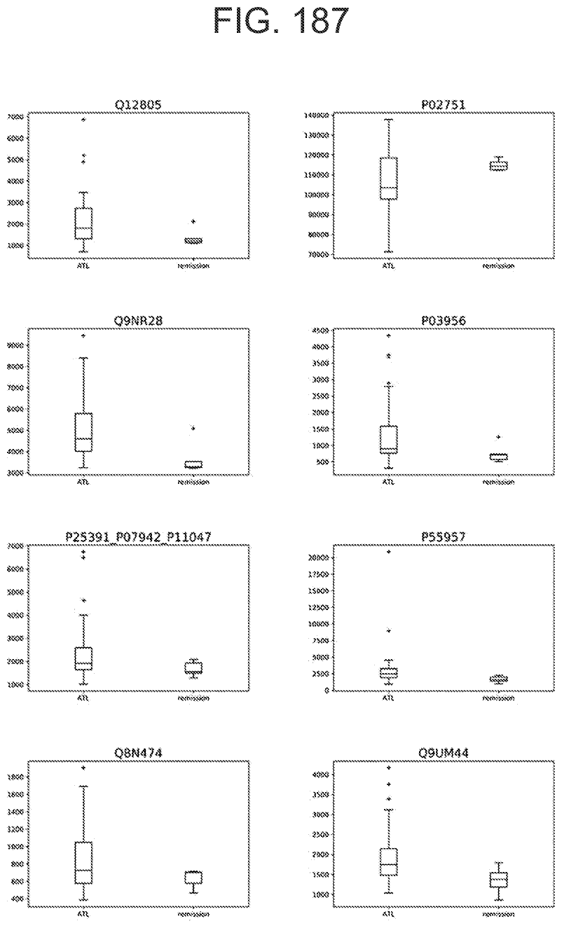

FIG. 188 The figure is a graph showing measurement results of the amount of a protein in plasmas of ATL patients and ATL patients in remission.

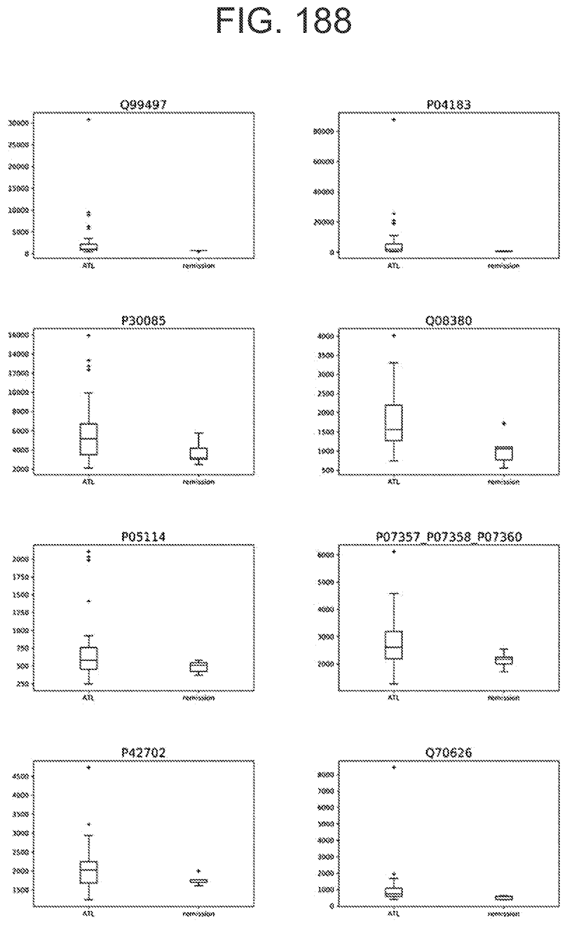

FIG. 189 The figure is a graph showing measurement results of the amount of a protein in plasmas of ATL patients and ATL patients in remission.

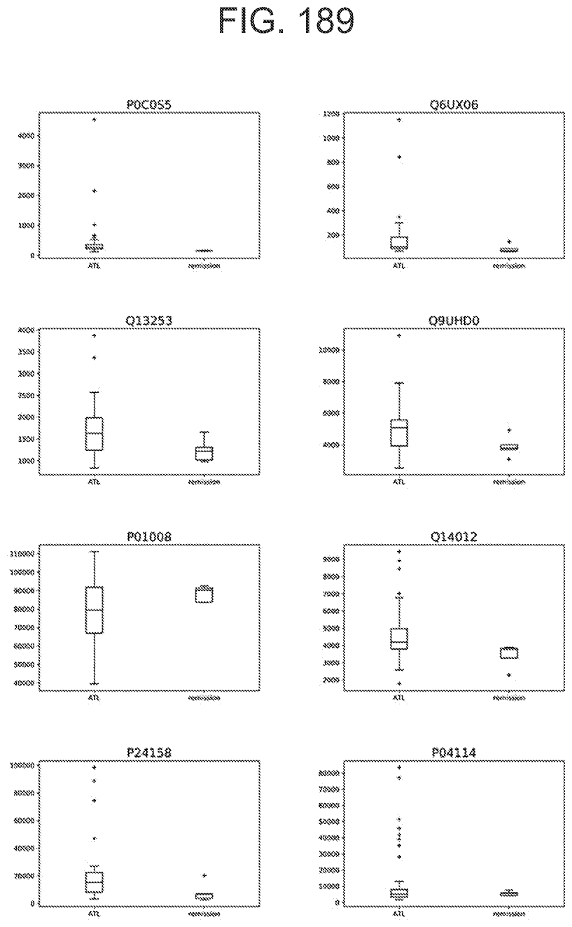

FIG. 190 The figure is a graph showing measurement results of the amount of a protein in plasmas of ATL patients and ATL patients in remission.

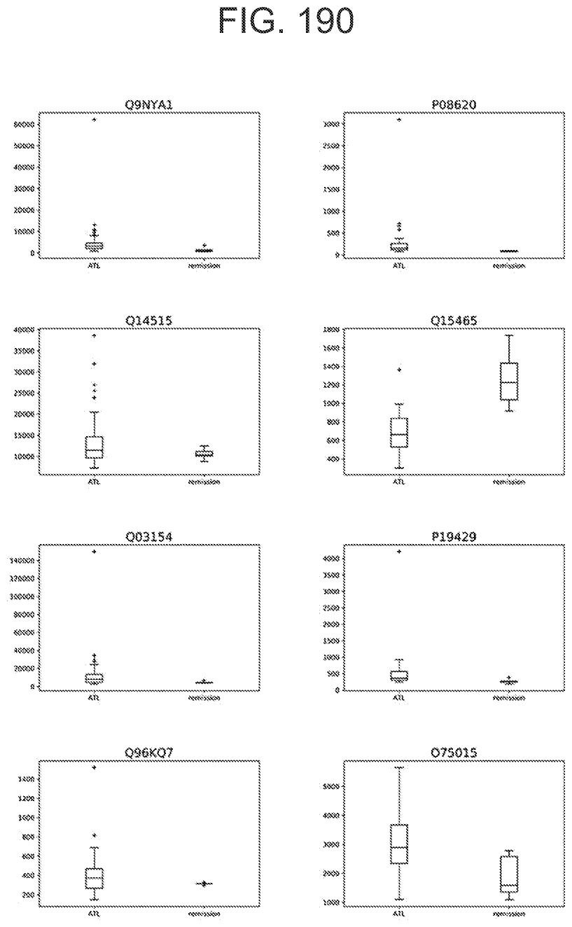

FIG. 191 The figure is a graph showing measurement results of the amount of a protein in plasmas of ATL patients and ATL patients in remission.

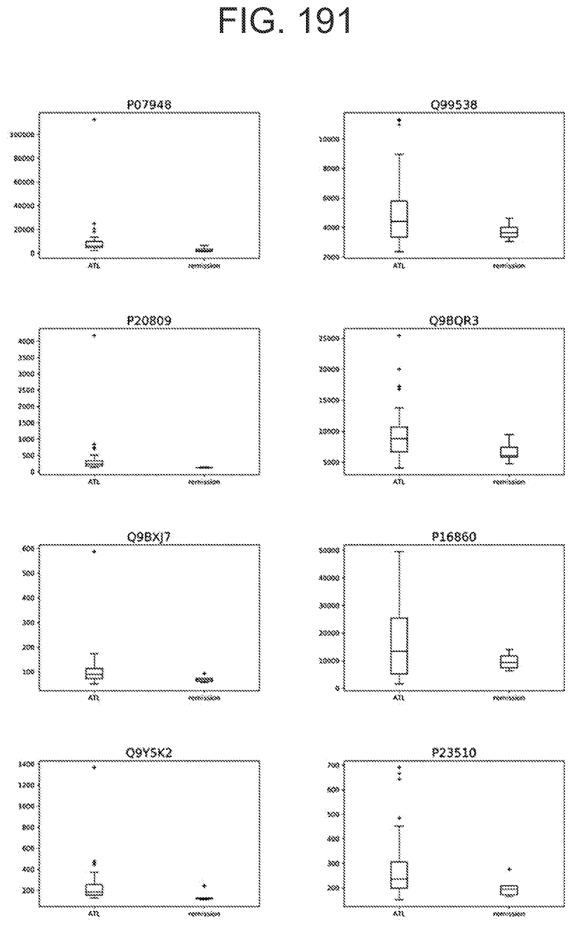

FIG. 192 The figure is a graph showing measurement results of the amount of a protein in plasmas of ATL patients and ATL patients in remission.

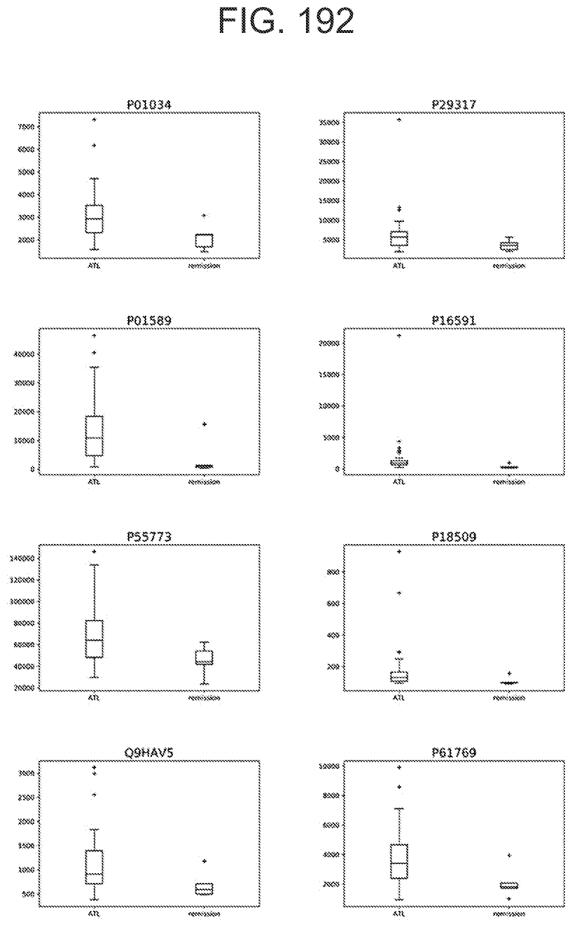

FIG. 193 The figure is a graph showing measurement results of the amount of a protein in plasmas of ATL patients and ATL patients in remission.

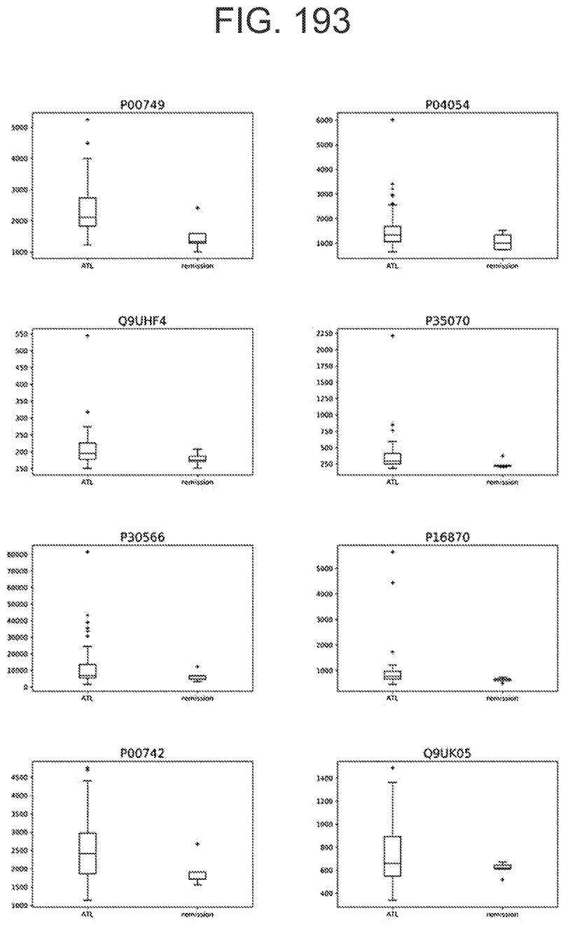

FIG. 194 The figure is a graph showing measurement results of the amount of a protein in plasmas of ATL patients and ATL patients in remission.

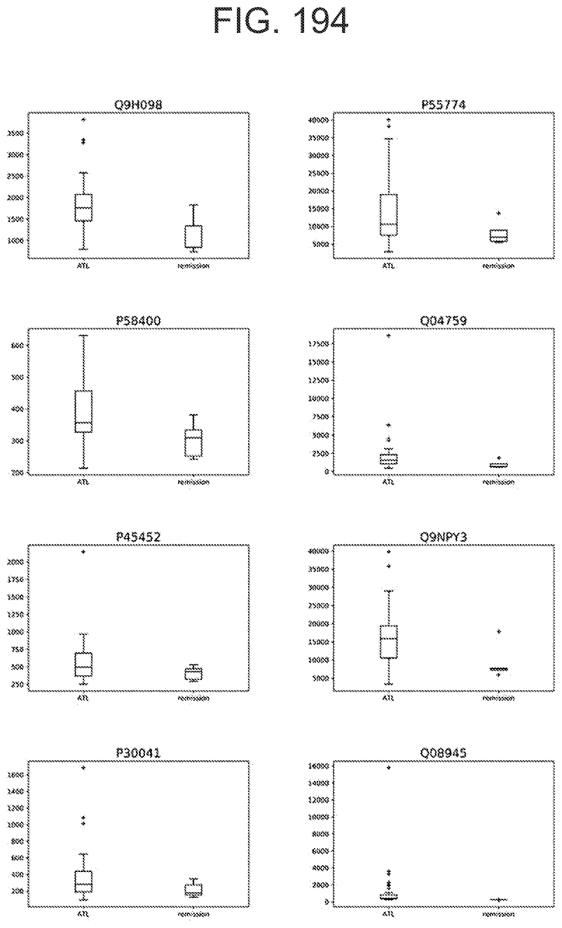

FIG. 195 The figure is a graph showing measurement results of the amount of a protein in plasmas of ATL patients and ATL patients in remission.

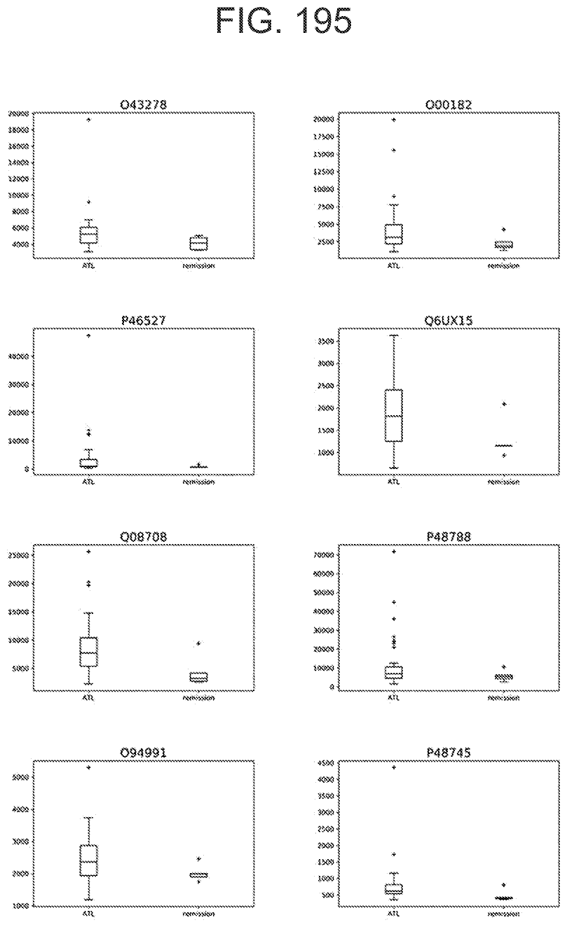

FIG. 196 The figure is a graph showing measurement results of the amount of a protein in plasmas of ATL patients and ATL patients in remission.

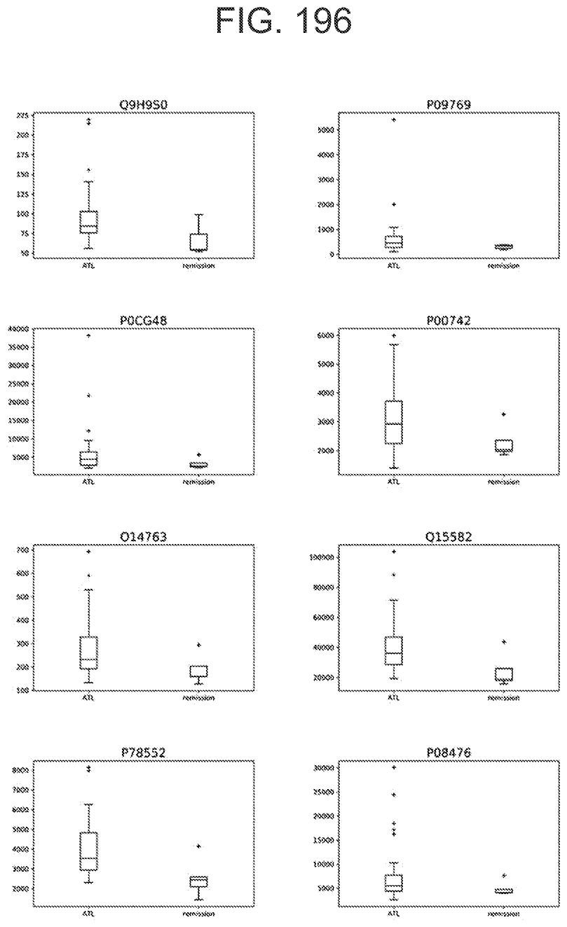

FIG. 197 The figure is a graph showing measurement results of the amount of a protein in plasmas of ATL patients and ATL patients in remission.

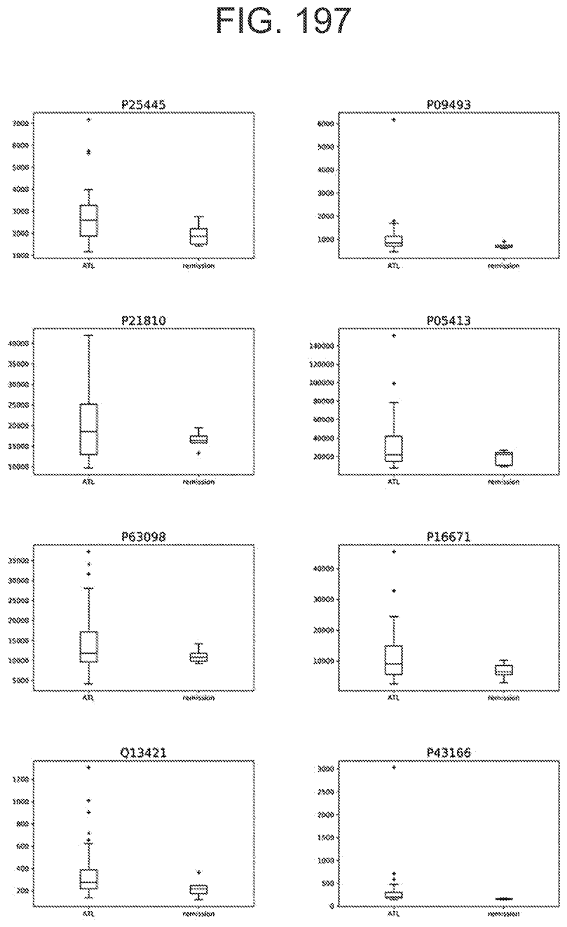

FIG. 198 The figure is a graph showing measurement results of the amount of a protein in plasmas of ATL patients and ATL patients in remission.

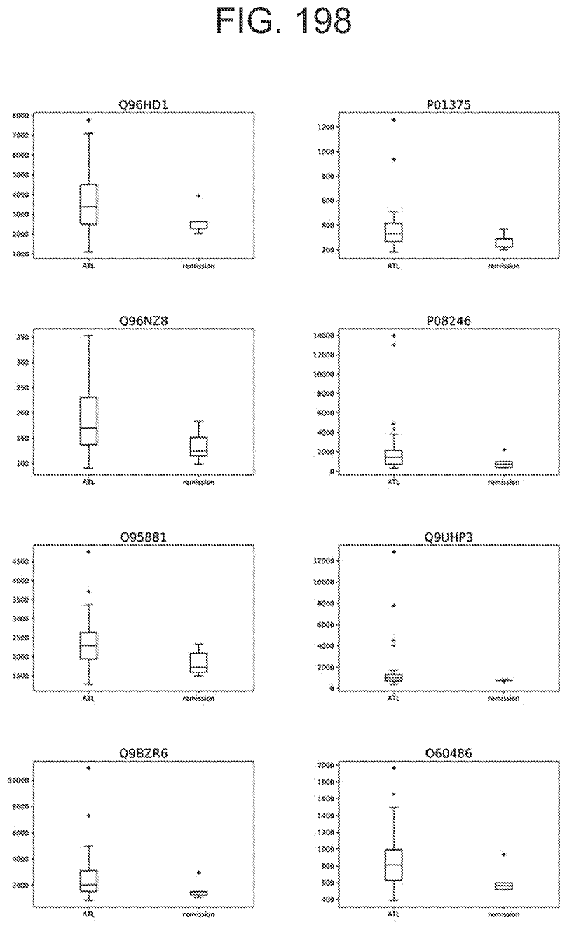

FIG. 199 The figure is a graph showing measurement results of the amount of a protein in plasmas of ATL patients and ATL patients in remission.

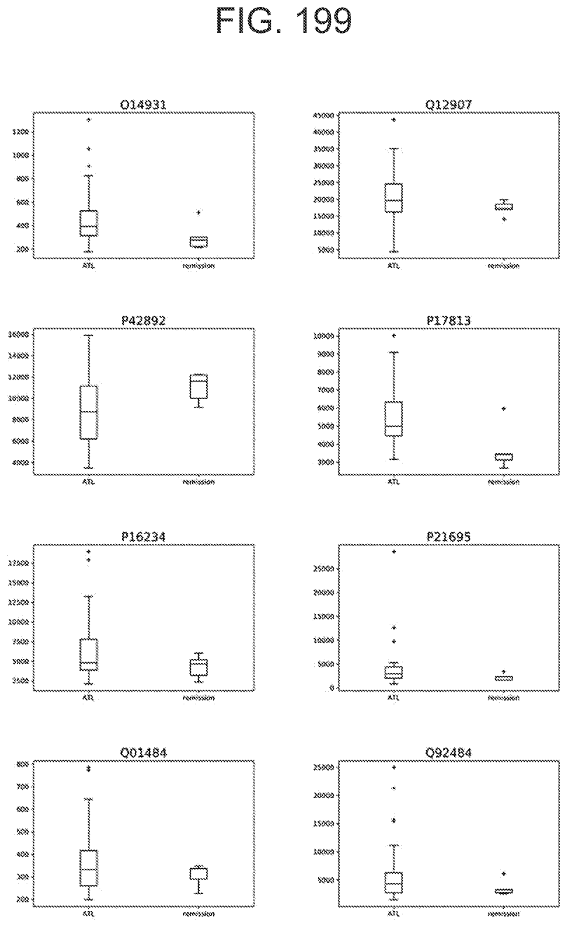

FIG. 200 The figure is a graph showing measurement results of the amount of a protein in plasmas of ATL patients and ATL patients in remission.

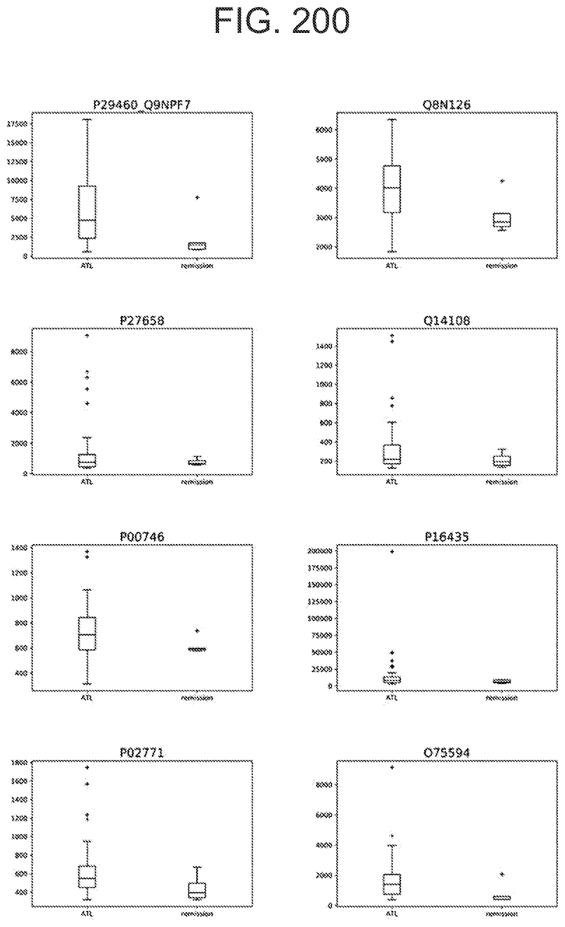

FIG. 201 The figure is a graph showing measurement results of the amount of a protein in plasmas of ATL patients and ATL patients in remission.

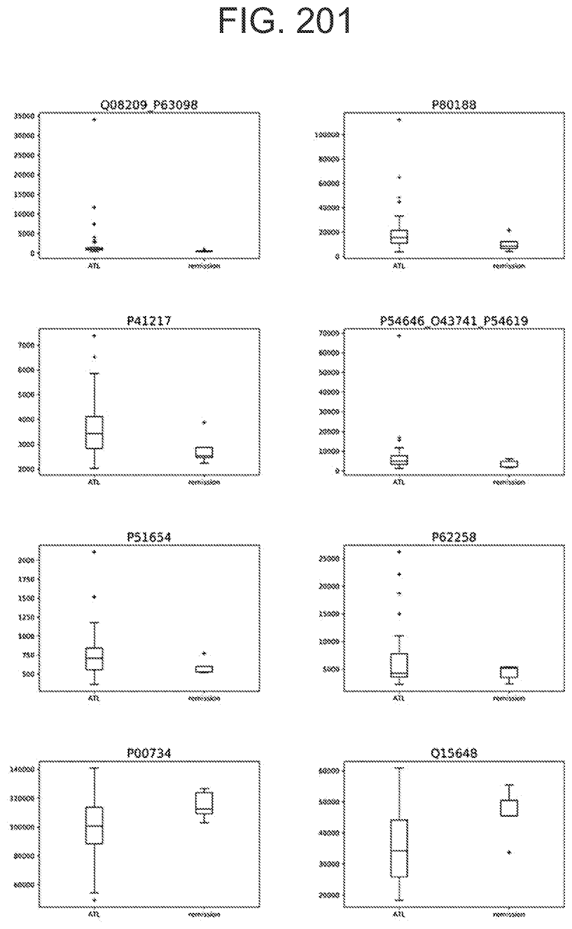

FIG. 202 The figure is a graph showing measurement results of the amount of a protein in plasmas of ATL patients and ATL patients in remission.

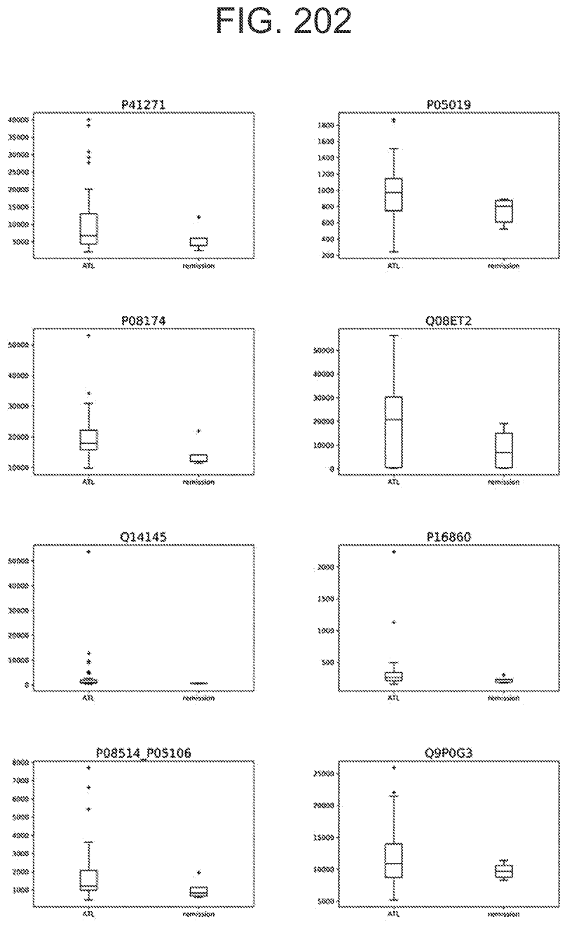

FIG. 203 The figure is a graph showing measurement results of the amount of a protein in plasmas of ATL patients and ATL patients in remission.

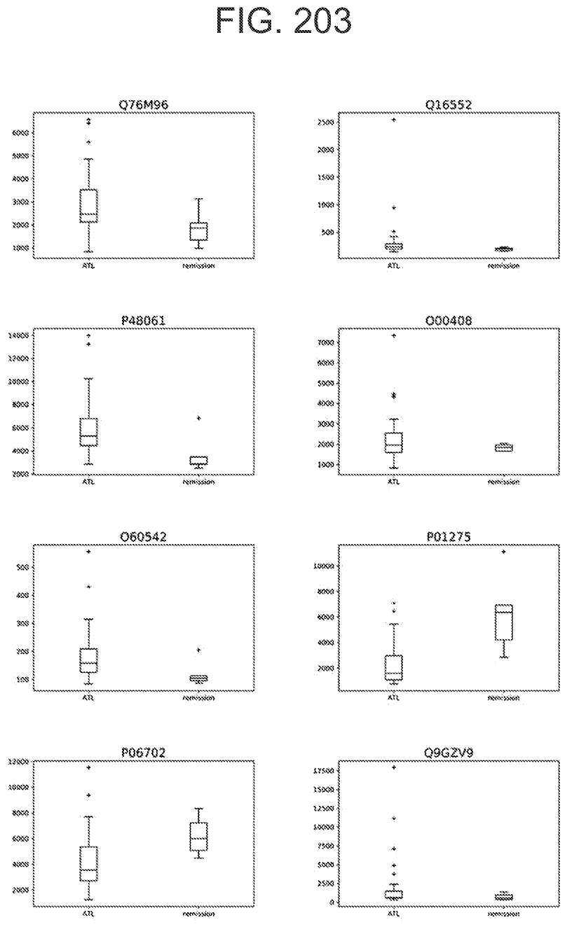

FIG. 204 The figure is a graph showing measurement results of the amount of a protein in plasmas of ATL patients and ATL patients in remission.

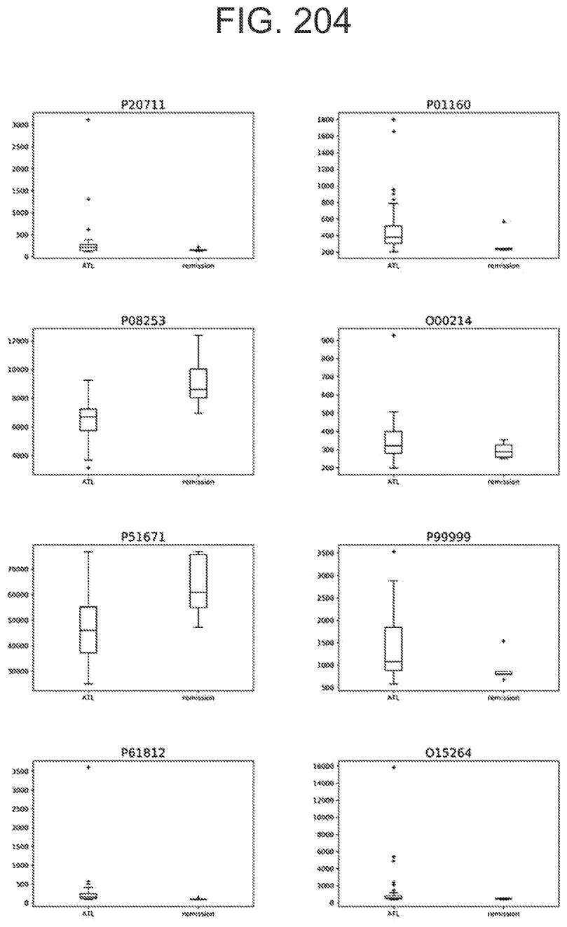

FIG. 205 The figure is a graph showing measurement results of the amount of a protein in plasmas of ATL patients and ATL patients in remission.

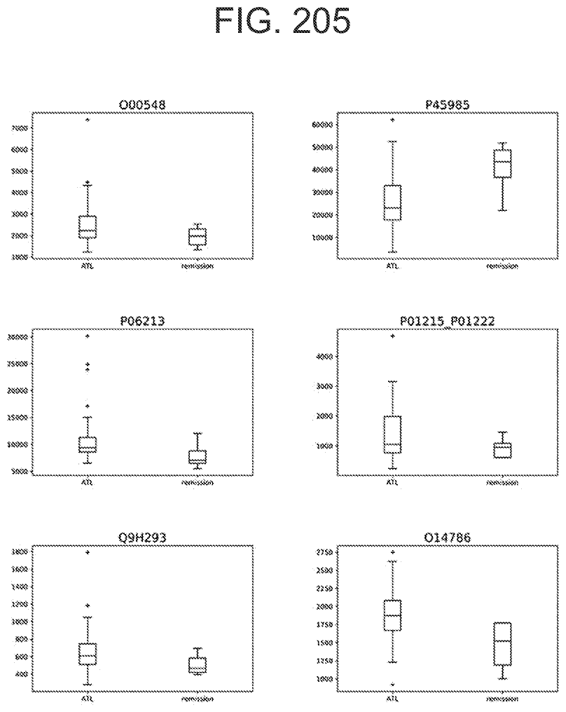

FIG. 206 The figure is a graph showing measurement results of the amount of a protein in plasmas of HTLV-1 careers, ATL patients and ATL patients in remission.

FIG. 207 The figure is a graph showing measurement results of the amount of a protein in plasmas of HTLV-1 careers, ATL patients and ATL patients in remission.

FIG. 208 The figure is a graph showing measurement results of the amount of a protein in plasmas of HTLV-1 careers, ATL patients and ATL patients in remission.

FIG. 209 The figure is a graph showing measurement results of the amount of a protein in plasmas of HTLV-1 careers, ATL patients and ATL patients in remission.

FIG. 210 The figure is a graph showing measurement results of the amount of a protein in plasmas of HTLV-1 careers, ATL patients and ATL patients in remission.

FIG. 211 The figure is a graph showing measurement results of the amount of a protein in plasmas of HTLV-1 careers, ATL patients and ATL patients in remission.

FIG. 212 The figure is a graph showing measurement results of the amount of a protein in plasmas of HTLV-1 careers, ATL patients and ATL patients in remission.

FIG. 213 The figure is a graph showing measurement results of the amount of a protein in plasmas of HTLV-1 careers, ATL patients and ATL patients in remission.

FIG. 214 The figure is a graph showing measurement results of the amount of a protein in plasmas of HTLV-1 careers, ATL patients and ATL patients in remission.

FIG. 215 The figure is a graph showing measurement results of the amount of a protein in plasmas of HTLV-1 careers, ATL patients and ATL patients in remission.

FIG. 216 The figure is a graph showing measurement results of the amount of a protein in plasmas of HTLV-1 careers, ATL patients and ATL patients in remission.

FIG. 217 The figure is a graph showing measurement results of the amount of a protein in plasmas of HTLV-1 careers, ATL patients and ATL patients in remission.

FIG. 218 The figure is a graph showing measurement results of the amount of a protein in plasmas of HTLV-1 careers, ATL patients and ATL patients in remission.

FIG. 219 The figure is a graph showing measurement results of the amount of a protein in plasmas of HTLV-1 careers, ATL patients and ATL patients in remission.

FIG. 220 The figure is a graph showing measurement results of the amount of a protein in plasmas of HTLV-1 careers, ATL patients and ATL patients in remission.

FIG. 221 The figure is a graph showing measurement results of the amount of a protein in plasmas of HTLV-1 careers, ATL patients and ATL patients in remission.

FIG. 222 The figure is a graph showing measurement results of the amount of a protein in plasmas of HTLV-1 careers, ATL patients and ATL patients in remission.

FIG. 223 The figure is a graph showing measurement results of the amount of a protein in plasmas of HTLV-1 careers, ATL patients and ATL patients in remission.

FIG. 224 The figure is a graph showing measurement results of the amount of a protein in plasmas of HTLV-1 careers, ATL patients and ATL patients in remission.

FIG. 225 The figure is a graph showing measurement results of the amount of a protein in plasmas of HTLV-1 careers, ATL patients and ATL patients in remission.

FIG. 226 The figure is a graph showing measurement results of the amount of a protein in plasmas of HTLV-1 careers, ATL patients and ATL patients in remission.

FIG. 227 The figure is a graph showing measurement results of the amount of a protein in plasmas of HTLV-1 careers, ATL patients and ATL patients in remission.

FIG. 228 The figure is a graph showing measurement results of the amount of a protein in plasmas of HTLV-1 careers, ATL patients and ATL patients in remission.

FIG. 229 The figure is a graph showing measurement results of the amount of a protein in plasmas of HTLV-1 careers, ATL patients and ATL patients in remission.

FIG. 230 The figure is a graph showing measurement results of the amount of a protein in plasmas of HTLV-1 careers, ATL patients and ATL patients in remission.

### DESCRIPTION OF EMBODIMENTS

Now, a preferred embodiment for carrying out the present invention will be described. Note that, the following embodiment is a typical embodiment of the present invention and should not be construed as limiting the range of the present invention.

1. Method for Diagnosing HTLV-1 Related Disease

The method for diagnosing an HTLV-1 related disease according to the present invention comprises the following steps (1) and (2):

(1) measuring an amount of a marker protein in a blood sample taken from a subject (measurement step); and

(2) if a measured value is a predetermined reference value or more or the predetermined reference value or less, determining that the subject has an HTLV-1 related disease or has a high possibility of developing the disease (onset diagnosis) in the case where the subject is not definitely diagnosed with the HTLV-1 related disease, and determining that the subject is in remission of an HTLV-1 related disease or has a high possibility of achieving a remission of the disease (remission diagnosis) in the case where the subject is definitely diagnosed with the HTLV-1 related disease (determination step).

In the present invention, the “onset” includes not only a first onset but also recurrence after remission, and the “remission” includes not only first remission but also re-remission after recurrence. Accordingly, step (2) can be defined as follows:

(2) if a measured value is a predetermined reference value or more or the predetermined reference value or less, determining, that the subject has an HTLV-1 related disease or has a high possibility of developing the disease in the case where the subject is an HTLV-1 career or an individual in remission of an HTLV-1 related disease (a person having a history with HTLV-1), and determining that the subject is in remission of an HTLV-1 related disease or has a possibility of achieving a remission of the disease, in the case where the subject is a patient with an HTLV-1 related disease.

[Measurement Step (Step (1))]

As described in Example, the present inventors have identified the proteins listed in Table 1 as the proteins (hereinafter referred to as “marker proteins”) that significantly change in amount in ATL patient's blood samples compared to HTLV-1 careers' blood samples. If the amounts of these marker proteins in blood samples taken from subjects are used as references, it is possible to identify that the subjects have an HTLV-1 related disease or have a high possibility of developing the disease.

When onset diagnosis is made, the subject is preferably an HTLV-1 career or an individual in remission of an HTLV-1 related disease. The HTLV-1 career can be selected in accordance with a known method in the technical field.

For example, the HTLV-1 career can be selected by detecting the presence or absence of an anti-HTLV-1 antibody in blood, for example, by PA (gelatin particle aggregation) method, CLEIA (chemiluminescent enzyme immunoassay) method or western blot.

The HTLV-1 career can be selected, for example, by detecting the presence or absence of insertion of HTLV-1 in the genome by southern blot.

In addition, the present inventors have identified the proteins listed in Table 2 as the marker proteins that significantly change in amount in blood samples of individuals in remission of ATL compared to blood samples of ATL patients. If the amounts of these marker proteins in blood samples taken from subjects are used as references, it is possible to identify that subjects are in remission of an HTLV-1 related disease or have a high possibility of achieving a remission of the disease.

When remission diagnosis is made, the subject is preferably a patient with an HTLV-1 related disease and more preferably a patient diagnosed to have an HTLV-1 related disease by the diagnostic method according to the present invention.

Examples of the HTLV-1 related disease include not only ATL but also HTLV-1 associated myelopathy (HAM) and HTLV-1 uveitis (HU). The HTLV-1 related disease is preferably ATL.

The blood sample taken from a subject may be whole blood, serum or plasma.

When onset diagnosis is made, the marker protein to be measured is defined as at least one selected from the proteins listed in Table 1.

The marker protein is preferably at least one selected from the proteins represented by Nos. 1 to 61 and 63 to 482 of Table 1, more preferably, at least one selected from the proteins represented by Nos. 1 to 61 and 63 to 333, and further preferably, at least one of the proteins represented by Nos. 1 and/or 2.

A blood coagulation factor X (UniProt ID: P00742) represented by No. 2 of Table 1 is activated to produce a blood coagulation factor Xa (UniProt ID: P00742) represented by No. 1. It is reported that factor Xa is involved in inflammation, wound healing and immunity through activation of PAR (protease-activated receptor) 1 and PAR2. As far as the present inventors know, it has not been reported that blood coagulation factors X and Xa are involved in HTLV-1 related diseases. Note that, the same UniProt ID is given to blood coagulation factor X and blood coagulation factor Xa.

When remission diagnosis is made, the marker protein to be measured is defined as at least one selected from the proteins listed in Table 2.

The marker protein is preferably at least one selected from the proteins represented by Nos. 1 to 633 listed in Table 2, more preferably at least one selected from the proteins represented by Nos. 1 to 440.

As the marker protein to be measured, 2 or more, preferably 3 or more, more preferably 4 or more, and further preferably 5 or more proteins are used in combination. The larger the number of marker proteins, the better the determination accuracy. For the reason, 6 or more proteins may be used.

The marker protein may be a full-length protein or a fragment thereof.

The “full-length protein” refers to a protein having a naturally occurring amino acid sequence, which is the same as expressed in vivo, or a mutant thereof. The fragment thereof may have a part of the amino acid sequence of a full-length protein, having a length of, for example, 5 to 10, 11 to 20, 21 to 30, 31 to 40, or 41 to 50 amino acids. The amino acid length may be 51 or more.

The amount of a marker protein in a blood sample can be measured by a method known in the technical field and using a detection reagent such as an antibody or a fragment thereof (e.g., F(ab), F(ab)2, F(ab)′ and Fv fragment), a nucleic acid or an aptamer (nucleic acid aptamer or peptide aptamer) that specifically binds to the protein.

The antibody may be a monoclonal or a polyclonal antibody and may belong to any one of the classes of IgG, IgM, IgA, IgD and IgE.

The methods for preparing these detection reagents are known in the technical field. Proper detection reagents are commercially available.

The detection reagent is preferably tagged with a label detectable by an optical, magnetic or electrical means.

The detection reagent is preferably labeled with a fluorescent substance having an excitation wavelength and emission wavelength suitably detected by a commercially available fluorescence analyzer. Examples of the fluorescence substance include phycoerythrin (PE), fluorescein isothiocyanate (FITC), rhodamine (RH), Texas Red (TX), Cy3, Hoechst 33258, and 4′,6-diamizino-2-phenyl indole (DAPI).

When a fluorescently labeled detection reagent is used, first, the detection reagent is allowed to bind to a solid phase. Then, a blood sample is brought into contact with the solid phase, reacted and washed, as needed. Fluorescence emitted from the fluorescent label of a marker protein held on the solid phase via the detection reagent, is detected. Based on the intensity thereof, the amount of protein can be determined.

The amount of the marker protein in a blood sample can be measured by, for example, LC/MS (liquid chromatography/mass spectrometry).

The amount of the marker protein can be measured by using an entrusted protein measurement service, for example, SOMAscan, provided by a company, SomaLogic.

A kit for diagnosing an HTLV-1 related disease according to the present invention can be contain not only a detection reagent as mentioned above but also various reagents for use in the measurement methods mentioned herein.

The amount of marker protein can be measured once or a plurality of times at intervals with respect to the same subject.

### [Determination Step (Step (2))]

In this step, the measured value obtained in the measurement step (step 1) is compared to a predetermined reference value. If the measured value is the reference value or more or the reference value or less, it is determined that the subject has the HTLV-1 related disease or has a high possibility of developing the disease (onset diagnosis), in the case where the subject is not definitely diagnosed with an HTLV-1 related disease (HTLV-1 career or an individual in remission of an HTLV-1 related disease), and it is determined that the subject is in remission of the HTLV-1 related disease or has a high possibility of achieving a remission of the disease (remission diagnosis) in the case where the subject is definitely diagnosed with an HTLV-1 related disease (a patient with an HTLV-1 related disease).

When onset diagnosis is made, an average, median or mode of measured values obtained in blood samples of, for example, an HTLV-1 carrier group or a healthy subject group (not HTLV-1 career), can be employed as the reference value.

With respect to the proteins represented by Nos. 1, 2, 4, 5, 7, 9, 10, 11, 16, 17, 19, 20, 21, 22, 25, 28, 31, 32, 33, 37, 39, 41, 42, 43, 44, 46, 47, 48, 49, 52, 56, 57, 58, 59, 60, 61, 62, 63, 64, 65, 66, 67, 68, 70, 72, 73, 74, 75, 76, 77, 79, 80, 81, 82, 88, 89, 90, 91, 92, 93, 94, 95, 96, 97, 98, 99, 100, 101, 102, 103, 104, 106, 108, 109, 112, 113, 115, 118, 119, 120, 121, 123, 124, 125, 128, 129, 130, 131, 133, 134, 135, 139, 141, 142, 143, 144, 149, 154, 155, 156, 157, 159, 161, 164, 165, 167, 168, 171, 172, 173, 174, 175, 176, 180, 181, 182, 183, 184, 185, 187, 189, 192, 195, 196, 198, 199, 200, 201, 203, 205, 206, 207, 208, 209, 211, 214, 215, 219, 220, 221, 222, 223, 224, 225, 226, 228, 230, 231, 232, 236, 237, 238, 240, 241, 242, 243, 244, 246, 247, 248, 249, 250, 251, 252, 253, 254, 255, 256, 259, 260, 263, 264, 267, 269, 271, 274, 279, 280, 283, 285, 288, 289, 290, 291, 293, 294, 295, 296, 298, 299, 300, 301, 302, 303, 305, 306, 307, 309, 310, 311, 312, 313, 315, 316, 317, 318, 321, 322, 323, 326, 327, 329, 330, 331, 332, 333, 334, 338, 339, 340, 341, 342, 343, 345, 346, 347, 348, 350, 352, 354, 355, 356, 357, 358, 359, 364, 365, 366, 367, 371, 373, 374, 375, 376, 378, 379, 382, 383, 384, 385, 386, 387, 388, 389, 390, 393, 394, 395, 396, 397, 398, 399, 400, 401, 405, 406, 407, 409, 411, 412, 416, 418, 420, 423, 425, 426, 427, 430, 431, 432, 433, 434, 435, 437, 438, 439, 440, 441, 443, 445, 446, 447, 450, 451, 452, 453, 454, 455, 456, 457, 460, 461, 463, 465, 466, 469, 470, 471, 472, 473, 474, 475, 477, 478, 480, 481, 482, 483, 484, 486, 487, 489, 492, 493, 494, 495, 498, 499, 500, 502, 503, 504, 505, 506, 507, 509, 510, 511, 512, 513, 514, 515, 516, 518, 523, 524, 529, 531, 532, 534, 537, 538, 540, 542, 543, 544, 546, 547, 548, 549, 551, 552, 554, 555, 556, 558, 560, 561, 565, 566, 568, 572, 573, 574, 575, 576, 579, 580, 583, 584, 585, 586, 587, 588, 589, 590, 591, 593, 594, 596, 597, 599, 600, 601, 602, 603, 605, 606, 607, 609, 610, 612, 613, 614, 615, 616, 617, 618, 619, 620, 622, 624, 627, 628, 629 and 631 of Table 1 in a patient group, a significant increase was observed compared to a carrier group.

With respect to the marker protein that has a significantly increased amount in these ATL patients, if the amount of the marker protein is increased to the reference value or more, preferably higher than the reference value by 1-5%, more preferably 6-10%, further preferably 11-25%, and most preferably 26-50%, it is determined that the subject suffers from, or is likely to develop, the HTLV-1 related disease. As the amount is significantly higher than the reference value, the determination accuracy improves. Because of this, if desired, a value higher than the reference value by 51% or more, 100% or more, 150% or more, or 200% or more may be used as an indicator.

With respect to the proteins represented by Nos. 3, 6, 8, 12, 13, 14, 15, 18, 23, 24, 26, 27, 29, 30, 34, 35, 36, 38, 40, 45, 50, 51, 53, 54, 55, 69, 71, 78, 83, 84, 85, 86, 87, 105, 107, 110, 111, 114, 116, 117, 122, 126, 127, 132, 136, 137, 138, 140, 145, 146, 147, 148, 150, 151, 152, 153, 158, 160, 162, 163, 166, 169, 170, 177, 178, 179, 186, 188, 190, 191, 193, 194, 197, 202, 204, 210, 212, 213, 216, 217, 218, 227, 229, 233, 234, 235, 239, 245, 257, 258, 261, 262, 265, 266, 268, 270, 272, 273, 275, 276, 277, 278, 281, 282, 284, 286, 287, 292, 297, 304, 308, 314, 319, 320, 324, 325, 328, 335, 336, 337, 344, 349, 351, 353, 360, 361, 362, 363, 368, 369, 370, 372, 377, 380, 381, 391, 392, 402, 403, 404, 408, 410, 413, 414, 415, 417, 419, 421, 422, 424, 428, 429, 436, 442, 444 448, 449, 458, 459, 462, 464, 467, 468, 476, 479, 485, 488, 490, 491, 496, 497, 501, 508, 517, 519, 520, 521, 522, 525, 526, 527, 528, 530, 533, 535, 536, 539, 541, 545, 550, 553, 557, 559, 562, 563, 564, 567, 569, 570, 571, 577, 578, 581, 582, 592, 595, 598, 604, 608, 611, 621, 623, 625, 626 and 630 in a patient group, a significant decrease was observed compared to a carrier group.

With respect to the marker protein that has a significantly decreased amount in these ATL patients, if the amount of the marker protein is decreased to the reference value or less, preferably lower than the reference value by 1-5%, more preferably 6-10%, further preferably 11-25%, and most preferably 26-50%, it is determined that the subject suffers from, or is likely to develop, the HTLV-1 related disease. As the amount is significantly lower than the reference value, the determination accuracy improves. Because of this, if desired, a value lower than the reference value by 51% or more, 100% or more, 150% or more, or 200% or more may be used as an indicator.

When the amount of a marker protein is measured a plurality of times at time intervals with respect to the same subject, the determination accuracy can be improved by taking a time-dependent change in the marker-protein amount into consideration.

More specifically, with respect to the marker protein that has a significantly increased amount in plasma of ATL patients compared to that in plasma of HTLV-1 carriers, if a time-dependent increase of the marker-protein in amount (for example, an increase from a value below the reference value to a value beyond the reference value) is observed, it is determined that the subject suffers from, or is more likely to develop, the HTLV-1 related disease. If the amount sharply increases with time, the subject can be diagnosed to develop a blast crisis.

With respect to the marker protein that has a significantly decreased amount in plasma of ATL patients compared to that in plasma of HTLV-1 carriers, if a time-dependent decrease of the marker-protein in amount (for example, and a decrease from a value beyond the reference value to a value below the reference value) is observed, it is determined that the subject suffers from, or is more likely to develop, the HTLV-1 related disease. If the amount sharply decreases with time, the subject can be diagnosed to develop a blast crisis.

When remission diagnosis is made, an average, median or mode of the measured values obtained in blood samples of, for example, an HTLV-1 related disease group, can be employed as the reference value.

With respect to the proteins represented by Nos. 468, 595, 607, 642, 661, 668, 739, 759, 760, 774, 775, 779, 781 and 786 of Table 2, a significant increase was observed in individuals in remission compared to a patient group.

With respect to the marker protein that has a significantly increased amount in these individuals in remission of ATL, if the amount of the marker protein is increased to the reference value or more, preferably is higher than the reference value by 1-5%, more preferably 6-10%, further preferably 11-25%, and most preferably 26-50%, it is determined that the subject is in a remission, or is likely to be in remission, of the HTLV-1 related disease. As the amount is significantly higher than the reference value, the determination accuracy improves. Because of this, if desired, a value higher than the reference value by 51% or more, 100% or more, 150% or more, or 200% or more may be used as an indicator.

With respect to the proteins represented by Nos. 1 to 467, 469 to 594, 596 to 606, 608 to 641, 643 to 660, 662 to 667, 669 to 738, 740 to 758, 761 to 773, 776 to 778, 780, 782 to 785, 786 to 790, a significant decrease was observed in individuals in remission compared to a patient group.

With respect to the marker protein that has a significantly decreased amount in these individuals in remission with ATL, if the amount of the marker protein is decreased to the reference value or less, preferably lower than the reference value by 1-5%, more preferably 6-10%, further preferably 11-25%, and most preferably 26-50%, it is determined that the subject is in remission, or is likely to be in remission, of the HTLV-1 related disease. As the amount is significantly lower than the reference value, the determination accuracy improves. Because of this, if desired, a value lower than the reference value by 51% or more, 100% or more, 150% or more, or 200% or more may be used as an indicator.

When the amount of marker protein is measured a plurality of times at time intervals with respect to the same subject, the determination accuracy can be improved by taking a time-dependent change in the marker-protein amount into consideration.

More specifically, with respect to the marker protein that has a significantly increased amount in plasma of an individual in remission of ATL compared to that in plasma of ATL patients, if a time-dependent increase in the marker-protein amount (for example, an increase from a value below the reference value to a value beyond the reference value) is observed, it is determined that the subject is in a remission, or is more likely to be in remission, of the HTLV-1 related disease. If the subject received a treatment for an HTLV-1 related disease during this period, a significant decrease in the marker protein indicates the effectiveness of the treatment.

With respect to the marker protein that has a significantly decreased amount in plasma of an individual in remission of ATL compared to that in plasma of ATL patients, if a time-dependent decrease in the marker-protein amount (for example, and a decrease from a value beyond the reference value to a value below the reference value) is observed, it is determined that the subject is in remission, or is more likely to be in remission, of the HTLV-1 related disease. If the subject received a treatment for an HTLV-1 related disease during this period, a significant decrease in the marker protein indicates the effectiveness of the treatment.

A statistical analysis method or machine learning can be used for determination.

Particularly, discriminant analysis using training data is preferably used. When two groups (for example, the HTLV-1 carrier group and the ATL patient group of the present invention) are discriminated, linear discriminant analysis is mostly sufficient.

However, linear discriminant analysis is limited in application because it is based on the assumption that the population variances of individual groups are equal. Accordingly, when discriminant analysis is carried out using more complicated data, non-linear discriminant analysis or discriminant analysis using machine learning is desirably used. Examples of the non-linear discriminant analysis include discriminant analysis based on Mahalanobis' generalized distance. Examples of the discrimination analysis using machine learning include k-nearest neighbor algorithm, Naive Bayes classifier, decision tree, neural network, support vector machine, and ensemble learning such as a bagging method, a boosting method and a random forest method. Such a method has been developed in the field of pattern recognition, and findings in the pattern recognition can be applied to the present invention.

In addition, even if an analysis method using no training data, such as principal component analysis, hierarchical clustering, non-hierarchical clustering, and self-organizing map, is employed, discrimination can be made by performing analysis in combination with the subject's data on status of an HTLV-1 related disease previously known.

2. Method for Treating HTLV-1 Related Disease

### [Treatment Step (Step (3))]

The method for diagnosing an HTLV-1 related disease according to the present invention can be applied as a method for treating a subject who has been determined to suffer from, or be likely to develop, an HTLV-1 related disease in the above step (2). The treatment method comprises the following step (3).

(3) step of applying a chemotherapy and/or a non-chemotherapy to the selected subject to be treated (treatment step).

More specifically, application of a treatment is quickly started for a treatment subject selected as having an HTLV-1 related disease or having a high possibility of developing the disease.

To a treatment subject determined to have an HTLV-1 related disease or have a higher possibility of developing the disease, or to a treatment subject determined to have a possibility of developing a blast crisis, based on the observation that a time dependent change in amount of a marker protein is large or sharp, an advanced therapy, such as allogeneic hematopoietic stem cell transplantation, T cell therapy, cancer vaccine therapy and cytokine therapy, is started in combination with or in place of a multi-drug chemotherapy.

In the cancer vaccine therapy, a peptide vaccine or a dendritic cell vaccine can be applied.

For these therapies, immune checkpoint inhibitors can be used in combination.

When the method for diagnosing an HTLV-1 related disease according to the present invention is applied as a therapeutic method, the application of a chemotherapy and/or a non-chemotherapy to a treatment subject, who has been determined to be in remission of an HTLV-1 related disease or have a high possibility of achieving a remission of the disease, can be reduced, stopped or terminated, or conversely increased, to induce a remission without fail. On the contrary, the application of a chemotherapy and/or a non-chemotherapy to a treatment subject, who has not yet been determined to be in remission of an HTLV-1 related disease or have a high possibility of achieving a remission of the disease, can be increased or another treatment can be applied in place. Note that, a radiation therapy is included in the non-chemotherapy.

### EXAMPLE

### Test Example 1: Analysis of Plasma Protein of ATL Patient

The amounts of proteins in plasmas of HTLV-1 careers (40 carriers), ATL patients (40 patients) and individuals having a remission of ATL (5 a patient in remission) were measured and compared.

The plasmas of HTLV-1 careers (hereinafter referred to as a “carrier group”), ATL patients (hereinafter referred to as a “patient group”) and individuals having a remission of ATL (hereinafter referred to as “remission group”) were provided by the Okinawa Bio Information Bank (ATL/HTLV-1 biobank) of Ryukyu University.

Using an entrusted protein measurement service, “SOMAscan” of a company, SomaLogic, the amounts of 1305 types of proteins were exhaustively measured. SOMAscan is a system that simultaneously quantifies more than 1000 types of proteins by use of aptamers SOMAmer (registered trademark) immobilized to a chip (see, Non Patent Literature 1). The outline of the procedure is as follows.

The plasma was diluted and incubated with beads, which were coated with streptavidin (SA) and having an SOMAmer reagent mixture previously immobilized thereto. The beads were washed to remove proteins non-specifically bound. The protein specifically bound to the SOMAmer reagent was labeled by use of an NHS-biotin reagent. The biotin-labeled protein-SOMAmer reagent complex and an unbound SOMAmer reagent were released from the beads by cutting a photocleaving linker with ultraviolet irradiation.

The released biotin-labeled protein-SOMAmer reagent complex and unbound SOMAmer reagent were incubated with second streptavidin coated beads. In this manner, the biotin-labeled protein-SOMAmer reagent complex was allowed to bind to the second streptavidin coated beads. The SOMAmer reagent unbound was removed by washing the beads. The SOMAmer reagent was released from the second streptavidin coated beads having the biotin-labeled protein-SOMAmer reagent complex bound thereto, in denaturation conditions.

The SOMAmer reagent was hybridized with a DNA microarray and fluorescence from cyanine 3 in the SOMAmer reagent was detected. The detected fluorescence intensity value was normalized and corrected. In this manner, the amount of a protein was determined as a relative fluorescence unit (RFU).

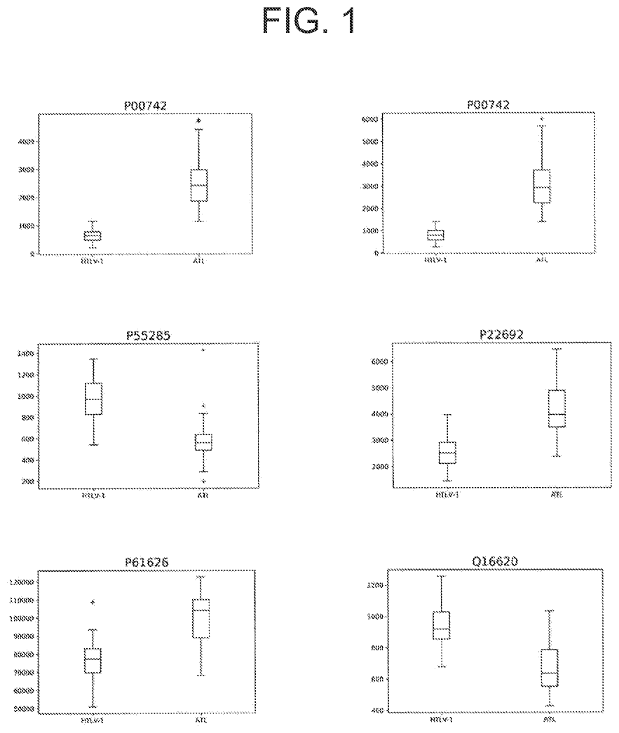

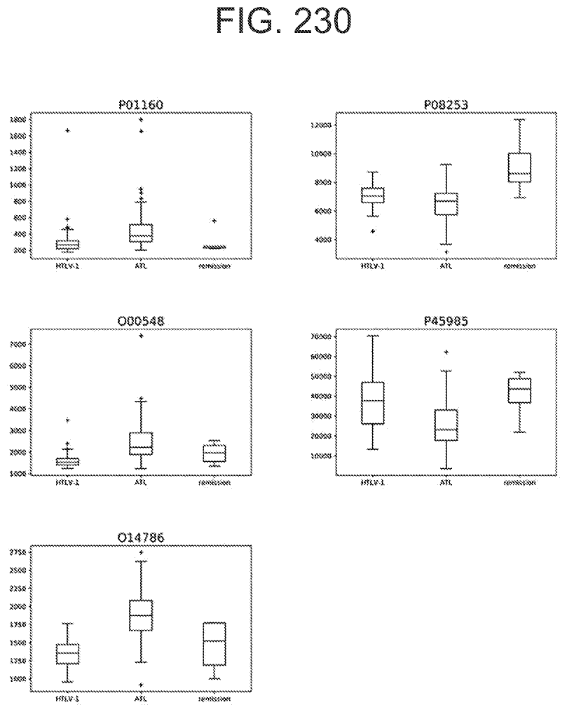

The results are shown in Tables 1 and 2 and FIGS. 1 to 230. At the top of each graph in figures, UniProt ID of a protein is shown. In the case of a protein having 2 or more UniProt IDs, all of them were described next to each other.

TABLE 1

| | | | | Average | Average | Median | Median |
|---|---|---|---|---|---|---|---|
| | | | | value | value | value | value |
| | | | | in | in | in | in |
| Protein | | UnitProt | | carrier | patient | carrier | patient |
| No. | Name of Protein | ID | p value | group | group | group | group |
| 1 | Coagulation_Factor_Xa | P00742 | 1.82E−16 | 618 | 2602 | 642 | 2419 |
| 2 | Coagulation_Factor_X | P00742 | 4.13E−16 | 786 | 3122 | 786 | 2921 |
| 3 | Cadherin-8 | P55286 | 9.32E−14 | 975 | 582 | 989 | 657 |
| 4 | IGFBP-4 | P22692 | 2.98E−12 | 2683 | 4181 | 2505 | 3968 |
| 5 | Lysozyme | P63626 | 5.65E−12 | 77252 | 100043 | 77310 | 104047 |
| 6 | TrkB | Q16620 | 1.70E−11 | 938 | 667 | 919 | 636 |
| 7 | GRN | P28799 | 2.73E−11 | 24063 | 37334 | 22295 | 36814 |
| 8 | RASA1 | P20836 | 2.71E−11 | 6719 | 3028 | 6406 | 2789 |
| 9 | IL-1B, BPa | O95998 | 5.03E−31 | 4965 | 12057 | 4567 | 10985 |
| 10 | TBF, sR-II | P20338 | 6.85E−11 | 11129 | 51875 | 9993 | 47932 |
| 11 | vWF | P04275 | 8.25E−11 | 13800 | 44646 | 12941 | 39515 |
| 12 | Apo_A-I | P02547 | 8.79E−11 | 8527 | 5094 | 8392 | 4356 |
| 13 | LYNB | P07948 | 9.20E−11 | 127208 | 43072 | 137770 | 32389 |
| 14 | FYN | P08241 | 9.30E−11 | 54557 | 11344 | 54431 | 7392 |
| 15 | ApoM | O95445 | 2.24E−10 | 11500 | 8732 | 11225 | 6490 |
| 16 | CRP | P02741 | 2.29E−10 | 21948 | 67638 | 20273 | 77904 |
| 17 | CD48 | P09326 | 4.59E−10 | 713 | 1844 | 687 | 1745 |
| 18 | ON | P09486 | 5.02E−10 | 60842 | 27996 | 56194 | 22916 |
| 19 | ST486 | Q7LFX6 | 5.03E−10 | 3408 | 5879 | 3403 | 5497 |
| 20 | NRP1 | Q14786 | 6.65E−10 | 1352 | 1875 | 1355 | 1874 |
| 21 | Ostenpondin | P10461 | 8.02E−10 | 18375 | 49729 | 17983 | 42709 |
| 22 | TIMDS | Q8TOQ0 | 8.09E−10 | 7572 | 18301 | 6742 | 17838 |
| 23 | TrkC | Q16286 | 9.69E−10 | 13276 | 10256 | 13325 | 10811 |
| 24 | RANTES | P13501 | 1.33E−09 | 59815 | 27994 | 56731 | 22737 |
| 25 | XTP3A | Q9H773 | 1.62E−09 | 21818 | 111104 | 21283 | 87965 |
| 26 | Cathepain_V | Q60911 | 1.95E−09 | 984 | 565 | 905 | 542 |
| 27 | Tromomypsin_4 | P67936 | 2.93E−09 | 106535 | 45669 | 111221 | 32924 |
| 28 | LG38P | Q08380 | 3.04E−09 | 886 | 1790 | 889 | 1559 |
| 29 | Carbonix_anhydrose_XIII | Q8N1Q1 | 3.13E−09 | 56423 | 13975 | 51630 | 7746 |
| 30 | ORG-1 | Q9NP79 | 3.74E−09 | 87913 | 27691 | 96133 | 19676 |
| 31 | FN1.4 | P02751 | 3.88E−09 | 80946 | 106550 | 80780 | 103495 |
| 32 | Endostatin | P39050 | 3.98E−09 | 47505 | 67973 | 46224 | 66189 |
| 33 | sCD163 | Q86V87 | 4.94E−09 | 3606 | 7608 | 3347 | 7419 |
| 34 | Alamin | P43652 | 6.76E−09 | 27398 | 18435 | 27106 | 17161 |
| 35 | Carbonix_anhydrose_II | P23280 | 6.76E−09 | 10695 | 3217 | 9093 | 1994 |
| 36 | Endothelin_converting_ | P42892 | 6.84E−09 | 13022 | 9062 | 12730 | 8752 |
| | enzyme_1 | | | | | | |
| 37 | FCG38 | O76016 | 7.39E−09 | 1683 | 3109 | 1487 | 2892 |
| 38 | Angiopoistin-1 | Q15389 | 7.66E−09 | 2572 | 893 | 2059 | 737 |
| 39 | Notch_1 | P46533 | 8.03E−09 | 18994 | 28986 | 19047 | 26793 |
| 40 | VAV | P15498 | 8.43E−09 | 56013 | 15041 | 56525 | 10974 |
| 41 | Tenesoin | P24821 | 8.87E−09 | 17280 | 40264 | 17028 | 35605 |
| 42 | IGFBP-7 | Q16270 | 9.52E−09 | 42549 | 66232 | 41469 | 67309 |
| 43 | b-2-Microglobulin | P61769 | 1.42-E08 | 1573 | 3797 | 1397 | 3400 |
| 44 | VCAM-1 | P19320 | 1.44E−08 | 10748 | 22797 | 10571 | 21005 |
| 45 | CTAP-III | P02775 | 1.47E−08 | 36483 | 15674 | 34495 | 12758 |
| 46 | [symbol] -b-8-1 | P55773 | 1.66E−08 | 35464 | 68059 | 35629 | 34163 |
| 47 | LAG-3 | P18627 | 2.51E−08 | 12158 | 64829 | 11652 | 45759 |
| 48 | TNF, sR-1 | P19438 | 2.37E−08 | 1080 | 2831 | 979 | 2612 |
| 49 | [symbol] | O60243 | 2.37E−08 | 1810 | 2209 | 1334 | 2094 |
| 50 | GPVI | Q9HCN6 | 2.39E−08 | 31656 | 11840 | 29765 | 9825 |
| 51 | LYN | P07948 | 2.46E−08 | 51489 | 10289 | 48708 | 6029 |
| 52 | LBP | P18428 | 2.51E−08 | 59687 | 51828 | 69582 | 78280 |
| 53 | CLC1B | Q9P126 | 2.58E−08 | 27317 | 9118 | 21196 | 8172 |
| 54 | Lactin_mannose-binding_2 | Q12967 | 0.000000028 | 30224 | 20793 | 29326 | 19690 |
| 55 | NAP-2 | P02775 | 3.34E−08 | 12643 | 5529 | 12075 | 4331 |
| 56 | CD30 | P28098 | 3.46E−08 | 1083 | 23615 | 973 | 18909 |
| 57 | TSP2 | P35442 | 3.51E−08 | 11480 | 76765 | 10152 | 64305 |
| 58 | Endovan | Q9NQ30 | 4.27E−08 | 424 | 1430 | 359 | 1130 |
| 59 | CSa | P01031 | 4.58E−08 | 4612 | 9090 | 8403 | 8015 |
| 60 | EPHB2 | P29328 | 5.39E−08 | 11162 | 22602 | 10454 | 20467 |
| 61 | P014_11_-23 | P25460 | 8.67E−08 | 1075 | 6495 | 897 | 4751 |
| | | (Q9NPF7) | | | | | |
| 62 | [symbol] _protein_2 | Q98Y67 | 5.69E−08 | 3852 | 21267 | 3359 | 17315 |
| 63 | Fibrogen_chain_dense | P62679 | 6.86E−08 | 27545 | 40703 | 25678 | 39872 |
| 64 | TNFSF15 | O85150 | 7.99E−08 | 2526 | 7169 | 2375 | 6800 |
| 65 | Ephrin-A4 | P52798 | 8.15E−08 | 1488 | 3341 | 1428 | 2746 |
| 66 | MPIE-1 | P65733 | 6.67E−08 | 1915 | 4013 | 1352 | 3567 |
| 67 | [symbol] | P00749 | 9.17E−08 | 1415 | 2358 | 1378 | 2115 |
| 68 | Macrophage_ [symbol] _receptor | P22897 | 9.21E−08 | 9297 | 19419 | 9291 | 16031 |
| 69 | PDGF-AA | P04085 | 0.000000115 | 19362 | 10151 | 18516 | 8192 |
| 70 | sL-Selectin | P14151 | 0.000000129 | 3782 | 14521 | 3647 | 13032 |
| 71 | P-Cadherin | P22223 | 0.000000129 | 19504 | 14559 | 19408 | 14719 |
| 72 | [symbol] heavy_chain, [symbol] | Q14624 | 0.000000131 | 22620 | 32685 | 25828 | 33158 |
| 73 | FST [symbol] | Q12841 | 0.000000139 | 23867 | 38451 | 23163 | 37096 |
| 74 | LO78-beta | P16819 | 0.000000159 | 1716 | 3756 | 1653 | 3165 |
| 75 | TCCR | [symbol] | 0.000000171 | 1099 | 3906 | 1082 | 3121 |
| 76 | FN1.3 | P52781 | 0.000000207 | 2623 | 4181 | 2458 | 4239 |
| 77 | Angio- [symbol] | Q15123 | 0.000000211 | 135 | 208 | 113 | 185 |
| 78 | EDAR | Q9ON52 | 0.000000213 | 5314 | 1231 | 3925 | 961 |
| 79 | Siglec-1 | Q9Y256 | 0.000000234 | 1250 | 1577 | 1264 | 1797 |
| 80 | Cystein_C | P01024 | 0.000000229 | 1902 | 3110 | 1747 | 2928 |
| 81 | PCSK7 | Q16549 | 0.000000276 | 1956 | 3653 | 2002 | 3487 |
| 82 | PARC | P56714 | 0.000000305 | 4419 | 38695 | 3754 | 10627 |
| 83 | PF-4 | P62776 | 0.00000024 | 4803 | 2358 | 4402 | 1896 |
| 84 | ER881 | P00533 | 0.000000354 | 22130 | 17998 | 22159 | 17735 |
| 85 | CHKB | Q9Y259 | 0.000000365 | 13398 | 2714 | 12087 | 6768 |
| 86 | 0230_GOF-13_8 | O95390 | 0.000000358 | 1148 | 728 | 1127 | 710 |
| | | (O14793) | | | | | |
| 87 | a2-143-Glycoprotein | P02765 | 0.000000375 | 2066 | 1572 | 2094 | 1530 |
| 88 | sE-Selectin | P16981 | 0.000000404 | 28099 | 70072 | 27988 | 61256 |
| 89 | TIMP-1 | P01033 | 0.000000412 | 278 | 712 | 264 | 614 |
| 90 | GAS1 | P64826 | 0.00000042 | 562 | 839 | 546 | 783 |
| 91 | DKK3 | Q9U8P4 | 0.000000421 | 5827 | 8437 | 6488 | 8433 |
| 92 | [symbol] | Q03605 | 0.000000426 | 12623 | 20641 | 12802 | 18294 |
| 93 | ALCAM | Q13740 | 0.000000434 | 13507 | 21624 | 12927 | 19556 |
| 94 | BCGF-beta | Q9Z240 | 0.000005454 | 1260 | 1514 | 1179 | 2575 |
| 95 | MC-1 | Q999988 | 0.000000462 | 1036 | 4137 | 921 | 3431 |
| 96 | N-terminal_pro_BNP | P18860 | 0.000000478 | 3889 | 15980 | 2737 | 13394 |
| 97 | ECGF-alpha | Q9Y240 | 0.000000489 | 3137 | 9354 | 3194 | 7496 |
| 98 | 0247_Fibrogen | P02873 | 0.000000498 | 13234 | 112850 | 85423 | 111585 |
| | | (P02675/ | | | | | |
| | | P02679 | | | | | |
| 99 | LRP8 | Q16114 | 0.000000504 | 2727 | 7659 | 2661 | 6194 |
| 100 | [symbol] K35 | P00843 | 0.000000615 | 790 | 2336 | 865 | 1722 |
| 101 | CLM6 | O08708 | 0.000000621 | 3712 | 3624 | 3457 | 7724 |
| 102 | IL-28- [symbol] | Q13478 | 0.000000559 | 8087 | 19642 | 7764 | 17346 |
| 103 | CREL3 | Q96HD3 | 0.000000803 | 2685 | 3757 | 1869 | 3579 |
| 104 | BSP | P21815 | 0.000000805 | 2616 | 14878 | 1901 | 10492 |
| 105 | BTK | Q06187 | 0.000000608 | | | | |
| 106 | IL [symbol] | P08637 | 0.000000608 | 12607 | 24838 | 12423 | 19647 |
| 107 | Kalistatin | P29622 | 0.000000667 | 33264 | 24304 | 34318 | 24164 |
| 108 | RELT | Q96924 | 0.000000668 | 3866 | 5248 | 1661 | 4144 |
| 109 | UNCSH3 | Q95185 | 0.000000678 | 2621 | 4176 | 2454 | 4061 |
| 110 | EPISR | Q9BBC2 | 0.000000734 | 9792 | 1311 | 3632 | 913 |
| 111 | PDPK3 | O15530 | 0.000000736 | 9421 | 2712 | 8877 | 1724 |
| 112 | tPA | P00750 | 0.0000000897 | 355 | 779 | 368 | 650 |
| 113 | IGFBF-2 | P18065 | 0.000000968 | 258 | 1008 | 180 | 749 |
| 114 | [symbol] | P53552 | 0.00000101 | 15683 | 11696 | 15452 | 10909 |
| 115 | Lay | Q8UX16 | 0.00000108 | 1180 | 1960 | 1108 | 1813 |
| 116 | IEC | P42680 | 0.0000012 | 8526 | 1693 | 5491 | 1133 |
| 117 | GPIBA | P97369 | 0.00000127 | 2196 | 1448 | 1936 | 1311 |
| 118 | [symbol] | Q8N423 | 0.00000129 | 63144 | 11285 | 6422 | 9598 |
| 119 | C9 | P02748 | 0.00000183 | 53767 | 73262 | 63808 | 73789 |
| 120 | Fas_soluble | P26445 | 0.00000146 | 1596 | 2753 | 1653 | 2594 |
| 121 | TGF-63 | P10600 | 0.00000148 | 269 | 434 | 247 | 358 |
| 122 | FEB | P18691 | 0.00000153 | 6334 | 1873 | 5794 | 932 |
| 123 | Q1QR1 | Q9NPY3 | 0.00000154 | 9800 | 16556 | 9378 | 15923 |
| 124 | LY9 | Q9M8G7 | 0.00000154 | 9368 | 17254 | 9070 | 19164 |
| 125 | GFRe-2 | O00452 | 0.00000188 | 4048 | 7646 | 3918 | 6788 |
| 126 | [symbol] _Hedging | Q15456 | 0.00000167 | 1012 | 594 | 983 | 686 |
| 127 | DUS3 | P81352 | 0.00000157 | 6282 | 1696 | 8788 | 1262 |
| 128 | [symbol] | Q13561 | 0.00000189 | 1041 | 1863 | 1002 | 1412 |
| 129 | PIANP | Q88Y10 | 0.00000175 | 3158 | 4868 | 2899 | 4554 |
| 130 | DR6 | O75509 | 0.00000176 | 6413 | 9956 | 6082 | 9157 |
| 131 | FBLN3 | Q12808 | 0.00000177 | 1037 | 2178 | 945 | 1808 |
| 132 | CAMK2B | Q13657 | 0.00000188 | 27132 | 8705 | 27940 | 4652 |
| 133 | LTBP4 | Q8N253 | 0.00000394 | 1372 | 2276 | 1127 | 1342 |
| 134 | [symbol] | P78423 | 0.00000186 | 1565 | 2900 | 1471 | 2718 |
| 135 | FLRTZ | O43156 | 0.00000199 | 13194 | 17263 | 12619 | 17248 |
| 136 | SRCN1 | P12931 | 0.00000208 | 78079 | 28542 | 76765 | 13831 |
| 137 | GRB2_adapter_protein | P52993 | 0.00000212 | 122881 | 73895 | 130648 | 68586 |
| 138 | Thrombospondin-1 | P07996 | 0.00000214 | 4302 | 1710 | 3957 | 1515 |
| 139 | CDGR | P13987 | 0.00000279 | 1805 | 2652 | 1724 | 2445 |
| 140 | NDP, kinase, B | P22392 | 0.00000229 | 62698 | 29297 | 80071 | 28592 |
| 141 | GFRs-1 | P56159 | 0.00000233 | 338 | 729 | 300 | 687 |
| 142 | D943 [symbol] -dimer | P02671 | 0.00000236 | 7195 | 9633 | 7043 | 9402 |
| | | (P02575/ | | | | | |
| | | P02670) | | | | | |
| 143 | a1-Antochymorrpsin [symbol] | P03011 | 0.000000244 | 196126 | 217260 | 188203 | 218536 |
| 144 | CATZ | O9U8R2 | 0.000000254 | 4080 | 7089 | 3845 | 5252 |
| 145 | NSF1C | Q9UNZ2 | 0.000000257 | 13757 | 5127 | 13067 | 3581 |
| 146 | Mynstatin | O14793 | 0.000000264 | 4920 | 2781 | 3767 | 2161 |
| 147 | CAMK2A | Q9UQM7 | 0.000000276 | 5484 | 1607 | 6377 | 903 |
| 148 | 1106_AMPK_a2b2g1 | P54546 | 0.000000278 | 22864 | 7390 | 23345 | 5042 |
| | | (Q43741/ | | | | | |
| | | F54619) | | | | | |
| 149 | PLXB2 | O15031 | 0.00000289 | 4142 | 8034 | 4065 | 5431 |
| 150 | POGF-88 | P01127 | 0.00000294 | 62328 | 32971 | 54398 | 28262 |
| 151 | [symbol] | P0059 | 0.00000303 | 32519 | 34214 | 31062 | 10424 |
| 152 | IFAG2 | P78344 | 0.00000316 | 29869 | 4735 | 6249 | 4434 |
| 153 | [symbol] | O60289 | 0.00000323 | 6460 | 4735 | 6249 | 4434 |
| 154 | TFPI | P10646 | 0.00000336 | 17825 | 27732 | 17824 | 24907 |
| 155 | CYCL16_ [symbol] | Q89H2A7 | 0.00000363 | 8635 | 14073 | 8608 | 11890 |
| 156 | Carhepsin_B | P07858 | 0.00000382 | 1313 | 3185 | 1298 | 2786 |
| 157 | 0222_C1q | P02745 | 0.00000421 | 27738 | 33862 | 27044 | 32650 |
| | | (P02746/ | | | | | |
| | | P02747) | | | | | |
| 158 | CAMK28 | Q13564 | 0.00000456 | 11206 | 3576 | 10893 | 1920 |
| 159 | [symbol] | P22306 | 0.00000672 | 5251 | 7382 | 4975 | 6033 |
| 160 | Heparin_cofactor_II | P05546 | 0.00000477 | 2782 | 2013 | 2783 | 1986 |
| 161 | FSTL3 | O95633 | 0.00000484 | 5891 | 17397 | 5398 | 13652 |
| 162 | RPS6RA3 | P51812 | 0.00000631 | 15456 | 61201 | 34015 | 4804 |
| 163 | SGTA | Q43768 | 0.00000638 | 21368 | 7163 | 20584 | 5353 |
| 164 | ILE−2 | Q8NHL5 | 0.00000679 | 3390 | 5021 | 3182 | 5017 |
| 165 | [symbol] | P62823 | 0.00000604 | 2536 | 3923 | 1391 | 2983 |
| 166 | 0652_CK-MB | P12277 | | | | | |
| | | (P06732) | 0.00000621 | 1206 | 529 | 1084 | 425 |
| 167 | M-CSF_8 | P07833 | 0.00000658 | 122 | 218 | 132 | 189 |
| 168 | [symbol] | Q14625 | 0.00000799 | 33599 | 68198 | 24817 | 59929 |
| 169 | [symbol] | Q14162 | 0.00000173 | 15143 | 8611 | 14177 | 7862 |
| 170 | MED-1 | Q18648 | 0.00000808 | 45159 | 36818 | 44762 | 34247 |
| 171 | OMD | Q980183 | 0.00000835 | 8784 | 14333 | 6431 | 12642 |
| 172 | [symbol] | Q80731 | 0.00000849 | 957 | 4608 | 888 | 3423 |
| 173 | DLL1 | O00548 | 0.00000864 | 1645 | 2578 | 1628 | 2222 |
| 174 | IL-2, sRe | P01589 | 0.00000943 | 2488 | 34187 | 634 | 10897 |
| 175 | MMP-7 | P09231 | 0.00000967 | 1916 | 4838 | 1841 | 3617 |
| 176 | [symbol] CAM-1 | P06362 | 0.00000984 | 4213 | 7312 | 4412 | 2057 |
| 177 | RAC1 | P63000 | 0.0000 [symbol] | 48434 | 21753 | 41684 | 15010 |
| 178 | [symbol] | P16721 | 0.0000106 | 10866 | 7868 | 10382 | 7867 |
| 179 | [symbol] | P00747 | 0.0000106 | 3607 | 2744 | 3536 | 3845 |
| 180 | P-16 | P02778 | 0.000011 | 2891 | 10124 | 2125 | 7806 |
| 181 | BMP10 | Q95393 | 0.000012 | 1816 | 3443 | 1872 | 2834 |
| 182 | [symbol] | P94165 | 0.0000129 | 446 | 2423 | 243 | 1386 |
| 183 | PAPP-A | Q13219 | 0.0000132 | 5206 | 18359 | 4283 | 14258 |
| 184 | AM328 | Q82688 | 0.0000136 | 111 | 245 | 129 | 203 |
| 185 | BGH3 | Q15682 | 0.0000136 | 26543 | 40693 | 26954 | 35002 |
| 186 | CD325 | Q15762 | 0.0000139 | 2807 | 1248 | 2062 | 1123 |
| 187 | URB | Q768M96 | 0.0000142 | 1835 | 2927 | 1698 | 2484 |
| 188 | [symbol] | Q9NYA1 | 0.0000154 | 18429 | 5492 | 16524 | 3129 |
| 189 | [symbol] | Q07829 | 0.0000161 | 900 | 2374 | 868 | 1085 |
| 190 | [symbol] | [symbol] | 0.0000162 | 9210 | 2167 | 3016 | 1882 |
| 191 | [symbol] | O95388 | 0.0000172 | 10990 | 7919 | 10578 | 7268 |
| 192 | [symbol] | P01736 | 0.0000219 | 248 | 489 | 232 | 365 |
| 193 | DKK1 | O94907 | 0.0000238 | 35702 | 21314 | 31923 | 18407 |
| 194 | [symbol] | P49862 | 0.0000241 | 1498 | 989 | 1444 | [symbol] |
| 195 | IL-16 | Q14006 | 0.000026 | [symbol] | 1140 | 323 | 729 |
| 196 | MCC-1 | Q16627 | 0.0000261 | 7769 | 11880 | 7744 | 11224 |
| 197 | PDESA | O78074 | 0.0000263 | 30593 | 8271 | 10990 | 6262 |
| 198 | IL-16_Ra | Q13261 | 0.0000265 | 1007 | 3088 | 948 | 2026 |
| 199 | DL14 | Q9NR63 | 0.0000265 | [symbol] | 1254 | 925 | 1193 |
| 200 | IAG [symbol] | P78504 | 0.000027 | 454 | 580 | 423 | 559 |
| 201 | BMFR1A | P30894 | 0.0000282 | 608 | 878 | 572 | 286 |
| 202 | EDA | Q92838 | 0.0000285 | 3087 | 852 | 3053 | 320 |
| 203 | [symbol] MOC1 | Q9M4F8 | 0.0000286 | 2798 | 4538 | 2685 | 3970 |
| 204 | EGF | P01138 | 0.0000288 | 1474 | 893 | 1343 | 811 |
| 205 | [symbol] | Q18591 | 0.0000304 | 4913 | 7756 | 4481 | 7187 |
| 206 | CO39 | P49981 | 0.0000312 | 1861 | 3596 | 1721 | 2608 |
| 207 | IL-10_ [symbol] | Q08334 | 0.0000322 | 1266 | 1750 | 1246 | 1548 |
| 208 | BANK | Q9Y6Q6 | 0.0000332 | 919 | 2287 | 868 | 1697 |
| 209 | [symbol] | Q [symbol] | 0.0000359 | 483 | 1189 | 439 | 638 |
| 210 | [symbol] | P05067 | 0.0000378 | 57979 | 35364 | 48651 | 30862 |
| 211 | [symbol] RNP_A2_B1 | P23626 | 0.000018 | 11788 | 51483 | 11337 | 28985 |
| 212 | PNB_beta | P31781 | 0.0000303 | 40022 | 18970 | 42966 | 11730 |
| 213 | [symbol] | P05684 | 0.0000406 | 20141 | 10958 | 18841 | 9409 |
| 214 | [symbol] | P20783 | 0.0000 [symbol] | 103 | 142 | 84 | 129 |
| 215 | JAK2 | Q60574 | 0.0000467 | 86246 | 105173 | 94972 | 107132 |
| 216 | METAP1 | [symbol] | 0.0000473 | 11854 | 5154 | 11815 | 3628 |
| 217 | UPM1 | P63980 | 0.0000 [symbol] | 33274 | 17440 | 34214 | 14138 |
| 218 | EPI | Q14944 | 0.0000503 | 2358 | 395 | 1972 | 876 |
| 219 | MMP-12 | P39500 | 0.0000512 | 1529 | 6366 | 1445 | 3905 |
| 220 | [symbol] | P07585 | 0.0000525 | 4385 | 8278 | 3945 | 5598 |
| 221 | LEAP-1 | P81372 | 0.0000534 | 12002 | 24042 | 10443 | 23785 |
| 222 | [symbol] | [symbol] | 0.0000538 | 4248 | 18696 | 3815 | 10033 |
| 223 | [symbol] | O43599 | 0.0000653 | 138 | 222 | 120 | 178 |
| 224 | ASGR1 | P07305 | 0.0000738 | 3907 | 11296 | 3804 | 9348 |
| 225 | YES | P67947 | 0.0000742 | 1472 | 9249 | 1364 | 3871 |
| 226 | [symbol] CAM-2 | P13598 | 0.0000747 | 1452 | 3676 | 1431 | 2962 |
| 227 | [symbol] | P36405 | 0.0000749 | 89685 | 24068 | 52592 | 12930 |
| 228 | TFF3 | Q07654 | 0.0000781 | 8836 | 13729 | 6083 | 12314 |
| 229 | [symbol] | P00741 | 0.0000803 | 23506 | 18922 | 23829 | 19823 |
| 230 | LRIG3 | [symbol] | 0.0000834 | 3315 | 8653 | 3314 | 5063 |
| 231 | [symbol] | P36888 | 0.0000868 | 1437 | 16433 | 1253 | 4541 |
| 232 | SP-D | P35247 | 0.0000874 | 34806 | 86912 | 34008 | 85092 |
| 233 | [symbol] | [symbol] | 0.0000946 | 2516 | 1587 | 2285 | 1393 |
| 234 | [symbol] | P41240 | 0.0000953 | 24146 | 11134 | 22395 | 2681 |
| 235 | MP2K4 | P45986 | 0.000102533 | 37781 | 26031 | 37634 | 25321 |
| 236 | N8T4 | Q [symbol] | 0.000103807 | 703 | 1197 | 668 | 897 |
| 237 | MIP-6 | Q18863 | 0.000106434 | 4979 | 8304 | 3939 | 6372 |
| 238 | FLR13 | [symbol] | 0.000112325 | 6403 | 8436 | 5236 | 7311 |
| 239 | MSP_4 [symbol] | P26858 | 0.000138362 | 1294 | 520 | 1112 | 336 |
| 240 | DAN | P41271 | 0.000114184 | 4024 | 10871 | 3285 | 6792 |
| 241 | CLC7A | [symbol] | 0.000117696 | 425 | 731 | 390 | 688 |
| 242 | Factor_D | P00746 | 0.000122723 | 382 | 123 | 658 | 705 |
| 243 | [symbol] | [symbol] | 0.000128207 | 703 | 4585 | 514 | 2692 |
| 244 | [symbol] | P29317 | 0.000130162 | 2598 | 6302 | 2457 | 6724 |
| 245 | [symbol] | P07384 | 0.000136365 | 45722 | 24715 | 44974 | 36307 |
| | | (P04832) | | | | | |
| 246 | [symbol] _Reception | [symbol] | 0.000145431 | 1440 | 2735 | 1283 | 2028 |
| 247 | POGFRA | P16234 | 0.000148032 | 3616 | 6459 | 3050 | 4785 |
| 248 | [symbol] | P00736 | 0.000162419 | 148 | 308 | 139 | 183 |
| 249 | [symbol] | P01298 | 0.000163207 | 2475 | 8735 | 2035 | 3323 |
| 250 | NLGNX | Q8N0W4 | 0.000169802 | 884 | 2851 | 823 | 1708 |
| 251 | PKC-G | P05129 | 0.000170371 | 238 | 392 | 228 | 316 |
| 252 | [symbol] | Q15197 | 0.000170187 | 1879 | 2773 | 1289 | 2193 |
| 253 | FCN2 | Q16485 | 0.000175439 | 802 | 1148 | 740 | 979 |
| 254 | EFNB2 | P82799 | 0.000177482 | 18212 | 24069 | 17890 | 22587 |
| 255 | IL-23_R | [symbol] | 0.00017987 | 2636 | 21977 | 2143 | 7781 |
| 256 | [symbol] | [symbol] | 0.000181993 | 6886 | 17662 | 4956 | 18020 |
| 257 | P-Solestin | P35305 | 0.000182083 | 31798 | 20915 | 27516 | 18423 |
| 258 | RACS | P60763 | 0.000153679 | 10416 | 5151 | 30462 | 3402 |
| 259 | [symbol] | O00182 | 0.000180248 | 1736 | 4238 | 1507 | 3118 |
| 260 | IMOH1 | P20835 | 0.000200976 | 3608 | 13261 | 5511 | 10092 |
| 261 | CCL28 | [symbol] | 0.000203074 | 14225 | 9334 | 13774 | 8826 |
| 262 | HRG | P04196 | 0.000204481 | 4104 | 3026 | 4197 | 2825 |
| 263 | [symbol] | O43927 | 0.000205393 | 1896 | 9083 | 1715 | 6585 |
| 264 | TECK | O15444 | 0.000213908 | 1519 | 3149 | 1352 | 2251 |
| 265 | [symbol] | P04070 | 0.000216450 | 1465 | [symbol] | 1482 | 1377 |
| 266 | [symbol] | P42574 | 0.000217056 | 83841 | 37407 | 64719 | 26365 |
| 267 | [symbol] | P20158 | 0.000219035 | 2248 | 3341 | 2303 | 2028 |
| 268 | Thrombin | P00734 | 0.000225439 | 4229 | 1450 | 2232 | 1036 |
| 269 | [symbol] | Q92583 | 0.000226735 | 802 | 11807 | 793 | 3746 |
| 270 | [symbol] | P00734 | 0.000232728 | 115835 | 191592 | 115827 | 100680 |
| 271 | [symbol] | P08681 | 0.000247161 | 6258 | 8647 | 5173 | 7387 |
| 272 | Transferrin | P02787 | 0.000250308 | 176989 | 145356 | 175151 | 188119 |
| 273 | SAP | P02743 | 0.000258713 | 30132 | 22049 | 28875 | 19678 |
| 274 | [symbol] | Q01361 | 0.000269179 | 398 | 1179 | 314 | 891 |
| 275 | [symbol] -4H | Q15056 | 0.000261365 | [symbol] | 59880 | 101339 | 48231 |
| 276 | [symbol] | P06398 | 0.000262882 | 1200 | 801 | 1097 | 685 |
| 277 | IBFEF-3 | P17936 | 0.000263904 | 2137 | 1684 | 2049 | 1645 |
| 278 | MK12 | P63778 | 0.000276489 | 1688 | 871 | 1420 | 666 |
| 279 | CYIF | O76096 | 0.000286389 | 1198 | 8103 | 1327 | 4268 |
| 280 | [symbol] ECTM1 | Q8WVN6 | 0.000291828 | 1144 | 1678 | 1067 | 1382 |
| 281 | RET | P07949 | 0.000293789 | 1352 | 965 | 1288 | 852 |
| 282 | [symbol] | P29353 | 0.000297374 | 48524 | 28302 | 49967 | 26221 |
| 283 | Trypsin_2 | P02478 | 0.000304986 | 2658 | 8224 | 2459 | 8486 |
| 284 | GOF2 | [symbol] | 0.000313398 | 1488 | 733 | 836 | 650 |
| 285 | [symbol] | P40189 | 0.000333147 | 6965 | 8755 | 6809 | 8123 |
| 286 | PTF-1C | P29350 | 0.000334392 | 22137 | 12283 | 18360 | 6364 |
| 287 | [symbol] | O43485 | 0.000336157 | 6081 | 2402 | 5117 | 1694 |
| 288 | [symbol] | P01009 | 0.000341517 | 600 | 781 | 683 | 731 |
| 289 | [symbol] | P14543 | 0.000354619 | 5676 | 7655 | 5279 | 7193 |
| 290 | DSC2 | Q02487 | 0.000357972 | 2600 | 3553 | 2368 | 3125 |
| 291 | PBEF | P43490 | 0.000360018 | 1770 | 4142 | 1679 | 2461 |
| 292 | ABMEL | Q49AH0 | 0.000632722 | 2963 | 2018 | 2809 | 1934 |
| 293 | [symbol] | Q9BD89 | 0.000316215 | 1930 | 3251 | 1823 | 2713 |
| 294 | INRA | P43489 | 0.000319437 | 867 | 2958 | 415 | 1404 |
| 295 | BAFF | Q9Y275 | 0.000382088 | 971 | 3244 | 856 | 2603 |
| 296 | INF_ [symbol] | P15260 | 0.000383076 | 943 | 1635 | 868 | 1234 |
| 297 | PRKACA | P17612 | 0.000412431 | 65365 | 31021 | 60173 | 21468 |
| 298 | [symbol] | P16403 | 0.000420845 | 6256 | 17180 | 5654 | 8423 |
| 299 | [symbol] | Q01638 | 0.000462752 | 3042 | 21278 | 2787 | 8185 |
| 300 | UCBP | P05161 | 0.000470284 | 8829 | 23715 | 5965 | 13767 |
| 301 | O5K21 | P65400 | 0.000476703 | 196 | 315 | 181 | 235 |
| 302 | FABP | P05413 | 0.000512089 | 14812 | 32494 | 12494 | 21796 |
| 303 | [symbol] | Q [symbol] | 0.000545428 | 870 | 1156 | 837 | 1056 |
| 304 | CNOP1 | Q [symbol] | 0.000549319 | 5262 | 3948 | 5303 | 3895 |
| 305 | [symbol] | Q03403 | 0.000573756 | 18269 | 26349 | 16612 | 23287 |
| 306 | [symbol] | P05164 | 0.000592045 | 26557 | 43557 | 23069 | 37229 |
| 307 | HXK2 | P52789 | 0.000601449 | 2126 | 6433 | 2013 | 3172 |
| 308 | CSM | P00ML2 | 0.000605524 | 3839 | 2613 | 1707 | 2101 |
| | | (P00ML3) | | | | | |
| 309 | PLXO1 | O60486 | 0.000868548 | 862 | 872 | 689 | 813 |
| 310 | ADAMTS-4 | Q75173 | 0.005619156 | 94 | 142 | 90 | 105 |
| 311 | [symbol] | P03973 | 0.000623518 | 22419 | 28395 | 21865 | 25692 |
| 312 | IL-34 | Q6ZM14 | 0.000623742 | 538 | 658 | 593 | 650 |
| 313 | BMP- [symbol] | Q13873 | 0.000642785 | 685 | 859 | 645 | 794 |
| 314 | [symbol] | Q92843 | 0.005645162 | 884 | 543 | 740 | 504 |
| 315 | PFO5 | Q93471 | 0.005684572 | 1462 | 4785 | 3305 | 1987 |
| 316 | HSP_ [symbol] | P08238 | 0.000713794 | 67070 | 110003 | 84016 | 90903 |
| 317 | ADAM_9 | Q13443 | 0.000719795 | 1607 | 2216 | 1410 | 1830 |
| 318 | [symbol] | Q09685 | 0.000743081 | 8371 | 13890 | 8136 | 10689 |
| 319 | BOMA | Q02233 | 0.006745871 | 8341 | 6502 | 8238 | 6595 |
| 320 | DAPK2 | Q [symbol] | 0.000745644 | 8637 | 3794 | 6989 | 2527 |
| 321 | C7 | P10643 | 0.000775785 | 1097 | 1351 | 1060 | 1397 |
| 322 | [symbol] | P48357 | 0.00032689 | 2348 | 3790 | 2227 | 2976 |
| 323 | IL-178 | Q9OHF3 | 0.006802968 | 73 | 112 | 69 | 84 |
| 324 | [symbol] | P83104 | 0.000605237 | 173257 | 127102 | 183783 | 111485 |
| 325 | [symbol] | P37252 | 0.000825271 | 16084 | 8724 | 13577 | 6743 |
| 326 | CgA | P10645 | 0.000842921 | 4842 | 12449 | 3212 | 7808 |
| 327 | IL-12_RB2 | Q99665 | 0.000652688 | 985 | 1253 | 615 | 794 |
| 328 | BMP-14 | P43026 | 0.006872297 | 2752 | 1625 | 2240 | 1368 |
| 329 | PTN | P21245 | 0.000875108 | 517 | 728 | 457 | 539 |
| 330 | PYHrP | P12272 | 0.000899598 | 541 | 4839 | 538 | 1901 |
| 331 | [symbol] | O95861 | 0.0006971475 | 1879 | 2360 | 1738 | 2285 |
| 332 | [symbol] | P61247 | 0.00035404 | 442 | 2093 | 375 | 381 |
| 333 | [symbol] | P12644 | 0.000091485 | 2590 | 8146 | 2734 | 4052 |
| 334 | [symbol] | P07900 | 0.001098707 | 17780 | 49289 | 15909 | 22574 |
| | | (P08238) | | | | | |
| 335 | 0520_PKB [symbol] | P31789 | 0.001113929 | 14614 | 7840 | 14368 | 4781 |
| | | (P31751/ | | | | | |
| | | Q8Y242 | | | | | |
| 336 | [symbol] | P37802 | 0.001160122 | 13816 | 7561 | 32380 | 6069 |
| 337 | PTF-18 | P18931 | 0.001192604 | 4073 | 2400 | 3960 | 1796 |
| 338 | ES8P4 | Q8GZN4 | 0.003254113 | 3181 | 1718 | 1197 | 1642 |
| 339 | [symbol] | Q89729 | 0.001265875 | 1921 | 12125 | 1763 | 4597 |
| 340 | SEM65 | Q9H3T3 | 0.001276838 | 6313 | 8372 | 6344 | 7380 |
| 341 | CHK1 | Q34757 | 0.003276091 | 299 | 986 | 251 | 422 |
| 342 | PC6K9 | Q6N8P7 | 0.001295953 | 428 | 565 | 415 | 513 |
| 343 | DAF | P08174 | 0.001313836 | 16424 | 19351 | 14454 | 17853 |
| 344 | TCTP | P [symbol] | 0.0013524 | 34426 | 20523 | 32456 | 15439 |
| 345 | HGF | P14216 | 0.001365618 | 712 | 1623 | 730 | 1054 |
| 346 | [symbol] | P09658 | 0.001392496 | 1517 | 2496 | 1353 | 2050 |
| 347 | [symbol] | P62258 | 0.001428972 | 3685 | 6514 | 3560 | 4282 |
| 348 | [symbol] | Q1449S | 0.051465912 | 1389 | 6450 | 1325 | 2711 |
| 349 | PK3CG | P48438 | 0.001872296 | 2181 | [symbol] | 2002 | 1269 |
| 350 | [symbol] | P17183 | 0.001476238 | 3630 | 5313 | 3516 | 4382 |
| 351 | KPCT | Q84753 | 0.001497368 | 4875 | 2237 | 3493 | 1530 |
| 352 | RS3 | P23396 | 0.001624799 | 448 | 1756 | 284 | 675 |
| 353 | [symbol] | P30041 | 0.001585019 | 621 | 372 | 567 | 381 |
| 354 | [symbol] | P27930 | 0.00171678 | 10879 | 18407 | 10434 | 13041 |
| 355 | CROL1 | Q [symbol] | 0.001878486 | 1990 | 2474 | 1846 | 2362 |
| 356 | [symbol] | PA7992 | 0.00185063 | 233 | 308 | 220 | 264 |
| 357 | [symbol] | Q8N125 | 0.001681921 | 3338 | 4011 | 3267 | 4017 |
| 358 | Trypsin | P07377 | 0.001890437 | 7004 | 16675 | 7275 | 8607 |
| 359 | [symbol] | P18586 | 0.001937987 | 3708 | 9620 | 3565 | 4226 |
| 360 | BMF-γ | P18075 | 0.001060934 | 683 | 427 | 840 | 365 |
| 361 | IL-8 | P10345 | 0.001873709 | 25823 | 5954 | 13977 | 1643 |
| 362 | SMAD3 | P84622 | 0.001583072 | 28589 | 17603 | 27522 | 13271 |
| 363 | CSRP3 | P50461 | 0.002010377 | 3815 | 1618 | 2456 | 1451 |
| 364 | [symbol] | P22749 | 0.002084425 | 2293 | 12649 | 2116 | 3788 |
| 365 | PESC | O00541 | 0.002175429 | 185 | 684 | 165 | 281 |
| 366 | HSP_76 | P0DMV8 | 0.002198822 | 11145 | 24644 | 1357 | 14313 |
| 367 | [symbol] | P62306 | 0.002317412 | 1795 | 4056 | 1648 | 2267 |
| 368 | Growth_hormone_receptor | P10312 | 0.002228453 | 1491 | 1131 | 1338 | 1070 |
| 369 | IL_1_R_AcP | Q9NPH3 | 0.002308033 | 23819 | 20307 | 24042 | 20787 |
| 370 | [symbol] EPR | Q12884 | 0.002328784 | 900 | 731 | 842 | 692 |
| 371 | IMBB2 | P12258 | 0.002391131 | 3130 | 6197 | 2862 | 3629 |
| 372 | [symbol] | P04179 | 0.002474346 | 34062 | 28765 | 31992 | 28967 |
| 373 | [symbol] | P25774 | 0.00253037 | 1112 | 1702 | 3130 | 1456 |
| 374 | SIG14 | Q06812 | 0.002688335 | 10803 | 19449 | 8839 | 20505 |
| 375 | IL-32_Bb3 | P42701 | 0.002521541 | 341 | 473 | 328 | 358 |
| 376 | LCK | P06238 | 0.002639534 | 471 | 1692 | 448 | 676 |
| 377 | Contactin-4 | Q8IWY2 | 0.002548618 | 3668 | 3117 | 3863 | 3105 |
| 378 | LKHA8 | P03960 | 0.00266779 | 7623 | 37743 | 4035 | 4828 |
| 379 | BOC | Q3BWVS | 0.002653581 | 1335 | 1224 | 1306 | 1678 |
| 380 | COMMDT | Q86VX2 | 0.002710768 | 9313 | 4799 | 7798 | 2906 |
| 381 | RBF | P02763 | 0.002718522 | 206 | 165 | 201 | 170 |
| 382 | BGN | P21810 | 0.002131636 | 18935 | 20412 | 16393 | 18980 |
| 383 | Fibropectin | P02761 | 0.002914316 | 31913 | 26945 | 10935 | 17226 |
| 384 | Ephrin-A2 | Q43921 | 0.002938931 | 869 | 1028 | 803 | 986 |
| 385 | GREM1 | O60865 | 0.002990713 | 289 | 625 | 267 | 348 |
| 386 | DRR1 | O95990 | 0.003027104 | 180 | 158 | 154 | 375 |
| 387 | [symbol] | P46378 | 0.003041802 | 2608 | 7521 | 2391 | 3506 |
| 388 | [symbol] | P62081 | 0.0003064287 | 1381 | 9254 | 1182 | 2367 |
| 389 | P3A2 | P29785 | 0.003083563 | 477 | 904 | 436 | 879 |
| 390 | SPARCL1 | Q14516 | 0.003137142 | 10300 | 13820 | 10420 | 13392 |
| 391 | [symbol] | Q [symbol] | 0.00323362 | 22494 | 17873 | 20105 | 17000 |
| 392 | PD63A | Q14432 | 0.00326166 | 1861 | 1316 | 1659 | 757 |
| 393 | MCP-4 | Q93516 | 0.003245407 | 280 | 643 | 237 | 365 |
| 394 | ENPP7 | Q6UWV6 | 0.00327762 | 6874 | 16056 | 5597 | 9068 |
| 395 | [symbol] | P10141 | 0.003305714 | 704 | 1420 | 689 | 1052 |
| 396 | MFGM | Q08431 | 0.003312438 | 961 | 1571 | 302 | 1175 |
| 397 | [symbol] | P31717 | 0.003393308 | 23367 | 26649 | 23119 | 26402 |
| 398 | [symbol] | P35690 | 0.003638067 | 8988 | [symbol] | 7068 | 8003 |
| 399 | [symbol] | Q9Y258 | 0.003565977 | 1511 | 2763 | 1268 | 1949 |
| 400 | DC-SIGNR | Q9M2X3 | 0.003567063 | 39794 | 44136 | 39133 | 43786 |
| 401 | [symbol] | P01374 | 0.003602968 | 1293 | 1912 | 1291 | 1365 |
| | | (Q06643) | | | | | |
| 402 | [symbol] | P23528 | 0.003659837 | 13718 | 8453 | 11378 | 6506 |
| 403 | [symbol] | P26401 | 0.00367898 | 82128 | 56936 | 32487 | 43488 |
| 404 | NSE | P09104 | 0.003681778 | 60649 | 37098 | 60482 | 26833 |
| 405 | [symbol] | Q99706 | 0.003236144 | 854 | 7418 | 651 | 2362 |
| 406 | [symbol] | Q18674 | 0.003835276 | 2329 | 3803 | 2145 | 2657 |
| 407 | SAA | [symbol] | 0.003896364 | 1335 | 9182 | 328 | 1384 |
| 408 | [symbol] | P0509 | 0.003816835 | 561 | 458 | 544 | 416 |
| 409 | [symbol] | Q75636 | 0.003917419 | 349 | 450 | 326 | 438 |
| 410 | SMAD2 | Q16796 | 0.003943018 | 21173 | 10710 | 18480 | 6413 |
| 411 | [symbol] | P62892 | 0.004059609 | 442 | 1804 | 428 | 1915 |
| 412 | CSF-1 | P08503 | 0.0040852 | 4135 | 8042 | 3577 | 4640 |
| 413 | SMAA | P54980 | 0.004128864 | 12605 | 7618 | 11407 | 4089 |
| 414 | AT533 | Q78 [symbol] | 0.004439458 | 3928 | 3145 | 3826 | 3196 |
| 415 | SHP-2 | Q68124 | 0.004513806 | 37265 | 23858 | 30338 | 12030 |
| 416 | PPa [symbol] | Q15181 | 0.004673612 | 30475 | 46571 | 30399 | 32985 |
| 417 | Protein_ [symbol] _A3 | P80103 | 0.004780658 | 7525 | 4963 | 8751 | 4030 |
| 418 | [symbol] _MD-2_complex | O50206 | 0.00492834 | 3585 | 4666 | 3785 | 4272 |
| | | (Q9Y8Y9) | | | | | |
| 419 | [symbol] | P08697 | 0.004744612 | 1607 | 1300 | 1342 | 1265 |
| 420 | RGM [symbol] | Q6NW40 | 0.004987725 | 2065 | 2524 | 1945 | 2266 |
| 421 | BCI2-I8e_1_protein | Q67817 | 0.006024294 | 3635 | 1896 | 2000 | 1439 |
| 422 | BDNF | P23560 | 0.005092632 | 1126 | 767 | 1035 | 524 |
| 423 | HDGH2 | Q724V5 | 0.006093068 | 418 | 941 | 380 | 811 |
| 424 | Sorting [symbol] | O96218 | 0.005093384 | 20694 | 10682 | 17820 | 6185 |
| 425 | B7-2 | P42081 | 0.00617096 | 1422 | 2364 | 1384 | 1785 |
| 426 | OHC2 | Q9M1K4 | 0.005239078 | 237 | 298 | 224 | 252 |
| 427 | 5H21A | O60830 | 0.006243165 | 8103 | 16833 | 4064 | 5854 |
| 428 | PAI-1 | P05123 | 0.00529658 | 2428 | 1717 | 2265 | 1267 |
| 429 | [symbol] | P28992 | 0.005359933 | 7636 | 6366 | 7514 | 5826 |
| 430 | [symbol] | P10144 | 0.006841363 | 132 | 212 | 102 | 133 |
| 431 | [symbol] | Q07011 | 0.005674188 | 272 | 1263 | 263 | 584 |
| 432 | HMG-1 | P05429 | 0.005876313 | 3765 | 7223 | 3382 | 4893 |
| 433 | REPO2 | Q [symbol] | 0.005683838 | 194 | 872 | 171 | 324 |
| 434 | COX-2 | P35364 | 0.005814634 | 454 | 712 | 386 | 562 |
| 435 | [symbol] | P11632 | 0.005816061 | 653 | 851 | 612 | 729 |
| 436 | [symbol] _isomerase | P60174 | 0.008046932 | 24309 | 15841 | 24198 | 9712 |
| 437 | MIP_3a | P76566 | 0.00606173 | 350 | 362 | 134 | 187 |
| 438 | TBP | P20226 | 0.006085849 | 600 | 3113 | 608 | 1058 |
| 439 | MIG | Q07326 | 0.006185356 | 1132 | 3831 | 468 | 2337 |
| 440 | NID2 | Q34132 | 0.006227373 | 2383 | 2969 | 2373 | 2704 |
| 441 | [symbol] | Q13642 | 0.008242365 | 157 | 218 | 145 | 376 |
| 442 | EIF-6A-1 | P63241 | 0.006292143 | 38152 | 36532 | 37969 | 18910 |
| 443 | [symbol] CAM-3 | P32942 | 0.006411249 | 649 | 942 | 612 | 593 |
| 444 | NCC27 | Q00295 | 0.006445978 | 8325 | 4653 | 7958 | 2875 |
| 445 | [symbol] | P12956 | 0.00658031 | 1112 | 5468 | 872 | 1802 |
| 446 | BMON3 | P05114 | 0.005618386 | 478 | 595 | 443 | 577 |
| 447 | SDF-1 | P48061 | 0.006868329 | 4761 | 5947 | 4692 | 5300 |
| 448 | IGF-1 | P06019 | 0.006374876 | 1150 | 957 | 1030 | 972 |
| 449 | VEGF_ [symbol] | P36968 | 0.006904128 | 6507 | 6725 | 6200 | 8736 |
| 450 | Chymase | P23946 | 0.005951732 | 932 | 4865 | 324 | 3513 |
| 451 | IL-20 | Q9NYY1 | 0.00710398 | 219 | 276 | 220 | 223 |
| 452 | αFRP-3 | Q92785 | 0.00719027 | 1075 | 1310 | 1056 | 1249 |
| 453 | PLCG1 | P19174 | 0.007191642 | 859 | 1771 | 725 | 936 |
| 454 | IL-1-sRI | P14778 | 0.0073368 | 10261 | 11879 | 9065 | 11080 |
| 455 | PIGR | P01833 | 0.007425376 | 2385 | 5044 | 2199 | 3465 |
| 456 | H2A3 | Q7L7L0 | 0.007498931 | 797 | 1532 | 539 | 938 |
| 457 | B7 | P33681 | 0.00760046 | 599 | 767 | 534 | 672 |
| 458 | CKAP2 | Q8WWK9 | 0.007580358 | 2611 | 18109 | 23408 | 15384 |
| 459 | ARI3A | Q99856 | 0.007833606 | 1773 | 1084 | 1844 | 768 |
| 460 | IL-22BP | Q96915 | 0.00785657 | 3068 | 6195 | 2779 | 3730 |
| 461 | Factor_I | P06365 | 0.008163322 | 2821 | 31630 | 27976 | 31322 |
| 462 | PAFAM_beta_ [symbol] | P98402 | 0.00819764 | 11104 | 7572 | 10934 | 8894 |
| 463 | [symbol] | P06412 | 0.005243514 | 4036 | 5501 | 3684 | 4527 |
| 464 | MK01 | P28482 | 0.008275633 | 6702 | 3870 | 6141 | 2795 |
| 465 | NPS-PLA2 | P34555 | 0.008307397 | 1673 | 7671 | 1589 | 3342 |
| 466 | [symbol] | [symbol] | 0.008520351 | 4532 | 5337 | 8495 | 5206 |
| 467 | KPC1 | P41743 | 0.008526498 | 6506 | 3242 | 5072 | 1862 |
| 468 | Cathepsin_A | P30619 | 0.008523099 | 10536 | 8258 | 10747 | 7613 |
| 469 | CTACK | Q8V4X3 | 0.008385941 | 795 | 329 | 702 | 826 |
| 470 | IL-30 | P22301 | 0.008076447 | 154 | 151 | 108 | 128 |
| 471 | PDGF-Rb | P03519 | 0.009088795 | 7707 | 15730 | 4954 | 10997 |
| 472 | RSPO3 | Q9BXY4 | 0.009147983 | 1714 | 2129 | 1512 | 2033 |
| 473 | MMP-10 | P09238 | 0.009205119 | 3182 | 4794 | 2894 | 3714 |
| 474 | TCPTP | P17706 | 0.009539443 | 239 | 342 | 227 | 245 |
| 475 | MTRA2 | O43464 | 0.009851527 | 6335 | 13343 | 5636 | 7679 |
| 476 | [symbol] | P01374 | | | | | |
| | | (Q06643) | 0.00958734 | 109 | 333 | 202 | 199 |
| 477 | EG-VEGF | P58284 | 0.008652765 | 136 | 293 | 128 | 159 |
| 478 | Protease_ [symbol] | P07093 | 0.009671343 | 1288 | 3398 | 1120 | 1651 |
| 479 | [symbol] | Q1497A | 0.009928434 | 13642 | 50268 | 76368 | 37454 |
| 480 | ANP | P01160 | 0.003936602 | 326 | 494 | 256 | 379 |
| 481 | FGF-12 | P01328 | 0.009980395 | 186 | 418 | 183 | 235 |
| 482 | PSMA | Q04809 | 0.00899171 | 843 | 1914 | 587 | 831 |
| 483 | [symbol] | P03950 | 0.010126884 | 2519 | 2078 | 2427 | 2891 |
| 484 | Ep [symbol] | P21709 | 0.010126959 | 24486 | 28675 | 25106 | 27443 |
| 485 | [symbol] | P02758 | 0.010175612 | 7798 | 8596 | 7569 | 8760 |
| 486 | LOH-B_1 | P07195 | 0.010276101 | 1440 | 3958 | 1313 | 1933 |
| 487 | Thymidine_kinase | P04183 | 0.010403125 | 414 | 5623 | 368 | 1929 |
| 488 | FETUB | [symbol] | 0.01048642 | 4717 | 3721 | 4785 | 3304 |
| 489 | CBK5 | P45973 | 0.010486889 | 301 | 535 | 256 | 309 |
| 490 | VEGF-D | O43935 | 0.10592915 | 758 | 618 | 672 | 383 |
| 491 | Phosphoglycarate_mutase_1 | P18669 | 0.01063914 | 10278 | 3498 | 3488 | 207 |
| 492 | [symbol] | P63278 | 0.010680394 | 877 | 895 | 632 | 857 |
| 493 | HSP70, [symbol] | P11142 | 0.010727377 | 4224 | 8518 | 3648 | 4745 |
| 494 | Cystetin_ [symbol] | Q16828 | 0.010738429 | 6797 | 7390 | 5499 | 6518 |
| 495 | TGF- [symbol] | P03157 | 0.010843233 | 963 | 1445 | [symbol] | 948 |
| 496 | TAF1 | Q86 [symbol] | 0.011168375 | 8687 | 7086 | 7958 | 7012 |
| 497 | S100A8 | P06703 | 0.013174054 | 4078 | 2840 | 4004 | 2180 |
| 498 | ILR2 | O60603 | 0.11322568 | 1165 | 3196 | 1103 | [symbol] |
| 499 | [symbol] | Q9N2Q7 | 0.011406343 | 1357 | 2284 | 1023 | 1843 |
| 500 | SHP51 | P76324 | 0.012047092 | 2767 | 5348 | 521 | 6095 |
| 501 | [symbol] | P49236 | | | | | |
| | | (P24863 | 0.012340445 | 2072 | 1872 | 1896 | 3024 |
| 502 | [symbol] _Factor_V | P12259 | 0.012296098 | 27312 | 31620 | 27358 | 31907 |
| 503 | alpha-1- [symbol] _complex | P01011 | 0.012744812 | 12190 | 18778 | 11681 | 14118 |
| 504 | C2 | P08681 | 0.1262476 | 3378 | 4339 | 3028 | 3848 |
| 505 | PKC-Z | Q06513 | 0.13093847 | 783 | 8020 | 863 | 1649 |
| 506 | [symbol] | O | 0.013418425 | 183 | 216 | 178 | 191 |
| 507 | Ephrin-A5 | P62803 | 0.013637141 | 4334 | 4947 | 4328 | 4862 |
| 508 | [symbol] | P85010 | 0.013787186 | 4802 | 1958 | 2136 | 1280 |
| 509 | PSP | P56481 | 0.01390108 | 484 | 773 | 985 | 562 |
| 510 | [symbol] | P00787 | 0.013982907 | 688 | 1286 | 562 | 776 |
| 511 | [symbol] | P0CG48 | 0.014319808 | 3433 | 5952 | 3256 | 4472 |
| 512 | LYVE1 | Q9Y5Y7 | 0.014208095 | 127 | 168 | 129 | 363 |
| 513 | MOP-2 | P80075 | 0.014884421 | 389 | 169 | 128 | 158 |
| 514 | PDKK | Q00756 | 0.014794565 | 3226 | 4989 | 3215 | 3569 |
| 515 | H2B2E | Q16728 | 0.014946275 | 7584 | 17531 | 4962 | 5672 |
| 516 | MOZ | Q62794 | 0.01527425 | 3021 | 1376 | 935 | 1148 |
| 517 | RSF-50 | P10809 | 0.015331497 | 30562 | 48896 | 30569 | 29905 |
| 518 | WFK [symbol] | Q [symbol] | 0.15353153 | 4085 | 6597 | 3830 | 4853 |
| 519 | [symbol] | P40238 | 0.016501567 | 185 | 193 | 149 | 117 |
| 520 | [symbol] | Q9U8T3 | 0.015840084 | 12065 | 9281 | 10584 | 7817 |
| 521 | PPAC | P24686 | 0.036434399 | 5732 | 4453 | 4832 | 3250 |
| 522 | Protein_8 | P07225 | 0.018620993 | 3545 | 3289 | 3512 | 3249 |
| 523 | MMP-15 | P51512 | 0.016881358 | 694 | 1464 | 678 | 676 |
| 524 | PIAS4 | Q8NZW9 | 0.017658654 | 687 | 993 | 527 | 702 |
| 525 | [symbol] | P03951 | 0.017251556 | 1309 | 1165 | 1311 | 1181 |
| 526 | [symbol] | O43818 | 0.02791097 | 3191 | 1845 | 2509 | 1229 |
| | | (Q15750) | | | | | |
| 527 | GPDA | P21695 | 0.017908090 | 8965 | 3984 | 4853 | 2922 |
| 528 | GPO5 | P78333 | 0.018133409 | 790 | 462 | 807 | 420 |
| 529 | PVY | P10082 | 0.1842469 | 742 | 977 | 685 | 898 |
| 530 | SBDS | Q9Y3A5 | 0.018502784 | 9013 | 5064 | 7183 | 3172 |
| 531 | IL-13_ [symbol] | P78652 | 0.018505363 | 3133 | 3983 | 2649 | 3632 |
| 532 | [symbol] | P06702 | 0.015818687 | 3708 | 4259 | 3725 | 3553 |
| 533 | Carbonic_Anhydrose, IV | P22748 | 0.03901373 | 3340 | 2962 | 3237 | 2968 |
| 534 | ZAP [symbol] | P43403 | 0.018190832 | 8135 | 10356 | 8125 | 9677 |
| 535 | MAPKAPKI | Q16644 | 0.019576347 | 1609 | 1038 | 1431 | 738 |
| 536 | NCK1 | P15353 | 0.019612018 | 2183 | 1026 | 1623 | 842 |
| 537 | GPNMB | Q13656 | 0.039841325 | 3872 | 4203 | 3776 | 4286 |
| 538 | DYRN3 | O43781 | 0.020230463 | 1878 | 2135 | 1610 | 1813 |
| 539 | [symbol] | Q02413 | 0.020322197 | 3062 | 2643 | 2933 | 2788 |
| 540 | [symbol] | [symbol] | 0.020424485 | 1837 | 3278 | 1058 | 1383 |
| 541 | PA2G4 | Q9UQ80 | 0.020624369 | 4025 | 2586 | 3625 | 1670 |
| 542 | [symbol] | Q70626 | 0.020888378 | 585 | 1048 | 537 | 740 |
| 543 | MSP_R | Q04912 | 0.021691718 | 464 | 581 | 423 | 492 |
| 544 | ADAM12 | Q43184 | 0.021719882 | 675 | 1242 | 638 | 902 |
| 545 | RAD61 | Q05603 | 0.021881971 | 2951 | 2494 | 2623 | 2331 |
| 546 | HCC-$ | O35467 | 0.023889563 | 24475 | 29273 | 25341 | 27785 |
| 547 | CLCAK | Q [symbol] | 0.021934001 | 110 | 132 | 100 | 139 |
| 548 | IL-S [symbol] | Q01346 | 0.022156529 | 2749 | 3681 | 2420 | 3157 |
| 549 | [symbol] | P31947 | 0.022325356 | 931 | 1265 | 881 | 3050 |
| 550 | [symbol] | Q00538 | 0.00489023 | 11476 | 10431 | 11365 | 10232 |
| 551 | [symbol] | P34130 | 0.22713973 | 111 | 143 | 303 | 320 |
| 552 | 1235_ [symbol] | P02794 | 0.02272763 | 18677 | 31831 | 13178 | 23616 |
| | | (P02792) | | | | | |
| 553 | NACA | Q13786 | 0.023049798 | 3905 | 2395 | 2964 | 1527 |
| 554 | IL [symbol] | Q8N6P7 | 0.023068379 | 404 | 571 | 360 | 423 |
| 555 | DRAK2 | O94768 | 0.023676193 | 6905 | 1028 | 5769 | 6440 |
| 556 | [symbol] | Q96 [symbol] | 0.023838237 | 458 | 584 | 414 | 470 |
| 557 | ING1 | O9UK63 | 0.02386179 | [symbol] | 6135 | 7153 | 3258 |
| 558 | NAGK | Q9 [symbol] | 0.023960289 | 1276 | 1942 | 1167 | 1210 |
| 559 | [symbol] | P03013 | 0.024328408 | 4625 | 3990 | 4464 | 3960 |
| 560 | Activin_R18 | P36895 | 0.024387978 | 199 | 653 | 185 | 272 |
| 561 | [symbol] | Q14929 | 0.024396057 | 214 | 407 | 183 | 213 |
| 562 | UFD1 | Q9Y3C8 | 0.02447791 | 48742 | 35886 | 48088 | 30538 |
| 563 | SPINTZ | O43291 | 0.024501396 | 919 | 765 | 864 | 700 |
| 564 | INK2 | P45984 | 0.024508632 | 1914 | 1335 | 1904 | 1807 |
| 565 | ROBO2 | [symbol] | 0.024298531 | 3280 | 2672 | 2194 | 2489 |
| 566 | C3b | P1024 | 0.026506423 | 35265 | 38962 | 2283 | 7251 |
| 567 | [symbol] | Q15619 | 0.025822267 | 749 | 6974 | 632 | 548 |
| 568 | [symbol] | Q3Y6W8 | 0.025919203 | 549 | 972 | 467 | 527 |
| 569 | NEGR1 | Q72363 | 0.025965894 | 4059 | 3649 | 3885 | 3658 |
| 570 | [symbol] | P61088 | 0.026430692 | 13165 | 5767 | 11827 | 5107 |
| 571 | MAPX14 | Q15839 | 0.025633241 | 8200 | 3857 | 4978 | 2285 |
| 572 | ERAB | Q99714 | 0.027331688 | 1514 | 2688 | 1305 | 1309 |
| 573 | [symbol] | P48783 | 0.027681056 | 6729 | 13782 | 6809 | 6921 |
| 574 | [symbol] | P05053 | 0.027857005 | 3832 | 2429 | 1808 | 1987 |
| 575 | MSP | P36927 | 0.028174706 | 3489 | 4338 | 4083 | 4616 |
| 576 | TIG2 | Q99969 | 0.02824644 | 2420 | 2741 | 2365 | 2744 |
| 577 | B7-B2 | O7514 [symbol] | 0.025623336 | 9134 | 5346 | 5100 | 3391 |
| 578 | BID | P55967 | 0.028444858 | 4715 | 3097 | 4236 | 2454 |
| 579 | FCG2A | P32358 | 0.028466156 | 964 | 2029 | 74 | 878 |
| 580 | [symbol] | P0CDL4 | 0.025373719 | 322 | 737 | 191 | 205 |
| | | (P0CDL5) | | | | | |
| 581 | Noggin | Q13253 | 0.028134056 | 2209 | 1698 | 1953 | 1633 |
| 582 | contactin-1 | Q12860 | 0.029107973 | 286 | 242 | 264 | 212 |
| 583 | VEGF_sRT | P35316 | 0.029752607 | 6240 | 7118 | 6245 | 6525 |
| 584 | FCGR1 | P32314 | 0.028921756 | 230 | 343 | 180 | 230 |
| 585 | LIN78 | [symbol] | 0.02976771 | 329 | 485 | 311 | 316 |
| 586 | THAIL_R4 | Q9UBN6 | 0.029829654 | 798 | 1142 | 847 | 584 |
| 587 | COC37 | Q18543 | 0.029920679 | 375 | 707 | 333 | 354 |
| 588 | NRX3B | Q9HDB5 | 0.030227339 | 708 | 843 | 862 | 802 |
| 589 | IGFSP-1 | P08833 | 0.030483589 | 2103 | 2934 | 1819 | 2271 |
| 590 | [symbol] | Q16392 | 0.030499649 | 412 | 601 | 394 | 462 |
| 591 | Cadherin-5 | P33151 | 0.03110628 | 11234 | 13062 | 31562 | 12576 |
| 592 | CK-MM | P06732 | 0.031361237 | 908 | 645 | 896 | 425 |
| 593 | BCAM | P50895 | 0.031442102 | 1560 | 1793 | 1623 | 1629 |
| 594 | p27 [symbol] | P46527 | 0.031536569 | 1007 | 3786 | 753 | 1125 |
| 595 | DBNL | Q9 [symbol] | 0.031736481 | 4438 | 3442 | 3614 | 2889 |
| 596 | MATN3 | Q16232 | 0.032344613 | 370 | 451 | 341 | 372 |
| 597 | PCNA | P12008 | 0.032763494 | 128 | 167 | 124 | 129 |
| 598 | NMT1 | P39419 | 0.033306568 | 2975 | 1900 | 2239 | 1450 |
| 599 | [symbol] | P18957 | 0.033885412 | 10585 | 14658 | 9505 | 12030 |
| 600 | ATS16 | Q87668 | 0.034730945 | 235 | 277 | 220 | 239 |
| 601 | TIMP-2 | P16035 | 0.035646857 | 343 | 380 | 315 | 387 |
| 602 | PARK7 | Q99497 | 0.035730463 | 1024 | 2276 | 876 | 1120 |
| 603 | BRF-1 | Q92994 | 0.036041185 | 1680 | 2840 | 1267 | 1806 |
| 604 | PSP9-5 | P09336 | 0.036418003 | 2953 | 2175 | 2732 | 1589 |
| 605 | CYTT | P08228 | 0.036434765 | 16481 | 23656 | 15107 | 18173 |
| 606 | Activin, A | P08476 | 0.03676409 | 5575 | 7606 | 5451 | 5457 |
| 607 | [symbol] | Q92823 | 0.036783687 | 12953 | 16383 | 12365 | 14779 |
| 608 | MP2K2 | P36507 | 0.036847254 | 781 | 634 | 743 | 610 |
| 609 | GCP-2 | P80162 | 0.037187957 | 2235 | 3538 | 1570 | 1838 |
| 610 | [symbol] | P35813 | 0.37983595 | 406 | 528 | 323 | 427 |
| 611 | FGR | P09768 | 0.038362057 | 3543 | 876 | 984 | 445 |
| 612 | [symbol] | P48050 | 0.030591468 | 1443 | 1898 | 1356 | 1727 |
| 613 | IL-3 | P08700 | 0.03928097 | 983 | 1239 | 903 | 1037 |
| 614 | [symbol] | Q9Y4O5 | 0.038836127 | 135 | 251 | 170 | 190 |
| 615 | [symbol] | [symbol] | 0.039916476 | 5390 | 7998 | 4771 | 5858 |
| 616 | [symbol] | P54780 | 0.040585757 | 5254 | 8749 | 4345 | 5637 |
| 617 | [symbol] | P34836 | 0.041484643 | 248 | 363 | 284 | [symbol] |
| 618 | [symbol] | P01878 | 0.041716727 | 3473 | 4627 | 3368 | 3769 |
| | | (P01877) | | | | | |
| 619 | [symbol] | 901037 | 0.04270275 | 1075 | 1808 | 1001 | 1372 |
| 620 | FON1 | O00602 | 0.043132816 | 4295 | 6258 | 3986 | 4341 |
| 621 | MMP-2 | P05253 | 0.043563651 | 7079 | 5602 | 7089 | 5692 |
| 622 | [symbol] | P27348 | 0.0442 [symbol] | 1960 | 3068 | 1698 | 2078 |
| 623 | [symbol] | O00408 | 0.044629986 | 2239 | 2269 | 2691 | 1958 |
| 624 | [symbol] | Q29980 | 0.04862974 | 1523 | 15895 | 1453 | 4336 |
| 625 | [symbol] | P35831 | 0.045860583 | 354 | 695 | 364 | 541 |
| 626 | [symbol] | P62797 | [symbol] | 1469 | 1124 | 1896 | 1358 |
| 627 | [symbol] | P24532 | 0.046267489 | 457 | 510 | 445 | 494 |
| 628 | PAKS | Q9NQUS | 0.047364061 | 369 | 736 | [symbol] | 187 |
| 629 | [symbol] | Q14145 | 0.047727433 | 537 | 3411 | 561 | 984 |
| 630 | [symbol] | [symbol] | 0.0488606117 | 468 | 486 | 374 | |
| 631 | [symbol] | [symbol] | 0.048972054 | 689 | 1293 | 769 | 771 |

[symbol]

indicates data missing or illegible when filed

With respect to the proteins represented by Nos. 1, 2, 4, 5, 7, 9, 10, 11, 16, 17, 19, 20, 21, 22, 25, 28, 31, 32, 33, 37, 39, 41, 42, 43, 44, 46, 47, 48, 49, 52, 56, 57, 58, 59, 60, 61, 62, 63, 64, 65, 66, 67, 68, 70, 72, 73, 74, 75, 76, 77, 79, 80, 81, 82, 88, 89, 90, 91, 92, 93, 94, 95, 96, 97, 98, 99, 100, 101, 102, 103, 104, 106, 108, 109, 112, 113, 115, 118, 119, 120, 121, 123, 124, 125, 128, 129, 130, 131, 133, 134, 135, 139, 141, 142, 143, 144, 149, 154, 155, 156, 157, 159, 161, 164, 165, 167, 168, 171, 172, 173, 174, 175, 176, 180, 181, 182, 183, 184, 185, 187, 189, 192, 195, 196, 198, 199, 200, 201, 203, 205, 206, 207, 208, 209, 211, 214, 215, 219, 220, 221, 222, 223, 224, 225, 226, 228, 230, 231, 232, 236, 237, 238, 240, 241, 242, 243, 244, 246, 247, 248, 249, 250, 251, 252, 253, 254, 255, 256, 259, 260, 263, 264, 267, 269, 271, 274, 279, 280, 283, 285, 288, 289, 290, 291, 293, 294, 295, 296, 298, 299, 300, 301, 302, 303, 305, 306, 307, 309, 310, 311, 312, 313, 315, 316, 317, 318, 321, 322, 323, 326, 327, 329, 330, 331, 332, 333, 334, 338, 339, 340, 341, 342, 343, 345, 346, 347, 348, 350, 352, 354, 355, 356, 357, 358, 359, 364, 365, 366, 367, 371, 373, 374, 375, 376, 378, 379, 382, 383, 384, 385, 386, 387, 388, 389, 390, 393, 394, 395, 396, 397, 398, 399, 400, 401, 405, 406, 407, 409, 411, 412, 416, 418, 420, 423, 425, 426, 427, 430, 431, 432, 433, 434, 435, 437, 438, 439, 440, 441, 443, 445, 446, 447, 450, 451, 452, 453, 454, 455, 456, 457, 460, 461, 463, 465, 466, 469, 470, 471, 472, 473, 474, 475, 477, 478, 480, 481, 482, 483, 484, 486, 487, 489, 492, 493, 494, 495, 498, 499, 500, 502, 503, 504, 505, 506, 507, 509, 510, 511, 512, 513, 514, 515, 516, 518, 523, 524, 529, 531, 532, 534, 537, 538, 540, 542, 543, 544, 546, 547, 548, 549, 551, 552, 554, 555, 556, 558, 560, 561, 565, 566, 568, 572, 573, 574, 575, 576, 579, 580, 583, 584, 585, 586, 587, 588, 589, 590, 591, 593, 594, 596, 597, 599, 600, 601, 602, 603, 605, 606, 607, 609, 610, 612, 613, 614, 615, 616, 617, 618, 619, 620, 622, 624, 627, 628, 629 and 631 of Table 1, a significant increase was observed in the patient group compared to the carrier group.

Note that, Nectin-like protein 2 (UniProt ID: Q9BY67) represented by No. 62, was identical with protein of TSLC1, which is reported as a gene specifically expressed in leukemia cells taken from ATL patients, in Patent Literature 1.

In contrast, with respect to the proteins represented by Nos. 3, 6, 8, 12, 13, 14, 15, 18, 23, 24, 26, 27, 29, 30, 34, 35, 36, 38, 40, 45, 50, 51, 53, 54, 55, 69, 71, 78, 83, 84, 85, 86, 87, 105, 107, 110, 111, 114, 116, 117, 122, 126, 127, 132, 136, 137, 138, 140, 145, 146, 147, 148, 150, 151, 152, 153, 158, 160, 162, 163, 166, 169, 170, 177, 178, 179, 186, 188, 190, 191, 193, 194, 197, 202, 204, 210, 212, 213, 216, 217, 218, 227, 229, 233, 234, 235, 239, 245, 257, 258, 261, 262, 265, 266, 268, 270, 272, 273, 275, 276, 277, 278, 281, 282, 284, 286, 287, 292, 297, 304, 308, 314, 319, 320, 324, 325, 328, 335, 336, 337, 344, 349, 351, 353, 360, 361, 362, 363, 368, 369, 370, 372, 377, 380, 381, 391, 392, 402, 403, 404, 408, 410, 413, 414, 415, 417, 419, 421, 422, 424, 428, 429, 436, 442, 444 448, 449, 458, 459, 462, 464, 467, 468, 476, 479, 485, 488, 490, 491, 496, 497, 501, 508, 517, 519, 520, 521, 522, 525, 526, 527, 528, 530, 533, 535, 536, 539, 541, 545, 550, 553, 557, 559, 562, 563, 564, 567, 569, 570, 571, 577, 578, 581, 582, 592, 595, 598, 604, 608, 611, 621, 623, 625, 626 and 630, a significant decrease was observed in the patient group compared to the carrier group.

The p values in the increase or decrease in amount of the proteins represented by Nos. 1 to 333 were less than 0.001.

The p values in the increase or decrease in amount of the proteins represented by Nos. 334 to 482 were less than 0.01.

The p values in the increase or decrease in amount of the proteins represented by Nos. 483 to 631 were less than 0.05.

The proteins represented by Nos. 1 and 2 can be particularly useful as markers for discriminating between a carrier group and a patient group because the ranges of the protein amounts in the carrier group are not overlapped with those in the patient group.

TABLE 2

| | | | | Average value | Average value | Median value | Median value |
|---|---|---|---|---|---|---|---|
| Protein | | | | in patient | in remission | in patient | in remission |
| No. | Name of protein | UniProtID | p value | group | group | group | group |
| 1 | elF-4H | Q15056 | 2.96E-08 | 59880 | 13083 | 46231 | 13562 |
| 2 | Siglec-3 | P20138 | 3.59E-08 | 3341 | 1475 | 3028 | 1483 |
| 3 | HSP_90b | P08238 | 4.55E-08 | 110003 | 30490 | 90903 | 31374 |
| 4 | NXPHI | P58417 | 5.33E-08 | 3671 | 2321 | 3241 | 2346 |
| 5 | HIV-2_Rey | P18093 | 8.96E-08 | 127 | 84 | 116 | 81 |
| 6 | Ficolin-3 | Q75536 | 9.57E-08 | 450 | 200 | 434 | 186 |
| 7 | sL-Selecton | P14151 | 9.98E-08 | 14521 | 3099 | 13032 | 2569 |
| 8 | Coflin-1 | P23528 | 1.01E-07 | 8453 | 3073 | 6506 | 3055 |
| 9 | Nectin-like_protein_2 | Q9BY67 | 1.26E-07 | 21267 | 3690 | 17315 | 2917 |
| 10 | CD39 | P49961 | 1.43E-07 | 3096 | 1445 | 2608 | 1459 |
| 11 | CD30 | P8908 | 1.62E-07 | 23615 | 2379 | 18909 | 1907 |
| 12 | C3 | P01024 | 1.71E-07 | 56604 | 19848 | 50716 | 16918 |
| 13 | GRB2_adapter_protein | P62993 | 1.77E-07 | 73895 | 21534 | 68556 | 21878 |
| 14 | CD38 | P28907 | 1.79E-07 | 356 | 267 | 339 | 272 |
| 15 | EDAR | Q9UNE0 | 2.18E-07 | 1231 | 466 | 961 | 507 |
| 16 | GFBP-1 | P08833 | 3.16E-07 | 2934 | 636 | 2271 | 564 |
| 17 | Cyclophilin_A | P62937 | 3.90E-07 | 133620 | 57907 | 126677 | 52657 |
| 18 | [symbol] | P46962 | 3.94E-07 | 5435 | 2202 | 4427 | 1975 |
| 19 | GPC6 | Q9Y625 | 3.98E-07 | 2115 | 1001 | 1724 | 940 |
| 20 | LRP8 | Q14114 | 6.03E-07 | 7609 | 2669 | 6194 | 2840 |
| 21 | LAG-3 | P18627 | 6.23E-07 | 64829 | 15117 | 45759 | 11577 |
| 22 | Thromopoietin_Receptor | P40238 | 6.87E-07 | 133 | 73 | 117 | 70 |
| 23 | PAI-1 | P05121 | 6.92E-07 | 1719 | 527 | 1267 | 602 |
| 24 | AN326 | Q92688 | 7.36E-07 | 245 | 107 | 203 | 101 |
| 25 | sCD4 | P01730 | 7.77E-07 | 480 | 178 | 368 | 156 |
| 26 | Enterkinase | P960073 | 7.92E-07 | 182 | 88 | 139 | 92 |
| 27 | CONA1 | Q86Y22 | 9.09E-07 | 471 | 132 | 293 | 138 |
| 28 | TIMP-1 | P01033 | 9.17E-07 | 771 | 265 | 614 | 235 |
| 29 | NOP_kinase_B | P22392 | 9.29E-07 | 29297 | 8133 | 23692 | 7605 |
| 30 | TGF-b1 | P01137 | 9.94E-07 | 1448 | 444 | 948 | 435 |
| 31 | LY9 | Q9H6G7 | 1.07E-06 | 17254 | 8651 | 15104 | 8180 |
| 32 | XTP3A | Q9H773 | 1.14E-06 | 111104 | 33364 | 87965 | 31013 |
| 33 | IL-18_Rb | O96256 | 1.24E-06 | 217 | 124 | 199 | 127 |
| 34 | BFL1 | Q16548 | 1.27E-06 | 947 | 518 | 780 | 523 |
| 35 | NSE | P09104 | 1.37E-06 | 37098 | 10606 | 26833 | 10849 |
| 36 | Testican | Q08629 | 1.40E-06 | 3152 | 1418 | 2543 | 1310 |
| 37 | ENTP3 | O75355 | 1.69E-06 | 953 | 535 | 846 | 529 |
| 38 | MATK | P42679 | 1.72E-06 | 339 | 171 | 252 | 177 |
| 39 | RNF43 | Q68DV7 | 1.73E-06 | 1110 | 728 | 1007 | 749 |
| 40 | Histone_H1.2 | P16403 | 1.83E-06 | 17180 | 1568 | 8429 | 1808 |
| 41 | PGP9.5 | P09936 | 1.85E-06 | 2178 | 834 | 1589 | 748 |
| 42 | P-Selectin | P16109 | 2.05E-06 | 20915 | 10095 | 18423 | 9778 |
| 43 | CSF-1 | P09603 | 2.08E-06 | 6042 | 2321 | 4640 | 2000 |
| 44 | FGF9 | P31371 | 2.13E-06 | 181 | 110 | 160 | 109 |
| 45 | IL-34 | Q6CM14 | 2.23E-06 | 665 | 476 | 660 | 464 |
| 46 | HPG- | P15428 | 2.42E-06 | 1964 | 940 | 1692 | 929 |
| 47 | TIMP-3 | P35625 | 2.52E-06 | 2172 | 692 | 1604 | 607 |
| 48 | Adrenomedullin | P35318 | 2.55E-06 | 2294 | 1324 | 2099 | 1284 |
| 49 | Angio [symbol] -4 | Q9Y264 | 2.60E-06 | 341 | 154 | 260 | 150 |
| 50 | SHC1 | P29353 | 2.66E-06 | 28302 | 7342 | 20221 | 6450 |
| 51 | TCCR | Q6UWB1 | 2.99E-06 | 3906 | 1074 | 3121 | 1009 |
| 52 | TCTP | P13693 | 3.10E-06 | 20521 | 6155 | 16439 | 4493 |
| 53 | RP56KA3 | P61812 | 3.16E-06 | 6104 | 1236 | 4804 | 1342 |
| 54 | MDM2 | Q00987 | 3.27E-06 | 223 | 96 | 175 | 93 |
| 55 | G-CSF-R | Q99062 | 3.32E-06 | 519 | 255 | 406 | 268 |
| 56 | PBEF | P43490 | 3.32E-06 | 4142 | 868 | 2461 | 900 |
| 57 | IL-4 | P06112 | 3.37E-06 | 408 | 217 | 342 | 222 |
| 58 | PKC-A | P17252 | 3.46E-06 | 6724 | 1506 | 6743 | 1640 |
| 59 | DEAD-box_protein_198 | Q9UMR2 | 3.48E-06 | 7070 | 1438 | 4413 | 1557 |
| 60 | Lymphotactin | P47992 | 3.72E-06 | 308 | 178 | 264 | 177 |
| 61 | IFN-lambda_2 | Q81210 | 3.89E-06 | 142 | 74 | 111 | 77 |
| 62 | PKC-B-II | P05771 | 3.92E-06 | 1744 | 399 | 1034 | 417 |
| 63 | NCK1 | P16333 | 3.96E-06 | 1026 | 493 | 843 | 470 |
| 64 | CHST6 | Q9GZX3 | 3.96E-06 | 224 | 141 | 191 | 142 |
| 65 | ARP19 | P56211 | 4.09E-06 | 833 | 267 | 585 | 254 |
| 66 | IMB1 | Q14975 | 4.10E-06 | 50268 | 14696 | 37464 | 11995 |
| 67 | Alpha_ [symbol] | P06733 | 4.26E-06 | 51192 | 11533 | 28201 | 11010 |
| 68 | Caspase-3 | P42574 | 4.36E-06 | 37407 | 10553 | 26365 | 10791 |
| 69 | TMA | P07202 | 4.44E-06 | 1360 | 823 | 1176 | 808 |
| 70 | UFC1 | Q9V3CB | 4.51E-06 | 35585 | 15725 | 30638 | 14277 |
| 71 | Ephrin-A4 | P62798 | 4.51E-06 | 3341 | 1428 | 2746 | 1232 |
| 72 | CD22 | P20273 | 4.43E-06 | 754 | 383 | 611 | 379 |
| 73 | STAB2 | Q8WWQ8 | 4.68E-06 | 731 | 436 | 628 | 430 |
| 74 | PDEAD | Q08499 | 4.73E-06 | 1184 | 639 | 1048 | 670 |
| 75 | Mammaglobin_2 | O76558 | 4.75E-06 | 12774 | 8105 | 11130 | 7750 |
| 76 | 14-3-3_protein_beta_alpha | P31945 | 4.95E-06 | 38736 | 8225 | 24130 | 7536 |
| 77 | CLC4K | Q9U171 | 5.43E-06 | 132 | 86 | 119 | 84 |
| 78 | SHP-2 | Q06124 | 5.67E-06 | 21858 | 4273 | 12030 | 3825 |
| 79 | B7 | P33681 | 5.77E-06 | 767 | 441 | 672 | 441 |
| 80 | elF-5A-1 | P63241 | 5.84E-06 | 25632 | 7902 | 18910 | 6937 |
| 81 | IL-I_sR9 | Q9NP60 | 5.87E-06 | 772 | 510 | 691 | 520 |
| 82 | HDAC8 | Q9BY41 | 6.22E-06 | 2318 | 1105 | 1754 | 1121 |
| 83 | eldolase_A | P04075 | 7.11E-06 | 89056 | 40465 | 65816 | 38958 |
| 84 | METAP1 | P63582 | 7.41E-06 | 5154 | 979 | 3528 | 740 |
| 85 | HSP_60 | P10809 | 7.51E-06 | 48890 | 11351 | 29906 | 11533 |
| 86 | BARK1 | P25098 | 7.71E-06 | 16311 | 2693 | 9061 | 2509 |
| 87 | PDGF-AA | P04085 | 8.02E-06 | 10151 | 3571 | 8192 | 3666 |
| 88 | Angio [symbol] -2 | O15123 | 8.05E-06 | 208 | 116 | 188 | 104 |
| 89 | PPase | Q15181 | 8.12E-06 | 48571 | 16612 | 32965 | 15512 |
| 90 | ATS15 | Q8TES8 | 8.22E-06 | 277 | 170 | 239 | 179 |
| 91 | GFRa-3 | O60609 | 8.40E-06 | 818 | 344 | 591 | 302 |
| 92 | Lamin-B1 | P20700 | 8.81E-06 | 780 | 330 | 566 | 319 |
| 93 | PDGF-BB | P01127 | 8.84E-06 | 32971 | 1171 | 28252 | 9721 |
| 94 | IL-1Ra | P18610 | 8.93E-06 | 10397 | 4299 | 9693 | 4411 |
| 95 | IL-1F6 | Q9UHA7 | 8.96E-06 | 5750 | 839 | 2975 | 779 |
| 96 | Glutamate_carboxypeptidase | Q96KP4 | 9.00E-06 | 929 | 448 | 686 | 432 |
| 97 | AMPM2 | P50579 | 9.12E-06 | 32565 | 6707 | 18381 | 6599 |
| 98 | Transketolase | P29401 | 9.16E-06 | 56936 | 20967 | 43488 | 17581 |
| 99 | GV | P39877 | 9.22E-06 | 302 | 155 | 256 | 150 |
| 100 | PGF-20 | Q9NP95 | 9.47E-06 | 279 | 169 | 250 | 164 |
| 101 | UFM1 | P61960 | 9.90E-06 | 17440 | 5527 | 14129 | 4699 |
| 102 | C3a | P01024 | 1.04E-05 | 1196 | 226 | 746 | 180 |
| 103 | NET4 | Q9HB63 | 1.09E-05 | 1107 | 598 | 897 | 634 |
| 104 | Gro-b_g | P19876_P | 1.09E-05 | 874 | 291 | 690 | 288 |
| 105 | 14-3-3_protein_zeta_delta | P63104 | 1.10E-05 | 127102 | 43703 | 111440 | 43314 |
| 106 | EPO-R | P19236 | 1.10E-05 | 223 | 114 | 187 | 116 |
| 107 | TrkA | P04629 | 1.12E-05 | 2249 | 1038 | 1850 | 999 |
| 108 | PRKACA | P17612 | 1.15E-05 | 31021 | 7621 | 21468 | 6680 |
| 109 | amyloid_precursor_protein | P05067 | 1.26E-05 | 35364 | 16014 | 30562 | 14292 |
| 110 | RAC3 | P60763 | 1.26E-05 | 6251 | 1183 | 3402 | 1042 |
| 111 | hnRNP_A2__B1 | P22625 | 1.33E-05 | 51483 | 8566 | 28985 | 8638 |
| 112 | TGF_b3 | P10600 | 1.35E-05 | 434 | 218 | 365 | 227 |
| 113 | PST | P19883 | 1.38E-05 | 691 | 259 | 467 | 240 |
| 114 | DBNL | Q9UJU6 | 1.39E-05 | 3442 | 1779 | 2889 | 1685 |
| 115 | GFRa-1 | P56159 | 1.41E-05 | 729 | 333 | 599 | 297 |
| 116 | OCAD1 | Q9NX40 | 1.42E-05 | 1337 | 693 | 1095 | 690 |
| 117 | DRR1 | O95990 | 1.42E-05 | 186 | 136 | 176 | 141 |
| 118 | Ephrin-B3 | Q15768 | 1.44E-05 | 1004 | 356 | 660 | 358 |
| 119 | HSP_90a__b | P07900_P | 1.47E-05 | 49289 | 5564 | 22674 | 5920 |
| 120 | [symbol] loadhesin | Q98ZZZ | 1.48E-05 | 170 | 90 | 136 | 90 |
| 121 | TSLP | Q969O9 | 1.56E-05 | 328 | 146 | 258 | 137 |
| 122 | annexin_VI | P08133 | 1.58E-05 | 1373 | 587 | 923 | 596 |
| 123 | CBG | P08185 | 1.59E-05 | 456 | 234 | 356 | 229 |
| 124 | XPNPEP1 | Q9NQW7 | 1.61E-05 | 25362 | 4856 | 12767 | 4733 |
| 125 | CRK | P46108 | 1.66E-05 | 24824 | 1195 | 20254 | 12563 |
| 126 | Mcl-1 | Q07820 | 1.73E-05 | 2374 | 850 | 1685 | 879 |
| 127 | alCAM-2 | P13598 | 1.74E-05 | 3676 | 1130 | 2962 | 1005 |
| 128 | Carbonic_Anhydrase_X | Q9NS85 | 1.79E-05 | 457 | 361 | 442 | 352 |
| 129 | Thyroglobulin | P10266 | 1.84E-05 | 139 | 88 | 124 | 89 |
| 130 | PCSk7 | Q16549 | 1.94E-05 | 3683 | 1864 | 3483 | 2004 |
| 131 | ERK-1 | P27361 | 2.00E-05 | 34390 | 9384 | 22959 | 9550 |
| 132 | Triosephosphate_isomerase | P60174 | 2.01E-05 | 15441 | 4399 | 9712 | 3785 |
| 133 | MATN3 | Q5232 | 2.09E-05 | 461 | 273 | 372 | 274 |
| 134 | MBD4 | O96243 | 2.15E-05 | 345 | 163 | 267 | 163 |
| 135 | CKS21 | P68400 | 2.22E-05 | 376 | 147 | 239 | 140 |
| 136 | FGF-17 | O60258 | 2.35E-05 | 168 | 111 | 153 | 107 |
| 137 | RXFP1 | Q9HBX9 | 2.35E-05 | 236 | 167 | 213 | 166 |
| 138 | TBK1 | Q9UHD2 | 2.36E-05 | 197 | 106 | 154 | 107 |
| 139 | HPV_E7_Type_16 | P03129 | 2.39E-05 | 254 | 120 | 175 | 124 |
| 140 | IL-10 | P22301 | 2.47E-05 | 151 | 67 | 128 | 91 |
| 141 | SMAD3 | P84022 | 2.52E-05 | 17803 | 5179 | 12271 | 423 |
| 142 | IAM-C | Q9BX67 | 2.52E-05 | 6316 | 2031 | 4563 | 1677 |
| 143 | Bone_proteoglycan_II | P07565 | 2.53E-05 | 6278 | 3593 | 5598 | 3724 |
| 144 | Cathepsin_G | P081311 | 2.57E-05 | 973 | 365 | 626 | 303 |
| 145 | TNF_sR-II | P20333 | 2.60E-05 | 51875 | 18432 | 47932 | 15777 |
| 146 | RAC1 | P63000 | 2.62E-05 | 21753 | 5888 | 15050 | 6017 |
| 147 | PHI | P06744 | 2.63E-05 | 63563 | 24015 | 44995 | 21688 |
| 148 | SMOC1 | Q9H4F3 | 2.77E-05 | 4539 | 2496 | 3970 | 2412 |
| 149 | CSK | P41240 | 2.82E-05 | 11134 | 1923 | 7851 | 1774 |
| 150 | VEGF-C | P49767 | 2.84E-05 | 2166 | 884 | 1633 | 937 |
| 151 | MIF | P14174 | 2.89E-05 | 3241 | 1003 | 2060 | 976 |
| 152 | B7-H2 | Q75144 | 2.94E-05 | 5346 | 1634 | 3393 | 1551 |
| 153 | BDNF | P23560 | 2.95E-05 | 752 | 266 | 624 | 254 |
| 154 | AURKB | Q96GO4 | 2.98E-05 | 584 | 353 | 471 | 350 |
| 155 | Transgelin-2 | P37802 | 3.05E-05 | 7661 | 2090 | 5069 | 2121 |
| 156 | ATS1 | Q9UHI8 | 3.18E-05 | 313 | 122 | 232 | 120 |
| 157 | NK [symbol] | O95944 | 3.18E-05 | 216 | 134 | 191 | 123 |
| 158 | Glutathione_S-transerase_Pl | P09211 | 3.25E-05 | 12831 | 6421 | 9783 | 6163 |
| 159 | CATC | P53634 | 3.29E-05 | 506 | 335 | 447 | 344 |
| 160 | MFRP | Q9BY79 | 3.32E-05 | 1917 | 916 | 1459 | 692 |
| 161 | GHC2 | Q9H1K4 | 3.35E-05 | 296 | 174 | 252 | 172 |
| 162 | YES | P07947 | 3.36E-05 | 9249 | 1010 | 3871 | 992 |
| 163 | GPC2 | Q8N156 | 3.44E-05 | 177 | 88 | 138 | 91 |
| 164 | DLRB1 | Q9NP97 | 3.45E-05 | 9687 | 1977 | 6135 | 2121 |
| 165 | EF-1-beta | P24534 | 3.60E-05 | 1024 | 325 | 707 | 310 |
| 166 | CTAP-III | P02775 | 3.71E-05 | 15674 | 6308 | 12758 | 6534 |
| 167 | IL22RA1 | Q8N6P7 | 3.83E-05 | 571 | 259 | 429 | 264 |
| 168 | EPI | 0O14944 | 4.04E-05 | 995 | 575 | 876 | 566 |
| 169 | Rab_GDP_dissociation_ | P50395 | 4.05E-05 | 30401 | 11672 | 20375 | 11863 |
| | inhibitor_bate | | | | | | |
| 170 | PECAM-1 | P16284 | 4.07E-05 | 847 | 441 | 650 | 444 |
| 171 | MMP-17 | Q9UL29 | 4.12E-05 | 1506 | 581 | 1004 | 536 |
| 172 | PSA1 | P25786 | 4.19E-05 | 772 | 290 | 536 | 291 |
| 173 | MIP-35 | Q99732 | 4.40E-05 | 4608 | 1264 | 3413 | 1423 |
| 174 | CD226 | Q15762 | 5.01E-05 | 1249 | 738 | 1123 | 794 |
| 175 | c-Jun | P06412 | 5.02E-05 | 5501 | 2672 | 4527 | 2588 |
| 176 | BCL2-like_1_protein | Q07817 | 5.07E-05 | 1896 | 711 | 1439 | 682 |
| 177 | GSK-3_alpha_beta | P49840_P | 5.12E-05 | 10979 | 2332 | 6683 | 2263 |
| 178 | ULBP-3 | Q9BZM4 | 5.12E-05 | 803 | 314 | 573 | 326 |
| 179 | GOT1 | P17174 | 5.26E-05 | 7872 | 4161 | 6144 | 3972 |
| 180 | TSLP_R | Q9HC73 | 5.33E-05 | 1700 | 609 | 1164 | 582 |
| 181 | IL-17D | Q8TAD2 | 5.36E-05 | 336 | 114 | 224 | 117 |
| 182 | CLC1B | Q9P126 | 5.63E-05 | 9118 | 4299 | 8172 | 4159 |
| 183 | IL-17B | Q9UHF5 | 5.63E-05 | 112 | 63 | 84 | 62 |
| 184 | MRCKB | Q9Y5S2 | 5.75E-05 | 1473 | 730 | 1070 | 694 |
| 185 | IL-2_sRg | P31785 | 5.76E-05 | 5307 | 1509 | 3552 | 1299 |
| 186 | SPTA2 | Q13813 | 5.85E-05 | 146 | 105 | 128 | 103 |
| 187 | Aggrecan | P16112 | 5.95E-05 | 996 | 596 | 886 | 538 |
| 188 | HSP_27 | P04792 | 6.29E-05 | 3465 | 827 | 2040 | 703 |
| 189 | MDZ | Q92794 | 6.40E-05 | 1376 | 702 | 1149 | 643 |
| 190 | PDXK | O00764 | 6.43E-05 | 4989 | 1888 | 3569 | 1833 |
| 191 | C1QBP | Q07021 | 6.43E-05 | 4817 | 3571 | 4492 | 3716 |
| 192 | DHH | O43323 | 6.45E-05 | 1175 | 379 | 820 | 344 |
| 193 | PSA2 | P25787 | 6.50E-05 | 904 | 299 | 579 | 276 |
| 194 | FGF7 | P21781 | 6.57E-05 | 1620 | 468 | 1133 | 421 |
| 195 | PPIB | P23284 | 6.83E-05 | 2552 | 1271 | 2154 | 3162 |
| 196 | Macrophage_scavenger_ | P21757 | 6.84E-05 | 809 | 400 | 624 | 379 |
| | receptor | | | | | | |
| 197 | CKAP2 | Q8WWK9 | 6.84E-05 | 18109 | 10908 | 15394 | 11424 |
| 198 | LYVE1 | Q9Y5Y7 | 6.86E-05 | 158 | 96 | 141 | 95 |
| 199 | MSP_R | Q04912 | 6.88E-05 | 583 | 341 | 492 | 321 |
| 200 | CPNE1 | Q99829 | 6.91E-05 | 2978 | 731 | 1503 | 680 |
| 201 | Clusterin | P10909 | 6.93E-05 | 1021 | 682 | 968 | 661 |
| 202 | IFN-aA | P01563 | 7.04E-05 | 91 | 56 | 84 | 68 |
| 203 | annexin_II | P07356 | 7.06E-05 | 18696 | 4432 | 10033 | 4257 |
| 204 | CD97 | P48960 | 7.10E-05 | 1896 | 1284 | 1727 | 1299 |
| 205 | IMDH2 | P12268 | 7.14E-05 | 6197 | 1956 | 3629 | 1773 |
| 206 | ING1 | Q9UK53 | 7.15E-05 | 5136 | 1212 | 3255 | 972 |
| 207 | IL-1b | P01584 | 7.16E-05 | 1095 | 443 | 780 | 417 |
| 208 | IL-23_ | Q5VWK6 | 7.20E-05 | 21977 | 1145 | 7781 | 1140 |
| 209 | Cystatin_M | Q15828 | 7.23E-05 | 7390 | 4364 | 6518 | 4415 |
| 210 | Vascartive_Intestinal_ | P01282 | 7.24E-05 | 304 | 128 | 227 | 123 |
| | Peptide | | | | | | |
| 211 | MK08 | P46983 | 7.43E-05 | 2230 | 452 | 1343 | 464 |
| 212 | H31 | P68431 | 7.47E-05 | 48429 | 20030 | 32732 | 19264 |
| 213 | IF4G2 | P78344 | 7.64E-05 | 11532 | 1994 | 8048 | 1614 |
| 214 | TCPTP | P17706 | 7.72E-05 | 342 | 176 | 346 | 169 |
| 215 | CCL2B | Q9NR13 | 7.74E-05 | 9334 | 6421 | 8826 | 6391 |
| 216 | SIRT2 | Q [symbol] | 8.00E-05 | 17662 | 4953 | 10020 | 4635 |
| 217 | IL-15_Ra | Q13261 | 8.07E-05 | 3088 | 996 | 20258 | 838 |
| 218 | PAFAH_beta_subunit | P68402 | 8.21E-05 | 7572 | 3185 | 5894 | 2994 |
| 219 | Fk-3 | P36888 | 8.22E-05 | 10432 | 1394 | 4541 | 1624 |
| 220 | Thrombospondin-1 | P07996 | 8.23E-05 | 1710 | 534 | 1515 | 465 |
| 221 | PSME1 | Q06323 | 8.23E-05 | 1861 | 690 | 1053 | 632 |
| 222 | phosphoglycerate_ | P00658 | 8.43E-05 | 11726 | 4548 | 8020 | 4860 |
| | kinase_1 | | | | | | |
| 223 | BAD | Q92934 | 8.52E-05 | 10828 | 2798 | 7425 | 2349 |
| 224 | CDK-2 | P35354 | 8.58E-05 | 772 | 300 | 562 | 277 |
| 225 | CaMKK_alpha | QBN5S9 | 8.64E-05 | 441 | 184 | 319 | 167 |
| 226 | STK16 | O75716 | 8.73E-05 | 1680 | 1172 | 1574 | 1137 |
| 227 | KREM2 | Q8NCW0 | 8.90E-05 | 3922 | 1248 | 2678 | 1163 |
| 228 | SUMO3 | P55864 | 9.01E-05 | 8300 | 2138 | 5149 | 2084 |
| 229 | PSA6 | P60900 | 9.57E-05 | 1026 | 303 | 702 | 265 |
| 230 | Angiopoietin-1 | Q15389 | 9.68E-05 | 893 | 450 | 737 | 489 |
| 231 | CTACK | Q9Y4X3 | 9.99E-05 | 829 | 540 | 824 | 533 |
| 232 | AK1A1 | P14550 | 1.01E-04 | 9545 | 2562 | 6281 | 2493 |
| 233 | Dna1_homolog | QB6DA6 | 1.03E-04 | 426 | 177 | 323 | 182 |
| 234 | PTP-1C | P29350 | 1.04E-04 | 12283 | 1707 | 6354 | 1808 |
| 235 | FGF-16 | O43320 | 1.06E-04 | 3417 | 884 | 2295 | 776 |
| 236 | Cardiotrophin-1 | Q16619 | 1.06E-04 | 604 | 394 | 548 | 364 |
| 237 | slCAM-3 | P32942 | 1.06E-04 | 942 | 363 | 593 | 350 |
| 238 | IL-6_sRa | P08687 | 1.07E-04 | 24836 | 11859 | 19547 | 11268 |
| 239 | IL-3_Ra | P26951 | 1.08E-04 | 1297 | 452 | 860 | 368 |
| 240 | ALT | P24298 | 1.09E-04 | 5831 | 2523 | 4096 | 2663 |
| 241 | CAMK2D | Q13567 | 1.09E-04 | 8706 | 1861 | 4562 | 1675 |
| 242 | Cadherin-12 | P65289 | 1.10E-04 | 769 | 334 | 592 | 369 |
| 243 | Bcl-2 | P10415 | 1.12E-04 | 1336 | 464 | 918 | 432 |
| 244 | PFD6 | Q99471 | 1.12E-04 | 4785 | 905 | 1987 | 804 |
| 245 | RELT | Q969Z4 | 1.16E-04 | 5248 | 2166 | 4144 | 2010 |
| 246 | OSM | P13725 | 1.18E-04 | 1555 | 1092 | 1531 | 1049 |
| 247 | MEPE | Q9NQ76 | 1.19E-04 | 204 | 109 | 150 | 110 |
| 248 | EP16R | Q9UBC2 | 1.21E-04 | 1311 | 310 | 913 | 277 |
| 249 | elF-5 | P55010 | 1.22E-04 | 1958 | 743 | 1280 | 690 |
| 250 | Protein_disulfide_ | P30101 | 1.23E-04 | 4963 | 2664 | 4030 | 2587 |
| | isomerase_A3 | | | | | | |
| 251 | RUXF | P62306 | 1.25E-04 | 4056 | 1127 | 2267 | 1040 |
| 252 | CAMKID | Q8IU65 | 1.25E-04 | 1690 | 1044 | 1457 | 1050 |
| 253 | CBX5 | P45973 | 1.25E-04 | 435 | 231 | 309 | 222 |
| 254 | Ubiquitin | P62979 | 1.29E-04 | 1678 | 796 | 1284 | 724 |
| 255 | UCRP | P05161 | 1.30E-04 | 23775 | 4394 | 13767 | 3413 |
| 256 | IC3b | P01024 | 1.30E-04 | 27030 | 9848 | 14769 | 9797 |
| 257 | PA2G4 | Q9UQ80 | 1.32E-04 | 2596 | 773 | 1670 | 768 |
| 258 | HEMK2 | Q9Y5N5 | 1.32E-04 | 4709 | 1404 | 3132 | 1153 |
| 259 | NAP-2 | P02775 | 1.35E-04 | 5529 | 2290 | 4331 | 2361 |
| 260 | UBC9 | P63279 | 1.36E-04 | 7345 | 1982 | 3967 | 1435 |
| 261 | AGR2 | O95994 | 1.41E-04 | 610 | 441 | 542 | 432 |
| 262 | CYTF | O76096 | 1.43E-04 | 8103 | 785 | 78668 | 617 |
| 263 | Cyclophilin_F | P30405 | 1.45E-04 | 24068 | 2970 | 12930 | 2616 |
| 264 | PLK-1 | P63350 | 1.46E-04 | 153 | 80 | 119 | 79 |
| 265 | ARI3A | Q99855 | 1.46E-04 | 1094 | 306 | 758 | 255 |
| 266 | 14-3-3_protein_theta | 1P27348 | 1.47E-04 | 3068 | 896 | 2079 | 845 |
| 267 | MP2K2 | P36507 | 1.49E-04 | 634 | 426 | 610 | 432 |
| 268 | ENA-78 | P42830 | 1.49E-04 | 580 | 246 | 385 | 243 |
| 269 | IL-1_sRII | P27930 | 1.53E-04 | 18407 | 8592 | 13041 | 8103 |
| 270 | HSP70_protein_8 | P11142 | 1.55E-04 | 8518 | 1828 | 4745 | 1542 |
| 271 | SLAF6 | Q96DU3 | 1.58E-04 | 4686 | 756 | 2562 | 727 |
| 272 | PAFAH | Q18003 | 1.59E-04 | 591 | 381 | 581 | 363 |
| 273 | BCAR3 | O75816 | 1.61E-04 | 1527 | 858 | 1390 | 798 |
| 274 | EGF | P01133 | 1.61E-04 | 893 | 591 | 8511 | 556 |
| 275 | UBE2N | P61086 | 1.61E-04 | 8757 | 2473 | 5107 | 2212 |
| 276 | GDF-9 | O60383 | 1.62E-04 | 329 | 168 | 238 | 163 |
| 277 | Nucleoside_diphosphate_ | P15631 | 1.63E-04 | 695 | 387 | 643 | 342 |
| | kinase_A | | | | | | |
| 278 | ILT-2 | Q8NHL6 | 1.65E-04 | 6023 | 2832 | 5017 | 2401 |
| 279 | HNRPQ | O60506 | 1.67E-04 | 5279 | 1608 | 8119 | 1542 |
| 280 | DRG-1 | Q9NP79 | 1.69E-04 | 27691 | 7686 | 19676 | 6544 |
| 281 | LIN78 | Q9HAP6 | 1.69E-04 | 496 | 193 | 316 | 198 |
| 282 | SNAA | P54920 | 1.75E-04 | 7618 | 2726 | 4888 | 2472 |
| 283 | AIP | O00170 | 1.75E-04 | 5465 | 974 | 3117 | 796 |
| 284 | PDE11 | QBHCRB | 1.76E-04 | 1784 | 1119 | 1424 | 1063 |
| 285 | Carbonic_anhydrase_9 | Q16790 | 1.77E-04 | 611 | 295 | 438 | 307 |
| 286 | SP-D | P35247 | 1.77E-04 | 86912 | 26885 | 55092 | 24995 |
| 287 | SOD | P00441 | 1.83E-04 | 1588 | 461 | 920 | 444 |
| 288 | MEK1 | Q02750 | 1.84E-04 | 1293 | 395 | 771 | 367 |
| 289 | KPCl | P41743 | 1.85E-04 | 3242 | 765 | 1852 | 656 |
| 290 | PIGF | P49763 | 1.87E-04 | 435 | 302 | 413 | 304 |
| 291 | CAMK28 | Q13564 | 1.87E-04 | 3576 | 742 | 1920 | 632 |
| 292 | SBFS | Q9Y3A5 | 1.88E-04 | 5064 | 943 | 3172 | 704 |
| 293 | CFCl | P0CG37 | 1.89E-04 | 3180 | 1326 | 2424 | 1165 |
| 294 | BAFF_Receptor | Q96R13 | 1.89E-04 | 2167 | 1485 | 1882 | 1494 |
| 295 | PCGR1 | P12314 | 1.90E-04 | 343 | 157 | 230 | 137 |
| 296 | CD83 | Q01151 | 1.91E-04 | 1179 | 392 | 891 | 396 |
| 297 | Testican-2 | Q92563 | 1.92E-04 | 11507 | 657 | 3746 | 542 |
| 298 | HDGR2 | Q7Z4V5 | 1.93E-04 | 941 | 220 | 511 | 225 |
| 299 | DPP2 | Q9UHL4 | 1.95E-04 | 1929 | 1287 | 1714 | 1206 |
| 300 | ABL2 | P42684 | 1.96E-04 | 2048 | 1081 | 1632 | 917 |
| 301 | Stress-induced- | P31948 | 2.01E-04 | 8766 | 2910 | 5855 | 2341 |
| | phosphoprotein_1 | | | | | | |
| 302 | RS3 | P23396 | 2.03E-04 | 1755 | 213 | 675 | 232 |
| 303 | SKP1 | P63208 | 2.03E-04 | 3671 | 1836 | 2550 | 1211 |
| 304 | Plasmin | P00747 | 2.03E-04 | 542 | 311 | 425 | 277 |
| 305 | LAG-1 | Q8NHW4 | 2.04E-04 | 3351 | 1237 | 2575 | 1006 |
| 306 | Midkine | P21741 | 2.07E-04 | 627 | 351 | 485 | 358 |
| 307 | SCGF-alpha | Q9Y240 | 2.07E-04 | 9954 | 3101 | 7496 | 2249 |
| 308 | PGM1 | P36873 | 2.16E-04 | 627 | 281 | 449 | 273 |
| 309 | SCGF-beta | Q9Y240 | 2.19E-04 | 3514 | 1173 | 2575 | 906 |
| 310 | NCC27 | O00299 | 2.23E-04 | 4663 | 965 | 9675 | 907 |
| 311 | U82L3 | P68036 | 2.24E-04 | 2578 | 693 | 1712 | 590 |
| 312 | STAT1 | P42224 | 2.26E-04 | 9781 | 1842 | 6702 | 1784 |
| 313 | PP [symbol] | Q9UNP9 | 2.27E-04 | 3279 | 507 | 1181 | 544 |
| 314 | SH21A | O60880 | 2.28E-04 | 16833 | 3056 | 3884 | 2713 |
| 315 | AREG | P15614 | 2.31E-04 | 3762 | 1618 | 2548 | 935 |
| 316 | Myeloperoxidase | P05164 | 2.32E-04 | 43657 | 18226 | 37229 | 19275 |
| 317 | RSK-like_protein_kinase | O75582 | 2.32E-04 | 235 | 75 | 157 | 71 |
| 318 | MMEL2 | Q495T6 | 2.32E-04 | 1026 | 618 | 907 | 589 |
| 319 | BMP-14 | P43026 | 2.32E-04 | 1628 | 779 | 1358 | 778 |
| 320 | CAMK2A | Q9UQM7 | 2.36E-04 | 1607 | 350 | 903 | 317 |
| 321 | PF-4 | P02776 | 2.38E-04 | 2358 | 1240 | 1895 | 1305 |
| 322 | IL-12_RB2 | Q99665 | 2.39E-04 | 1201 | 517 | 794 | 551 |
| 323 | LRIGS | Q6UXM1 | 2.42E-04 | 6659 | 3486 | 5063 | 3755 |
| 324 | b-ECGF | P05230 | 2.42E-04 | 102 | 67 | 95 | 69 |
| 325 | MDHC | P40925 | 2.44E-04 | 32745 | 13421 | 20041 | 13117 |
| 326 | PLPP | Q96GD0 | 2.55E-04 | 5908 | 1387 | 3750 | 1198 |
| 327 | IFN-lambda_1 | Q8IU54 | 2.56E-04 | 406 | 199 | 321 | 183 |
| 328 | H6ST1 | O60243 | 2.56E-04 | 2200 | 1305 | 2094 | 1381 |
| 329 | STX1 [symbol] | Q16623 | 2.60E-04 | 567 | 197 | 324 | 186 |
| 330 | IL-16 | Q14005 | 2.66E-04 | 1140 | 399 | 729 | 374 |
| 331 | ERBB2 | P04525 | 2.70E-04 | 370 | 212 | 295 | 198 |
| 332 | KIF23 | Q02241 | 2.74E-04 | 1408 | 464 | 912 | 413 |
| 333 | Myoglobin | P02144 | 2.75E-04 | 2008 | 943 | 1610 | 924 |
| 334 | M2-PK | P14618 | 2.77E-04 | 31274 | 8626 | 19958 | 8412 |
| 335 | Keratin_1B | P05783 | 2.86E-04 | 874 | 425 | 682 | 381 |
| 336 | PLCg1 | P19174 | 2.97E-04 | 1771 | 499 | 936 | 487 |
| 337 | HMG-1 | P09429 | 2.97E-04 | 7223 | 2409 | 4693 | 2340 |
| 338 | HGF | P14210 | 2.99E-04 | 1623 | 484 | 1054 | 413 |
| 339 | ART | O00253 | 2.99E-04 | 3077 | 1305 | 2321 | 1143 |
| 340 | PAK3 | O75914 | 3.04E-04 | 544 | 422 | 514 | 424 |
| 341 | MAPK14 | Q16539 | 3.13E-04 | 3567 | 1211 | 2285 | 898 |
| 342 | MO2R1 | Q87D46 | 3.23E-04 | 1967 | 1178 | 1942 | 1106 |
| 343 | HO-2 | P30519 | 3.24E-04 | 726 | 155 | 418 | 161 |
| 344 | ON | P09486 | 3.32E-04 | 27995 | 14784 | 22916 | 16860 |
| 345 | Hatl | O14929 | 3.37E-04 | 407 | 95 | 213 | 96 |
| 346 | LCK | P06239 | 3.38E-04 | 1692 | 200 | 676 | 184 |
| 347 | CHK1 | O14757 | 3.42E-04 | 985 | 212 | 422 | 224 |
| 348 | FAK1 | Q05397 | 3.43E-04 | 281 | 193 | 237 | 187 |
| 349 | Semaphorin_3A | Q14563 | 3.43E-04 | 1019 | 436 | 756 | 427 |
| 350 | MIC-1 | Q99988 | 3.43E-04 | 4137 | 1674 | 3431 | 1197 |
| 351 | NMT1 | P30419 | 3.55E-04 | 1900 | 732 | 1450 | 734 |
| 352 | EPHA3 | P29320 | 3.63E-04 | 157 | 71 | 109 | 70 |
| 353 | CDC37 | Q16543 | 3.65E-04 | 707 | 149 | 364 | 142 |
| 354 | PKB_beta | P31751 | 3.70E-04 | 18970 | 5785 | 11739 | 6014 |
| 355 | D [symbol] | Q9UBT3 | 3.79E-04 | 9261 | 5803 | 7817 | 6017 |
| 356 | [symbol] | Q9N2K7 | 3.79E-04 | 195 | 110 | 155 | 116 |
| 357 | NSP_70 | PBDMV8 | 3.81E-04 | 24644 | 7537 | 14113 | 6000 |
| 358 | RBM39 | Q14498 | 3.88E-04 | 6450 | 716 | 2711 | 653 |
| 359 | [symbol] | Q92876 | 3.90E-04 | 2075 | 1400 | 1809 | 1391 |
| 360 | TSP2 | P35442 | 3.92E-04 | 76766 | 20845 | 64305 | 13654 |
| 361 | CHIP | QSUNET | 3.92E-04 | 5201 | 825 | 2685 | 715 |
| 362 | WNK3 | Q9BYP7 | 3.97E-04 | 3503 | 614 | 1513 | 568 |
| 363 | C3b | P01024 | 4.04E-04 | 38962 | 2363 | 7287 | 2307 |
| 364 | PESC | O00541 | 4.11E-04 | 654 | 108 | 281 | 111 |
| 365 | TNR4 | P43489 | 4.21E-04 | 2958 | 578 | 1404 | 522 |
| 366 | PKC-G | P05129 | 4.27E-04 | 392 | 248 | 316 | 257 |
| 367 | Moesin | P26038 | 4.27E-04 | 2600 | 744 | 1490 | 691 |
| 368 | GAPDH_liver | P04406 | 4.29E-04 | 82299 | 35464 | 58066 | 32269 |
| 369 | ACE2 | Q9BYF1 | 4.34E-04 | 515 | 258 | 391 | 269 |
| 370 | Osteocaloin | P02818 | 4.49E-04 | 2412 | 1094 | 1626 | 1148 |
| 371 | CD40_ligand_soluble | P29965 | 4.56E-04 | 331 | 151 | 262 | 128 |
| 372 | Tropomyisin_4 | P67936 | 4.58E-04 | 45569 | 12022 | 32924 | 6580 |
| 373 | PNB_a__b__8 | P31749_P | 4.64E-04 | 7840 | 2411 | 4781 | 2297 |
| 374 | DKK1 | O94907 | 4.71E-04 | 21314 | 11500 | 18407 | 11828 |
| 375 | FLRT1 | Q9NZU1 | 4.79E-04 | 953 | 515 | 767 | 564 |
| 376 | Lymophotoxin_a2__b1 | P01374_Q | 4.80E-04 | 1912 | 1030 | 1356 | 984 |
| 377 | IL-3 | P0870 | 4.83E-04 | 1239 | 719 | 1017 | 634 |
| 378 | Casoase-10 | Q92851 | 4.86E-04 | 4040 | 2705 | 3447 | 2667 |
| 379 | ANGL4 | Q9BY76 | 4.87E-04 | 3359 | 861 | 2019 | 730 |
| 380 | Cyclin_B1 | P14635 | 4.99E-04 | 363 | 178 | 279 | 178 |
| 381 | DLC8 | P63167 | 5.06E-04 | 394 | 162 | 248 | 161 |
| 382 | Lymphotoxin_b_R | P36941 | 5.08E-04 | 7039 | 5286 | 7098 | 4974 |
| 383 | PDE7A | Q13946 | 5.14E-04 | 395 | 221 | 309 | 205 |
| 384 | Notch_1 | P46531 | 5.17E-04 | 28986 | 17955 | 26793 | 16912 |
| 385 | Siglec-6 | O43699 | 5.19E-04 | 222 | 112 | 178 | 103 |
| 386 | MAPKAPK3 | Q16644 | 5.35E-04 | 1038 | 433 | 736 | 411 |
| 387 | RS3A | P61247 | 5.38E-04 | 2093 | 333 | 881 | 264 |
| 388 | PDE1A | P54750 | 5.42E-04 | 1328 | 466 | 886 | 407 |
| 389 | HXK2 | P62789 | 5.55E-04 | 5433 | 3773 | 3172 | 3622 |
| 390 | ERAB | Q99714 | 5.58E-04 | 2688 | 682 | 1309 | 585 |
| 391 | COMMD7 | Q86VX2 | 5.60E-04 | 47799 | 1763 | 2906 | 2068 |
| 392 | slCAM-5 | Q9UMF0 | 5.77E-04 | 800 | 536 | 743 | 556 |
| 393 | [symbol] | P11387 | 5.93E-04 | 525 | 255 | 332 | 251 |
| 394 | [symbol] | P62979 | 5.94E-04 | 5857 | 1389 | 2637 | 1306 |
| 395 | NSF1C | O9UN22 | 5.96E-04 | 6127 | 1516 | 3551 | 1246 |
| 396 | alpha-1-entichymotrypain_ | P01011 | 5.99E-04 | 18778 | 7309 | 14118 | 6532 |
| | complex | | | | | | |
| 397 | MPIF-1 | P55773 | 6.16E-04 | 4013 | 2261 | 3557 | 2287 |
| 398 | FGF-6 | P12034 | 6.26E-04 | 602 | 317 | 430 | 307 |
| 399 | SGTA | Q43765 | 6.32E-04 | 7163 | 2076 | 5363 | 1603 |
| 400 | ASGR1 | P07306 | 6.42E-04 | 11296 | 4701 | 9349 | 4114 |
| 401 | Esterase_D | P10768 | 6.48E-04 | 19092 | 8907 | 13720 | 8417 |
| 402 | TRAIL_R4 | Q9UBN6 | 6.52E-04 | 1142 | 681 | 854 | 732 |
| 403 | PTP-1B | P18031 | 6.53E-04 | 2400 | 1098 | 1796 | 1060 |
| 404 | HTRA2 | O43464 | 6.54E-04 | 13343 | 3836 | 7579 | 3563 |
| 405 | [symbol] FRP-3 | Q92765 | 6.55E-04 | 1310 | 974 | 1249 | 925 |
| 406 | Eotaxin-3 | Q9Y258 | 6.57E-04 | 2763 | 1243 | 1949 | 1224 |
| 407 | Stanniocalcin-1 | P52823 | 6.58E-04 | 3923 | 1944 | 2988 | 2050 |
| 408 | Calpain_1 | P07384_P | 6.69E-04 | 24715 | 11135 | 16307 | 9931 |
| 409 | HMGR | P04035 | 6.81E-04 | 220 | 145 | 191 | 137 |
| 410 | Gro-a | P09341 | 6.87E-04 | 6161 | 2142 | 4494 | 1842 |
| 411 | CLC7A | Q9BXN2 | 6.98E-04 | 731 | 355 | 588 | 297 |
| 412 | C3adesArg | P01024 | 7.08E-04 | 146004 | 88831 | 145867 | 77440 |
| 413 | GM-CSF | P04141 | 7.15E-04 | 349 | 187 | 255 | 162 |
| 414 | SEMSA | Q13593 | 7.38E-04 | 7795 | 4021 | 7187 | 3670 |
| 415 | 14-3-3 | P31946_P | 7.41E-04 | 8254 | 1381 | 4054 | 1184 |
| 416 | SPINTZ | O43291 | 7.52E-04 | 765 | 427 | 700 | 383 |
| 417 | STAT3 | P40753 | 7.67E-04 | 2801 | 714 | 1681 | 687 |
| 418 | IL-IF7 | Q9NZH [symbol] | 7.73E-04 | 2127 | 1398 | 1870 | 1233 |
| 419 | Carbonic_anhydrase_XIII | Q8NIQ1 | 7.83E-04 | 13975 | 4081 | 7746 | 2942 |
| 420 | DYRK3 | O43781 | 7.84E-04 | 2198 | 1324 | 1813 | 1331 |
| 421 | Cytochrome_P450_3A4 | P08684 | 7.88E-04 | 10956 | 6571 | 9409 | 6346 |
| 422 | POE3A | Q14432 | 8.04E-04 | 1118 | 433 | 757 | 339 |
| 423 | IMDH1 | P20839 | 8.06E-04 | 13261 | 4917 | 10092 | 4835 |
| 424 | GPVI | Q9HCNS | 8.16E-04 | 11840 | 4456 | 9825 | 3439 |
| 425 | PTHIP | P12272 | 8.18E-04 | 4819 | 497 | 1901 | 441 |
| 426 | IL-7 | P13232 | 8.24E-04 | 99 | 69 | 87 | 65 |
| 427 | Integrin_a1b1 | P56199_P | 8.52E-04 | 7443 | 993 | 2633 | 562 |
| 428 | DUS3 | P51452 | 8.61E-04 | 1696 | 598 | 1269 | 596 |
| 429 | sE-Selection | P16581 | 8.83E-04 | 70072 | 31853 | 61266 | 29209 |
| 430 | TNF_sR-1 | P19438 | 9.00E-04 | 2831 | 1317 | 2512 | 1270 |
| 431 | NAGK | Q9UJ70 | 9.08E-04 | 1942 | 803 | 1210 | 756 |
| 432 | Siglec-7 | Q9Y286 | 9.14E-04 | 1877 | 1069 | 1797 | 1001 |
| 433 | NLGNX | Q8N0W4 | 9.42E-04 | 2861 | 985 | 1708 | 839 |
| 434 | TACl | O14836 | 9.52E-04 | 1417 | 856 | 1164 | 774 |
| 435 | CNTF | P26441 | 9.54E-04 | 12 | 89 | 118 | 84 |
| 436 | M-CSF_R | P07333 | 9.56E-04 | 218 | 111 | 189 | 107 |
| 437 | Eph86 | O15197 | 9.60E-04 | 2773 | 1441 | 2193 | 1413 |
| 438 | Peroxiredoxin-1 | Q06830 | 9.81E-04 | 3767 | 1322 | 2136 | 1129 |
| 439 | ENPP7 | Q6UWV6 | 9.83E-04 | 16062 | 5012 | 9068 | 4893 |
| 440 | Azurocidin | P20160 | 9.89E-04 | 443 | 139 | 279 | 107 |
| 441 | MK01 | P28482 | 1.00E-03 | 3870 | 1379 | 2796 | 1379 |
| 442 | JAG1 | P78504 | 1.04E-03 | 880 | 448 | 659 | 370 |
| 443 | SRCN1 | P12931 | 1.06E-03 | 26542 | 6767 | 18881 | 4197 |
| 444 | hnRNP_A__B | Q99729 | 1.06E-03 | 12125 | 1735 | 4597 | 1657 |
| 445 | RAD51 | Q06609 | 1.10E-03 | 2494 | 1848 | 2331 | 1894 |
| 446 | ALCAM | Q13740 | 1.11E-03 | 21624 | 12230 | 19555 | 10577 |
| 447 | FCN1 | O00602 | 1.13E-03 | 5258 | 2935 | 4341 | 2821 |
| 448 | IL-10_Ra | Q13651 | 1.13E-03 | 1056 | 895 | 1043 | 911 |
| 449 | Tropomyosin_2 | P07951 | 1.15E-03 | 1534 | 1161 | 1439 | 1149 |
| 450 | Granzyme_H | P20718 | 1.15E-03 | 174 | 110 | 142 | 116 |
| 451 | C4b | P0C0L4_P | 1.18E-03 | 737 | 114 | 208 | 106 |
| 452 | IL-10_Ra | Q13478 | 1.18E-03 | 19642 | 9436 | 17346 | 9228 |
| 453 | Granzyme_B | P10144 | 1.20E-03 | 212 | 94 | 133 | 90 |
| 454 | SLAF5 | Q9UIB8 | 1.21E-03 | 17873 | 8768 | 17000 | 7090 |
| 455 | TNF-b | P01374 | 1.21E-03 | 532 | 230 | 357 | 227 |
| 456 | CAD15 | P55291 | 1.22E-03 | 9448 | 5457 | 8022 | 5208 |
| 457 | LKHA4 | P09960 | 1.23E-03 | 37743 | 4208 | 4828 | 1423 |
| 458 | H282E | Q16778 | 1.26E-03 | 17531 | 4074 | 5672 | 3695 |
| 459 | SE6L2 | QUXDD5 | 1.26E-03 | 1618 | 574 | 1022 | 421 |
| 460 | MAPK2 | P49137 | 1.27E-03 | 3772 | 1564 | 2774 | 1353 |
| 461 | EFNB1 | P98172 | 1.29E-03 | 2773 | 1447 | 2569 | 1160 |
| 462 | HSP_40 | P25685 | 1.32E-03 | 520 | 160 | 330 | 128 |
| 463 | NUDC3 | Q8IVO9 | 1.34E-03 | 1867 | 335 | 974 | 323 |
| 464 | LD78-beta | P16619 | 1.34E-03 | 3765 | 2105 | 3165 | 1939 |
| 465 | prostatic_binding_protein | P30086 | 1.35E-03 | 7591 | 3116 | 4769 | 3994 |
| 466 | RS7 | P62081 | 1.36E-03 | 9254 | 653 | 2357 | 611 |
| 467 | AIF1 | P55008 | 1.36E-03 | 4150 | 2397 | 3567 | 2647 |
| 468 | Kallistatin | P29622 | 1.39E-03 | 24304 | 29753 | 24164 | 29686 |
| 469 | GFRa-2 | O00451 | 1.40E-03 | 7646 | 3809 | 6745 | 314 |
| 470 | Survivin | O15392 | 1.42E-03 | 601 | 301 | 462 | 307 |
| 471 | MICA | Q29983 | 1.43E-03 | 583 | 156 | 495 | 76 |
| 472 | EphB4 | P54760 | 1.43E-03 | 6749 | 3991 | 6537 | 3705 |
| 473 | ICOS | Q9Y5W8 | 1.46E-03 | 972 | 347 | 527 | 304 |
| 474 | Periostin | Q15053 | 1.47E-03 | 2916 | 2043 | 2662 | 2010 |
| 475 | FYN | P06241 | 1.50E-03 | 11344 | 2955 | 7392 | 2450 |
| 476 | BASI | P35613 | 1.52E-03 | 528 | 326 | 427 | 323 |
| 477 | FCN2 | Q15485 | 1.52E-03 | 1148 | 681 | 979 | 608 |
| 478 | DC-SIGNB | Q9H2X3 | 1.55E-03 | 44138 | 36127 | 43786 | 36712 |
| 479 | RANTES | P13501 | 1.56E-03 | 27994 | 15643 | 22737 | 16877 |
| 480 | SPHK2 | Q9NRAC | 1.60E-03 | 583 | 322 | 469 | 308 |
| 481 | LDH-H_1 | P07195 | 1.63E-03 | 3965 | 802 | 1933 | 805 |
| 482 | ROBO3 | Q96MS0 | 1.65E-03 | 283 | 201 | 244 | 188 |
| 483 | a1-Antitrypsin | P01009 | 1.66E-03 | 781 | 546 | 731 | 547 |
| 484 | Ferritin | P02794 | 1.67E-03 | 31837 | 13854 | 23616 | 12080 |
| 485 | CRIS3 | P54108 | 1.68E-03 | 372 | 149 | 202 | 161 |
| 486 | GCP-2 | P80152 | 1.71E-03 | 3588 | 1305 | 1835 | 1324 |
| 487 | HPV_E&_Type18 | P06788 | 1.73E-03 | 1990 | 1276 | 1685 | 1164 |
| 488 | Galectin-2 | P05162 | 1.75E-03 | 915 | 627 | 816 | 466 |
| 489 | TIMD3 | Q8TDQ0 | 1.76E-03 | 18301 | 9012 | 17638 | 8435 |
| 490 | Protease_nextin_1 | P07003 | 1.77E-03 | 3398 | 802 | 1651 | 785 |
| 491 | MMP-7 | P09237 | 1.81E-03 | 4638 | 2509 | 3617 | 2311 |
| 492 | IL-1_R4 | Q0163S | 1.82E-03 | 21278 | 5080 | 8185 | 4171 |
| 493 | ZAP70 | P43403 | 1.84E-03 | 10366 | 6944 | 9677 | 6592 |
| 494 | SMAD2 | Q15796 | 1.85E-03 | 10710 | 2376 | 6413 | 1949 |
| 495 | FSTL1 | Q12841 | 1.86E-03 | 38461 | 22593 | 37095 | 20478 |
| 496 | IF4A3 | P38919 | 1.93E-03 | 828 | 316 | 419 | 294 |
| 497 | ERBB4 | Q15606 | 1.96E-03 | 113 | 65 | 93 | 68 |
| 498 | ETHE1 | O95571 | 1.99E-03 | 1672 | 1325 | 1656 | 1268 |
| 499 | DcR3 | O96407 | 1.99E-03 | 467 | 332 | 418 | 330 |
| 500 | B7-2 | P42081 | 2.00E-03 | 2364 | 1258 | 1786 | 1246 |
| 501 | CD48 | P09326 | 2.03E-03 | 1844 | 875 | 1745 | 740 |
| 502 | PIK3CA_PIK3R1 | P42336_P | 2.03E-03 | 2069 | 592 | 1032 | 507 |
| 503 | Granolysin | P22749 | 2.04E-03 | 12649 | 2272 | 3788 | 2388 |
| 504 | Cathepsin_B | P07858 | 2.09E-03 | 3185 | 1847 | 2765 | 1873 |
| 505 | PIAS4 | Q8N2W9 | 2.11E-03 | 993 | 518 | 702 | 437 |
| 506 | NACA | Q13765 | 2.13E-03 | 2395 | 971 | 1537 | 902 |
| 507 | FLRT3 | Q9NZU0 | 2.15E-03 | 8436 | 5049 | 7111 | 4875 |
| 508 | Neurotrophin-5 | P34130 | 2.28E-03 | 143 | 94 | 120 | 90 |
| 509 | FCRL3 | Q96P31 | 2.33E-03 | 783 | 322 | 550 | 334 |
| 510 | GP114 | Q8IZF4 | 2.34E-03 | 670 | 310 | 438 | 310 |
| 511 | Cathepsin_H | P09668 | 2.35E-03 | 2495 | 1567 | 2000 | 1560 |
| 512 | a2-Macroglobulin | P01023 | 2.37E-03 | 24438 | 12026 | 14058 | 10984 |
| 513 | VAV | P15498 | 2.40E-03 | 15041 | 3843 | 10974 | 3774 |
| 514 | ATP_synthase_beta_chain | P06576 | 2.41E-03 | 3430 | 963 | 1600 | 848 |
| 515 | MP2K3 | P46734 | 2.44E-03 | 1352 | 660 | 989 | 630 |
| 516 | [symbol] Leptin_R | P48357 | 2.45E-03 | 3790 | 1982 | 2976 | 1680 |
| 517 | tPA | P00750 | 2.47E-03 | 719 | 477 | 650 | 477 |
| 518 | CD59 | P13987 | 2.49E-03 | 2652 | 1564 | 2445 | 1349 |
| 519 | Ku70 | P12956 | 2.49E-03 | 5459 | 574 | 1802 | 539 |
| 520 | CSRP3 | P50461 | 2.53E-03 | 1618 | 966 | 1451 | 848 |
| 521 | H2A3 | Q7L7L0 | 2.62E-03 | 1632 | 684 | 918 | 674 |
| 522 | QORL1 | O95825 | 2.67E-03 | 654 | 291 | 401 | 286 |
| 523 | Contactin-5 | O94779 | 2.70E-03 | 388 | 297 | 370 | 312 |
| 524 | Chitotrio [symbol] | Q13231 | 2.73E-03 | 2109 | 1241 | 1826 | 1231 |
| 525 | PKC-D | Q05656 | 2.78E-03 | 6917 | 2479 | 4256 | 2469 |
| 526 | BPI | P17213 | 2.78E-03 | 9449 | 2776 | 7836 | 1254 |
| 527 | PDPK1 | O15530 | 2.80E-03 | 2712 | 503 | 1724 | 453 |
| 528 | GRN | P28799 | 2.83E-03 | 37334 | 23478 | 36614 | 26816 |
| 529 | [symbol] CD163 | Q86VB7 | 2.83E-03 | 7606 | 4160 | 7419 | 4233 |
| 530 | GI24 | Q9H7M9 | 2.87E-03 | 1050 | 380 | 637 | 332 |
| 531 | LY56 | O95711 | 2.98E-03 | 6045 | 4008 | 5154 | 3688 |
| 532 | RAP | P30533 | 2.98E-03 | 1031 | 663 | 799 | 635 |
| 533 | STAT5 | P42226 | 2.98E-03 | 1224 | 470 | 766 | 396 |
| 534 | HVEM | Q92956 | 3.03E-03 | 544 | 326 | 431 | 283 |
| 535 | RSPO2 | Q6UXX9 | 3.12E-03 | 672 | 142 | 324 | 144 |
| 536 | Karyophetin-a2 | P52292 | 3.13E-03 | 1804 | 390 | 1015 | 403 |
| 537 | BTK | Q06187 | 3.20E-03 | 6791 | 1573 | 3854 | 1261 |
| 538 | TNFSF15 | O96150 | 3.20E-03 | 7169 | 3063 | 6800 | 2181 |
| 539 | ADAM_9 | Q13443 | 3.33E-03 | 2216 | 1448 | 1830 | 1363 |
| 540 | GNS | P15566 | 3.35E-03 | 5820 | 3403 | 4225 | 2921 |
| 541 | FGFR4 | P22455 | 3.35E-03 | 1433 | 625 | 901 | 603 |
| 542 | MK11 | Q15759 | 3.36E-03 | 653 | 481 | 615 | 455 |
| 543 | PPID | Q06752 | 3.40E-03 | 3899 | 370 | 1938 | 288 |
| 544 | TAK1-TAB1 | O43318_Q | 3.45E-03 | 1845 | 582 | 1229 | 592 |
| 545 | aTie-2 | Q02763 | 3.50E-03 | 1901 | 1206 | 1733 | 1115 |
| 546 | DRAK2 | O94768 | 3.58E-03 | 10256 | 4278 | 6440 | 4235 |
| 547 | ALK-1 | P37023 | 3.67E-03 | 1645 | 689 | 1134 | 656 |
| 548 | Collectin_Kidney_1 | Q9BWP8 | 3.67E-03 | 5886 | 3496 | 4747 | 3258 |
| 549 | CAPG | P40121 | 3.69E-03 | 3656 | 1504 | 1999 | 1497 |
| 550 | LRRT3 | Q86VH5 | 3.72E-03 | 177 | 103 | 141 | 99 |
| 551 | FGF-6 | P10767 | 3.74E-03 | 663 | 144 | 322 | 143 |
| 552 | PDESA | O76074 | 3.83E-03 | 8278 | 3018 | 5262 | 2105 |
| 553 | TF | P13726 | 3.91E-03 | 1714 | 1094 | 1608 | 1032 |
| 554 | TBP | P20226 | 3.91E-03 | 3133 | 459 | 1068 | 607 |
| 555 | FGF-7 | O15520 | 4.92E-03 | 254 | 105 | 171 | 102 |
| 556 | CTGF | P29279 | 4.04E-03 | 1879 | 1620 | 1309 | 1051 |
| 557 | CDK8_cyclin_C | P49836_P | 4.06E-03 | 1372 | 810 | 1024 | 760 |
| 558 | CD53 | P08962 | 4.08E-03 | 813 | 390 | 548 | 358 |
| 559 | ADAM12 | O43184 | 4.09E-03 | 1242 | 523 | 902 | 494 |
| 560 | 6Ckine | O00585 | 4.19E-03 | 13890 | 8313 | 10667 | 7993 |
| 561 | Lymphotoxin_a1__b2 | P01374_Q | 4.20E-03 | 333 | 163 | 199 | 164 |
| 562 | EMR2 | Q9UMK3 | 4.29E-03 | 1159 | 511 | 838 | 396 |
| 563 | IAM-B | P57087 | 4.48E-03 | 7764 | 4661 | 6620 | 4293 |
| 564 | PAK6 | Q9NQUS | 4.50E-03 | 710 | 217 | 337 | 208 |
| 565 | EG-VEGF | P58294 | 4.55E-03 | 293 | 119 | 159 | 110 |
| 566 | IL-20 | Q9NYY1 | 4.56E-03 | 276 | 191 | 223 | 183 |
| 567 | Met | P08581 | 4.60E-03 | 8647 | 5342 | 7397 | 4852 |
| 568 | VCAM-1 | P19320 | 4.67E-03 | 22797 | 11821 | 21005 | 9359 |
| 569 | VEGF | P15592 | 4.68E-03 | 10202 | 7761 | 10088 | 8168 |
| 570 | [symbol] | Q9UKR3 | 4.81E-03 | 853 | 616 | 733 | 586 |
| 571 | PIANP | Q8IYJ0 | 4.93E-03 | 4886 | 3394 | 4654 | 3394 |
| 572 | SLIK1 | Q96PX8 | 5.00E-03 | 1475 | 867 | 1203 | 808 |
| 573 | IL24 | Q13007 | 5.21E-03 | 336 | 88 | 176 | 88 |
| 574 | Troponin_T | P45379 | 5.24E-03 | 7521 | 2500 | 3506 | 2069 |
| 575 | MCP-4 | Q99616 | 5.28E-03 | 843 | 278 | 365 | 204 |
| 576 | C5s | P01031 | 5.33E-03 | 8090 | 5894 | 8015 | 5625 |
| 577 | PolyUbiquitin_K48 | P0CG47 | 5.37E-03 | 2336 | 1333 | 1722 | 1274 |
| 578 | I-309 | P22362 | 5.51E-03 | 1543 | 1315 | 1550 | 1327 |
| 579 | Chymase | P23946 | 5.57E-03 | 4855 | 812 | 1513 | 814 |
| 580 | FSTL3 | O95633 | 5.60E-03 | 17397 | 9274 | 13552 | 9903 |
| 581 | kallikrein_5 | Q9Y337 | 5.69E-03 | 103 | 77 | 95 | 84 |
| 582 | IL-5_Ra | Q01344 | 5.73E-03 | 3651 | 1838 | 3157 | 1542 |
| 583 | IL-27 | Q8NEV9_ | 5.73E-03 | 567 | 453 | 531 | 477 |
| 584 | S100A4 | P26447 | 5.74E-03 | 5416 | 2364 | 2905 | 2220 |
| 585 | BMP10 | O95393 | 5.95E-03 | 3443 | 2034 | 2834 | 1896 |
| 586 | LEAP-1 | P81172 | 6.10E-03 | 24042 | 11085 | 23780 | 9498 |
| 587 | CK2-A1-B | P68400_P | 6.13E-03 | 2297 | 601 | 1067 | 560 |
| 588 | LYNB | P07946 | 6.15E-03 | 43072 | 17545 | 32330 | 13323 |
| 589 | FGF-12 | P61328 | 6.26E-03 | 416 | 182 | 236 | 183 |
| 590 | Cathepsin_S | P25774 | 6.33E-03 | 1707 | 956 | 1466 | 827 |
| 591 | AMGO2 | Q86512 | 6.36E-03 | 2231 | 1362 | 1856 | 1212 |
| 592 | Sorting_nexin_4 | O95219 | 6.50E-03 | 10682 | 2713 | 6185 | 1571 |
| 593 | RNase_H1 | O60930 | 6.71E-03 | 427 | 173 | 201 | 140 |
| 594 | 4-1BB | Q07011 | 6.71E-03 | 1263 | 285 | 684 | 224 |
| 595 | PCl | P05154 | 6.77E-03 | 33122 | 53518 | 31322 | 50794 |
| 596 | [symbol] | P15289 | 6.87E-03 | 2074 | 1531 | 1875 | 1540 |
| 597 | NKp46 | O76035 | 6.89E-03 | 3456 | 2168 | 2754 | 2272 |
| 598 | PK3CG | P48736 | 6.91E-03 | 1409 | 960 | 1268 | 967 |
| 599 | CD47 | Q08722 | 6.95E-03 | 740 | 352 | 538 | 327 |
| 600 | IL-12_Rb1 | P42701 | 6.99E-03 | 473 | 265 | 368 | 261 |
| 601 | INK2 | P45984 | 7.03E-03 | 1735 | 876 | 1807 | 889 |
| 602 | VEGF121 | P15692 | 7.06E-03 | 1001 | 647 | 843 | 602 |
| 603 | RSPO4 | QIDM5 | 7.15E-03 | 490 | 293 | 385 | 275 |
| 604 | FGF-18 | O76093 | 7.17E-03 | 123 | 82 | 106 | 80 |
| 605 | a-Synuclein | P37540 | 7.20E-03 | 4868 | 2187 | 3185 | 1886 |
| 606 | IL-18_BP [symbol] | O95998 | 7.30E-03 | 12067 | 7298 | 10985 | 7356 |
| 607 | Fibronectin | P02741 | 7.55E-03 | 16946 | 21457 | 17220 | 21675 |
| 608 | IL-5 | P06113 | 7.60E-03 | 192 | 111 | 151 | 117 |
| 609 | IL-7_Ra | P16871 | 7.65E-03 | 246 | 181 | 215 | 170 |
| 610 | CXCL16_soluble | Q9H2A7 | 7.72E-03 | 14073 | 8769 | 11890 | 7781 |
| 611 | TLR4_MD-2_complex | O00206_Q | 7.77E-03 | 4666 | 3311 | 4272 | 3242 |
| 612 | ERBB3 | P21560 | 7.93E-03 | 290 | 207 | 268 | 185 |
| 613 | RAN | P62826 | 8.05E-03 | 3130 | 1430 | 1704 | 1313 |
| 614 | IGFBP-7 | Q16270 | 8.07E-03 | 66232 | 42954 | 67309 | 39232 |
| 615 | NID2 | Q14112 | 8.28E-03 | 2957 | 1921 | 2704 | 1921 |
| 616 | Apo_D | P05090 | 8.42E-03 | 4818 | 3975 | 5010 | 4011 |
| 617 | IL-22BP | Q96915 | 8.42E-03 | 6198 | 2446 | 3730 | 3180 |
| 618 | [symbol] | P10451 | 8.58E-03 | 49729 | 22902 | 42709 | 19256 |
| 619 | soPAR | Q03405 | 8.60E-03 | 20841 | 13431 | 18794 | 13931 |
| 620 | IL-10_Rb | Q08334 | 8.66E-03 | 1750 | 1109 | 1548 | 594 |
| 621 | KI2L4 | Q99706 | 8.87E-03 | 7418 | 1443 | 2362 | 988 |
| 622 | TEC | P42680 | 8.88E-03 | 1693 | 509 | 1133 | 464 |
| 623 | PKC-2 | Q05513 | 8.94E-03 | 8070 | 358 | 1649 | 346 |
| 624 | IDE | P14735 | 8.99E-03 | 3001 | 1640 | 1894 | 1619 |
| 625 | IFN-a__B_R1 | P17181 | 9.41E-03 | 6311 | 3832 | 4352 | 3622 |
| 626 | Nidogen | P14543 | 9.43E-03 | 7656 | 4499 | 7193 | 4406 |
| 627 | SREC-1 | Q14162 | 9.49E-03 | 8611 | 4615 | 7682 | 3473 |
| 628 | Galectin-4 | P56470 | 9.54E-03 | 479 | 282 | 412 | 231 |
| 629 | C2 | P05681 | 9.68E-03 | 4335 | 2578 | 3545 | 2180 |
| 630 | EMAP-2 | Q12904 | 9.74E-03 | 2379 | 1706 | 1947 | 1617 |
| 631 | CSH | P0DML2_ | 9.77E-03 | 2519 | 1672 | 2101 | 1497 |
| 632 | DLL4 | Q9NR51 | 9.93E-03 | 1254 | 916 | 1193 | 864 |
| 633 | Ephrin-A3 | P52797 | 9.96E-03 | 1124 | 968 | 1139 | 970 |
| 634 | DAPK2 | Q9UIK4 | 1.01E-02 | 3794 | 2028 | 2827 | 1840 |
| 635 | Aflatoxin_B1_aldehyde_ | O43488 | 1.02E-02 | 2402 | 702 | 1694 | 537 |
| | reductase | | | | | | |
| 636 | Dynactin_subunit_2 | Q13561 | 1.01E-02 | 1563 | 995 | 1412 | 864 |
| 637 | IL-17_RD | Q8NFM7 | 1.02E-02 | 363 | 234 | 276 | 250 |
| 638 | protein_Z_inhibitor | Q9UK56 | 1.03E-02 | 9241 | 7101 | 8378 | 6860 |
| 639 | IFN [symbol] | P15260 | 1.04E-02 | 1533 | 927 | 1294 | 751 |
| 640 | Growth_hormone_ | P10912 | 1.06E-02 | 1111 | 812 | 1070 | 744 |
| | receptor | | | | | | |
| 641 | FBLN3 | Q12805 | 1.07E-02 | 2178 | 1374 | 1806 | 1209 |
| 642 | FN1.4 | P02751 | 1.07E-02 | 106550 | 114894 | 103495 | 114256 |
| 643 | SMAC | Q9NR28 | 1.12E-02 | 5085 | 3678 | 4606 | 3295 |
| 644 | MMP-1 | P03956 | 1.14E-02 | 1328 | 759 | 950 | 720 |
| 645 | Laminin | P26391_P | 1.17E-02 | 2353 | 1671 | 1908 | 1560 |
| 646 | BID | P55957 | 1.19E-02 | 3097 | 1644 | 2484 | 1617 |
| 647 | SARP-2 | Q8N474 | 1.23E-02 | 843 | 635 | 725 | 702 |
| 648 | HHLA2 | Q9UM44 | 1.24E-02 | 1964 | 1349 | 1749 | 1371 |
| 649 | PARK7 | Q99497 | 1.24E-02 | 2776 | 578 | 1120 | 713 |
| 650 | Thymidine_kinase | P04183 | 1.24E-02 | 5621 | 576 | 1929 | 557 |
| 651 | Cytidylate_kinase | P30085 | 1.27E-02 | 6009 | 3736 | 5177 | 3199 |
| 652 | LG3BP | Q08380 | 1.28E-02 | 1790 | 1042 | 1559 | 1057 |
| 653 | HMGN1 | P05114 | 1.30E-02 | 696 | 486 | 577 | 611 |
| 654 | C8 | P07357_P | 1.31E-02 | 3696 | 2134 | 2612 | 2172 |
| 655 | LIF_sR | P42702 | 1.33E-02 | 2074 | 1755 | 2028 | 1702 |
| 656 | C34_go41_HIV_Fragment | Q70626 | 1.34E-02 | 1044 | 513 | 740 | 681 |
| 657 | Histone_H2A.z | P0C065 | 1.34E-02 | 457 | 152 | 250 | 154 |
| 658 | Ollactomedin-4 | Q6UX06 | 1.35E-02 | 178 | 85 | 103 | 70 |
| 659 | Noggin | Q13253 | 1.36E-02 | 1696 | 1237 | 1633 | 1226 |
| 660 | IL-19 | Q9UHD0 | 1.40E-02 | 5037 | 3903 | 5094 | 3797 |
| 661 | Antithrombin_III | P01008 | 1.40E-02 | 79108 | 88300 | 79532 | 90222 |
| 662 | CAMK1 | Q14012 | 1.40E-02 | 4572 | 3416 | 4192 | 3773 |
| 663 | Proteinese-3 | P24158 | 1.41E-02 | 20677 | 8074 | 15463 | 6704 |
| 664 | Apo_B | P04114 | 1.41E-02 | 13897 | 5483 | 5154 | 5570 |
| 665 | Sphingosine_kinase_1 | Q9NYA1 | 1.43E-02 | 5492 | 1366 | 3129 | 837 |
| 666 | FGF-4 | P08620 | 1.46E-02 | 279 | 84 | 157 | 82 |
| 667 | SPARCL1 | Q14515 | 1.47E-02 | 13520 | 10588 | 11392 | 10353 |
| 668 | Sonic_Hedgehog | Q15465 | 1.51E-02 | 694 | 1269 | 666 | 1225 |
| 669 | Aminoacylase-1 | Q03154 | 1.52E-02 | 13995 | 4485 | 8002 | 4171 |
| 670 | Troponin_1 | P19429 | 1.53E-02 | 531 | 268 | 364 | 255 |
| 671 | NG36 | Q96XQ7 | 1.55E-02 | 408 | 316 | 374 | 317 |
| 672 | FCG3B | O75015 | 1.59E-02 | 3109 | 1876 | 2892 | 1687 |
| 673 | LYN | P07948 | 1.61E-02 | 10289 | 3019 | 6029 | 2235 |
| 674 | LGMN | Q99538 | 1.62E-02 | 4938 | 3745 | 4412 | 3661 |
| 675 | IL-11 | P20809 | 1.66E-02 | 380 | 127 | 230 | 122 |
| 676 | Maraspsin | Q9BQR3 | 1.67E-02 | 9720 | 6720 | 8822 | 6070 |
| 677 | AMNLS | Q9BXJ7 | 1.73E-02 | 106 | 70 | 88 | 67 |
| 678 | N-terminal_pro-BNP | P16860 | 1.74E-02 | 15980 | 9763 | 13394 | 9384 |
| 679 | Kallikrein [symbol] | Q9Y5K2 | 1.75E-02 | 249 | 145 | 187 | 120 |
| 680 | OX40_Ligand | P23510 | 1.81E-02 | 2279 | 203 | 236 | 194 |
| 681 | Cystatin_C | P01034 | 1.81E-02 | 3119 | 2139 | 2928 | 2204 |
| 682 | Epithelial)cell_kinase | P29317 | 1.83E-02 | 6302 | 3590 | 5724 | 3508 |
| 683 | IL-2_sRs | P01589 | 1.85E-02 | 14187 | 3821 | 10897 | 1057 |
| 684 | FER | P16591 | 1.87E-02 | 1673 | 350 | 932 | 255 |
| 685 | Ck-b-B-1 | P55773 | 1.91E-02 | 68059 | 45201 | 64163 | 44113 |
| 686 | PACAP-36 | P18509 | 1.95E-02 | 175 | 108 | 130 | 108 |
| 687 | XEDAR | Q9HAV5 | 1.97E-02 | 1142 | 693 | 914 | 593 |
| 688 | b2-Microglobulin | P61769 | 2.01E-02 | 3797 | 2104 | 3400 | 1819 |
| 689 | uPA | P06749 | 2.01E-02 | 2358 | 1525 | 2115 | 1329 |
| 690 | GIB | P04054 | 2.05E-02 | 1649 | 1067 | 1330 | 998 |
| 691 | IL-20_Rs | Q9UHF4 | 2.06E-02 | 214 | 178 | 195 | 175 |
| 692 | BTC | P35070 | 2.08E-02 | 398 | 246 | 295 | 216 |
| 693 | PUR8 | P30566 | 2.09E-02 | 13453 | 6307 | 6808 | 4689 |
| 694 | CBPE | P15870 | 2.09E-02 | 1015 | 634 | 763 | 653 |
| 695 | Coagulation_Factor_Xa | P00742 | 2.11E-02 | 2602 | 1916 | 2419 | 1717 |
| 696 | GDF2 | Q9UK05 | 2.14E-02 | 733 | 513 | 660 | 618 |
| 697 | FAM1078 | Q9H098 | 2.15E-02 | 1824 | 1116 | 1761 | 842 |
| 698 | PARC | P55774 | 2.16E-02 | 13696 | 8211 | 10627 | 6989 |
| 699 | NRX1B | P68400 | 2.18E-02 | 392 | 304 | 356 | 310 |
| 700 | KPCT | Q04759 | 2.19E-02 | 2217 | 971 | 1530 | 711 |
| 701 | MMP-13 | P45452 | 2.20E-02 | 579 | 409 | 492 | 429 |
| 702 | C1QR1 | Q9NPY3 | 2.23E-02 | 16555 | 9263 | 15923 | 7598 |
| 703 | [symbol] | P30041 | 2.24E-02 | 372 | 215 | 281 | 178 |
| 704 | SSRP1 | Q08945 | 2.35E-02 | 1185 | 251 | 447 | 251 |
| 705 | HAI-1 | O43278 | 2.40E-02 | 5472 | 4118 | 5259 | 4155 |
| 706 | LEG9 | O00182 | 2.42E-02 | 4238 | 2311 | 3118 | 1910 |
| 707 | p27Kip1 | P46527 | 2.44E-02 | 3786 | 555 | 1125 | 869 |
| 708 | [symbol] | Q6UK15 | 2.47E-02 | 1960 | 1298 | 1819 | 1157 |
| 709 | CLM6 | Q08708 | 2.47E-02 | 8624 | 4427 | 7724 | 3205 |
| 710 | Troponin_I__skeletal__fast_twitch | P48788 | 2.48E-02 | 11782 | 5809 | 6921 | 5298 |
| 711 | SLIKS | O94997 | 2.51E-02 | 2452 | 2017 | 2366 | 1987 |
| 712 | NovH | P48745 | 2.51E-02 | 793 | 478 | 517 | 419 |
| 713 | NANOG | Q9H980 | 2.52E-02 | 96 | 67 | 84 | 65 |
| 714 | FGR | P09769 | 2.65E-02 | 526 | 303 | 446 | 321 |
| 715 | PolyUbiquitin_K63 | P0CG48 | 2.66E-02 | 5992 | 3216 | 4472 | 2854 |
| 716 | Coagulation_Factor_X | P00742 | 2.70E-02 | 3122 | 2296 | 2921 | 2035 |
| 717 | TRAIL_R2 | O14763 | 2.74E-02 | 282 | 189 | 233 | 160 |
| 718 | BGH3 | Q15682 | 2.76E-02 | 40693 | 24378 | 36082 | 18838 |
| 719 | IL-13_Ra1 | P73552 | 2.76E-02 | 3983 | 2544 | 3521 | 2441 |
| 720 | Activin_A | P08476 | 2.77E-02 | 7605 | 4903 | 5457 | 44136 |
| 721 | Fas__soluble | P25445 | 2.78E-02 | 2753 | 1946 | 2594 | 1851 |
| 722 | Tropomysin_1_alpha_chain | P09493 | 2.78E-02 | 1058 | 717 | 845 | 665 |
| 723 | BGN | P21810 | 2.78E-02 | 20412 | 16499 | 18590 | 15332 |
| 724 | FABP | P05413 | 2.79E-02 | 32494 | 18578 | 21796 | 22317 |
| 725 | Calcineirin_B_a | P63098 | 2.83E-02 | 14630 | 11180 | 11803 | 10800 |
| 726 | CD36_ANTIGEN | P16671 | 2.84E-02 | 11119 | 6650 | 9036 | 5399 |
| 727 | Mesothelin | Q13421 | 2.86E-02 | 362 | 223 | 375 | 214 |
| 728 | Carbonic_anhydrase_VII | P41166 | 2.87E-02 | 321 | 156 | 199 | 156 |
| 729 | CREL1 | Q96HDI | 2.88E-02 | 3737 | 2643 | 3379 | 2302 |
| 730 | TNF-a | P01375 | 2.92E-02 | 375 | 275 | 331 | 287 |
| 731 | WFKN1 | Q95NZ8 | 2.94E-02 | 181 | 134 | 170 | 124 |
| 732 | Elatase | P08246 | 2.97E-02 | 2231 | 914 | 1433 | 712 |
| 733 | TXD12 | O95881 | 2.99E-02 | 2350 | 1844 | 2285 | 1723 |
| 734 | UBP25 | Q9UHP3 | 2.99E-02 | 1583 | 778 | 975 | 793 |
| 735 | Nogo_Receptor | Q9BZR6 | 3.06E-02 | 2735 | 1606 | 2028 | 1254 |
| 736 | PLXC1 | O60486 | 3.08E-02 | 872 | 626 | 813 | 567 |
| 737 | NK [symbol] 30 | O14931 | 3.11E-02 | 474 | 305 | 394 | 275 |
| 738 | Lectin_mannose-binding_2 | Q12907 | 3.17E-02 | 20793 | 17331 | 19690 | 17322 |
| 739 | Endothelin-converting_enzyme_1 | P42892 | 3.25E-02 | 9062 | 11034 | 8752 | 11606 |
| 740 | Endogiin | P17813 | 3.27E-02 | 5496 | 3729 | 4994 | 3429 |
| 741 | PDGFRA | P16234 | 3.29E-02 | 6469 | 4279 | 4786 | 4674 |
| 742 | GPDA | P21695 | 3.36E-02 | 3984 | 2250 | 2922 | 2293 |
| 743 | ANK2 | Q01484 | 3.37E-02 | 372 | 298 | 331 | 290 |
| 744 | ASM3A | Q92484 | 3.38E-02 | 5904 | 3491 | 4333 | 2480 |
| 745 | IL-23 | P29460_Q | 3.49E-02 | 6496 | 2549 | 4751 | 1496 |
| 746 | Nectin-like_protein_1 | Q8N126 | 3.52E-02 | 4013 | 3096 | 4017 | 2853 |
| 747 | CO8A1 | P27658 | 3.54E-02 | 1497 | 766 | 756 | 663 |
| 748 | LIMP_II | Q14108 | 3.64E-02 | 345 | 211 | 219 | 191 |
| 749 | Factor_D | P00746 | 3.66E-02 | 723 | 618 | 705 | 592 |
| 750 | NADPH-P450_Oxidoredictase | P145435 | 3.70E-02 | 17437 | 6380 | 8332 | 5937 |
| 751 | AFP | P02771 | 3.73E-02 | 639 | 444 | 547 | 396 |
| 752 | PGRP-S | O75594 | 3.75E-02 | 1762 | 773 | 1391 | 406 |
| 753 | Calcinurin | Q08209_P | 3.75E-02 | 2479 | 582 | 1021 | 494 |
| 754 | Lipocatin_2 | P80158 | 3.79E-02 | 20416 | 10500 | 16425 | 8474 |
| 755 | OX2G | P41217 | 3.81E-02 | 3644 | 2795 | 3433 | 2538 |
| 756 | AMPX_a3b2g1 | P54646_O | 3.81E-02 | 7390 | 3291 | 5042 | 2120 |
| 757 | Glypican_3 | P51654 | 3.83E-02 | 759 | 504 | 709 | 598 |
| 758 | 14-3-3E | P62258 | 3.85E-02 | 6614 | 4345 | 4252 | 5138 |
| 759 | Prothrombin | P00734 | 3.88E-02 | 101692 | 115113 | 100580 | |
| 760 | MED-1 | Q15648 | 3.94E-02 | 35616 | 46117 | 34247 | 46516 |
| 761 | DAN | P41271 | 4.06E-02 | 10871 | 5650 | 6792 | 3904 |
| 762 | IGF-1 | P05019 | 4.07E-02 | 957 | 739 | 972 | 803 |
| 763 | GAF | P08174 | 4.14E-02 | 19961 | 14235 | 17881 | 12018 |
| 764 | SIG14 | Q08ET2 | 4.19E-02 | 19449 | 8372 | 20606 | 8914 |
| 765 | KEAP1 | Q14145 | 4.19E-02 | 3411 | 550 | 994 | 534 |
| 766 | BNP-32 | P16860 | 4.22E-02 | 347 | 223 | 262 | 226 |
| 767 | gol [symbol] | P08514_P | 4.26E-02 | 1808 | 1037 | 1211 | 841 |
| 768 | kallikrein_14 | Q9P0G3 | 4.28E-02 | 11774 | 9747 | 10856 | 9596 |
| 769 | URB | Q75M96 | 4.29E-02 | 2927 | 1882 | 2464 | 1864 |
| 770 | IL-17 | Q16552 | 4.32E-02 | 321 | 191 | 234 | 195 |
| 771 | SDF-1 | P48061 | 4.33E-02 | 5947 | 3701 | 5300 | 2876 |
| 772 | cGMF-stimulated_PDE | Q00408 | 4.45E-02 | 2259 | 1834 | 1955 | 1838 |
| 773 | Persephin | O60542 | 4.49E-02 | 182 | 121 | 158 | 105 |
| 774 | Glucsgon | P01275 | 4.50E-02 | 2275 | 6302 | 1586 | 6369 |
| 775 | calgranulin_8 | P06702 | 4.52E-02 | 4269 | 6219 | 3593 | 6006 |
| 776 | FGF23 | Q9G2VG | 4.54E-02 | 1867 | 710 | 626 | 588 |
| 777 | dope_decarboxylase | P20731 | 4.58E-02 | 323 | 159 | 209 | 158 |
| 778 | ANP | P01160 | 4.62E-02 | 494 | 303 | 379 | 247 |
| 779 | MMP-2 | P08253 | 4.63E-02 | 6552 | 9196 | 6692 | 8603 |
| 780 | Galectin_8 | O00214 | 4.71E-02 | 356 | 295 | 321 | 287 |
| 781 | Exotaxin | P51671 | 4.73E-02 | 47139 | 63123 | 46050 | 60905 |
| 782 | Cytochrome_c | P99999 | 4.75E-02 | 1358 | 934 | 1078 | 809 |
| 783 | TGF-b2 | P61832 | 4.75E-02 | 279 | 100 | 150 | 91 |
| 784 | MK13 | O15264 | 4.77E-02 | 1296 | 456 | 573 | 432 |
| 785 | OLL1 | O00548 | 4.86E-02 | 2578 | 1945 | 2222 | 1973 |
| 786 | MP2K4 | P45985 | 4.89E-02 | 26031 | 40552 | 23121 | 43610 |
| 787 | IR | P06213 | 4.91E-02 | 11211 | 7984 | 3398 | 7048 |
| 788 | TSH | P01215_P | 4.92E-02 | 1427 | 945 | 1051 | 951 |
| 789 | IL-17E | Q9H293 | 4.94E-02 | 658 | 510 | 607 | 465 |
| 790 | NRP1 | O14786 | 4.95E-02 | 1875 | 1451 | 1874 | 1522 |

[symbol]

indicates data missing or illegible when filed

With respect to the proteins represented by Nos. 468, 595, 607, 642, 661, 668, 739, 759, 760, 774, 775, 779, 781 and 786 of Table 2, a significant increase was observed in the remission group compared to the patient group.

In contrast, with respect to the proteins represented by Nos. 1 to 467, 469 to 594, 596 to 606, 608 to 641, 643 to 660, 662 to 667, 669 to 738, 740 to 758, 761 to 773, 776 to 778, 780, 782 to 785 and 786 to 790, a significant decrease was observed in the remission group compared to the patient group.

The p values in the increase or decrease in amount of the proteins represented by Nos. 1 to 440 were less than 0.001.

The p values in the increase or decrease in amount of the proteins represented by Nos. 441 to 633 were less than 0.01.

The p values in the increase or decrease in amount of the proteins represented by Nos. 634 to 790 were less than 0.05.

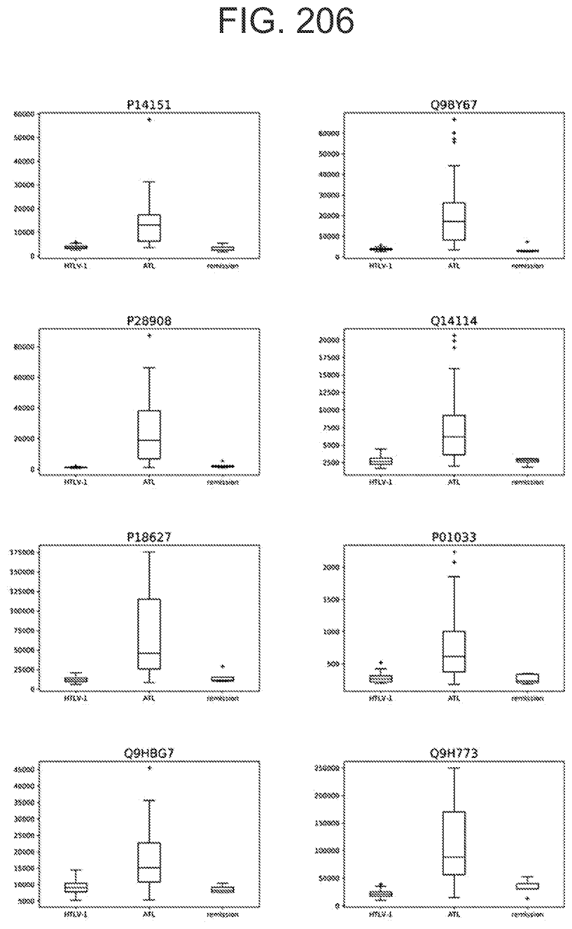

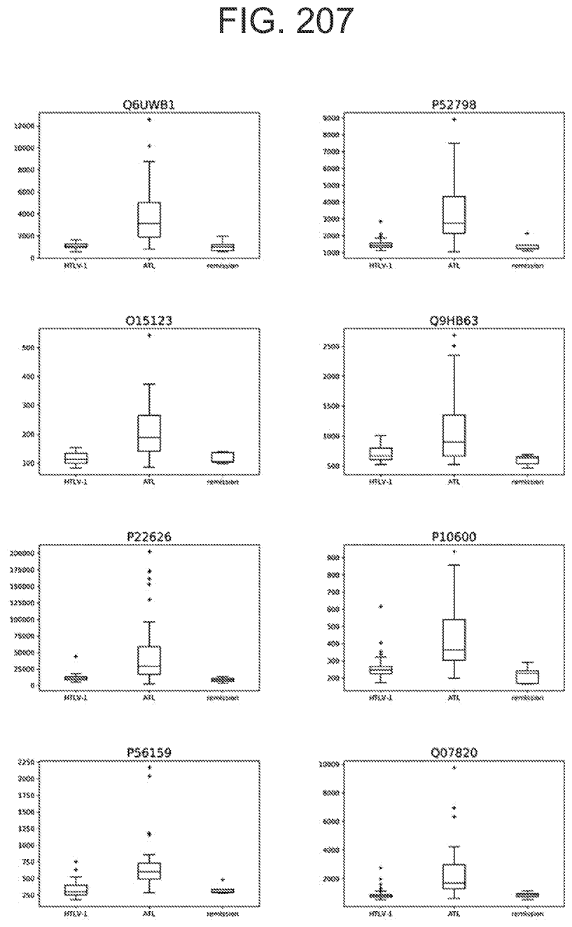

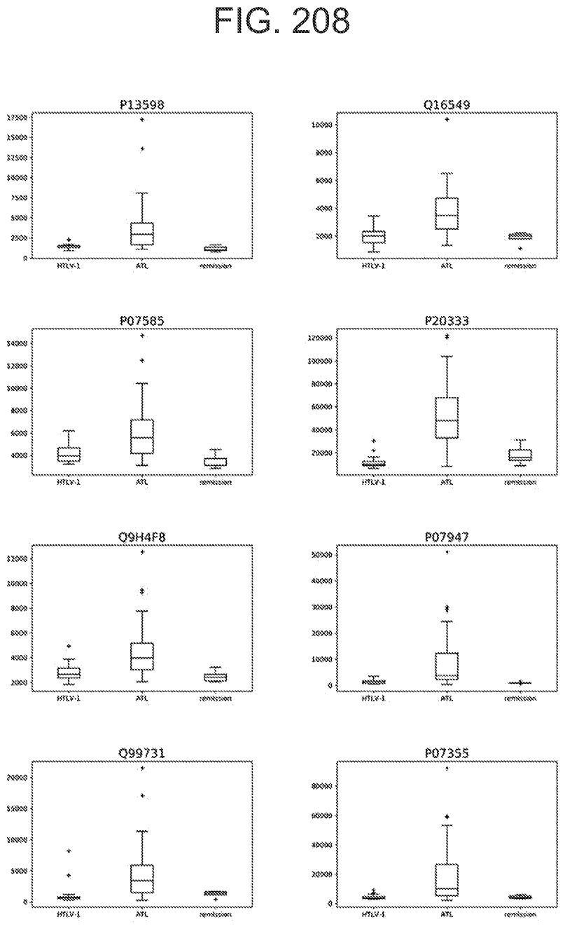

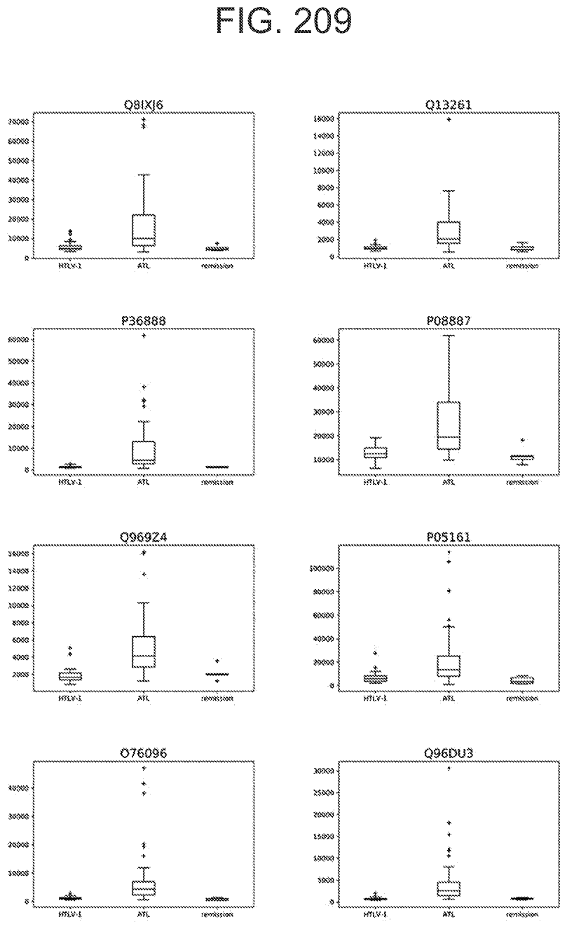

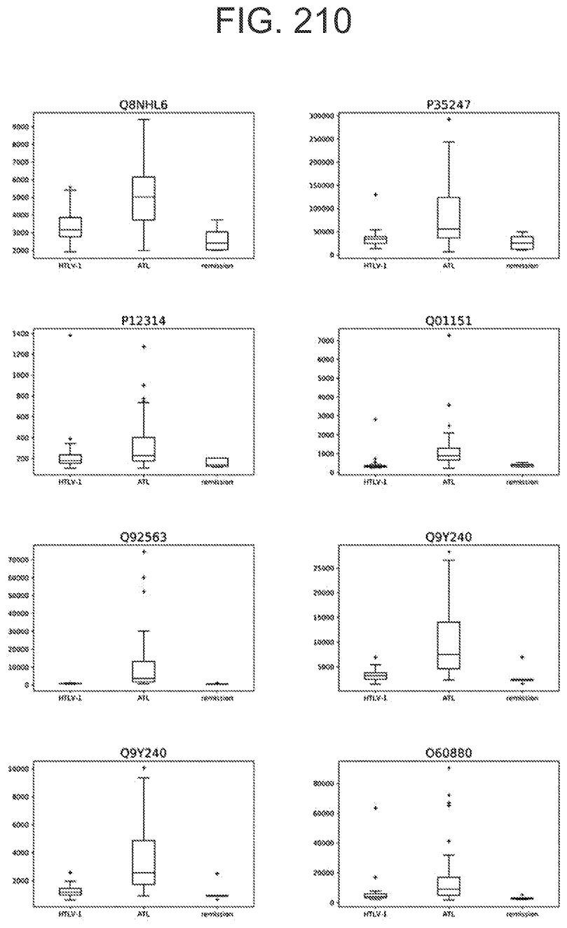

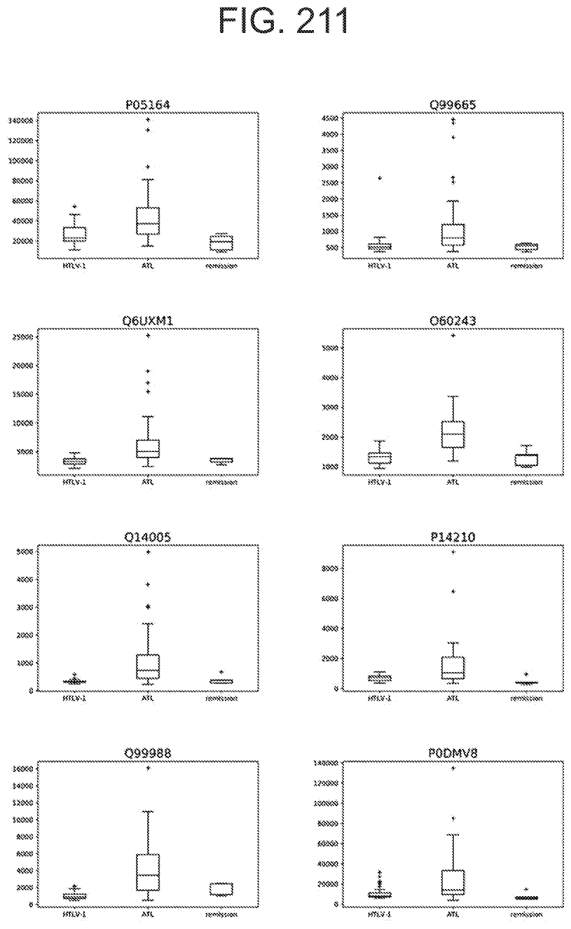

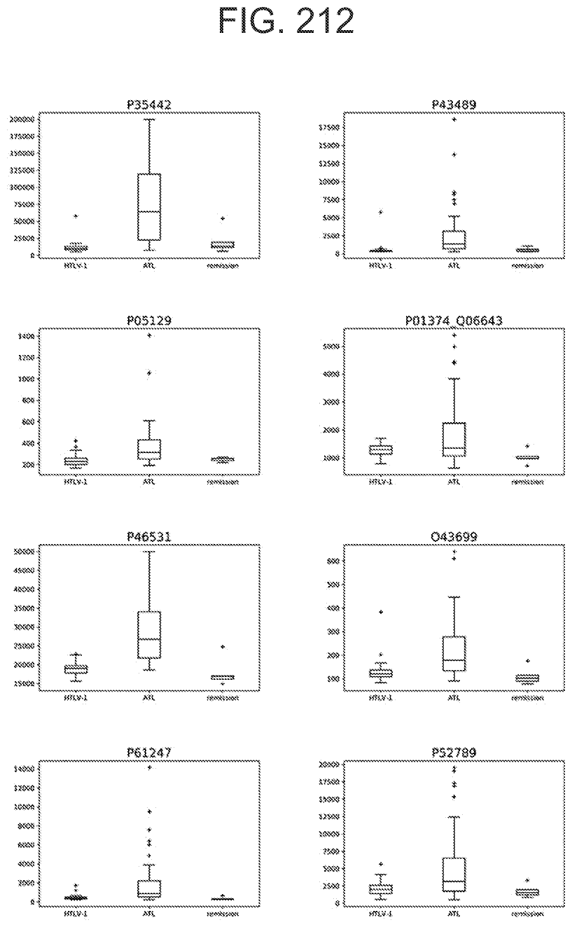

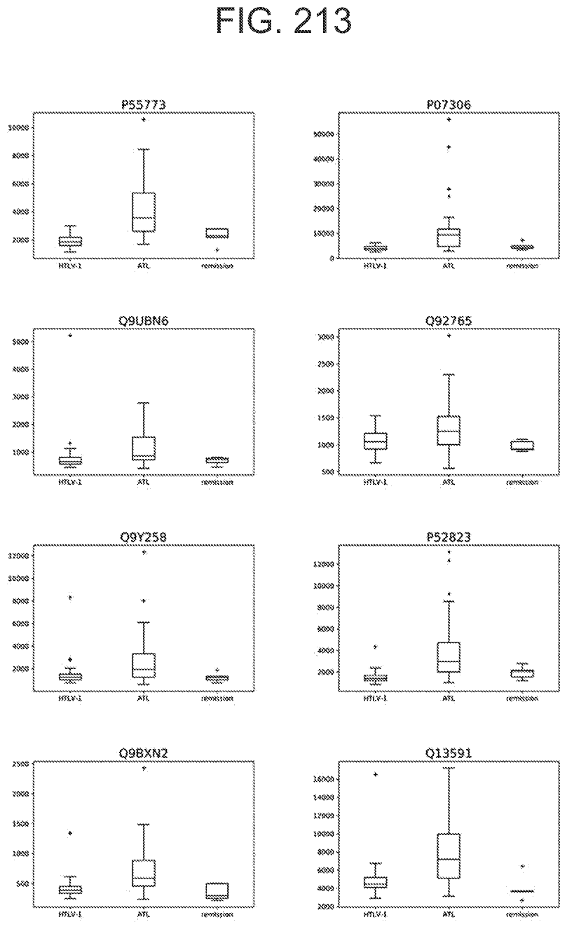

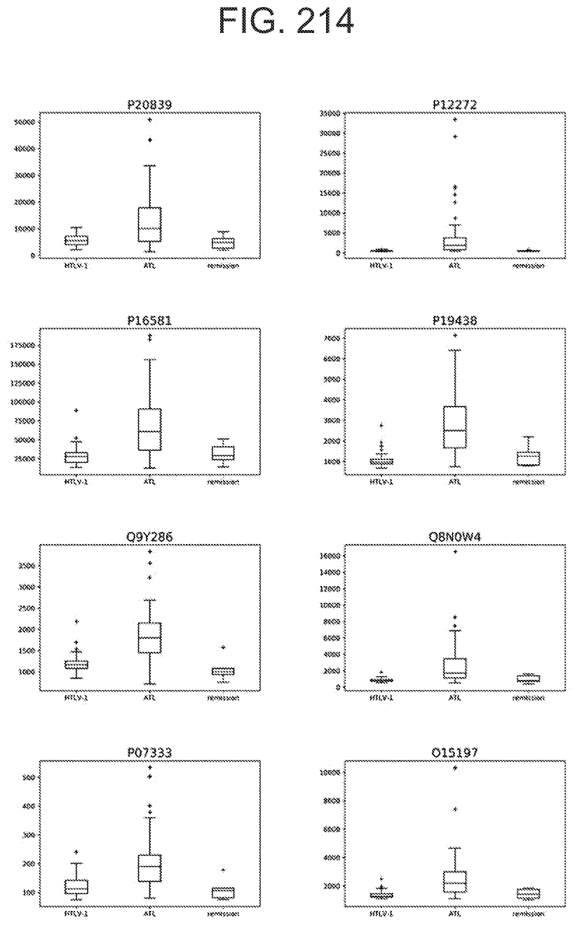

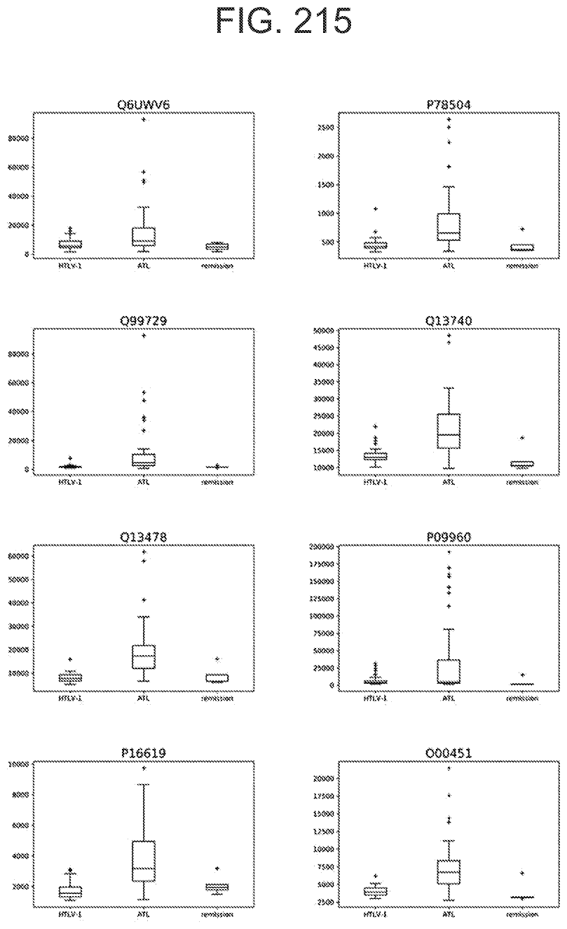

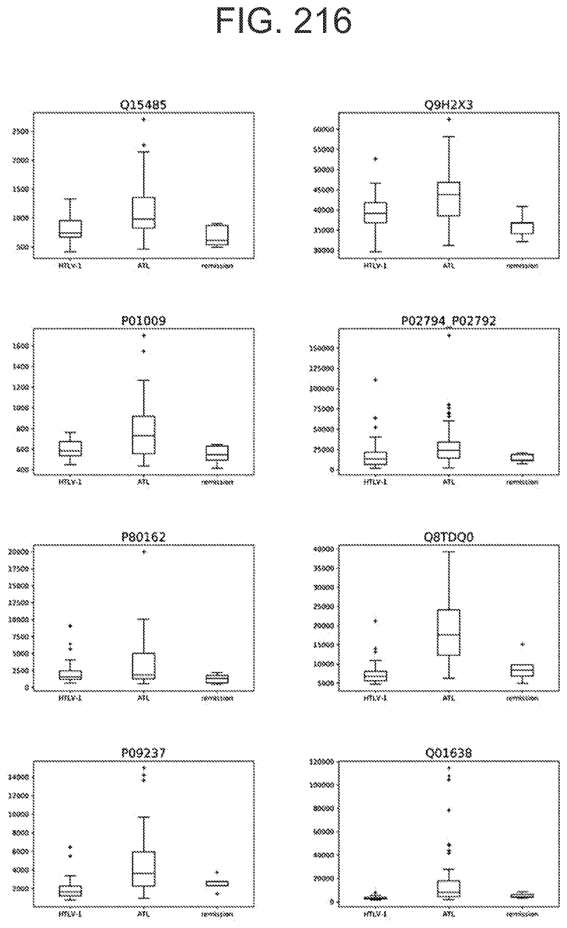

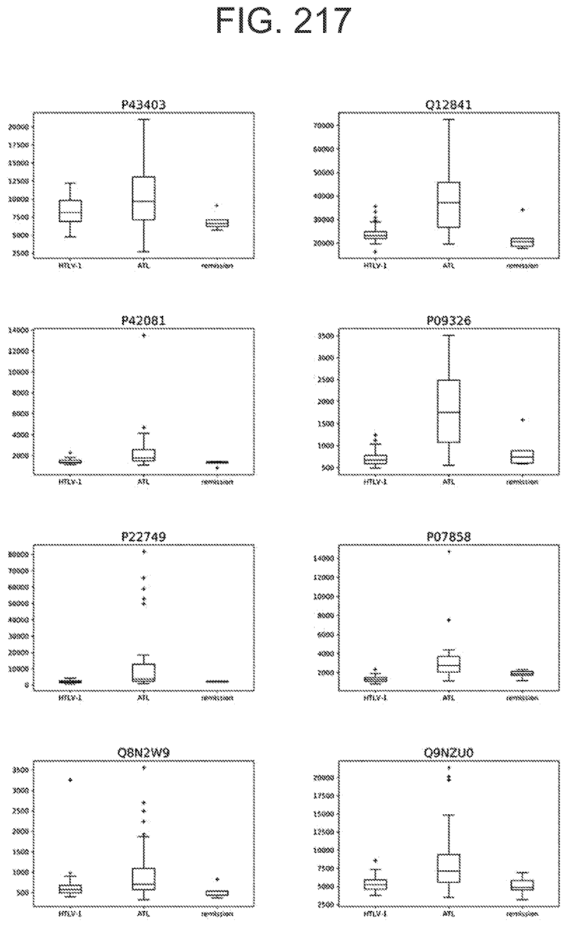

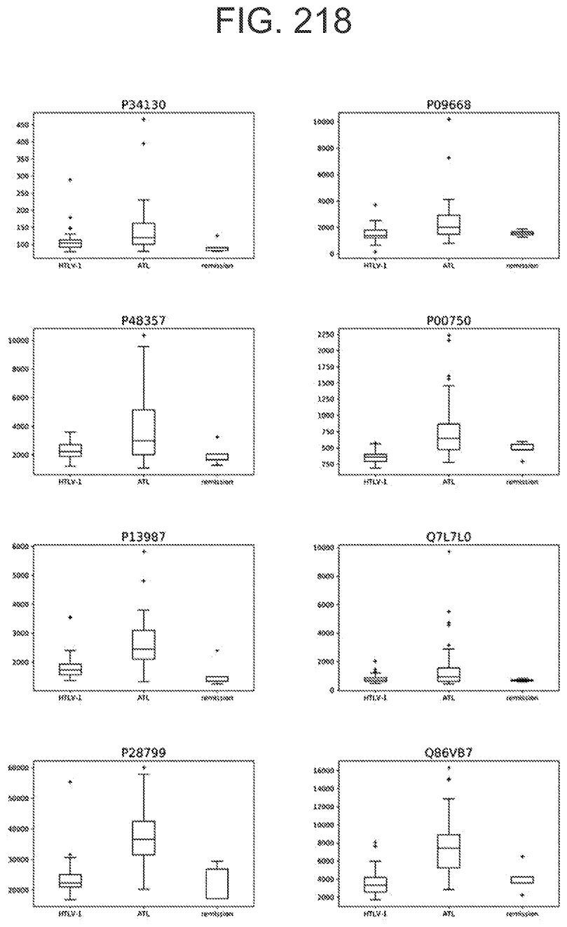

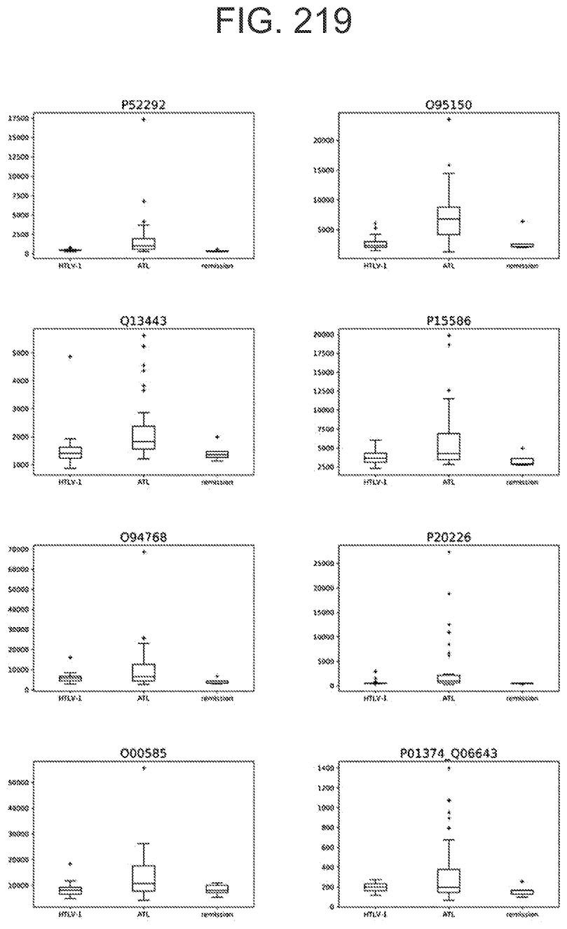

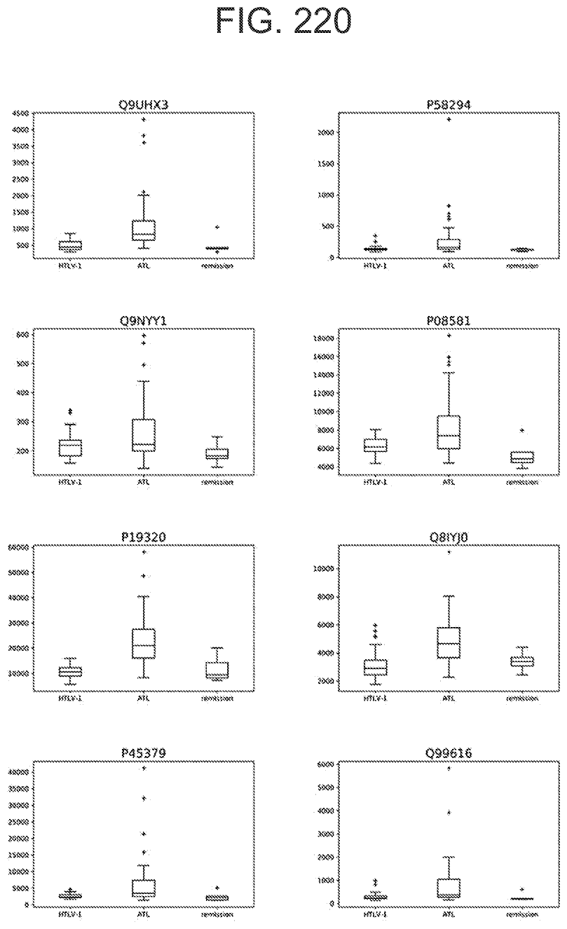

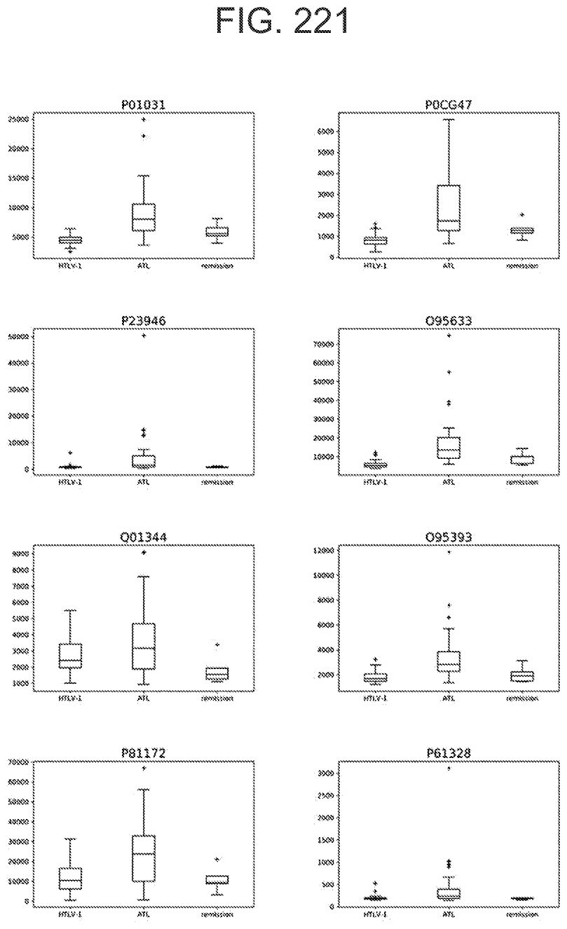

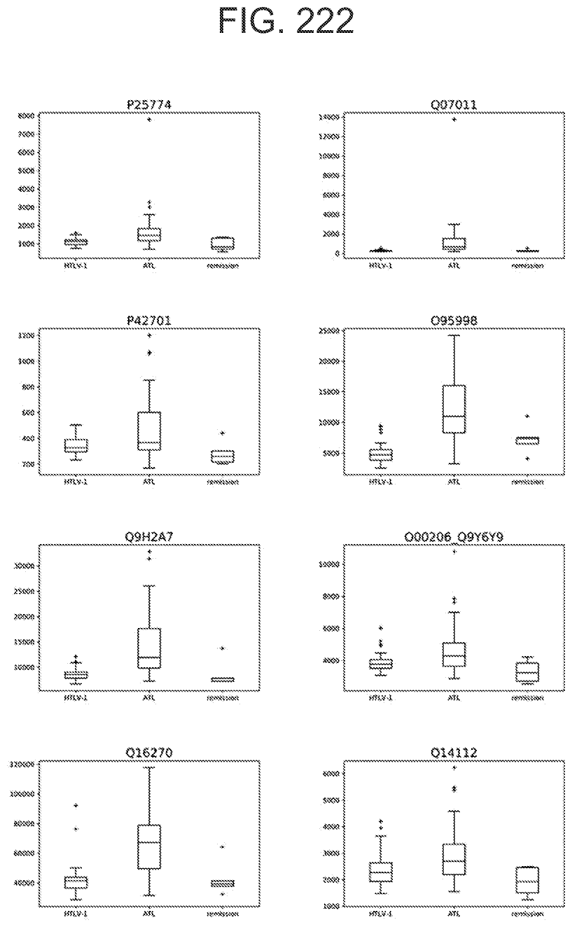

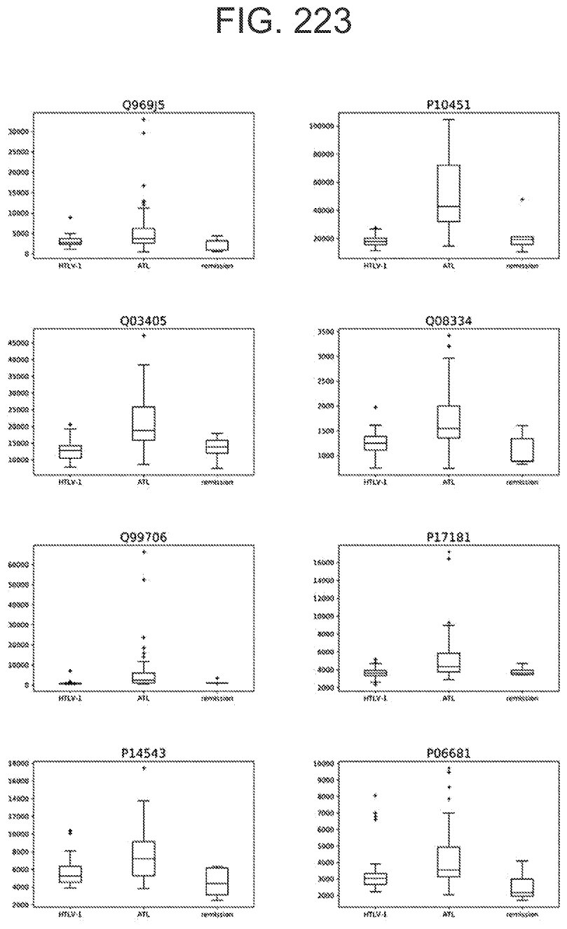

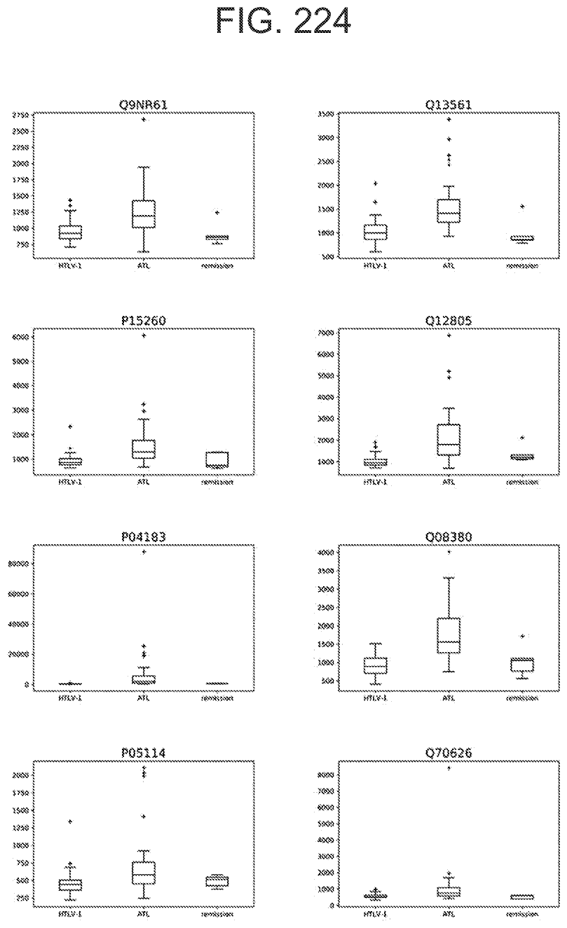

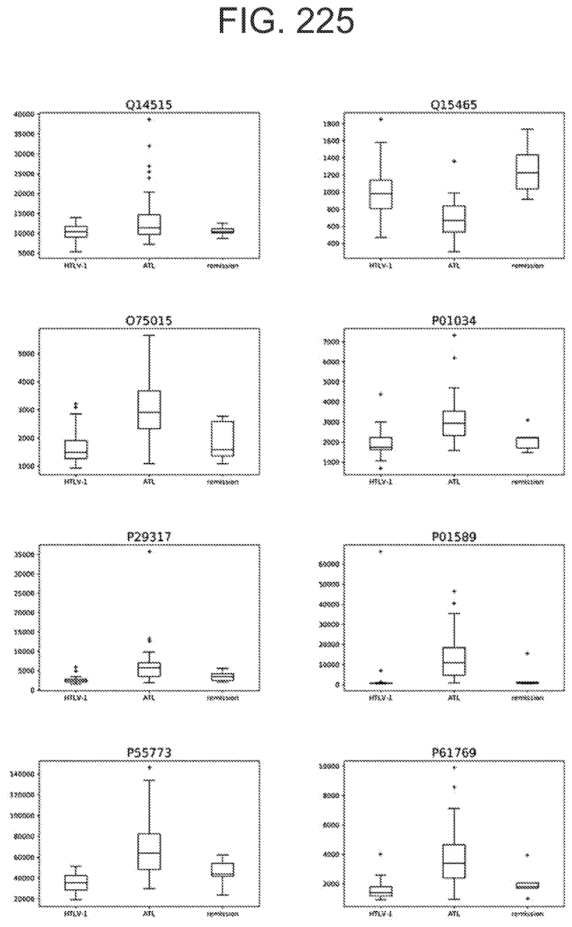

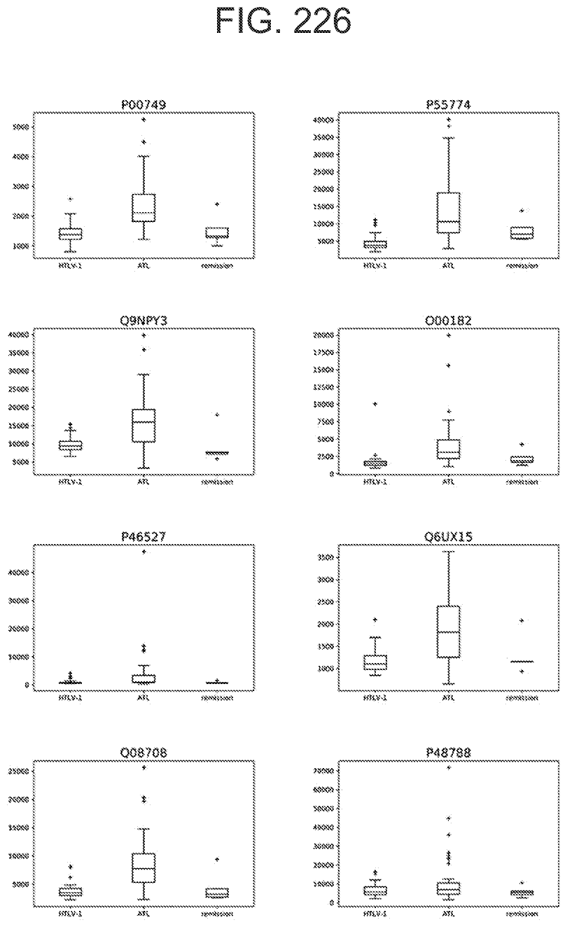

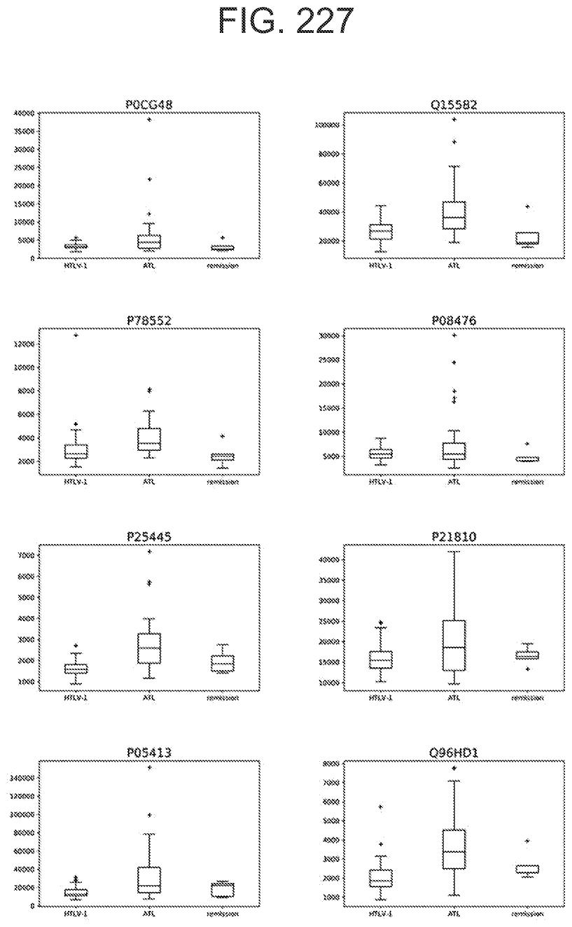

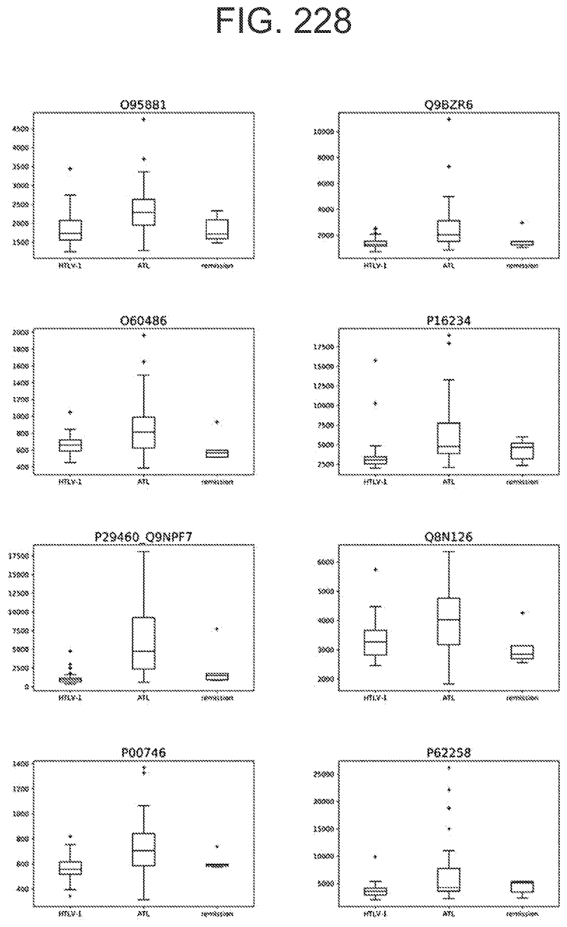

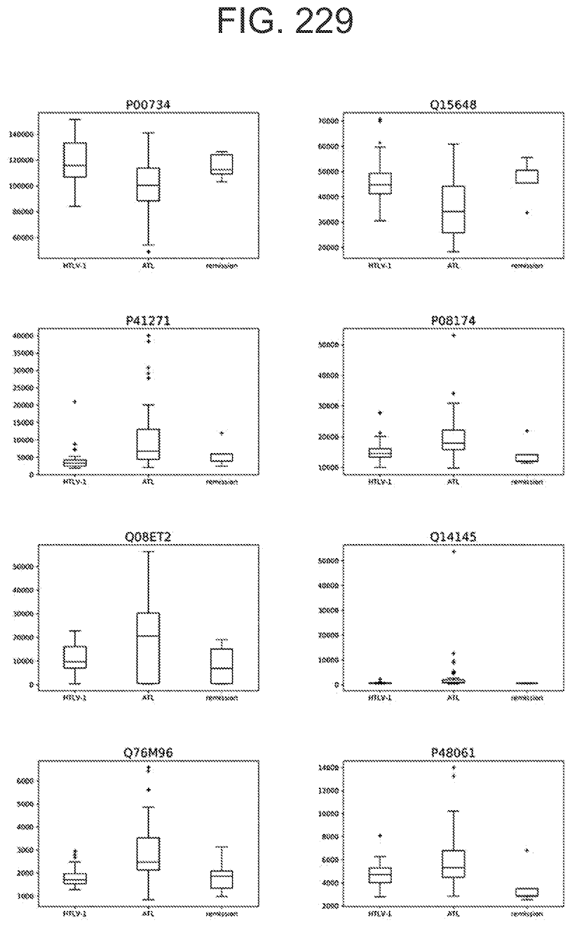

Of the proteins listed in Table 1 and Table 2, with respect to the proteins (represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 128, 129, 131, 139, 141, 155, 156, 161, 164, 165, 167, 172, 173, 174, 175, 181, 185, 187, 189, 195, 198, 199, 200, 203, 205, 207, 209, 211, 220, 221, 222, 223, 224, 225, 226, 230, 231, 232, 236, 238, 240, 241, 242, 243, 244, 246, 247, 250, 251, 252, 253, 256, 259, 260, 269, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609 and 629 of Table 1) showing a significant increase in the patient group compared to the carrier group and a significant decrease in the remission group compared to the patient group, or the proteins (represented by Nos. 126, 170, 235, 270 and 621 of Table 1) showing a significant decrease in the patient group compared to the carrier group and a significant increase in the remission group compared to the patient group, the measurement results are shown in FIGS. 206 to 230.

These proteins can serve as an onset marker for discriminating between a carrier group and a patient group and as a remission marker for discriminating between a patient group and a remission group; and can be a particularly useful marker for ATL diagnosis.

1-13. (canceled)

14. A method for treating an HTLV-1 related disease, comprising the steps of:
measuring an amount of a marker protein in a blood sample taken from a subject, wherein the marker protein is at least one selected from the proteins represented by Nos. 7, 9, 10, 17, 20, 21, 22, 25, 28, 33, 37, 39, 42, 43, 44, 46, 47, 48, 49, 56, 57, 59, 61, 62, 64, 65, 66, 67, 70, 73, 74, 75, 77, 79, 80, 81, 82, 88, 89, 92, 93, 94, 95, 97, 99, 100, 101, 102, 103, 106, 108, 112, 115, 120, 121, 123, 124, 125, 126, 128, 129, 131, 139, 141, 155, 156, 161, 164, 165, 167, 170, 172, 173, 174, 175, 181, 185, 187, 189, 195, 198, 199, 200, 203, 205, 207, 209, 211, 220, 221, 222, 223, 224, 225, 226, 230, 231, 232, 235, 236, 238, 240, 241, 242, 243, 244, 246, 247, 250, 251, 252, 253, 256, 259, 260, 269, 270, 271, 274, 279, 288, 289, 294, 296, 299, 300, 302, 306, 307, 309, 317, 318, 322, 327, 330, 331, 332, 339, 343, 345, 346, 347, 350, 357, 359, 364, 366, 373, 374, 375, 378, 382, 387, 390, 393, 394, 399, 400, 401, 405, 411, 418, 425, 427, 431, 438, 440, 446, 447, 450, 451, 452, 456, 460, 476, 477, 480, 481, 487, 504, 511, 524, 531, 534, 542, 548, 551, 552, 555, 573, 584, 586, 594, 606, 609, 621 and 629 of Table 1;
selecting the subject as a subject to be treated if the measured value is a predetermined reference value or more or the predetermined reference value or less;
applying a chemotherapy and/or a non-chemotherapy to the selected subject to be treated; and
thereafter, increasing, reducing, stopping or terminating application of a chemotherapy and/or non-chemotherapy to the subject if the measured value reaches a predetermined reference value or less or the predetermined reference or more.

15. The method according to claim 14, wherein the subject is a patient with an HTLV-1 related disease.

16. The method according to claim 14, wherein the HTLV-1 related disease is adult T-cell leukemia (ATL).

17. The method according to claim 14, wherein the non-chemotherapy is a cancer vaccine therapy.

18. A method for treating an HTLV-1 related disease, comprising the steps of:
measuring an amount of a marker protein in a blood sample taken from a subject, wherein the marker protein is at least one selected from the proteins listed in Table 1 (except the protein represented by No. 62);
selecting the subject as a subject to be treated if the measured value is a predetermined reference value or more or the predetermined reference value or less; and
applying a chemotherapy and/or a non-chemotherapy to the selected subject to be treated.

19. The method according to claim 18, wherein the subject is an HTLV-1 career.

20. The method according to claim 18, wherein the HTLV-1 related disease is adult T-cell leukemia (ATL).

21. The method according to claim 18, wherein the non-chemotherapy is a cancer vaccine therapy.

22. A method for treating an HTLV-1 related disease, comprising the steps of:
measuring an amount of a marker protein in a blood sample taken from a subject, wherein the marker protein is at least one selected from the proteins listed in Table 2; and
increasing, reducing, stopping or terminating application of a chemotherapy and/or non-chemotherapy to the subject if the measured value is a predetermined reference value or more or the predetermined reference or less.

23. The method according to claim 22, wherein the subject is a patient with an HTLV-1 related disease.

24. The method according to claim 22, wherein the HTLV-1 related disease is adult T-cell leukemia (ATL).

25. The method according to claim 22, wherein the non-chemotherapy is a cancer vaccine therapy.

26. The method according to claim 14, wherein the blood sample is plasma.

27. A diagnostic kit for an HTLV-1 related disease, comprising a reagent for detecting at least one protein selected from the proteins listed in Table 1 and Table 2.

28. Use of at least one protein selected from the proteins listed in Table 1 and Table 2, for diagnosing an HTLV-1 related disease.
